(19) United States
(12) Patent Application Publication
(10) Pub. No.: US 2025/0032454 A1
(43) Pub. Date: Jan. 30, 2025

(54) KDM1A INHIBITORS FOR THE TREATMENT OF DISEASE

(71) Applicant: Imago Biosciences, Inc., Rahway, NJ (US)

(72) Inventors: Hugh Y. Rienhoff, Jr., San Carlos, CA (US); Cassandra Celatka, Hull, MA (US); Amy E. Tapper, Boston, MA (US); Michael Clare, Skokie, IL (US); Nivedita Namdev, Westford, MA (US)

(73) Assignee: Imago Biosciences, Inc., Rahway, NJ (US)

(21) Appl. No.: 18/702,005

(22) PCT Filed: Oct. 17, 2022
(86) PCT No.: PCT/US2022/078187

§ 371 Date: Apr. 17, 2024

Related U.S. Application Data
(60) Provisional application No. 63/256,850, filed on Oct. 18, 2021.

(51) Int. Cl. A61K 31/416; A61K 31/454; A61K 31/55; A61K 45/06; A61P 7/00; C07D 231/38; C07D 403/04; C07D 403/10; C07D 403/12; C07D 403/14; C07D 471/08

(52) U.S. Cl. CPC A61K 31/416; A61K 31/454; A61K 31/55; A61K 45/06; A61P 7/00; C07D 231/38; C07D 403/04; C07D 403/10; C07D 403/12; C07D 403/14; C07D 471/08

(57) ABSTRACT

Disclosed herein are new compounds and compositions and their application as pharmaceuticals for the treatment of diseases. Methods of inhibition of KDM1A, methods of increasing gamma globin gene expression, and methods to induce differentiation of cancer cells in a human or animal subject are also provided for the treatment of diseases such as acute myelogenous leukemia.

This application claims the benefit of priority of U.S. Provisional Application No. 63/256,850, filed Oct. 18, 2021, which is incorporated herein by reference in its entirety.

Inhibiting the enzyme KDM1A (also known as lysine-specific demethylase 1, LSD1, Flavin-containing Amine Oxidase Domain-Containing Protein, AOF2, BRAF35-HDAC Complex Protein BHC110, FAD-Binding Protein BRAF35-HDAC Complex) would be useful for the treatment of diseases such as cancer and heritable diseases such as Wilson disease, cardiomyopathies, and hemoglobinopathies. Accordingly, a need exists for new inhibitors of KDM1A.

Provided is a compound of structural Formula I:

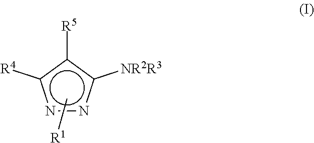

or a salt or tautomer thereof, wherein:
R¹ is chosen from cycloalkyl, heterocycloalkyl, aryl, and heteroaryl, any of which is optionally substituted with one R⁶, and one or more R⁷;
R² and R³ are independently chosen from H, cycloalkyl, heterocycloalkyl, aryl, heteroaryl, (cycloalkyl)alkyl, (heterocycloalkyl)alkyl, (aryl)alkyl, and (heteroaryl)alkyl, any of which is optionally substituted with one or more R⁸,
or R² and R³, together with the intervening nitrogen, combine to form heterocycloalkyl which is optionally substituted with one or more R⁸;
R⁴ is chosen from cycloalkyl, heterocycloalkyl, aryl, and heteroaryl, any of which is optionally substituted with one or more R⁹;
R⁵ is chosen from H, halo, and cyano;
R⁶ is chosen from cycloalkyl, heterocycloalkyl, aryl, heteroaryl, (cycloalkyl)carbonyl, (heterocycloalkyl)carbonyl, (aryl)carbonyl, and (heteroaryl)carbonyl, any of which is optionally substituted with one or more R¹⁰;
each R⁷ is independently chosen from halo, cyano, hydroxy, alkyl, alkoxy, (hydroxy)alkyl, haloalkoxy, and (hydroxy)alkoxy;
each R⁸ is independently chosen from —N(R¹¹)₂, —NHR¹¹, —C(NH)NHR¹¹, —NHC(═NH)—R¹¹, —NHC(═NH)NHR¹¹, —CONHR¹¹, —CH₂NHR¹¹, —CH₂C(═NH)NHR¹¹, —CH₂NHC(═NH)—R¹¹, —CH₂NHC(═NH)NHR¹¹, —CH₂CONHR¹¹, halo, cyano, hydroxy, alkyl, and alkoxy;
each R⁹ is independently chosen from halo, cyano, hydroxy, alkyl, and alkoxy; and
each R¹⁰ is independently chosen from halo, cyano, hydroxy, oxo, alkyl, alkoxy, (hydroxy)alkyl, (hydroxy)alkoxy, alkylthio, and alkylsulfonyl; and
each R¹¹ is independently chosen from H and alkyl.

Also provided are pharmaceutical compositions comprising one or more compounds, or a salt or tautomer thereof, disclosed herein together with a pharmaceutically acceptable carrier, as well as methods of making and using the compounds, salts, tautomers, and compositions.

Also provided are methods for inhibiting KDM1A and methods for treating a KDM1A-mediated disorder in a patient in need of such treatment, comprising administering to said patient a therapeutically effective amount of a compound, or a salt or tautomer thereof, or composition described herein. Also provided is the use of certain compounds, salts, or tautomers thereof, disclosed herein for use in the manufacture of a medicament for the treatment of a disease or condition ameliorated by the inhibition of KDM1A.

These and other aspects of the disclosure disclosed herein will be set forth in greater detail as the patent disclosure proceeds.

### DETAILED DESCRIPTION

Provided is a compound of structural Formula I:

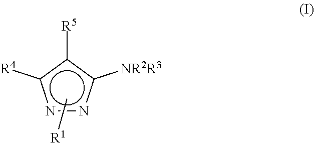

or a salt or tautomer thereof, wherein:
R¹ is chosen from cycloalkyl, heterocycloalkyl, aryl, and heteroaryl, any of which is optionally substituted with one R⁶, and one or more R⁷;
R² and R³ are independently chosen from H, cycloalkyl, heterocycloalkyl, aryl, heteroaryl, (cycloalkyl)alkyl, (heterocycloalkyl)alkyl, (aryl)alkyl, and (heteroaryl)alkyl, any of which is optionally substituted with one or more R⁸,
or R² and R³, together with the intervening nitrogen, combine to form heterocycloalkyl which is optionally substituted with one or more R⁸;
R⁴ is chosen from cycloalkyl, heterocycloalkyl, aryl, and heteroaryl, any of which is optionally substituted with one or more R⁹;
R⁵ is chosen from H, halo, and cyano;
R⁶ is chosen from cycloalkyl, heterocycloalkyl, aryl, heteroaryl, (cycloalkyl)carbonyl, (heterocycloalkyl)carbonyl, (aryl)carbonyl, and (heteroaryl)carbonyl, any of which is optionally substituted with one or more R¹⁰;
each R⁷ is independently chosen from halo, cyano, hydroxy, alkyl, alkoxy, (hydroxy)alkyl, haloalkoxy, and (hydroxy)alkoxy;
each R⁸ is independently chosen from —N(R¹¹)₂, —NHR¹¹, —C(NH)NHR¹¹, —NHC(═NH)—R¹¹, —NHC(═NH)NHR¹¹, —CONHR¹¹, —CH₂NHR¹¹, —CH₂C(═NH)NHR¹¹, —CH₂NHC(═NH)—R¹¹, —CH₂NHC(═NH)NHR¹¹, —CH₂CONHR¹¹, halo, cyano, hydroxy, alkyl, and alkoxy;
each R⁹ is independently chosen from halo, cyano, hydroxy, alkyl, and alkoxy; and
each R¹⁰ is independently chosen from halo, cyano, hydroxy, oxo, alkyl, alkoxy, (hydroxy)alkyl, (hydroxy)alkoxy, alkylthio, and alkylsulfonyl; and
each R¹¹ is independently chosen from H and alkyl.

Also provided is a compound of structural Formula (I):

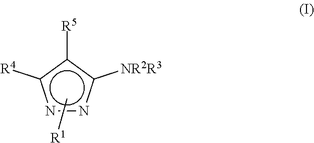

or a salt or tautomer thereof, wherein:
R¹ is chosen from cycloalkyl, heterocycloalkyl, aryl, and heteroaryl, any of which is optionally substituted with one R⁶, and one or more R⁷;
R² and R³ are independently chosen from H, cycloalkyl, heterocycloalkyl, aryl, heteroaryl, (cycloalkyl)alkyl, (heterocycloalkyl)alkyl, (aryl)alkyl, and (heteroaryl)alkyl, any of which is optionally substituted with one or more R⁸,
or R² and R³, together with the intervening nitrogen, combine to form heterocycloalkyl which is optionally substituted with one or more R⁸;
R⁴ is chosen from cycloalkyl, heterocycloalkyl, aryl, and heteroaryl, any of which is optionally substituted with one or more R⁹;
R⁵ is chosen from H, halo, and cyano;
R⁶ is chosen from cycloalkyl, heterocycloalkyl, aryl, heteroaryl, (cycloalkyl)carbonyl, (heterocycloalkyl)carbonyl, (aryl)carbonyl, and (heteroaryl)carbonyl, any of which is optionally substituted with one or more R¹⁰;
each R⁷ is independently chosen from halo, cyano, hydroxy, alkyl, and alkoxy;
each R⁸ is independently chosen from —NHR¹¹, —C(NH)NHR¹¹, —NHC(═NH)—R¹¹, —NHC(═NH)NHR¹¹, —CONHR¹¹, —CH₂NHR¹¹, —CH₂C(═NH)NHR¹¹, —CH₂NHC(═NH)—R¹¹, —CH₂NHC(═NH)NHR¹¹, —CH₂CONHR¹¹, halo, cyano, hydroxy, alkyl, and alkoxy;
each R⁹ is independently chosen from halo, cyano, hydroxy, alkyl, and alkoxy; and
each R¹⁰ is independently chosen from halo, cyano, hydroxy, oxo, alkyl, alkoxy, (hydroxy)alkyl, (hydroxy)alkoxy, alkylthio, and alkylsulfonyl; and
each R¹¹ is independently chosen from H and alkyl.

Also provided is a compound having structural Formula (IIa) or (IIb):

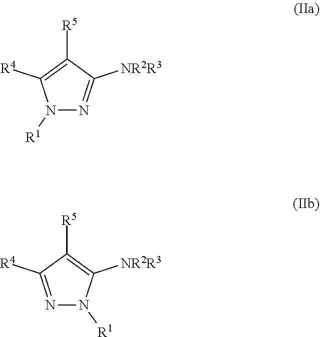

or a salt thereof wherein R¹, R², R³, R⁴, and R⁵ are as described herein.

Also provided herein is a compound having structural Formula (IIIa) or (IIIb):

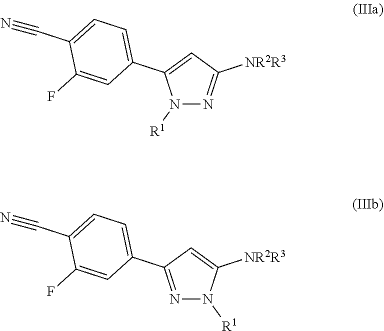

or a salt thereof wherein R¹, R², and R³ are as described herein.

In certain embodiments, R¹ is chosen from (C₃₋₇) cycloalkyl, 4- to 7-membered heterocycloalkyl, (C₆₋₁₀) aryl, and 5- to 10-membered heteroaryl, any of which is optionally substituted with one R⁶ and one or more R⁷ wherein R⁶ is optionally substituted with one or more R¹⁰.

In certain embodiments, R¹ is chosen from (C₃₋₇) cycloalkyl, 4- to 7-membered heterocycloalkyl, (C₆₋₁₀) aryl, and 5- to 10-membered heteroaryl, any of which is optionally substituted with one R⁶ and one or more R⁷.

In certain embodiments, R¹ is chosen from (C₆₋₁₀) aryl and 5- to 10-membered heteroaryl, either of which is optionally substituted with one R⁶ and one or more R⁷, wherein R⁶ is optionally substituted with one or more R¹⁰.

In certain embodiments, R¹ is chosen from (C₆₋₁₀) aryl, and 5- to 10-membered heteroaryl, any of which is optionally substituted with one R⁶ and one or more R⁷.

In certain embodiments, R¹ is phenyl optionally substituted with one R⁶ and one or more R⁷ wherein R⁶ is optionally substituted with one or more R¹⁰

In certain embodiments, R¹ is phenyl optionally substituted with one R⁶ and one or more R⁷.

In certain embodiments, R¹ optionally substituted with one R⁶ and one or more R⁷ wherein R⁶ is optionally substituted with one or more R¹⁰ is chosen from

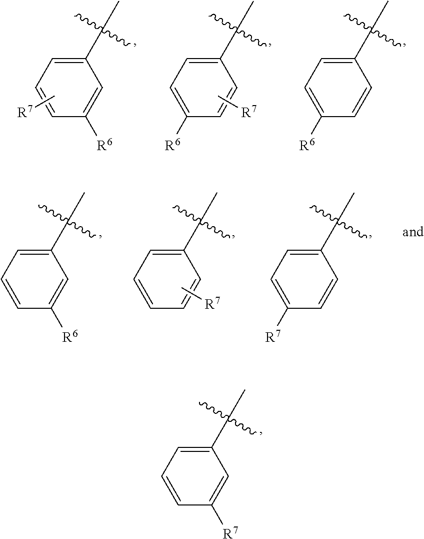

wherein
R⁶ is chosen from cycloalkyl, heterocycloalkyl, aryl, heteroaryl, (cycloalkyl)carbonyl, (heterocycloalkyl)carbonyl, (aryl)carbonyl, and (heteroaryl)carbonyl, any of which is optionally substituted with one or more R¹⁰; and
R⁷ is independently chosen from halo, cyano, hydroxy, alkyl, alkoxy, (hydroxy)alkyl, haloalkoxy, and (hydroxy)alkoxy.

In certain embodiments, R⁶ is chosen from cycloalkyl, heterocycloalkyl, aryl, and heteroaryl, any of which is optionally substituted with one or more R¹⁰.

In certain embodiments, R⁶ is heterocycloalkyl optionally substituted with one or more R¹⁰.

In certain embodiments, R⁶ is chosen from pyrrolidin-1-yl, piperidin-1-yl, piperazin-1-yl, thiomorpholin-4-yl, 1-oxidothiomorpholino, 1,1-dioxidothiomorpholino, 3,3-dioxido-3-thia-6-azabicyclo[3.1.1]heptan-6-yl, 1-oxa-8-azaspiro[4.5]decan-8-yl, and 3-oxopiperazin-1-yl, any of which is optionally substituted with one or more R¹⁰.

In certain embodiments, R⁶ is chosen from pyrrolidin-1-yl, piperidin-1-yl, piperazin-1-yl, thiomorpholin-4-yl, phenyl, pyrazol-4-yl, 1H-1,2,3-triazol-1-yl, pyridin-4-yl, (pyrrolidin-1-yl)carbonyl, any of which is optionally substituted with one or more R¹⁰.

In certain embodiments, each R¹⁰ is independently chosen from cyano, hydroxy, alkyl, (hydroxy)alkyl, and alkoxy.

In certain embodiments, each R¹⁰ is independently chosen from methyl, methoxy, ethoxy, isopropoxy, hydroxy, and —CH₂CH₂OH.

In certain embodiments, R¹ optionally substituted with one R⁶ and one or more R⁷ wherein R⁶ is optionally substituted with one or more R¹⁰ is chosen from

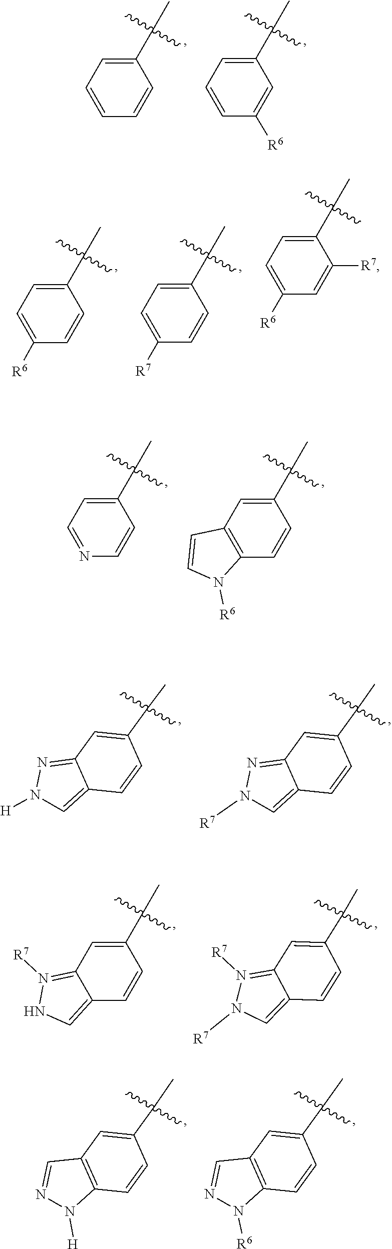

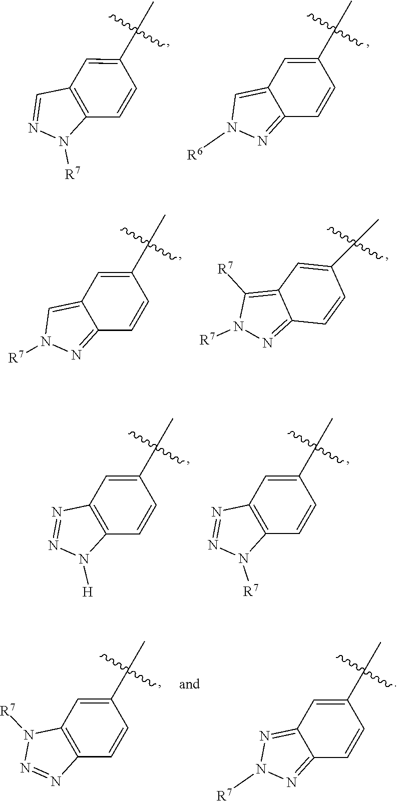

In certain embodiments, R¹ optionally substituted with one Re and one or more R⁷ wherein R⁶ is optionally substituted with one or more R¹⁰ is chosen from

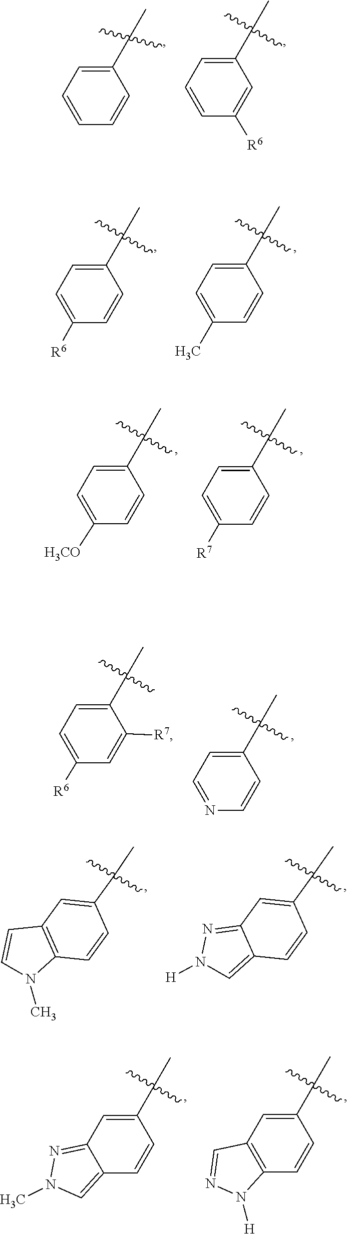

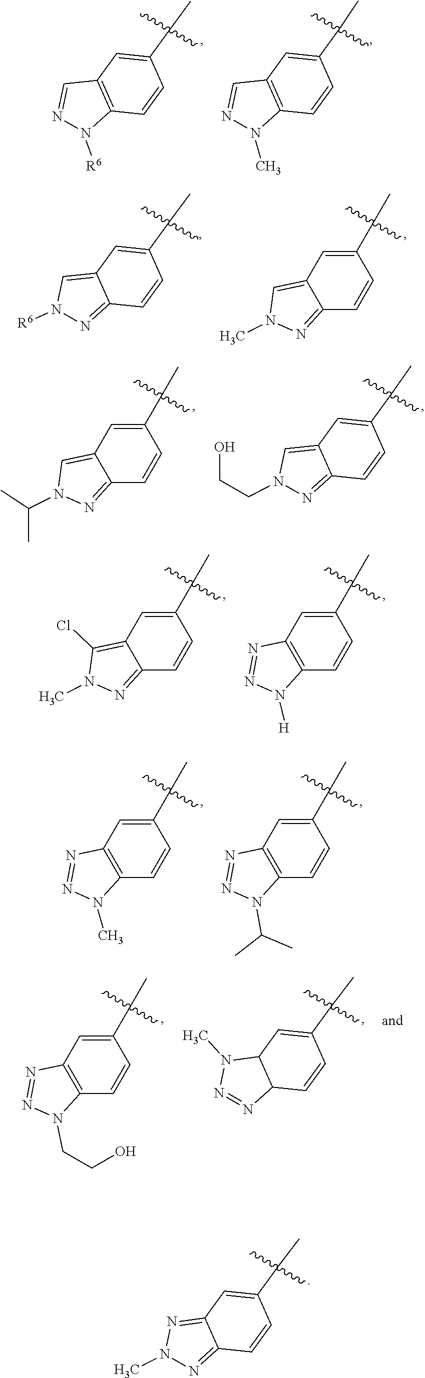

In certain embodiments, R¹ optionally substituted with one R⁶ and one or more R⁷ wherein R⁶ is optionally substituted with one or more R¹⁰ is chosen from

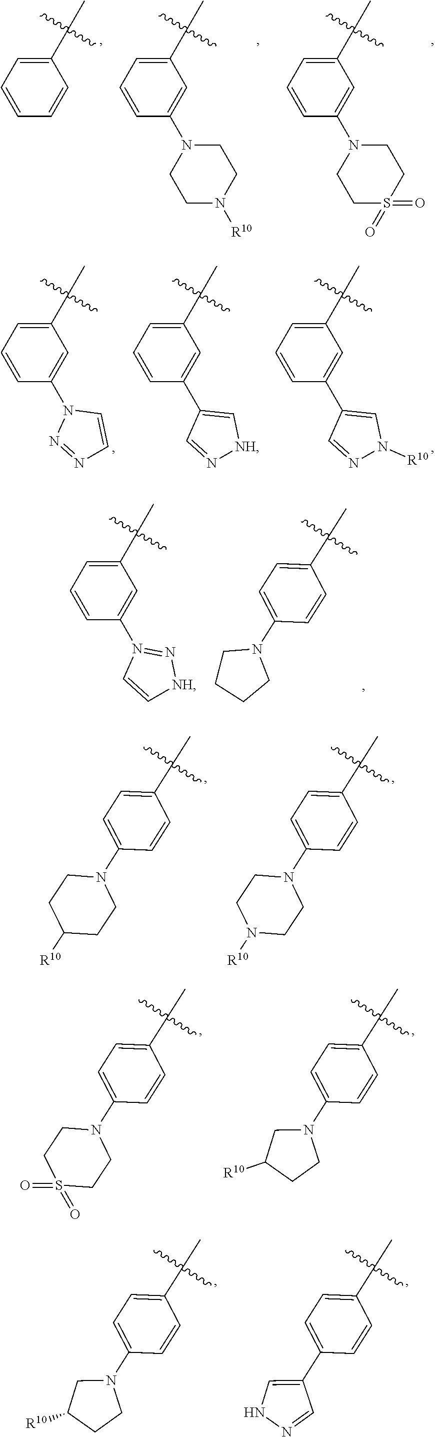

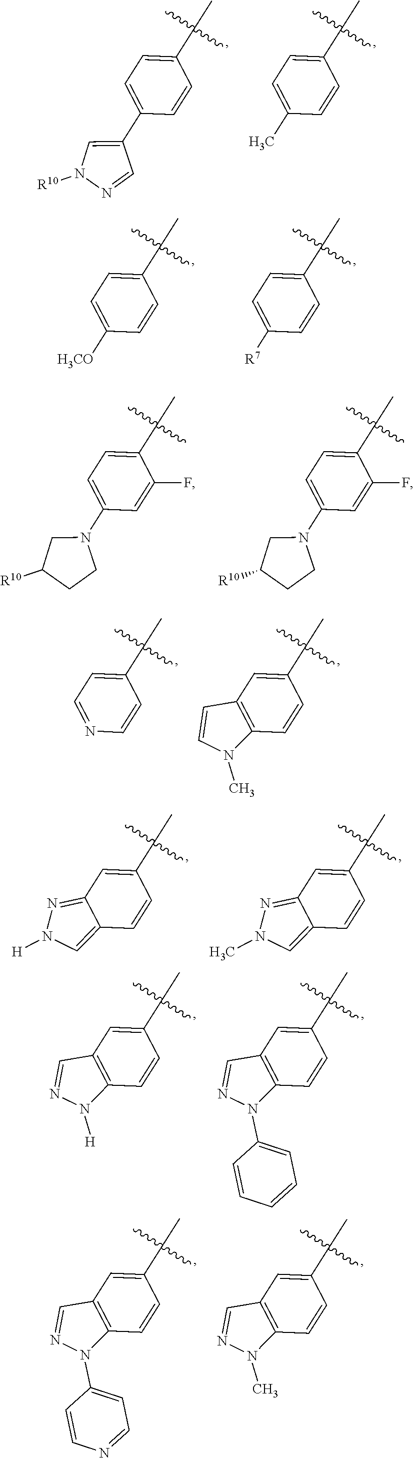

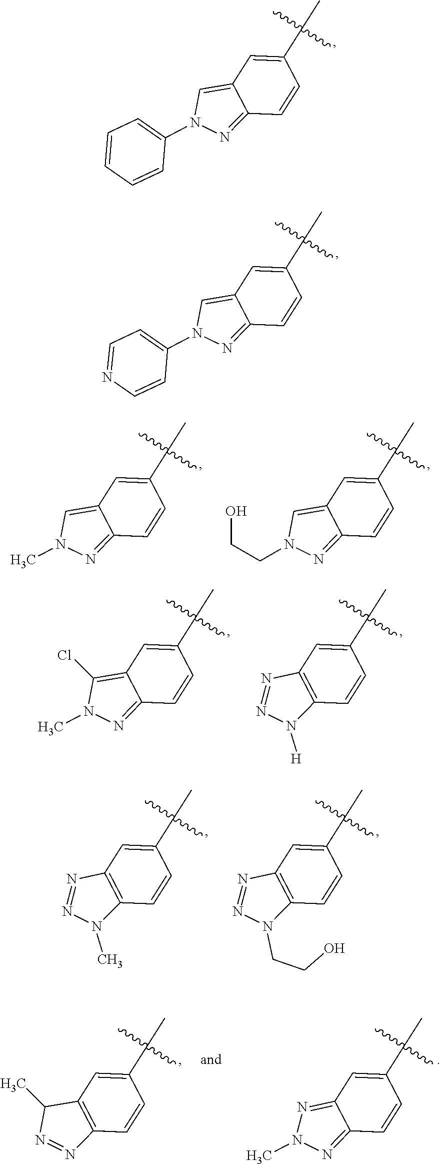

In certain embodiments. R¹ optionally substituted with one R⁶ and one or more R⁷ wherein R⁶ is optionally substituted with one or more R¹⁰ is chosen from

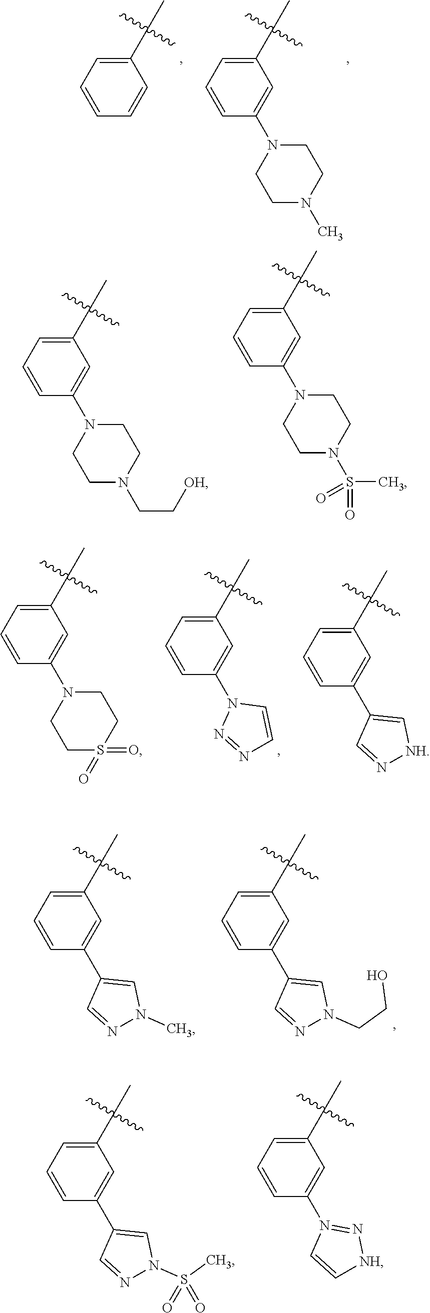

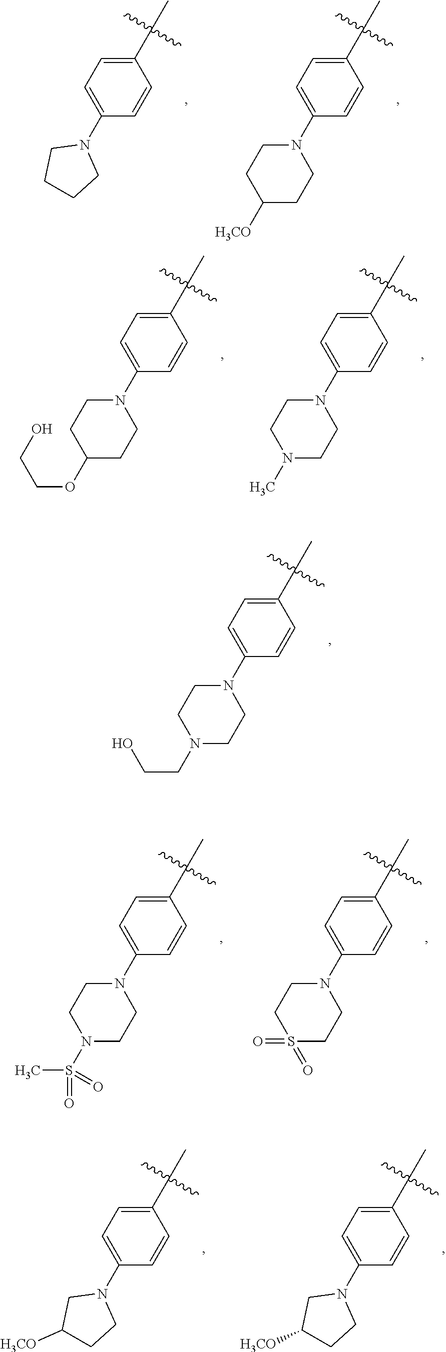

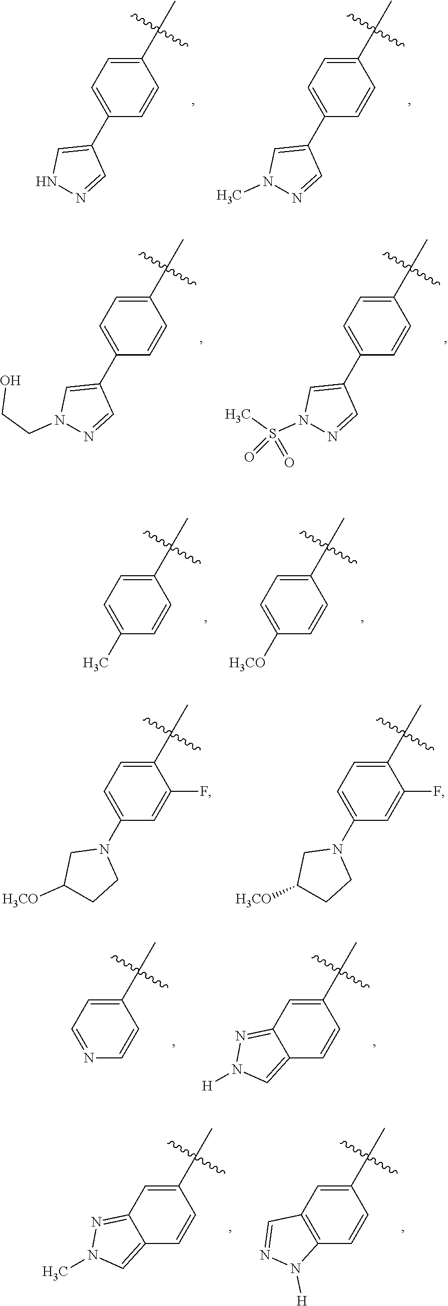

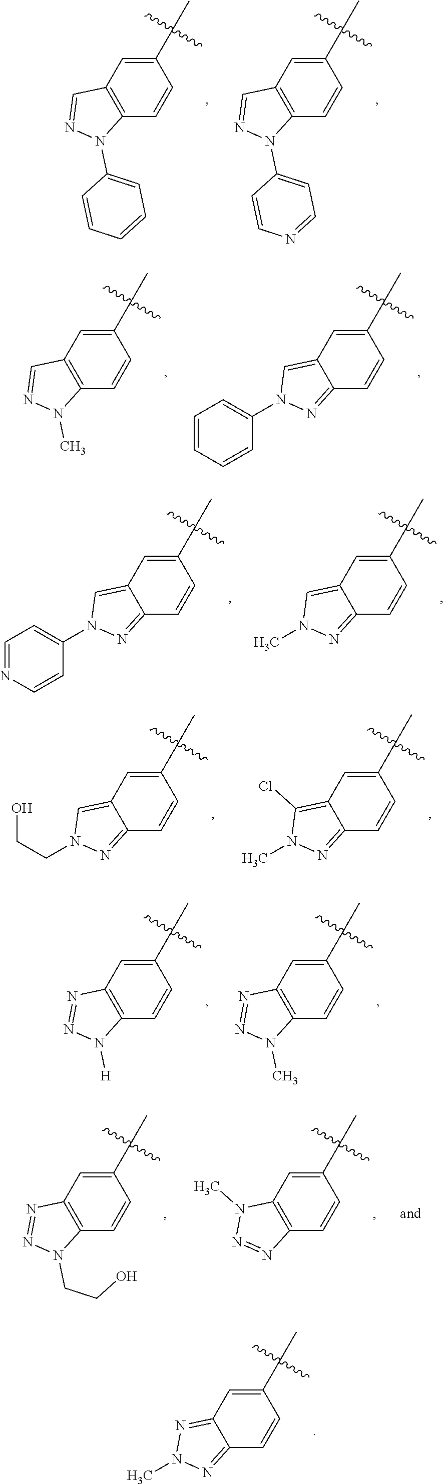

In certain embodiments, R¹ optionally substituted with one R⁶ and one or more R⁷ wherein R⁶ is optionally substituted with one or more R¹⁰ is chosen from

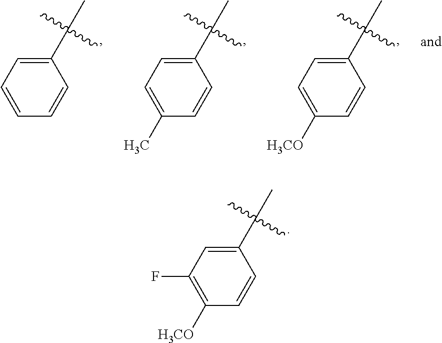

In certain embodiments, R² is H.

In certain embodiments, R³ is chosen from H, cycloalkyl, heterocycloalkyl, aryl, heteroaryl, (cycloalkyl)C₁₋₆alkyl, (heterocycloalkyl)C₁₋₆alkyl, (aryl)C₁₋₆alkyl, and (heteroaryl)C₁₋₆alkyl, any of which is optionally substituted with one or more R⁸. In certain embodiments, R³ is chosen from H, cycloalkyl, heterocycloalkyl, aryl, heteroaryl, (cycloalkyl)methyl, (heterocycloalkyl)methyl, (aryl)methyl, and (heteroaryl)methyl, any of which is optionally substituted with one or more R⁸.

In certain embodiments, R³ is chosen from H, C₃₋₁₀cycloalkyl, 4- to 10-membered heterocycloalkyl, C₆₋₁₀aryl, 5- to 10-membered heteroaryl, (C₃₋₁₀cycloalkyl)methyl, (4- to 10-membered heterocycloalkyl)methyl, (C₆₋₁₀aryl)methyl, and (5- to 10-membered heteroaryl)methyl, any of which is optionally substituted with one or more R⁸.

In certain embodiments, R³ is chosen from H, cycloalkyl, heterocycloalkyl, aryl, heteroaryl, (cyclopropyl)methyl, (cyclobutyl)methyl, (cyclopentyl)methyl, (cyclohexyl)methyl, (bicyclo[3.1.1]heptanyl)methyl, (pyrrolidinyl)methyl, (piperidinyl)methyl, (azepanyl)methyl, (quinuclidinyl)methyl, (phenyl)methyl, and (heteroaryl)methyl, any of which is optionally substituted with one or more R⁸.

In certain embodiments, R³ is chosen from (C₃₋₁₀cycloalkyl)methyl and (4- to 10-membered heterocycloalkyl)methyl, any of which is optionally substituted with one or more R⁸.

In certain embodiments, R³ is chosen from

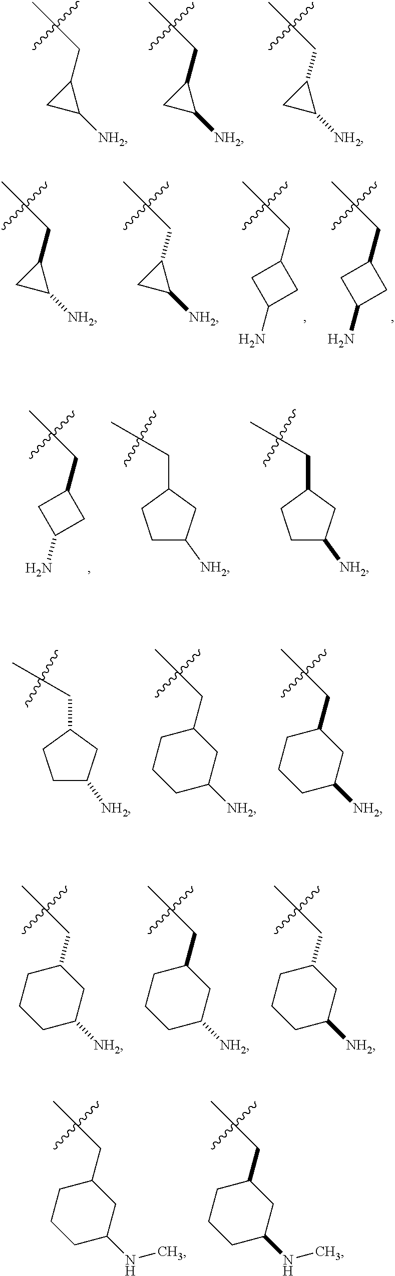

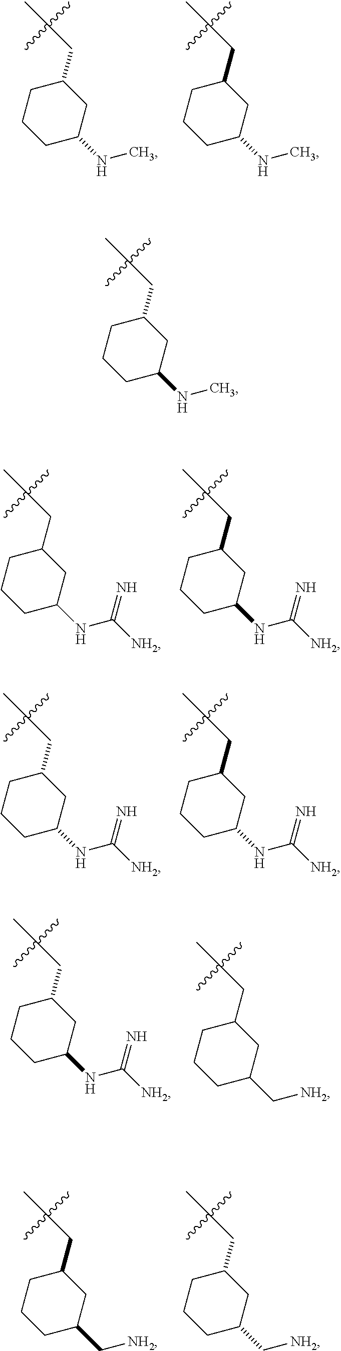

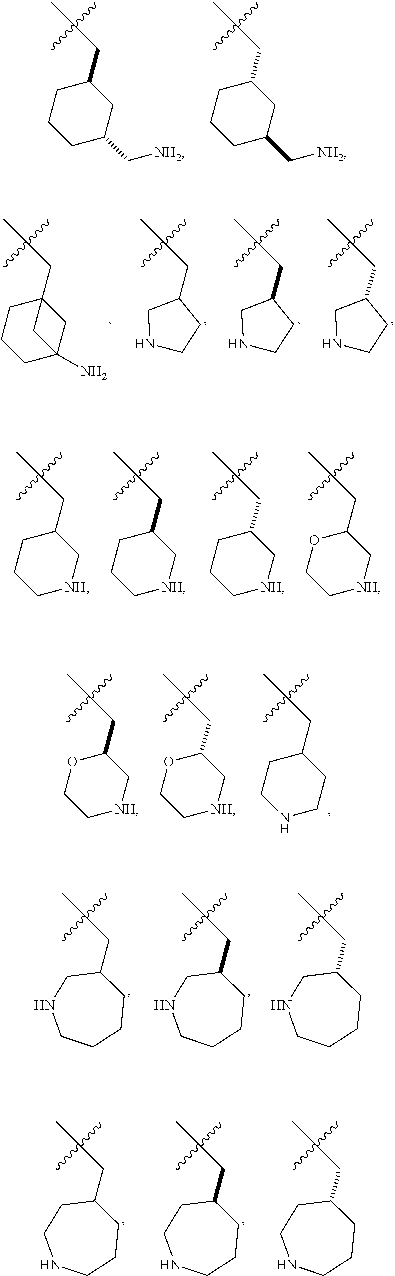

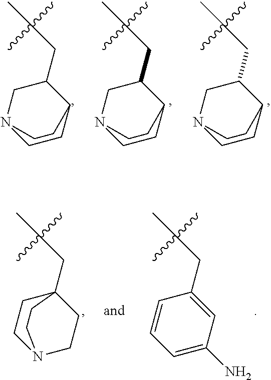

In certain embodiments. R³ is chosen from

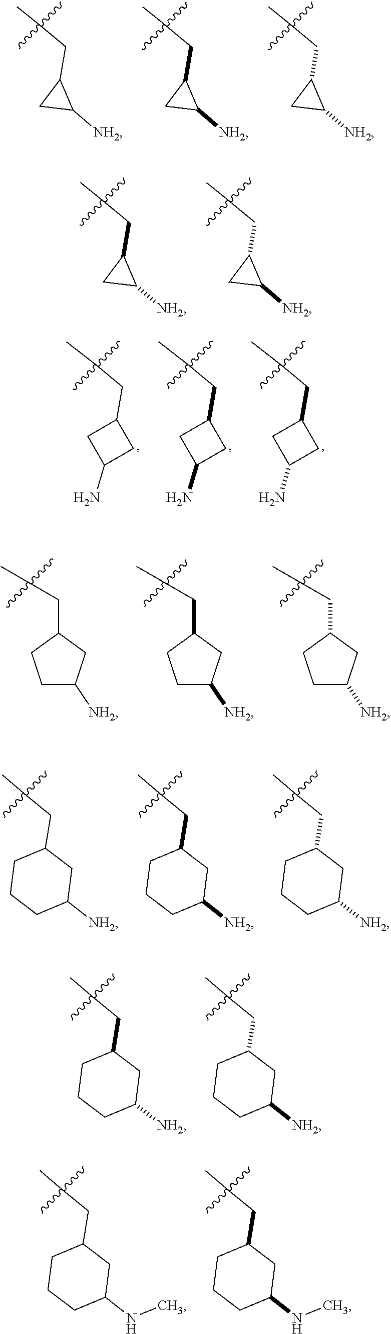

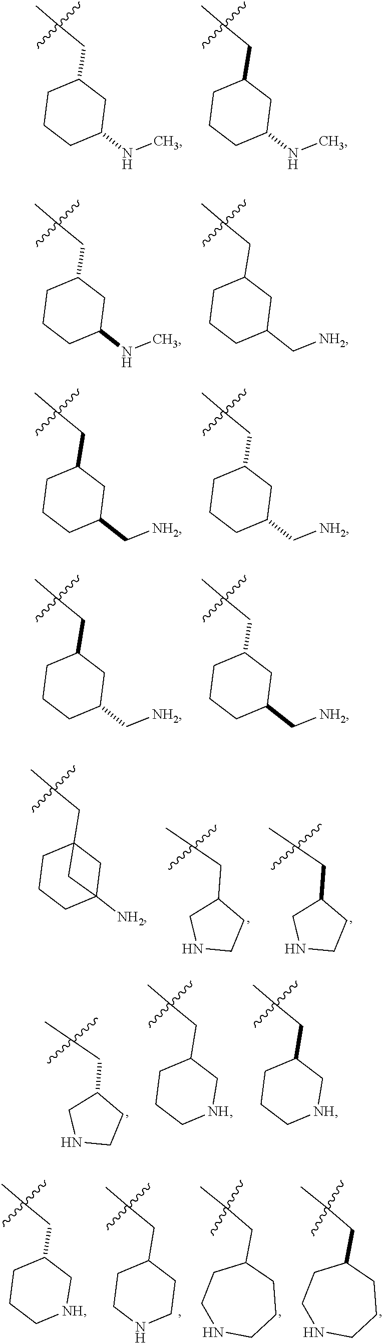

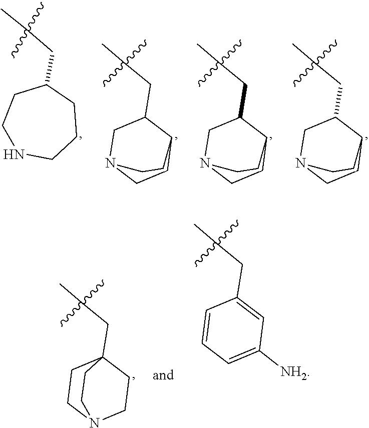

In some embodiments, R³ is chosen from

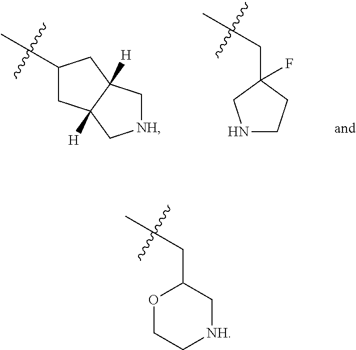 [SMILES: *C1C[C@H]2CNC[C@H]2C1 ; *CC1(F)CCNC1 ; "and" ; *CC1CNCCO1]

In certain embodiments, R² and R³, together with the intervening nitrogen, combine to form heterocycloalkyl chosen from pyrrolidinyl, piperidinyl, azepanyl, octahydroquinolinyl, octahydro-1H-pyrrolo[3,2-c]pyridin-5-yl, 2,6-diazaspiro[3.4]octan-6-yl, 2,7-diazaspiro[4.4]nonan-2-yl, 1,8-diazaspiro[4.5]decan-8-yl, 2,7-diazaspiro[4.5]decan-7-yl, 3,9-diazaspiro[5.5]undecan-3-yl, 9-oxa-3,7-diazabicyclo[3.3.1]nonan-3-yl, and (3aR, 8aS)-decahydropyrrolo[3,4-d]azepin-6-yl, any of which is optionally substituted with one or more R⁸.

In certain embodiments, R² and R³, together with the intervening nitrogen, combine to form heterocycloalkyl chosen from pyrrolidinyl, piperidinyl, azepanyl, and octahydroquinolinyl, any of which is optionally substituted with one or more R⁸.

In certain embodiments, R² and R³, together with the intervening nitrogen, combine to form

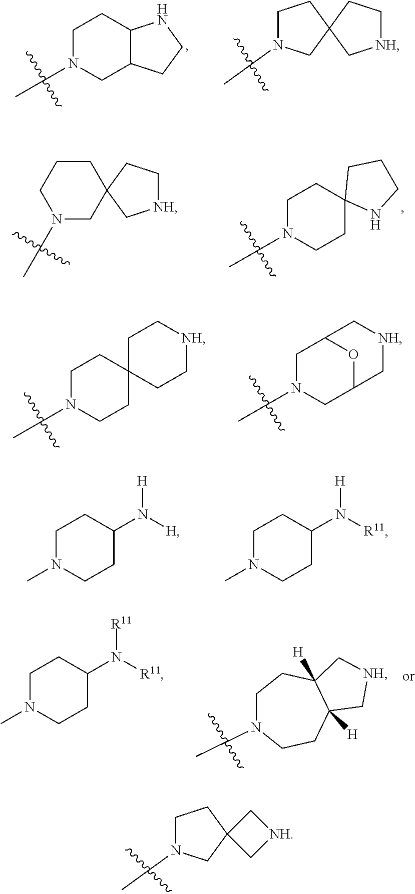

In certain embodiments, R⁴ is chosen from (C₃₋₇) cycloalkyl, 4- to 7-membered heterocycloalkyl, (C₆₋₁₀) aryl, and 5- to 10-membered heteroaryl, any of which is optionally substituted with one or more R⁹.

In certain embodiments, R⁴ is chosen from phenyl, pyridinyl, pyridazinyl, pyrimidinyl, and pyrazinyl, any of which is optionally substituted with one or more R⁹.

In certain embodiments:
R⁴ is

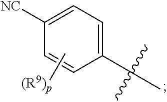

each R⁹ is independently chosen from cyano, halo, hydroxy, alkyl, and alkoxy; and
p is chosen from 0, 1, and 2.

In certain embodiments:
R⁴ is

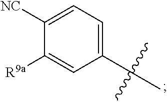

and
R^{7a} is chosen from H, cyano, halo, hydroxy, alkyl, and alkoxy.

In certain embodiments, R^{9a} is chosen from H, cyano, F, Cl, Br, hydroxy, C₁₋₆alkyl, and C₁₋₆alkoxy. In certain embodiments, R^{9a} is chosen from H, cyano, F, Cl, hydroxy, methyl, and methoxy. In certain embodiments, R^{9a} is chosen from H, F, Cl, and hydroxy. In certain embodiments, R^{9a} is chosen from H and F.

In certain embodiments, R⁴ is chosen from

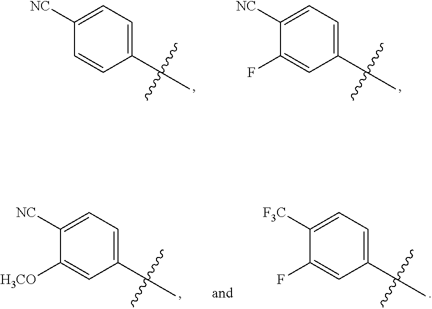 [SMILES: *c1ccc(C#N)cc1 ; *c1ccc(C#N)c(F)c1 ; *c1ccc(C#N)c(OC)c1 ; *c1ccc(C(F)(F)F)c(F)c1]

In certain embodiments, R⁴ is

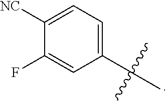 [SMILES: *c1ccc(C#N)c(F)c1]

In certain embodiments, R⁵ is chosen from H, F, and Cl. In certain embodiments, R⁵ is chosen from H and Cl. In certain embodiments, R⁵ is H.

Also provided is a compound chosen from:

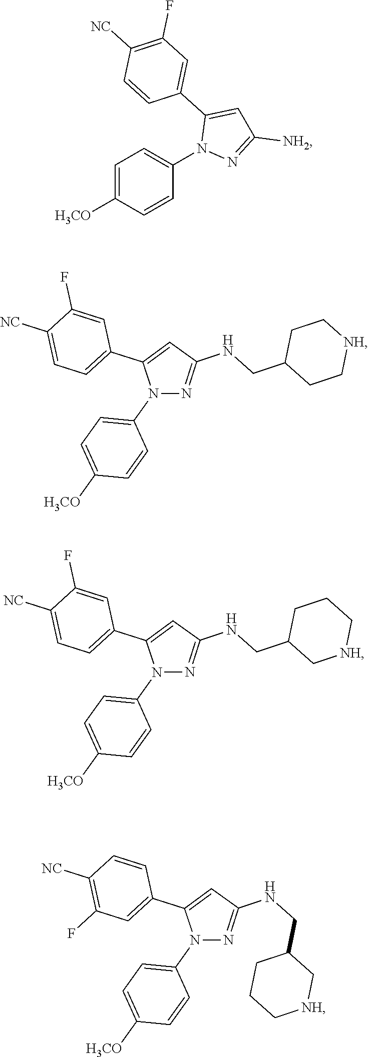

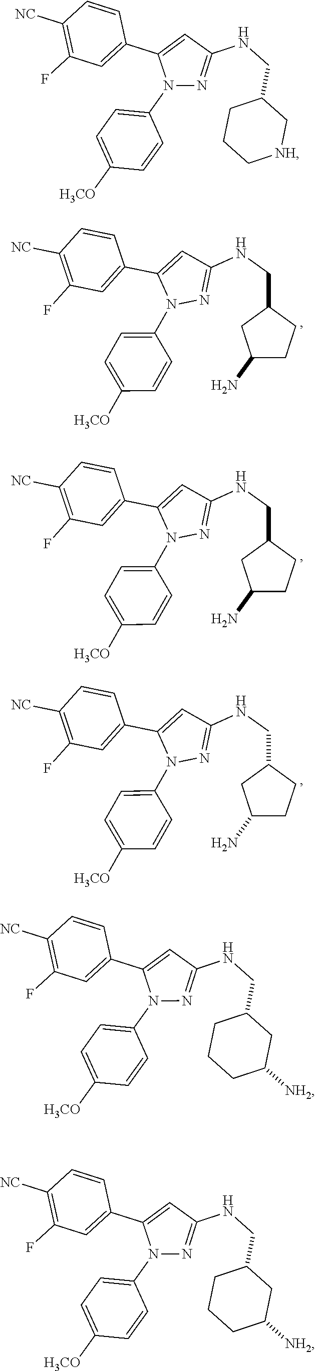

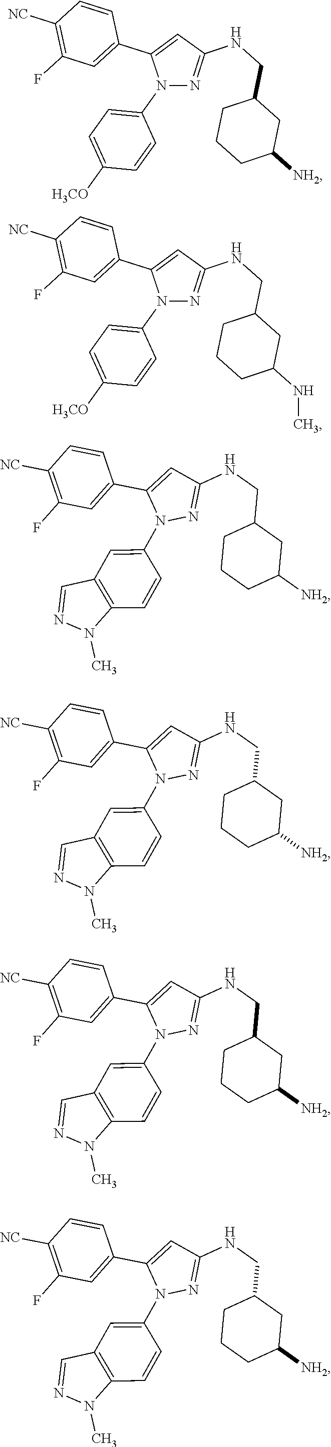

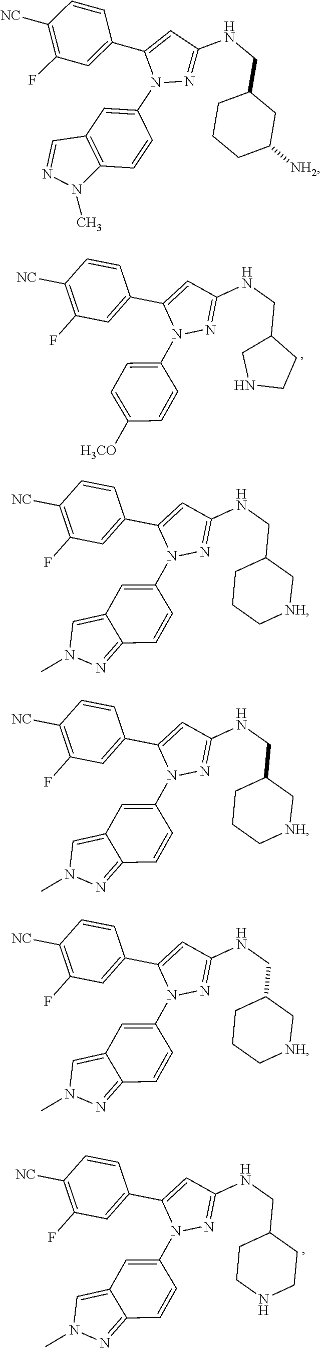

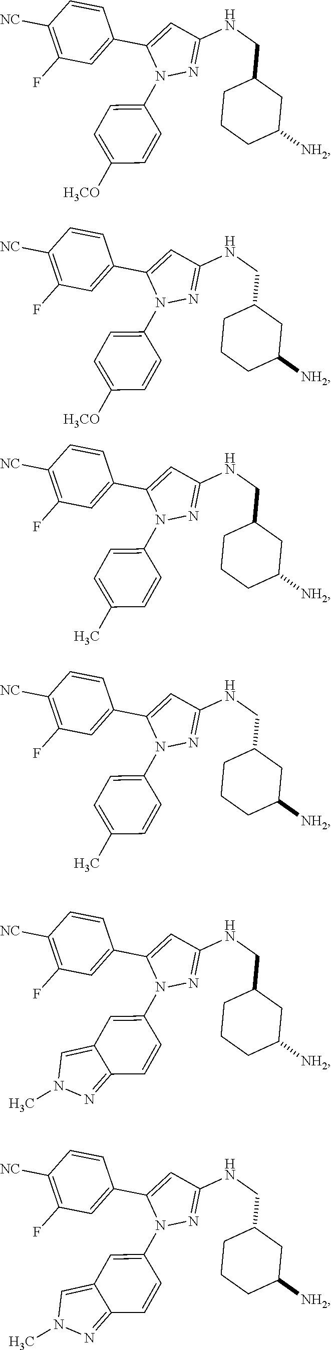

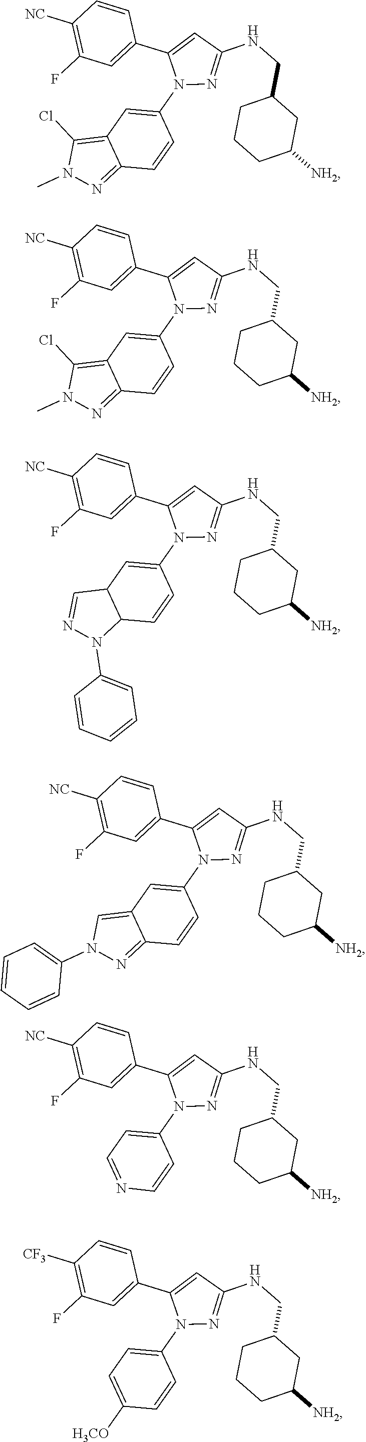

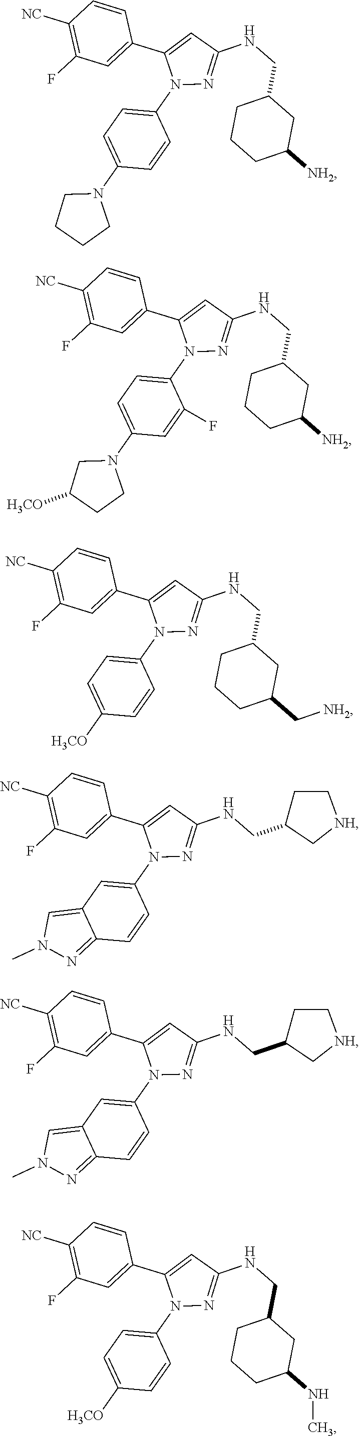

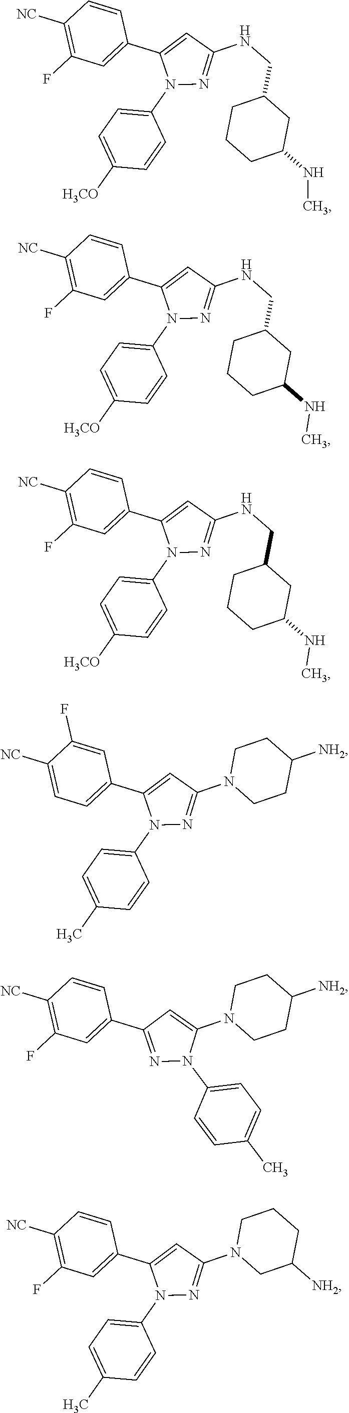

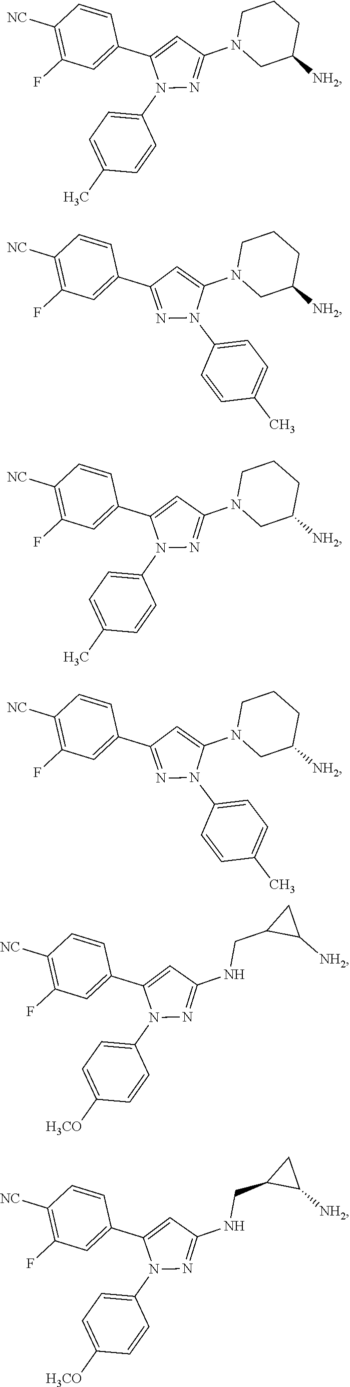

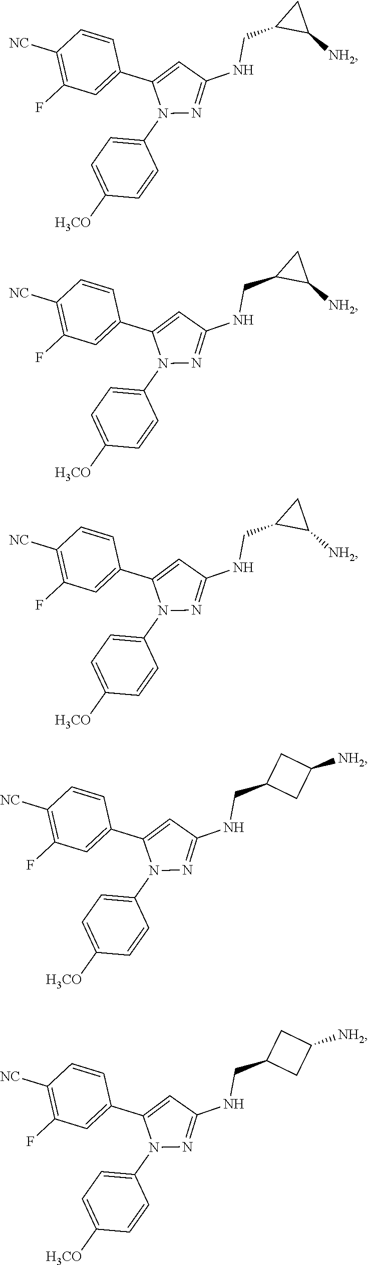 [SMILES: COc1ccc(-n2nc(NC[C@@H]3C[C@H]3N)cc2-c2ccc(C#N)c(F)c2)cc1 ; COc1ccc(-n2nc(NC[C@H]3C[C@H]3N)cc2-c2ccc(C#N)c(F)c2)cc1 ; COc1ccc(-n2nc(NC[C@@H]3C[C@@H]3N)cc2-c2ccc(C#N)c(F)c2)cc1 ; COc1ccc(-n2nc(NC[C@H]3C[C@@H](N)C3)cc2-c2ccc(C#N)c(F)c2)cc1 ; COc1ccc(-n2nc(NC[C@H]3C[C@H](N)C3)cc2-c2ccc(C#N)c(F)c2)cc1]

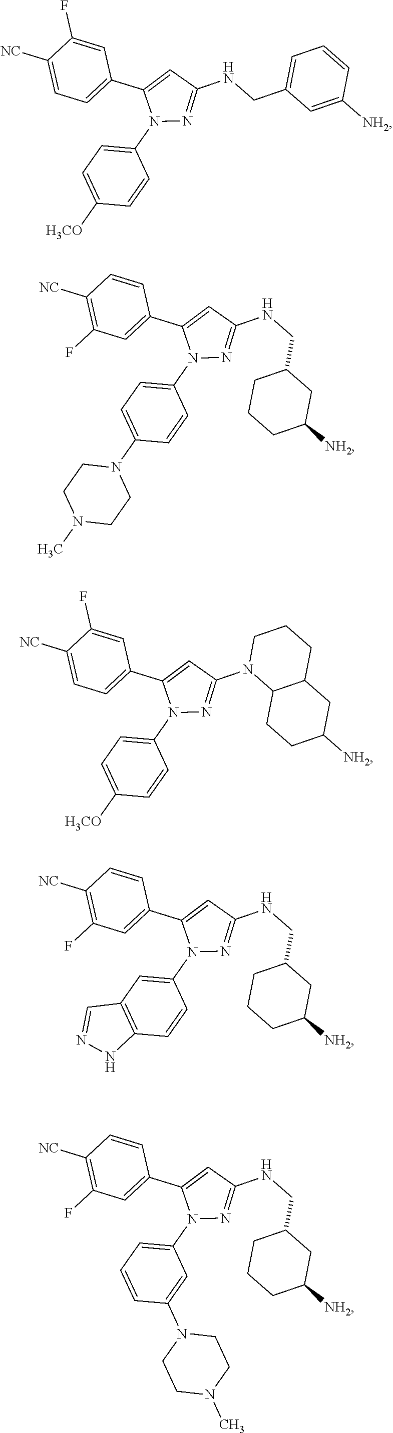

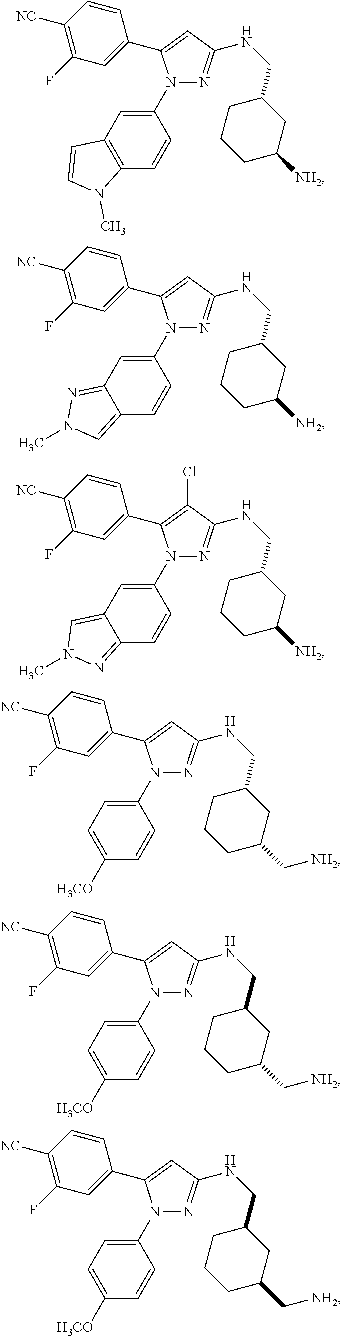

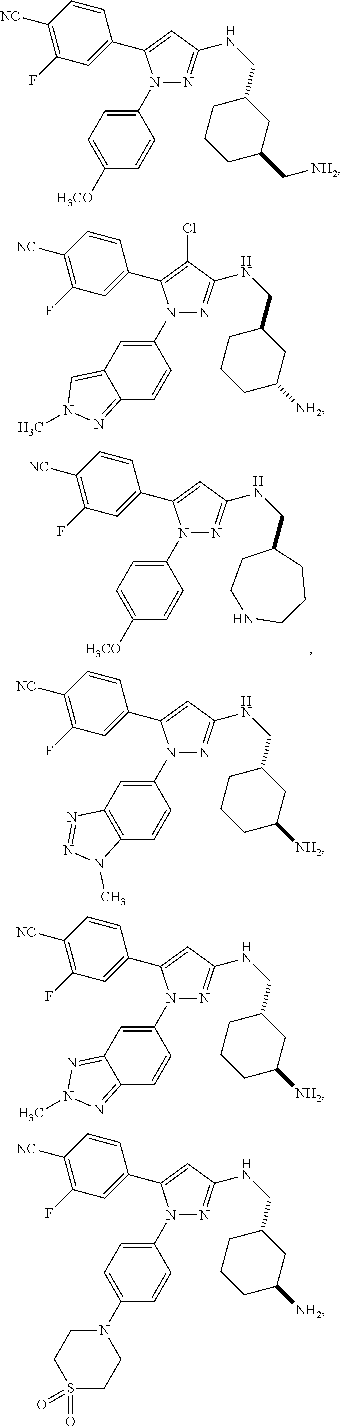

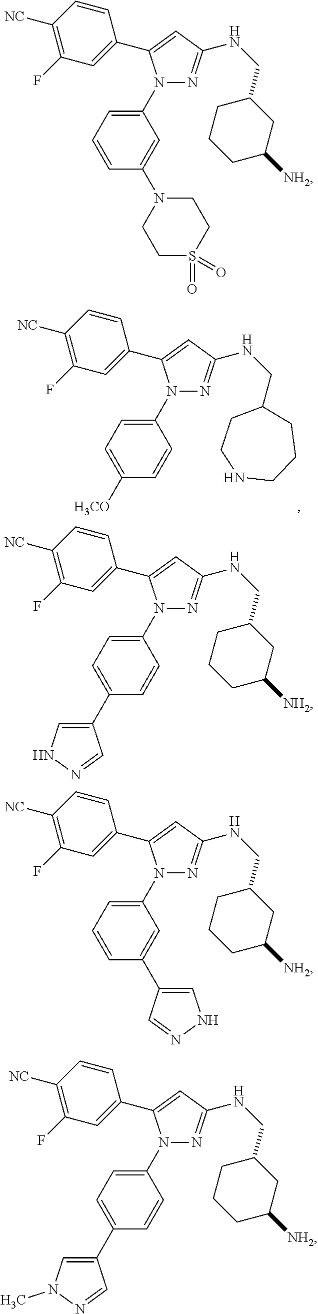

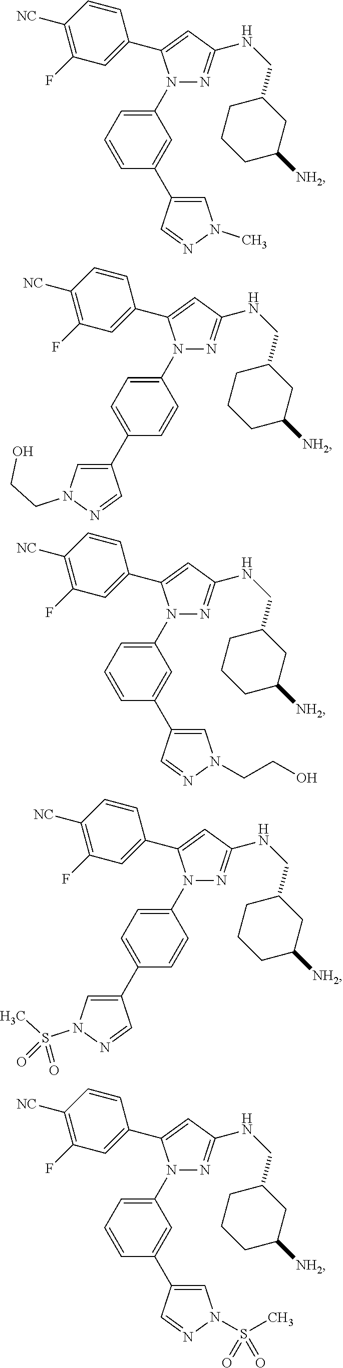

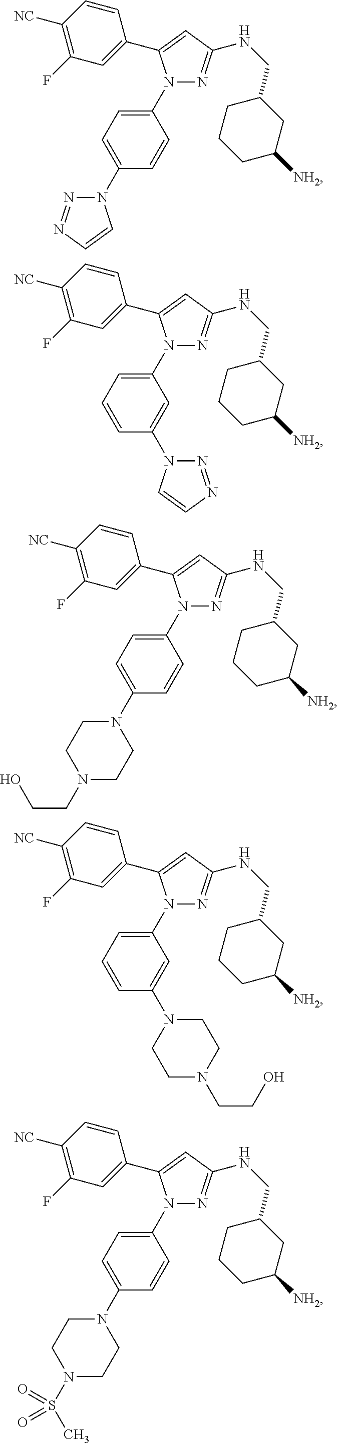

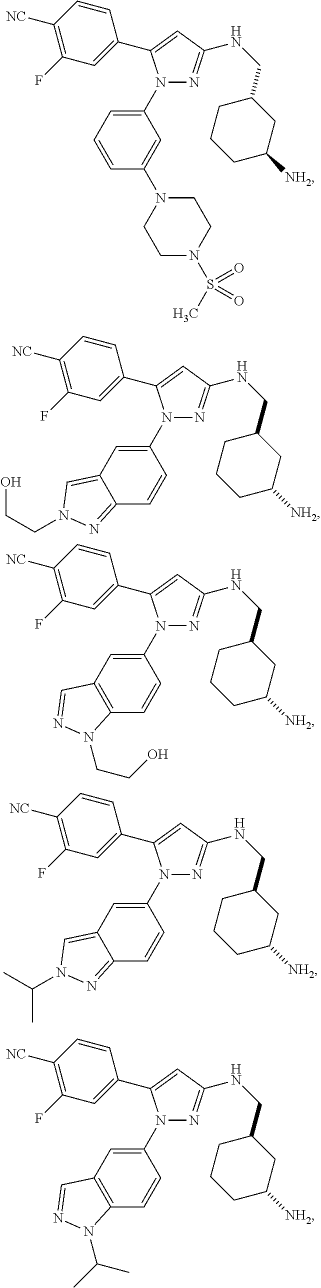

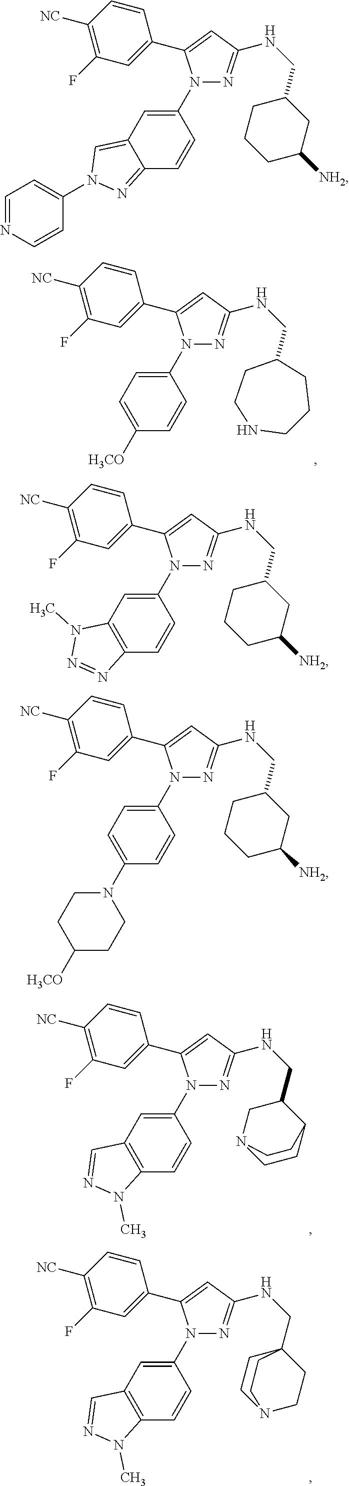

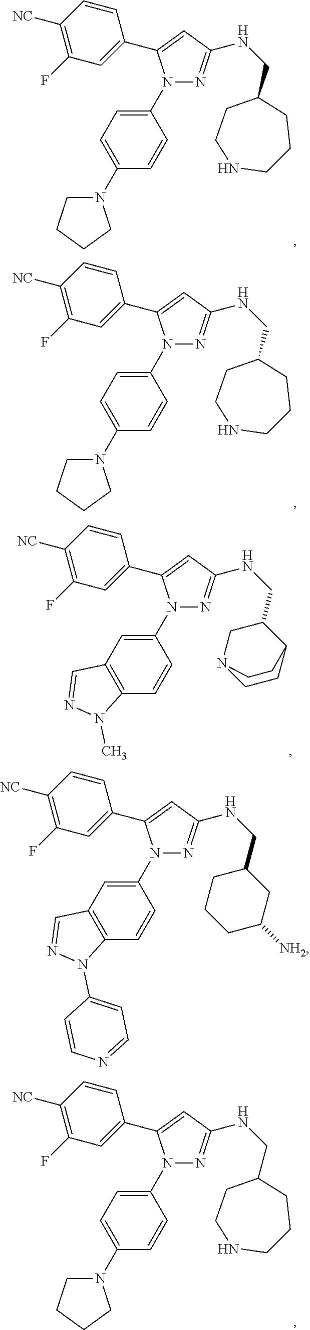

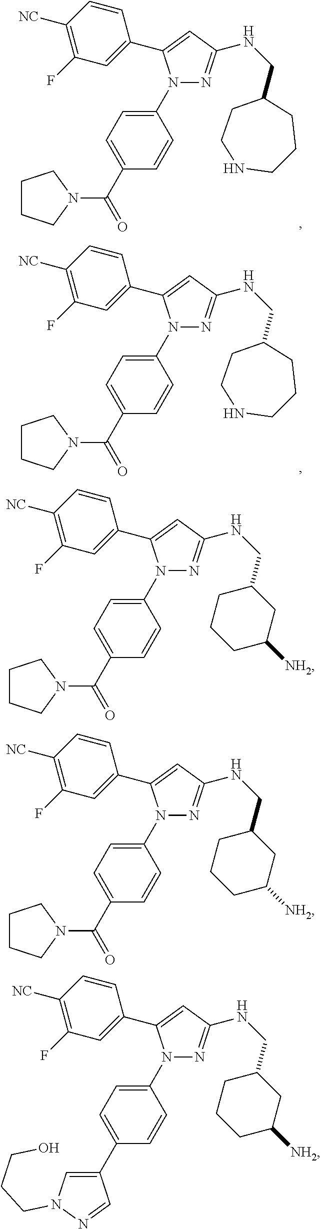

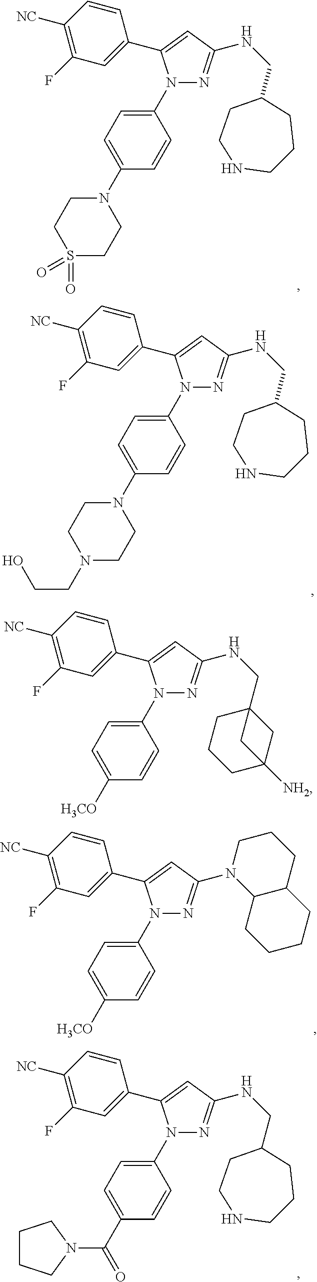

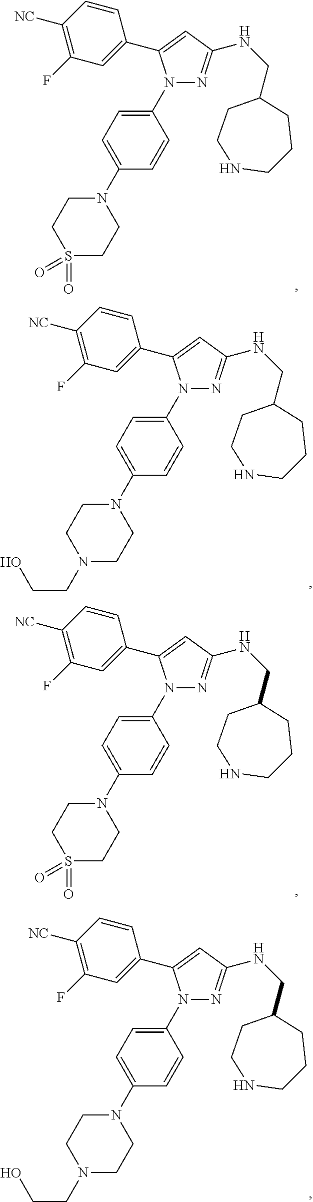

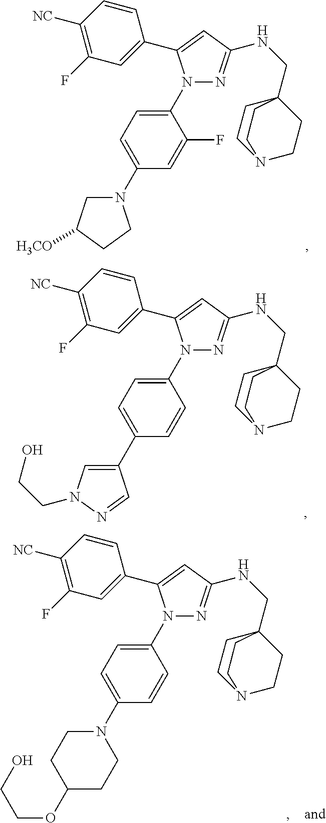

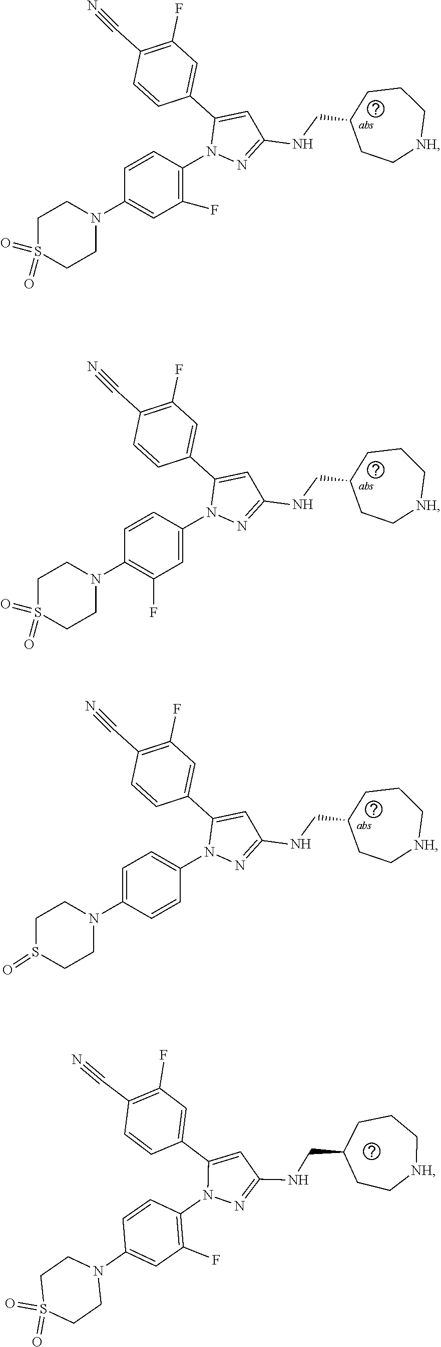 [SMILES: N#Cc1ccc(-c2cc(NC[C@@H]3CCCNCC3)nn2-c2ccc(N3CCS(=O)(=O)CC3)cc2F)cc1F ; N#Cc1ccc(-c2cc(NC[C@@H]3CCCNCC3)nn2-c2ccc(N3CCS(=O)(=O)CC3)c(F)c2)cc1F ; N#Cc1ccc(-c2cc(NC[C@@H]3CCCNCC3)nn2-c2ccc(N3CCS(=O)CC3)cc2)cc1F ; N#Cc1ccc(-c2cc(NC[C@H]3CCCNCC3)nn2-c2ccc(N3CCS(=O)(=O)CC3)cc2F)cc1F]

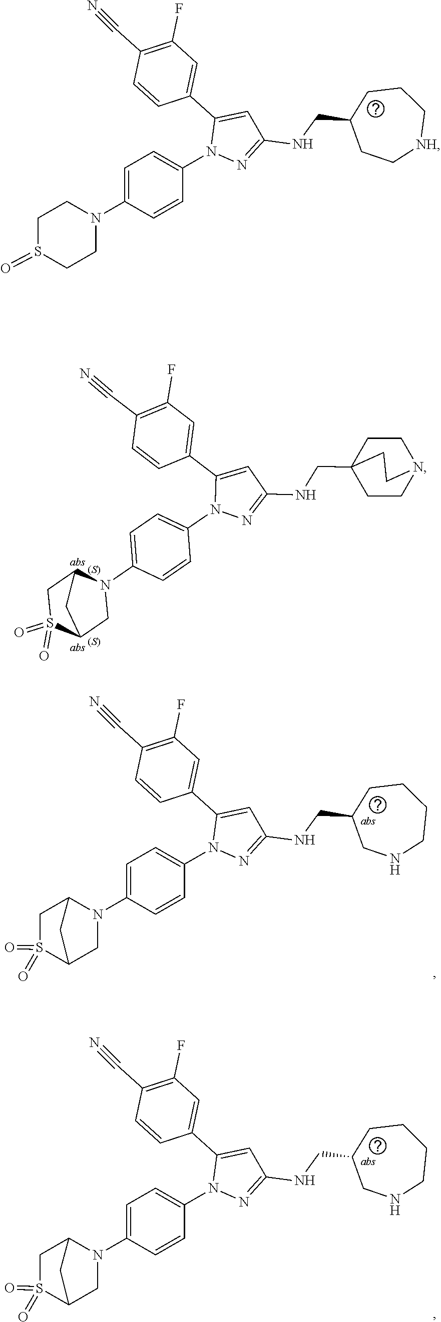

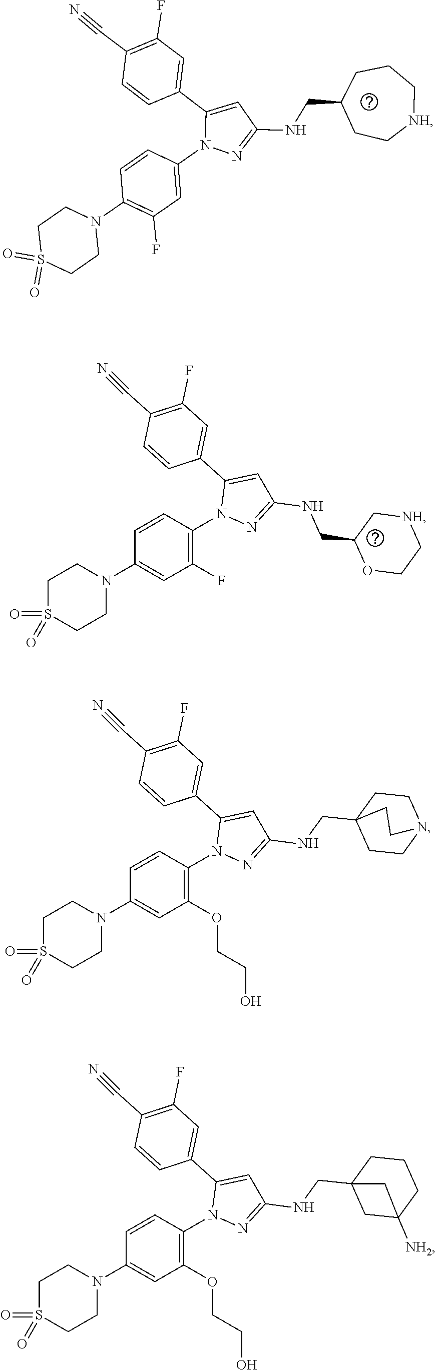 [SMILES: N#Cc1ccc(-c2cc(NC[C@H]3CCCNCC3)nn2-c2ccc(N3CCS(=O)(=O)CC3)c(F)c2)cc1F ; N#Cc1ccc(-c2cc(NC[C@H]3CNCCO3)nn2-c2ccc(N3CCS(=O)(=O)CC3)cc2F)cc1F ; N#Cc1ccc(-c2cc(NCC34CCN(CC3)CC4)nn2-c2ccc(N3CCS(=O)(=O)CC3)cc2OCCO)cc1F ; N#Cc1ccc(-c2cc(NCC34CCCC(N)(C3)C4)nn2-c2ccc(N3CCS(=O)(=O)CC3)cc2OCCO)cc1F]

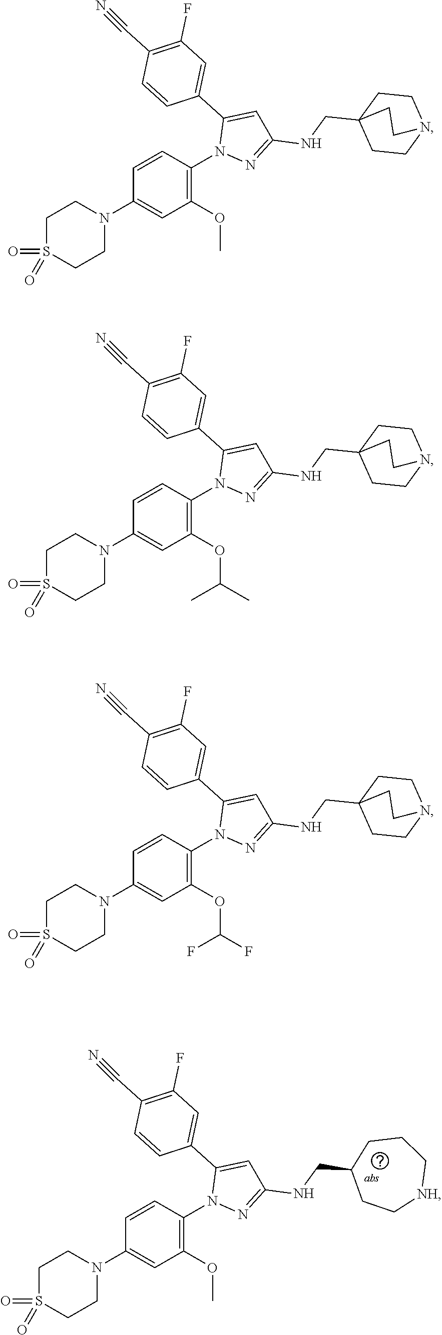 [SMILES: COc1cc(N2CCS(=O)(=O)CC2)ccc1-n1nc(NCC23CCN(CC2)CC3)cc1-c1ccc(C#N)c(F)c1 ; CC(C)Oc1cc(N2CCS(=O)(=O)CC2)ccc1-n1nc(NCC23CCN(CC2)CC3)cc1-c1ccc(C#N)c(F)c1 ; N#Cc1ccc(-c2cc(NCC34CCN(CC3)CC4)nn2-c2ccc(N3CCS(=O)(=O)CC3)cc2OC(F)F)cc1F ; COc1cc(N2CCS(=O)(=O)CC2)ccc1-n1nc(NC[C@H]2CCCNCC2)cc1-c1ccc(C#N)c(F)c1]

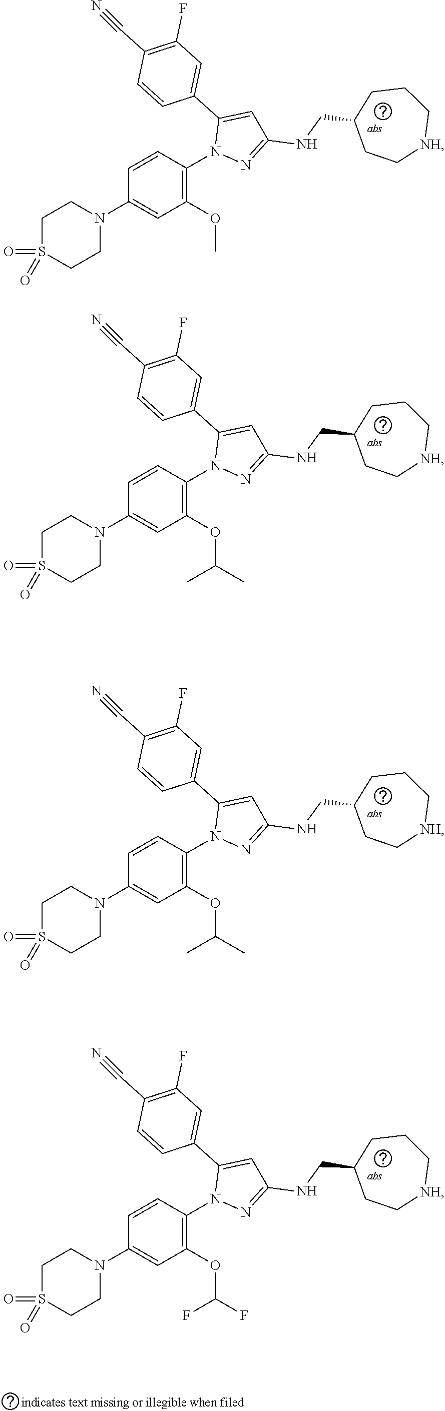 [SMILES: COc1cc(N2CCS(=O)(=O)CC2)ccc1-n1nc(NC[C@@H]2CCCNCC2)cc1-c1ccc(C#N)c(F)c1 ; CC(C)Oc1cc(N2CCS(=O)(=O)CC2)ccc1-n1nc(NC[C@H]2CCCNCC2)cc1-c1ccc(C#N)c(F)c1 ; CC(C)Oc1cc(N2CCS(=O)(=O)CC2)ccc1-n1nc(NC[C@@H]2CCCNCC2)cc1-c1ccc(C#N)c(F)c1 ; N#Cc1ccc(-c2cc(NC[C@H]3CCCNCC3)nn2-c2ccc(N3CCS(=O)(=O)CC3)cc2OC(F)F)cc1F]

or a salt or tautomer thereof.

Also provided is a compound chosen from:

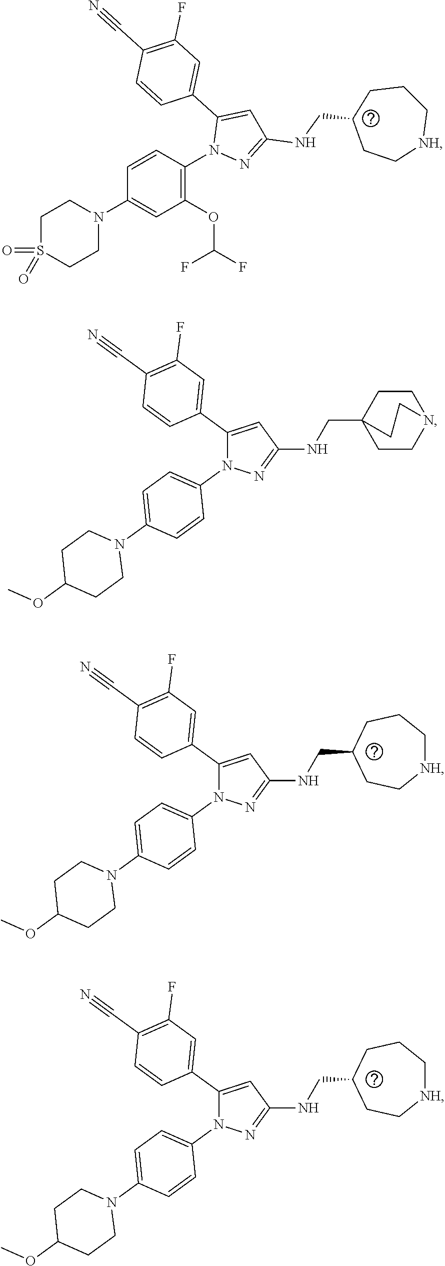 [SMILES: N#Cc1ccc(-c2cc(NC[C@@H]3CCCNCC3)nn2-c2ccc(N3CCS(=O)(=O)CC3)cc2OC(F)F)cc1F ; COC1CCN(c2ccc(-n3nc(NCC45CCN(CC4)CC5)cc3-c3ccc(C#N)c(F)c3)cc2)CC1 ; COC1CCN(c2ccc(-n3nc(NC[C@H]4CCCNCC4)cc3-c3ccc(C#N)c(F)c3)cc2)CC1 ; COC1CCN(c2ccc(-n3nc(NC[C@@H]4CCCNCC4)cc3-c3ccc(C#N)c(F)c3)cc2)CC1]

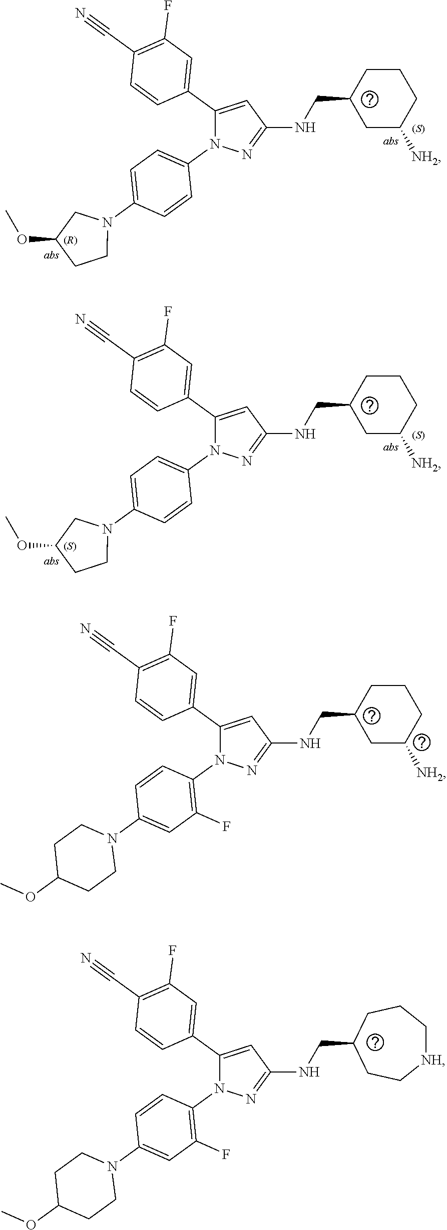

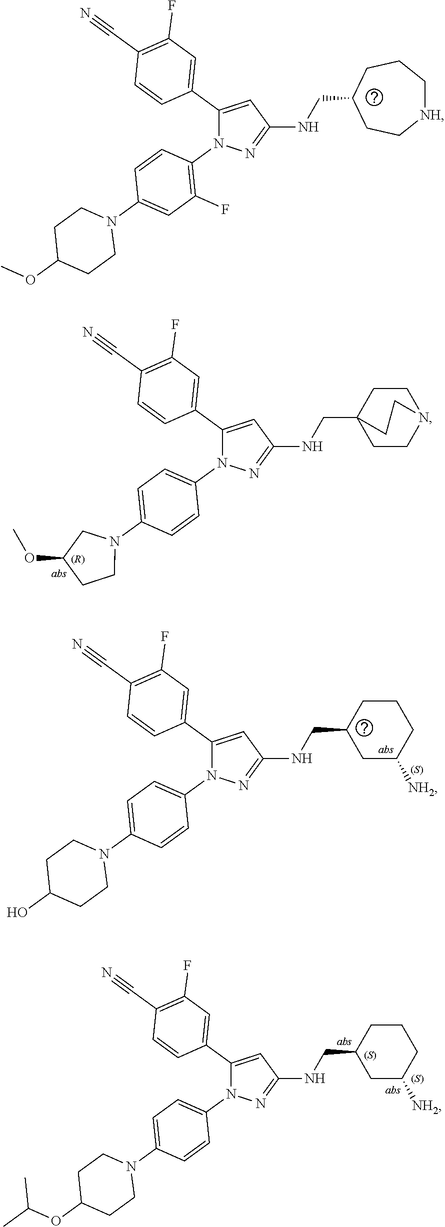

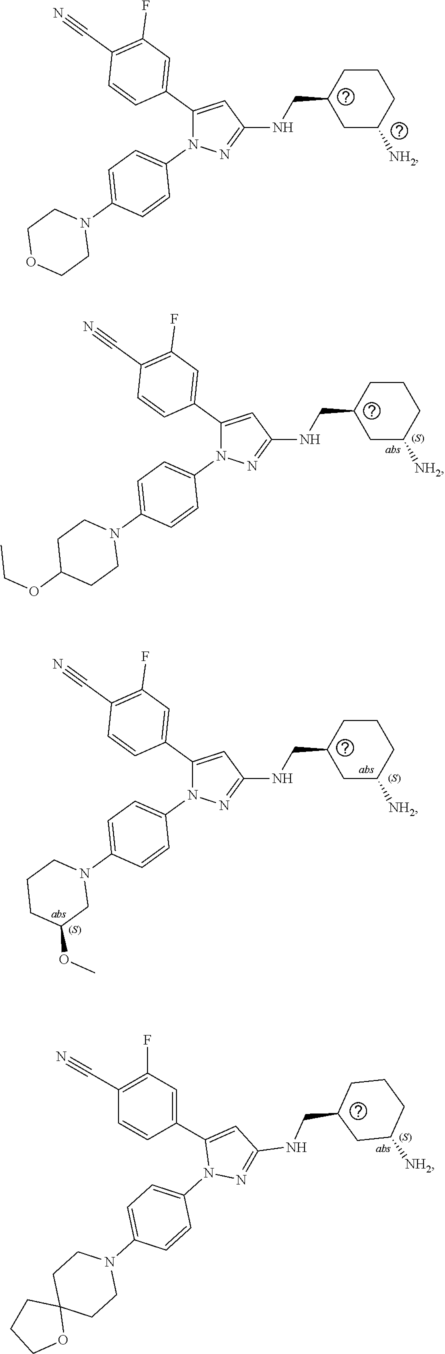

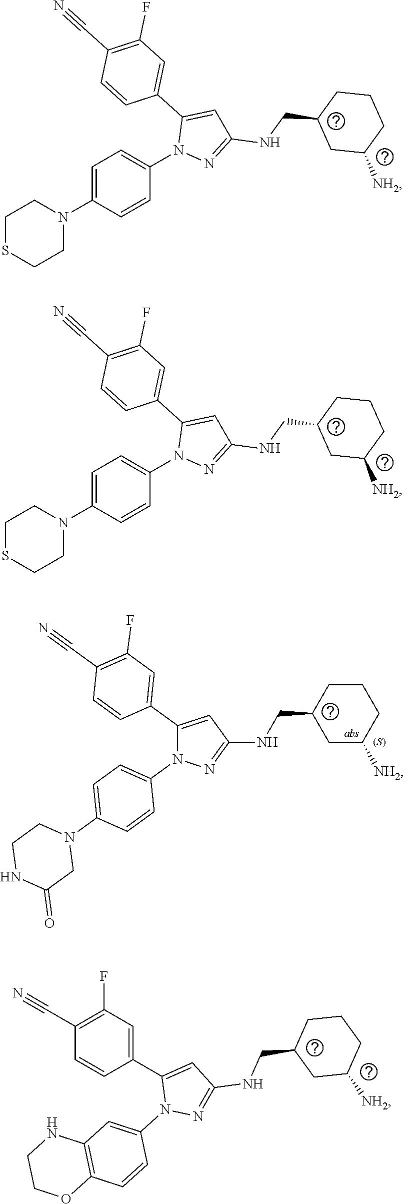

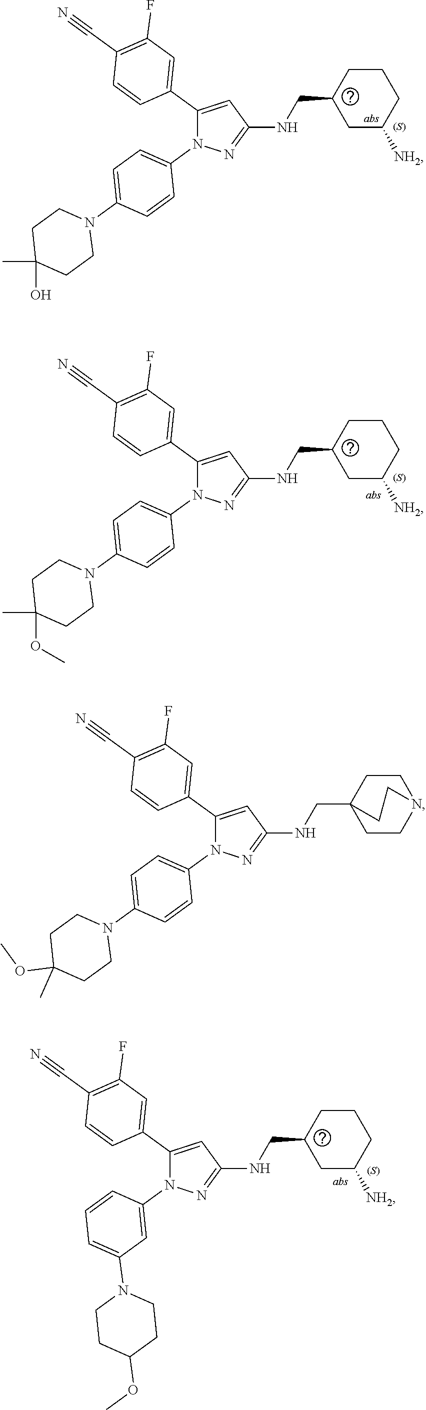

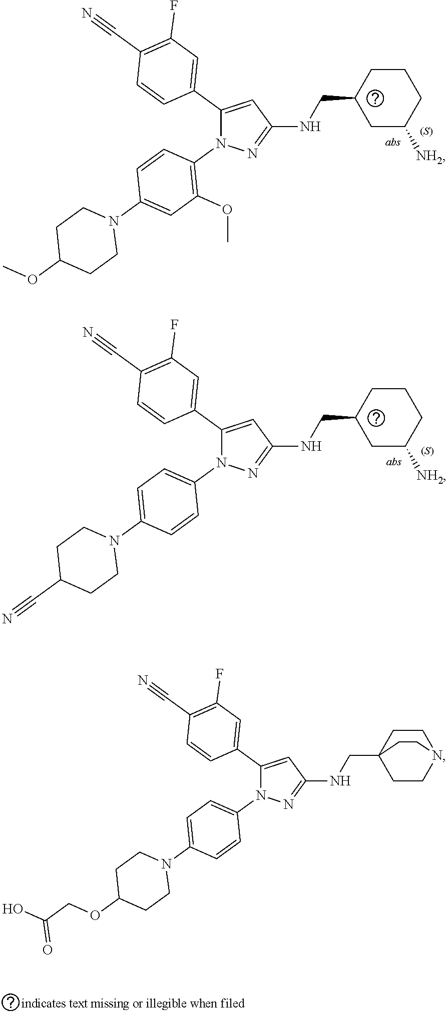

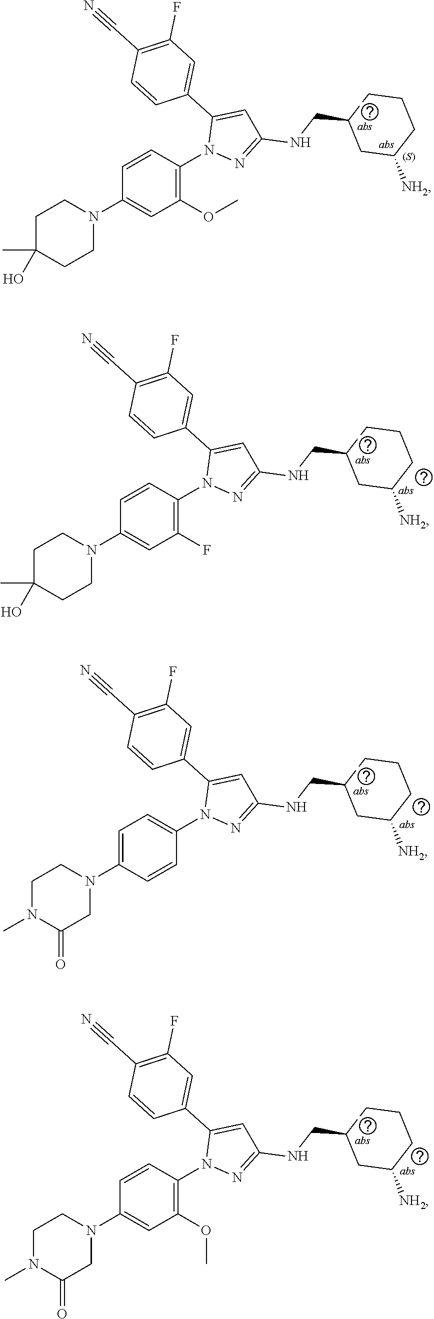

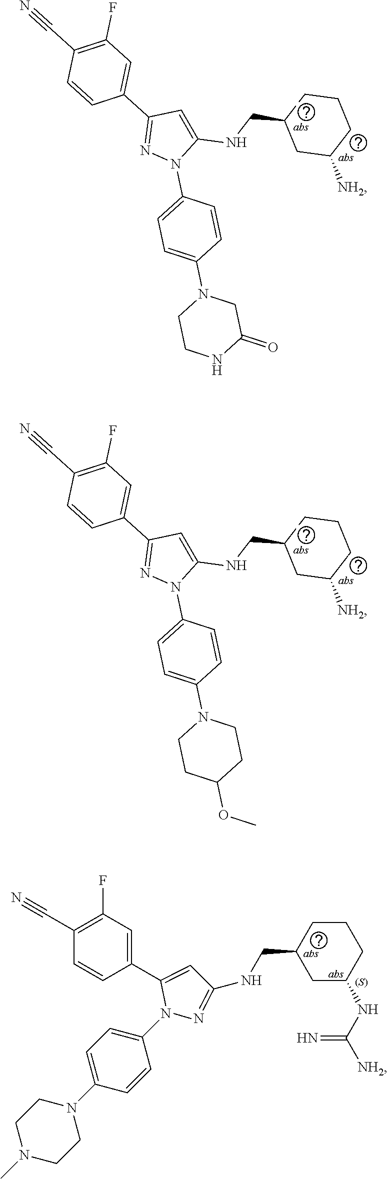

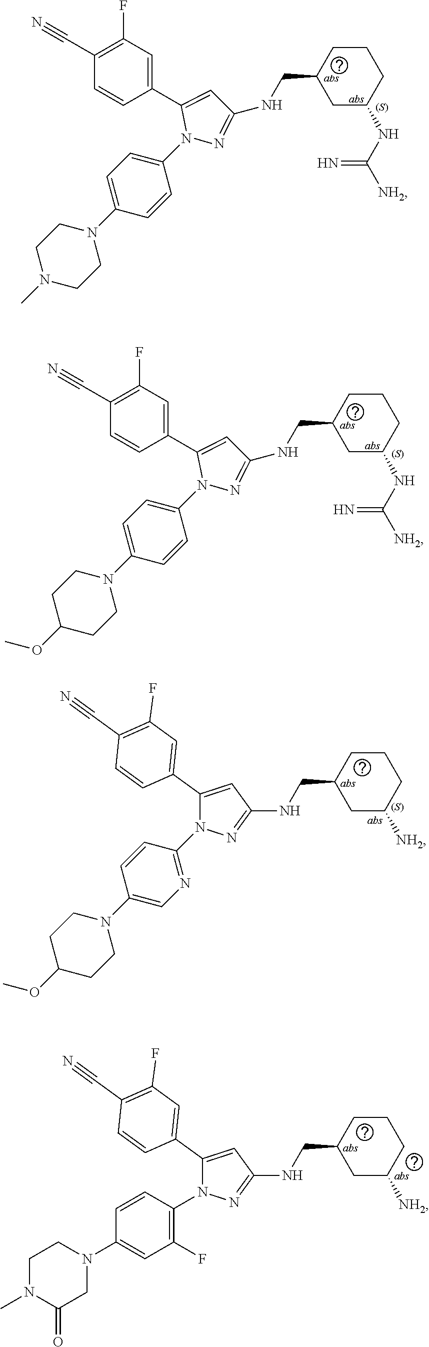

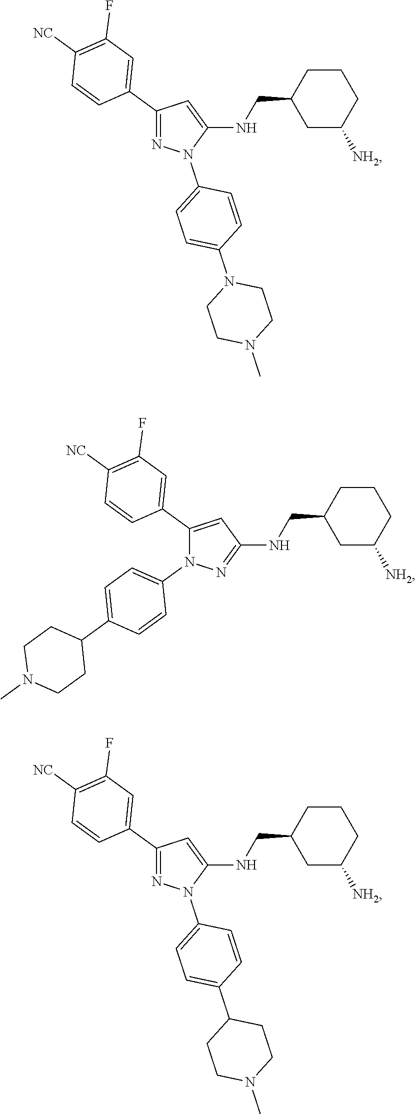 [SMILES: CN1CCN(c2ccc(-n3nc(-c4ccc(C#N)c(F)c4)cc3NC[C@H]3CCC[C@H](N)C3)cc2)CC1 ; CN1CCC(c2ccc(-n3nc(NC[C@H]4CCC[C@H](N)C4)cc3-c3ccc(C#N)c(F)c3)cc2)CC1 ; CN1CCC(c2ccc(-n3nc(-c4ccc(C#N)c(F)c4)cc3NC[C@H]3CCC[C@H](N)C3)cc2)CC1]

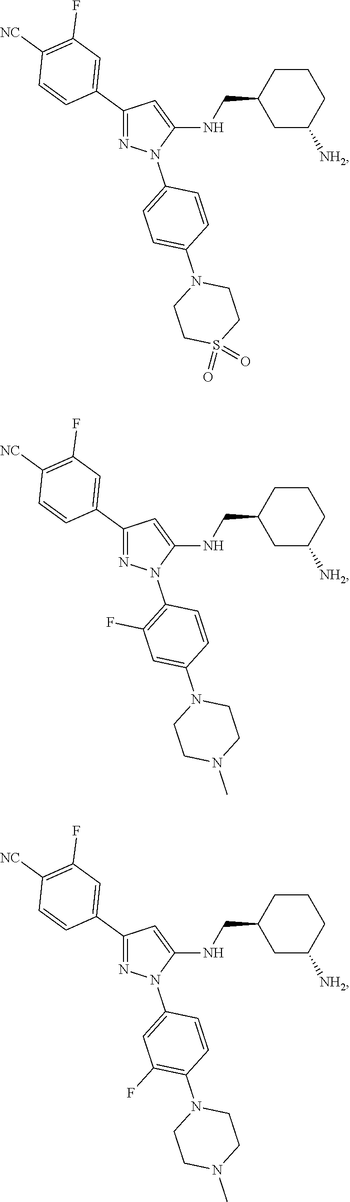 [SMILES: N#Cc1ccc(-c2cc(NC[C@H]3CCC[C@H](N)C3)n(-c3ccc(N4CCS(=O)(=O)CC4)cc3)n2)cc1F ; CN1CCN(c2ccc(-n3nc(-c4ccc(C#N)c(F)c4)cc3NC[C@H]3CCC[C@H](N)C3)c(F)c2)CC1 ; CN1CCN(c2ccc(-n3nc(-c4ccc(C#N)c(F)c4)cc3NC[C@H]3CCC[C@H](N)C3)cc2F)CC1]

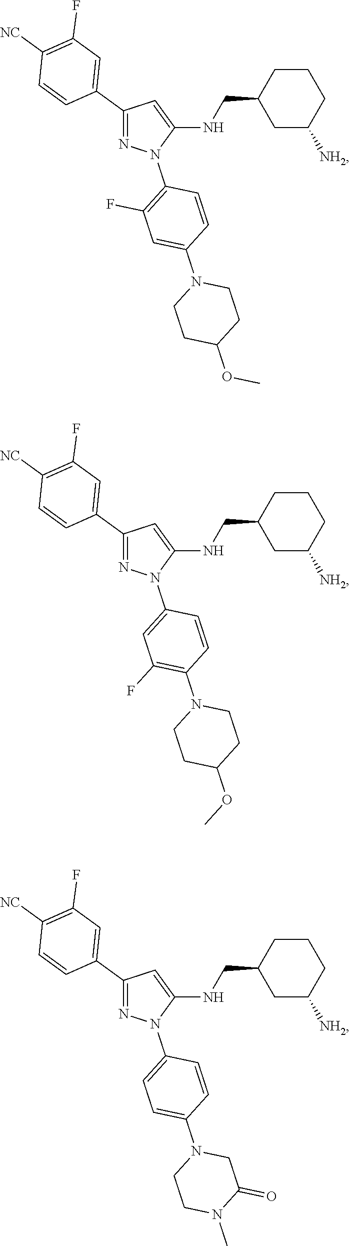 [SMILES: COC1CCN(c2ccc(-n3nc(-c4ccc(C#N)c(F)c4)cc3NC[C@H]3CCC[C@H](N)C3)c(F)c2)CC1 ; COC1CCN(c2ccc(-n3nc(-c4ccc(C#N)c(F)c4)cc3NC[C@H]3CCC[C@H](N)C3)cc2F)CC1 ; CN1CCN(c2ccc(-n3nc(-c4ccc(C#N)c(F)c4)cc3NC[C@H]3CCC[C@H](N)C3)cc2)CC1=O]

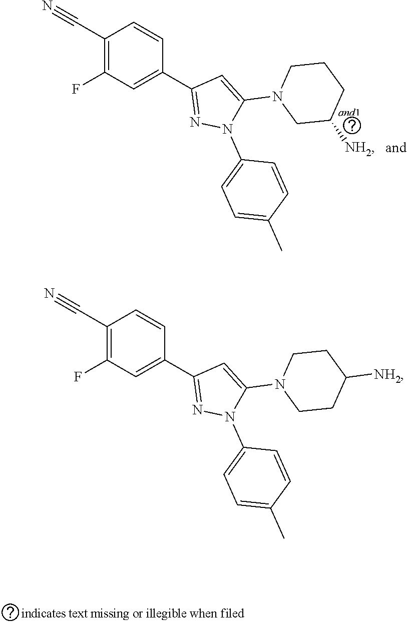 [SMILES: Cc1ccc(-n2nc(-c3ccc(C#N)c(F)c3)cc2N2CCC[C@H](N)C2)cc1 ; "and" ; Cc1ccc(-n2nc(-c3ccc(C#N)c(F)c3)cc2N2CCC(N)CC2)cc1]

or a salt or tautomer thereof.

Also provided herein is a compound, or a salt or tautomer thereof, as disclosed herein is provided for use as a medicament. Also provided herein is a compound as disclosed herein, or a salt or tautomer thereof, for use in the manufacture of a medicament for the prevention or treatment of a KDM1A-mediated disease.

Also provided herein is a compound as disclosed herein is for use in the manufacture of a medicament for the prevention or treatment of a disease or condition chosen from sickle cell disease, thalassemia major, and other beta-hemoglobinopathies.

Also provided herein is a pharmaceutical composition is provided which comprises a compound as disclosed herein, together with a pharmaceutically acceptable carrier.

In certain embodiments, the pharmaceutical composition is formulated for oral administration.

In certain embodiments, the pharmaceutical composition additionally comprises another therapeutic agent.

Also provided herein is a method of inhibiting KDM1A, comprising contacting KDM1A with a compound as disclosed herein.

Also provided herein is a method of treatment of a KDM1A-mediated disease comprising the administration of a therapeutically effective amount of a compound as disclosed herein, or a salt or tautomer thereof, to a patient in need thereof.

In certain embodiments, the disease is cancer.

In certain embodiments, the cancer is chosen from Ewing's sarcoma, multiple myeloma, T-cell leukemia, Wilm's tumor, small-cell lung cancer, bladder cancer, prostate cancer, breast cancer, head/neck cancer, colon cancer, and ovarian cancer.

In certain embodiments, the disease is a myeloid disease.

In certain embodiments, the myeloid disease is chosen from chronic neutrophilic leukemia, myelofibrosis, polycythemia vera, essential thrombocythemia, myelodysplastic syndrome (MDS), acute myelogenous leukemia (AML), and chronic myelogenous leukemia (CML).

In certain embodiments, the disease is an inflammatory disease.

In certain embodiments, the inflammatory disease is chosen from inflammatory bowel disease, rheumatoid arthritis, or systemic lupus erythematosus.

Also provided herein is a method of treatment of a globin-mediated disease comprising the administration of a therapeutically effective amount of a compound as disclosed herein, or a salt or tautomer thereof, to a patient in need thereof.

Also provided herein is a method of treatment of a disease mediated by beta-globin or a hemoglobinopathy comprising the administration of a therapeutically effective amount of a compound as disclosed herein, or a salt or tautomer thereof, to a patient in need thereof.

Also provided herein is a method for achieving an effect in a patient comprising the administration of a therapeutically effective amount of a compound as disclosed herein; wherein the effect is chosen from an elevation of red blood cell count, an elevation of the red blood cell count of red cells containing fetal hemoglobin, an elevation in the total concentration of fetal hemoglobin in red cells, an elevation in the total concentration of fetal hemoglobin in reticulocytes, an increase in the transcription of the gamma globin gene in bone marrow-derived red cell precursors, e.g., pro-erythroblasts, a reduction in the number of sickle cell crises a patient experiences over a unit period of time, a halt to or prevention of tissue damage e.g. in the heart, spleen, brain or kidney caused by sickling cells, a reduction in the proportion of red cells that undergo sickling under physiological conditions of relative hypoxia as measured using patient blood in an in vitro assay, an increase in the amount of histone 3 lysine methylation at lysine position 4 (H3K4me1 and H3K4me2), and/or a decrease in the amount of histone 3 methylation at lysine position 9 (H3K9me1 or H3K4me2) near or at the gamma globin promoter as assayed by ChIP using cells derived from a treated patient.

Also provided herein is a method of inhibiting at least one KDM1A function is provided; comprising the step of contacting KDM1A with a compound as disclosed herein; wherein the inhibition is measured by phenotype of red cells or their precursors either cultured or in vivo in humans or mouse or transgenic mice containing the human beta globin locus or portions thereof, the ability of cancer cells to proliferate, the expression of specific genes known to be regulated by KDM1A activity such as gamma globin, a change in the histone methylation states, a change in the methylation state of proteins known to be demethylated by KDM1A such as G9a or SUV39H1, expression of KDM1A-regulated genes, or binding of KDM1A with a natural binding partner such as CoREST, DNMT1 or HDACs.

Further provided is a method for suppressing proliferation of malignant myeloid cells in a subject in need thereof, the method comprising administering a therapeutically effective amount of a KDM1A inhibitor.

Also provided is a method for reducing reticulin and collagen bone marrow fibrosis in a subject in need thereof, the method comprising administering a therapeutically effective amount of a KDM1A inhibitor.

Also provided is a method for reducing plasma levels of one or more inflammatory cytokines in a subject in need thereof, the method comprising administering a therapeutically effective amount of a KDM1A inhibitor.

Also provided is a method for reducing the mass of malignant myeloid cells in a subject in need thereof, the method comprising administering a therapeutically effective amount of a KDM1A inhibitor.

Also provided is a method for reducing abnormal spleen size or volume in a subject in need thereof, the method comprising administering a therapeutically effective amount of a KDM1A inhibitor.

Also provided is a method for reducing the amount of extramedullary hematopoiesis in a subject in need thereof, the method comprising administering a therapeutically effective amount of a KDM1A inhibitor.

Also provided is a method for reducing the constitutional symptoms of myelofibrosis measured by patient-reported surveys in a subject in need thereof, the method comprising administering a therapeutically effective amount of a KDM1A inhibitor.

Also provided is a method for reducing platelet counts in a subject in need thereof, the method comprising administering a therapeutically effective amount of a KDM1A inhibitor.

Also provided is a method for reducing bone marrow cellularity to age-adjusted normocellularity with fewer than 5% blast cells in a subject in need thereof, the method comprising administering a therapeutically effective amount of a KDM1A inhibitor.

Also provided is a method for a) reducing hemoglobin level in a PV patient to <160 g/L, or b) decreasing red cell mass in a PV patient, wherein the decrease is inferred from hemoglobin levels Hb of <160 g/L, either comprising administering a therapeutically effective amount of a KDM1A inhibitor. Also provided is a method for increasing hemoglobin to >100 g/L in a MF patient, comprising administering a therapeutically effective amount of a KDM1A inhibitor. Also provided is a method for increasing hemoglobin to a value >100 g/L and less than the upper limit of age- and sex adjusted normal in a subject in need thereof, the method comprising administering a therapeutically effective amount of a KDM1A inhibitor.

Provided herein are methods for treating or preventing a myeloproliferative neoplasm, the method comprising administering to a subject in need thereof a KDM1A inhibitor compound as disclosed herein. In certain embodiments, the method effects or results in one or more of the following:
suppresses proliferation of malignant myeloid cells in a subject in need thereof;
reduces reticulin and/or collagen bone marrow fibrosis in a subject in need thereof;
reduces plasma levels of one or more inflammatory cytokines in a subject in need thereof;
reduces mutant allele burden in a subject in need thereof;
reduces a pathologically elevated red blood cell mass in a subject in need thereof;
reduces the mass of malignant myeloid cells in a subject in need thereof;
reduces abnormal spleen size or volume in a subject in need thereof;
reduces the amount of extramedullary hematopoiesis in a subject in need thereof;
reduces the constitutional symptoms of myelofibrosis measured by patient-reported surveys in a subject in need thereof;
reduces platelet counts in a subject in need thereof;
reduces elevated an elevated level of bone marrow cells of granulocytic lineage in a subject in a subject in need thereof;
reduces bone marrow cellularity to age-adjusted normocellularity with fewer than 5% blast cells in a subject in need thereof;
reduces hemoglobin level in a PV patient to <160 g/L in a PV patient;
decreases red cell mass in a PV patient, wherein the decrease is inferred from hemoglobin levels Hb of <160 g/L; or
increases hemoglobin to a value >100 g/L and less than the upper limit of age- and sex adjusted normal in a MF patient.

In certain embodiments, the method effects or results in two or more of the foregoing. In certain embodiments, the method effects or results in three or more of the foregoing. In certain embodiments, the method effects or results in two or more of the foregoing other than reduces platelet counts in a subject in need thereof. In certain embodiments, the one, two, three, or more of the foregoing is limited by a recitation below in paragraphs [0294]-[0314].

In certain embodiments, the myeloproliferative neoplasm is selected from the group consisting of polycythemia vera (PV), essential thrombocythemia (ET), myelofibrosis (MF), chronic myelogenous leukemia (CML), chronic neutrophilic leukemia (CNL), and chronic eosinophilic leukemia (CEL). In certain embodiments, the myeloproliferative neoplasm is selected from the group consisting of polycythemia vera (PV), essential thrombocythemia (ET), and myelofibrosis (MF). In certain embodiments, the myeloproliferative neoplasm is myelofibrosis selected from primary myelofibrosis (PMF) and post PV/ET myelofibrosis. In certain embodiments, the myeloproliferative neoplasm is primary myelofibrosis (PMF). In certain embodiments, the myeloproliferative neoplasm is post PV/ET myelofibrosis. In certain embodiments, the myeloproliferative neoplasm is essential thrombocythemia. In certain embodiments, the myeloproliferative neoplasm is polycythemia vera. In certain embodiments, the myeloproliferative neoplasm is chronic myelogenous leukemia. In certain embodiments, the myeloproliferative neoplasm is chronic neutrophilic leukemia. In certain embodiments, the myeloproliferative neoplasm is chronic eosinophilic leukemia. In certain embodiments, the patient is a human.

Provided herein is a method for suppressing proliferation of malignant myeloid cells, in a subject in need thereof, the method comprising administering a therapeutically effective amount of a KDM1A inhibitor. In certain embodiments, the malignant myeloid cells have mutations in one of the genes selected from the group consisting of Janus Kinase 2 (JAK2), myeloproliferative leukemia virus oncogene (MPL) and calreticulin (CALR). In certain embodiments, the method further comprises the step of determining whether said subject has mutations in one of the genes selected from the group consisting of Janus Kinase 2 (JAK2), myeloproliferative leukemia virus oncogene (MPL) and calreticulin (CALR). In certain embodiments, the malignant myeloid cells are malignant stem cells. In certain embodiments, reduction of the malignant myeloid cells is measured by the frequency of the mutant allele burden as measured by PCR or sequencing or other methods known in the art. In certain embodiments, the malignant myeloid cells are reduced by at least 50%. In certain embodiments, the malignant myeloid cells are reduced by 2 or more logs (100× or more).

Provided herein is a method for reducing reticulin and/or collagen bone marrow fibrosis in a subject in need thereof, the method comprising administering a therapeutically effective amount of a KDM1A inhibitor. In certain embodiments, the bone marrow fibrosis is reticulin bone marrow fibrosis. In certain embodiments, the bone marrow fibrosis is collagen bone marrow fibrosis. In certain embodiments, the bone marrow fibrosis is reticulin and collagen bone marrow fibrosis. In certain embodiments, the reticulin and/or collagen bone marrow fibrosis is reduced by at least one grade, e.g., from 3 to 2, or from 2 to 1, or from 1 to 0. In certain embodiments, the reticulin and/or collagen bone marrow fibrosis is reduced by at least two grades.

In certain embodiments, the subject has mutations in one of the genes selected from the group consisting of Janus Kinase 2 (JAK2), myeloproliferative leukemia virus oncogene (MPL) and calreticulin (CALR). In certain embodiments, the KDM1A inhibitor is a KDM1A inhibitor compound as disclosed herein. The mutations may be assessed by methods known in the art.

Provided herein is a method for reducing plasma levels of one or more inflammatory cytokines in a subject in need thereof, the method comprising administering a therapeutically effective amount of a KDM1A inhibitor. In certain embodiments, one or more of the inflammatory cytokines is selected from the group consisting of interferon gamma, interleukin 6, tumor necrosis factor alpha, and interleukin 8, interleukin 12, interleukin 15, interleukin 17 and CXCL5.

In certain embodiments, the measured cytokine or cytokines are reduced to about the following levels, or below:
IL-6 is reduced to below about 9 pg/ml;
IL-8 is reduced to below about 18 pg/mL;
IL-10 is reduced to below about 51 pg/mL;
IL-12 is reduced to below about 182 pg/mL;
IL-15 is reduced to below about 38 pg/mL;
TNF-alpha is reduced to below about 15 pg/mL; and/or
INF-gamma is reduced to below about 23 pg/mL.
   In certain embodiments, two, three, four, five, or more of the inflammatory cytokines are reduced.

Provided herein is a method for reducing the mass of malignant myeloid cells in a subject in need thereof, the method comprising administering a therapeutically effective amount of a KDM1A inhibitor. In certain embodiments, the mass of malignant myeloid cells is measured by flow cytometry immunophenotyping. In certain embodiments, the mass of malignant myeloid cells is measured by the frequency of the mutant allele, a ratio of the number of cells with the causative MPN mutations (MPL, CALR or JAK2) over the total number of cells that contain both the wild-type and mutant alleles.

Provided herein is a method for reducing mutant allele burden in a subject in need thereof, the method comprising a therapeutically effective amount of a KDM1A inhibitor. In certain embodiments, the mutant allele is an allele of one of the genes selected from the group consisting of Janus Kinase 2 (JAK2), myeloproliferative leukemia virus oncogene (MPL) and calreticulin (CALR). In certain embodiments, the KDM1A inhibitor is a KDM1A inhibitor compound as disclosed herein. In certain embodiments, the mutant allele burden is reduced by about 50% of a subject's (or the subject pool's average) mutant allele burden of mutated Janus Kinase 2 (JAK2), myeloproliferative leukemia virus oncogene (MPL) or calreticulin (CALR). In certain embodiments, the reduction in mutant allele burden is measured within patient(s) after treatment and compared to the level prior to treatment to the level after a course of treatment. In certain embodiments, the mutant allele burden is reduced to a level where mutant alleles of Janus Kinase 2 (JAK2), myeloproliferative leukemia virus oncogene (MPL) and calreticulin (CALR) are undetectable. Mutant allele burden may be assessed by methods known in the art, including those disclosed above.

Provided herein is a method for reducing a pathologically elevated red blood cell mass in a subject in need thereof, the method comprising administering a therapeutically effective amount of a KDM1A inhibitor. In certain embodiments, the subject has polycythemia vera. In certain embodiments, the subject has a mutation in Janus Kinase 2 (JAK2). In certain embodiments, the elevated red blood cell mass is inferred by the measure of the hematocrit or blood hemoglobin. In certain embodiments, measured the hematocrit or the hemoglobin should be reduced to the normal range appropriate to gender. For example, in certain embodiments:
blood hemoglobin will be reduced to less than 16.5 g/dL for a male PV patient or to less than 16.0 g/dL for a female PV patient;
hematocrit will be reduced to less than 49% for a male PV patient or to less than 48% for a female PV patient.
   In certain embodiments, the elevated red blood cell mass is measured by isotopic red cell mass measurement. In certain embodiments the increased red cell mass is greater than 25% above mean normal predicted value.

Provided herein is a method for reducing an elevated white blood cell count in a subject in need thereof, the method comprising administering a therapeutically effective amount of a KDM1A inhibitor. In certain embodiments, subject has chronic neutrophilic leukemia.

Also provided herein is a method for reducing an elevated level of bone marrow cells of granulocytic lineage in a subject in need thereof, the method comprising administering a therapeutically effective amount of a KDM1A inhibitor. In certain embodiments, the bone marrow cells of granulocytic lineage are reduced to a value within the normal range. Also provided herein is a method for, in a subject in need thereof, reducing bone marrow cellularity to age-adjusted normocellularity with fewer than 5% blast cells, the method comprising administering a therapeutically effective amount of a KDM1A inhibitor. In certain embodiments, subject has chronic neutrophilic leukemia.

Provided herein is a method for increasing hemoglobin to >100 g/L up to a level less than the upper limit of age- and sex adjusted normal in a subject in need thereof, the method comprising administering a therapeutically effective amount of a KDM1A inhibitor.

Also provided is a method for a) reducing hemoglobin level in a PV patient to <160 g/L, or b) decreasing red cell mass in a PV patient, wherein the decrease is inferred from hemoglobin levels Hb of <160 g/L, either comprising administering a therapeutically effective amount of a KDM1A inhibitor. Also provided is a method for increasing hemoglobin to >100 g/L in a MF patient, comprising administering a therapeutically effective amount of a KDM1A inhibitor. Also provided is a method for increasing hemoglobin to a value >100 g/L and less than the upper limit of age- and sex adjusted normal in a MF patient, the method comprising administering a therapeutically effective amount of a KDM1A inhibitor. In certain embodiments, said subject has a mutation in one of the genes selected from the group consisting of Janus Kinase 2 (JAK2), myeloproliferative leukemia virus oncogene (MPL) and calreticulin (CALR). In certain embodiments, said subject has essential thrombocythemia. In certain embodiments, the transfusion burden of said patient is reduced.

Provided herein is a method for reducing abnormal spleen size or volume in a subject in need thereof, the method comprising administering a therapeutically effective amount of a KDM1A inhibitor. In certain embodiments, said subject has a mutation in one of the genes selected from the group consisting of Janus Kinase 2 (JAK2), myeloproliferative leukemia virus oncogene (MPL) and calreticulin (CALR).

Provided herein is a method for reducing the amount of extramedullary hematopoiesis in a subject in need thereof, the method comprising administering a therapeutically effective amount of a KDM1A inhibitor. In certain embodiments, said subject has a mutation in one of the genes selected from the group consisting of Janus Kinase 2 (JAK2), myeloproliferative leukemia virus oncogene (MPL) and calreticulin (CALR). In certain embodiments, the amount of extramedullary hematopoiesis is measured by splenomegaly. In certain embodiments, splenomegaly in said subject is reduced by at least about 30%, at least about 35%, at least about 40%, or least about 45%. In certain embodiments, splenomegaly in said subject is reduced by at least 35%. In certain embodiments, splenomegaly in is reduced by at least 35% in about 50% of patients.

Provided herein is a method for reducing the constitutional symptoms of myelofibrosis, as measured by patient-reported surveys in a subject in need thereof, the method comprising administering a therapeutically effective amount of a KDM1A inhibitor. In certain embodiments, said constitutional symptoms comprise one or more symptoms selected from the group consisting of fatigue, early satiety, abdominal discomfort, inactivity, problems with concentration, numbness and/or tingling in the hands and feet, night sweats, pruritis, bone pain, fever greater than 100° F., and unintentional weight loss.

In certain embodiments, said patient-reported survey is the Myeloproliferative Neoplasm Symptom Assessment Form (MPN-SAF). The MPN-SAF is a validated clinical assessment form for the most common symptoms of myeloproliferative neoplasms, in which patients self-reports their score, on a scale of 1-10, of various common symptoms, where 1 is the most favorable or the symptom is absent, and 10 is the least favorable or the symptom is the worst imaginable. Either the full or abbreviated forms may be administered to the patient. In the abbreviated version, a “total symptom score” (TSS) may be calculated from the ten most clinically relevant symptoms from the 17-item MPN-SAF: worst fatigue, concentration, early satiety, inactivity, night sweats, itching, bone pain, abdominal discomfort, weight loss, and fever. The MPN-SAF TSS thus has a possible range of 0 to 100. Quality of life scores are defined as “clinically deficient” when they rate as at least 4 of 10; “moderate” if symptoms are rated as ≥4 of 10 or ≤6 of 10; and “severe” if symptoms are rated as ≥7 of 10. For patients who complete at least six of these 10 items on the BFI and MPN-SAF, the MPN TSS is computed as the average of the observed items multiplied by 10 to achieve a 0-to-100 scale.

In certain embodiments, the total symptom score (MPN-SAF: TSS) is reduced by at least 50%.

In certain embodiments, said patient-reported survey is the myelofibrosis Symptom Assessment Form (MF-SAF). In certain embodiments, the MF-SAF total symptom score is reduced by at least 50%.

In certain embodiments, the subject has a mutation in one of the genes selected from the group consisting of Janus Kinase 2 (JAK2), myeloproliferative leukemia virus oncogene (MPL) and calreticulin (CALR).

In certain embodiments, the subject has a myeloproliferative neoplasm.

In certain embodiments, the subject has a myeloproliferative neoplasm selected from polycythemia vera (PV), essential thrombocythemia (ET), and myelofibrosis.

In certain embodiments, the subject has myelofibrosis.

In certain embodiments, the subject has myelofibrosis selected from primary myelofibrosis (PMF) and post PV/ET myelofibrosis.

In certain embodiments, the subject has post PV/ET myelofibrosis (MF).

In certain embodiments, the subject has primary myelofibrosis (PMF).

In certain embodiments, the subject has polycythemia vera.

In certain embodiments, the subject has essential thrombocythemia.

In certain embodiments, the subject has chronic myelogenous leukemia.

In certain embodiments, the subject has chronic neutrophilic leukemia.

In certain embodiments, the subject has chronic eosinophilic leukemia.

In certain embodiments, the subject is a human.

In certain embodiments, the KDM1A inhibitor is a KDM1A inhibitor compound as disclosed herein.

Also provided are embodiments wherein any method embodiment above may be combined with any one or more of these embodiments, provided the combination is not mutually exclusive. As used herein, two embodiments are “mutually exclusive” when one is defined to be something which cannot overlap with the other. For example, an embodiment wherein the disorder to be treated is primary myelofibrosis (PMF) is mutually exclusive with an embodiment wherein the disorder to be treated is post PV/ET myelofibrosis (MF), because these classifications are the product of different diagnoses. However, an embodiment wherein the disorder to be treated is PMF is not mutually exclusive with an embodiment wherein reticulin and/or collagen bone marrow fibrosis is reduced, because reticulin and/or collagen bone marrow fibrosis occur in PMF.

Inhibition of KDM1A (KDM1A) activity alone may be sufficient therapy for the treatment of some diseases; for other such as cancer, combination therapies are often additive or synergistic in their therapeutic effects and may even be necessary to achieve the full clinical benefit desired. There is specific scientific evidence to rationalize the combination of an inhibitor of KDM1A with all-trans retinoic acid (ATRA), arsenic trioxide, inhibitors of DNA methyltransferases such as 5′-azacytidine or 5′-aza 2′-deoxycytidine, inhibitors of NFκB signaling such as sulindac or conventional anti-neoplastic agents such as anthracyclines or nucleoside analogues such as cytosine arabinoside. Likewise, agents that induce leukemia stem cells into the cell cycle (G-CSF, GM-CSF, stem cell factor, thrombopoietin (TPO)) or agents that negate the contributory role cytokines (TPO, CCL3 (MIP-1)) play in remodeling the niche of cancer stem cells may be useful as part of a combination including a KDM1A inhibitor.

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein when referring to a measurable value such as an amount of a compound, dose, time, temperature, and the like, is meant to encompass variations of 20%, 10%, 5%, 1%, 0.5%, or even 0.1% from the specified amount.

A “therapeutically effective amount” of a drug is an amount of drug or its pharmaceutically acceptable salt that eliminates, alleviates, or provides relief of the symptoms of the disease for which it is administered.

A “subject in need thereof” is a human or non-human animal that exhibits one or more symptoms or indicia of a disease.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched-chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, hydroxyalkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aminoalkyl,” as used herein, alone or in combination, refers to an amino group as defined herein linked through an alkyl group to the parent moiety.

The term “aryl”, as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkyl” or “aralkyl,” as used herein, alone or in combination, refers to an aryl group attached to the parent molecular moiety through an alkyl group.

The term “arylalkanoyl” or “aralkanoyl” or “aroyl,” as used herein, alone or in combination, refers to an acyl group derived from an aryl-substituted alkanecarboxylic acid such as benzoyl, naphthoyl, phenylacetyl, 3-phenylpropionyl (hydrocinnamoyl), 4-phenylbutyryl, (2-naphthyl) acetyl, 4-chlorohydrocinnamoyl, and the like.

The term “aryloxy” as used herein, alone or in combination, refers to an aryl group attached to the parent molecular moiety through an oxy.

The terms “benzo” and “benz,” as used herein, alone or in combination, refer to the divalent group C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “guanidine”, as used herein, alone or in combination, refers to —NHC(═NH)NH₂, or the corresponding guanidinium cation.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogen atoms are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms chosen from O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom chosen from O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings.

The term “heteroarylalkyl” as used herein alone or as part of another group refers to alkyl groups as defined above having a heteroaryl substituent.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. The heterocycle groups may be optionally substituted unless specifically prohibited.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The phrase “in the main chain” refers to the longest contiguous or adjacent chain of carbon atoms starting at the point of attachment of a group to the compounds of any one of the formulas disclosed herein. The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroaryl,” as used herein, alone or in combination, means either 1) monocyclic heteroaryl comprising five or six ring members, of which between one and four said members may be heteroatoms chosen from O, S, and N, or 2) bicyclic heteroaryl, wherein each of the fused rings comprises five or six ring members, comprising between them one to four heteroatoms chosen from O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphonate,” as used herein, alone or in combination, refers to a —P(═O)(OR)₂ group, wherein R is chosen from alkyl and aryl. The term “phosphonic acid”, as used herein, alone or in combination, refers to a —P(═O)(OH)₂ group.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. Similarly, when a designation such as “n” which may be chosen from a group or range of integers is designated to be 0, then the group which it designates is either absent, if in a terminal position, or condenses to form a bond, if it falls between two other groups.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

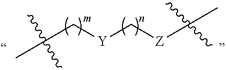

When the construction as used herein, the alkylene groups enclosed by ( )ₘ and ( )ₙ may be m or n carbons long.

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein the term “treat,” “treating,” or “treatment” refers to the administration of therapy to an individual (i.e., a human) who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reaction of the appropriate compound, in the form of the free base, with the appropriate acid.

The compounds disclosed herein can exist as polymorphs and other distinct solid forms such as solvates, hydrates, and the like. A compound may be a polymorph, solvate, or hydrate of a salt or of the free base or acid.

While it may be possible for the compounds disclosed herein to be administered as the raw chemical, it is also possible to present them as pharmaceutical formulations (equivalently, “pharmaceutical compositions”). Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, intraadiposal, intraarterial, intracranial, intralesional, intranasal, intraocular, intrapericardial, intraperitoneal, intrapleural, intraprostatical, intrarectal, intrathecal, intratracheal, intratumoral, intraumbilical, intravaginal, intravesicular, intravitreal, and intramedullary), intraperitoneal, rectal, topical (including, without limitation, dermal, buccal, sublingual, vaginal, rectal, nasal, otic, and ocular), local, mucosal, sublingual, subcutaneous, transmucosal, transdermal, transbuccal, transdermal, and vaginal; liposomal, in cremes, in lipid compositions, via a catheter, via a lavage, via continuous infusion, via infusion, via inhalation, via injection, via local delivery, via localized perfusion, bathing target cells directly, or any combination thereof. Administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as hard or soft capsules, wafers, cachets or tablets each containing a predetermined amount of the active ingredient; as a powder or granules; as a syrup, elixir, solution, or a suspension in an aqueous liquid or a non-aqueous liquid; or as an oil-in-water liquid emulsion, a water-in-oil liquid emulsion, or a compound dispersed in a liposome. The active ingredient may also be presented as a bolus, electuary or paste.

Pharmaceutical preparations that can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide delayed, slowed, or controlled release or absorption of the active ingredient therein. Compositions may further comprise an agent that enhances solubility or dispersability. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Depending on the route of administration, the compounds, or granules or particles thereof, may be coated in a material to protect the compounds from the action of acids and other natural conditions that may inactivate the compounds.

The compounds may be formulated for parenteral administration by injection, e.g., by bolus injection or continuous infusion, either to the body or to the site of a disease or wound. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. To administer the therapeutic compound by other than parenteral administration, it may be necessary to coat the compound with, or co-administer the compound with a material to prevent its inactivation (for example, via liposomal formulation).

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Topical ophthalmic, otic, and nasal formulations disclosed herein may comprise excipients in addition to the active ingredient. Excipients commonly used in such formulations include, but are not limited to, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants. Any of a variety of excipients may be used in formulations disclosed herein including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, guar gum, xanthan gum, carrageenan, agar and acacia, starch derivatives, such as starch acetate and hydroxypropyl starch, and also other synthetic products such as polyvinyl alcohol, polyvinylpyrrolidone, polyvinyl methyl ether, polyethylene oxide, such as cross-linked polyacrylic acid and mixtures of those products. The concentration of the excipient is, typically, from 1 to 100,000 times the concentration of the active ingredient. In certain embodiments, the excipients to be included in the formulations are typically selected because of their inertness towards the active ingredient component of the formulations.

Relative to ophthalmic, otic, and nasal formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants, RLM 100, POE 20 cetylstearyl ethers such as Procol® CS20 and poloxamers such as Pluronic® F68.

The formulations set forth herein may comprise one or more preservatives. Examples of such preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, amino alcohols such as AMP-95, or sorbic acid. In certain embodiments, the formulation may be self-preserved so that no preservation agent is required.

In certain topical embodiments, formulations are prepared using a buffering system that maintains the formulation at a pH of about 4.5 to a pH of about 8. In further embodiments, the pH is from 7 to 8.

Gels for topical or transdermal administration may comprise, generally, a mixture of volatile solvents, nonvolatile solvents, and water. In certain embodiments, the volatile solvent component of the buffered solvent system may include lower (C1-C6)alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. Several optional ingredients can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, galactomannan polymers (such as guar and derivatives thereof), and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example, a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

The therapeutic compound may also be administered intraspinally or intracerebrally. Dispersions for these types of administrations can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents will be included, for example, sugars, sodium chloride, or polyalcohols such as mannitol and sorbitol, in the composition.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and required other ingredients to be pharmacologically sound. In the case of sterile powders for the preparation of sterile injectable solutions, the methods of preparation are vacuum drying and freeze-drying which yields a powder of the active ingredient (i.e., the therapeutic compound) plus any additional desired ingredient from a previously sterile-filtered solution thereof.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

It should be understood that in addition to the ingredients particularly mentioned above, the formulations described above may include other agents conventional in the art having regard to the type of formulation in question, for example, those suitable for oral administration may include flavoring agents.

Compounds may be administered at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

Preferred unit dosage formulations are those containing an effective dose, as herein below recited, or an appropriate fraction thereof, of the active ingredient. In certain embodiments, a formulation disclosed herein is administered once a day. However, the formulations may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. The formulations are administered at varying dosages, but typical dosages are one to two drops at each administration, or a comparable amount of a gel or other formulation. One of ordinary skill in the art would be familiar with determining a therapeutic regimen for a specific indication.

The amount of active ingredient that may be combined with the carrier materials to produce a single dosage form will vary depending upon the host treated and the particular mode of administration. Similarly, the precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Alternatively, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). There is even the possibility that two compounds, one of the compounds described herein and a second compound may together achieve the desired therapeutic effect that neither alone could achieve. Alternatively, by way of example only, the benefit experienced by a patient may be increased by administering one of the compounds described herein with another therapeutic agent (which also includes a therapeutic regimen) that also has therapeutic benefit. By way of example only, in a treatment for acute myelogenous leukemia or sickle cell anemia involving administration of one of the compounds described herein, increased therapeutic benefit may result by also providing the patient with another therapeutic agent for sickle cell anemia or for acute myelogenous leukemia. In any case, regardless of the disease, disorder or condition being treated, the overall benefit experienced by the patient may simply be additive of the two therapeutic agents or the two agents may have synergistic therapeutic effects in a patient.

Effective combination therapy may be achieved with a single composition or pharmacological formulation that includes both agents, or with two distinct compositions or formulations, at the same time, wherein one composition includes a compound of the present disclosure, and the other includes the second agent(s). Alternatively, the therapy may precede or follow the other agent treatment by intervals ranging from minutes to months. Administration of the compounds of the present disclosure to a patient will follow general protocols for the administration of pharmaceuticals, taking into account the toxicity, if any, of the drug. It is expected that the treatment cycles would be repeated as necessary.

Specific, non-limiting examples of possible combination therapies include use of certain compounds of the disclosure with the following agents and classes of agents: agents that inhibit DNA methyltransferases such as decitabine or 5′-aza-cytadine; agents that inhibit the activity of histone deacetylases, histone de-sumoylases, histone de-ubiquitinases, or histone phosphatases such as hydroxyurea; antisense RNAs that might inhibit the expression of other components of the protein complex bound at the DR site in the gamma globin promoter; agents that inhibit the action of Klf1 or the expression of KLF1; agents that inhibit the action of Bcl11a or the expression of BCL11A; and agents that inhibit cell cycle progression such as hydroxyurea, ara-C or daunorubicin; agents that induce differentiation in leukemic cells such as all-trans retinoic acid (ATRA).

Thus, in another aspect, the present disclosure provides methods for treating diseases or disorders in a human or animal subject in need of such treatment comprising administering to said subject an amount of a compound disclosed herein effective to reduce or prevent said disorder in the subject, optionally in combination with at least one additional agent for the treatment of said disorder that is known in the art.

Used either as a monotherapy or in combination with other agents, the compounds disclosed herein are useful in the prevention and/or treatment of beta-hemoglobinopathies such as thalassemia major, sickle cell disease, hemoglobin E/thalassemia, and thalassemia intermedia.

The compounds disclosed herein can be used in the treatment of diseases in which an increase in transcription through the manipulation of epigenetic regulatory factors such as inhibition of KDM1A would be beneficial to the patient. This applies to diseases including but not limited to loss of function mutations, mutations resulting in haploinsufficiency, deletions and duplications of genetic material or epigenetic regulatory mechanisms have altered the normal expression pattern of a gene or genes that has the effect of altering the dose of a gene product(s). Such diseases may include diseases both acquired and hereditary in which the expression of, for example, cytokines affecting immune function, are altered, X-linked mental retardation and other forms of compromised cognitive or motor function such as Alzheimer and Parkinson disease whether they are the acquired or hereditary forms, lipid disorders such as elevated cholesterol, low density lipoprotein, very low density lipoprotein or triglycerides, both type one and type two diabetes, and Mendelian genetic diseases.

Other disorders or conditions that can be advantageously treated by the compounds disclosed herein include inflammation and inflammatory conditions. Inflammatory conditions include, without limitation: arthritis, including sub-types and related conditions such as rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis; osteoporosis, tendonitis, bursitis, and other related bone and joint disorders; gastrointestinal conditions such as reflux esophagitis, diarrhea, inflammatory bowel disease, Crohn's disease, gastritis, irritable bowel syndrome, ulcerative colitis, acute and chronic inflammation of the pancreas; pulmonary inflammation, such as that associated with viral infections and cystic fibrosis; skin-related conditions such as psoriasis, eczema, burns, sunburn, dermatitis (such as contact dermatitis, atopic dermatitis, and allergic dermatitis), and hives; pancreatitis, hepatitis, pruritus and vitiligo. In addition, compounds of disclosure are also useful in organ transplant patients either alone or in combination with conventional immunomodulators.

Autoimmune disorders may be ameliorated by the treatment with compounds disclosed herein. Autoimmune disorders include Crohn's disease, ulcerative colitis, dermatitis, dermatomyositis, diabetes mellitus type 1, Goodpasture's syndrome, Graves' disease, Guillain-Barré syndrome (GBS), autoimmune encephalomyelitis, Hashimoto's disease, idiopathic thrombocytopenia purpura, lupus erythematosus, mixed connective tissue disease, multiple sclerosis (MS), myasthenia gravis, narcolepsy, pemphigus vulgaris, pernicious anemia, psoriasis, psoriatic arthritis, polymyositis, primary biliary cirrhosis, rheumatoid arthritis, Sjögren's syndrome, scleroderma, temporal arteritis (also known as “giant cell arteritis”), vasculitis, and Wegener's granulomatosis.

The compounds disclosed herein are also useful for the treatment of organ and tissue injury associated with severe burns, sepsis, trauma, wounds, and hemorrhage- or resuscitation-induced hypotension, and also in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, swelling occurring after injury, ischemias including myocardial ischemia, cardiovascular ischemia, and ischemia secondary to cardiac arrest, and the like.

The compounds disclosed herein are also useful for the treatment of certain diseases and disorders of the nervous system. Central nervous system disorders in KDM1A inhibition is useful include cortical dementias including Alzheimer's disease, central nervous system damage resulting from stroke, ischemias including cerebral ischemia (both focal ischemia, thrombotic stroke and global ischemia (for example, secondary to cardiac arrest), and trauma. Neurodegenerative disorders in which KDM1A inhibition is useful include nerve degeneration or nerve necrosis in disorders such as hypoxia, hypoglycemia, epilepsy, and in cases of central nervous system (CNS) trauma (such as spinal cord and head injury), hyperbaric oxygen-induced convulsions and toxicity, dementia e.g., pre-senile dementia, and AIDS-related dementia, cachexia, Sydenham's chorea, Huntington's disease, Parkinson's Disease, amyotrophic lateral sclerosis (ALS), Korsakoff's disease, cognitive disorders relating to a cerebral vessel disorder, hypersensitivity, sleeping disorders, schizophrenia, depression, depression or other symptoms associated with Premenstrual Syndrome (PMS), and anxiety.

Still other disorders or conditions advantageously treated by the compounds disclosed herein include the prevention or treatment of hyperproliferative diseases, especially cancers, either alone or in combination with standards of care especially those agents that target tumor growth by re-instating tumor suppressor genes in the malignant cells. Hematological and non-hematological malignancies which may be treated or prevented include but are not limited to multiple myeloma, acute and chronic leukemias and hematopoietic proliferative and neoplastic disorders including myelodysplastic syndrome (MDS), acute myelogenous leukemia (AML), Acute Lymphocytic Leukemia (ALL), Chronic Lymphocytic Leukemia (CLL), and chronic myelogenous leukemia (CML), lymphomas, including Hodgkin's lymphoma and non-Hodgkin's lymphoma (low, intermediate, and high grade), as well as solid tumors and malignancies of the brain, head and neck, breast, lung (including non-small-cell lung cancer), reproductive tract, upper digestive tract, pancreas, liver, renal system, bladder, prostate and colorectal. The present compounds and methods can also be used to treat fibrosis, such as that which occurs with radiation therapy. The present compounds and methods can be used to treat subjects having or prevent the progression of adenomatous polyps, including those with familial adenomatous polyposis (FAP) or sarcoidosis. Non-cancerous proliferative disorders additionally include psoriasis, eczema, and dermatitis.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds disclosed herein may also be used to prevent tissue damage when therapeutically combined with antibacterial or antiviral agents.

The compounds disclosed herein are also useful for the treatment of treat metabolic disorders. KDM1A, using flavin adenosine dinucleotide (FAD) as a cofactor, epigenetically regulates energy-expenditure genes in adipocytes depending on the cellular FAD availability. Additionally, loss of KDM1A function induces a number of regulators of energy expenditure and mitochondrial metabolism resulting in the activation of mitochondrial respiration. Furthermore, in the adipose tissues from mice fed a high-fat diet, expression of KDM1A-target genes is reduced.

Metabolic syndrome (also known as metabolic syndrome X) is characterized by having at least three of the following symptoms: insulin resistance; abdominal fat—in men this is defined as a 40 inch waist or larger, in women 35 inches or larger; high blood sugar levels—at least 110 milligrams per deciliter (mg/dL) after fasting; high triglycerides—at least 150 mg/dL in the blood stream; low HDL—less than 40 mg/dL; pro-thrombotic state (e.g., high fibrinogen or plasminogen activator inhibitor in the blood); or blood pressure of 130/85 mmHg or higher. A connection has been found between metabolic syndrome and other conditions such as obesity, high blood pressure and high levels of LDL cholesterol, all of which are risk factors for cardiovascular diseases. For example, an increased link between metabolic syndrome and atherosclerosis has been shown. People with metabolic syndrome are also more prone to developing type 2 diabetes, as well as PCOS (polycystic ovarian syndrome) in women and prostate cancer in men.

As described above, insulin resistance can be manifested in several ways, including type 2 diabetes. Type 2 diabetes is the condition most obviously linked to insulin resistance. Compensatory hyperinsulinemia helps maintain normal glucose levels often for decades before overt diabetes develops. Eventually the beta cells of the pancreas are unable to overcome insulin resistance through hypersecretion. Glucose levels rise and a diagnosis of diabetes can be made. Patients with type 2 diabetes remain hyperinsulinemic until they are in an advanced stage of disease. As described above, insulin resistance can also correlate with hypertension. One half of patients with essential hypertension are insulin resistant and hyperinsulinemic, and there is evidence that blood pressure is linked to the degree of insulin resistance. Hyperlipidemia, too, is associated with insulin resistance. The lipid profile of patients with type 2 diabetes includes increased serum very-low-density lipoprotein (VLDL) cholesterol and triglyceride levels and, sometimes, a decreased low-density lipoprotein (LDL) cholesterol level. Insulin resistance has been found in persons with low levels of high-density lipoprotein HDL). Insulin levels have also been linked to VLDL synthesis and plasma triglyceride levels.

Specific metabolic diseases and symptoms to be treated by the compounds, compositions, and methods disclosed herein are those mediated at least in part by KDM1A. Accordingly, disclosed herein are methods: for treating insulin resistance in a subject; for reducing glycogen accumulation in a subject; for raising HDL or HDLc, lowering LDL or LDLc, shifting LDL particle size from small dense to normal LDL, lowering VLDL, lowering triglycerides, or inhibiting cholesterol absorption in a subject; for reducing insulin resistance, enhancing glucose utilization or lowering blood pressure in a subject; for reducing visceral fat in a subject; for reducing serum transaminases in a subject; for inducing mitochondrial respiration in a subject; or for treating disease; all comprising the administration of a therapeutic amount of a compound as described herein, to a patient in need thereof. In further embodiments, the disease to be treated may be a metabolic disease. In further embodiment, the metabolic disease may be selected from the group consisting of: obesity, diabetes mellitus, especially Type 2 diabetes, hyperinsulinemia, glucose intolerance, metabolic syndrome X, dyslipidemia, hypertriglyceridemia, hypercholesterolemia, and hepatic steatosis. In other embodiments, the disease to be treated may be selected from the group consisting of: cardiovascular diseases including vascular disease, atherosclerosis, coronary heart disease, cerebrovascular disease, heart failure and peripheral vessel disease. In some embodiments, the methods above do not result in the induction or maintenance of a hypoglycemic state.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### General Synthetic Methods for Preparing Compounds

The following schemes can be used to prepare the compounds disclosed herein.

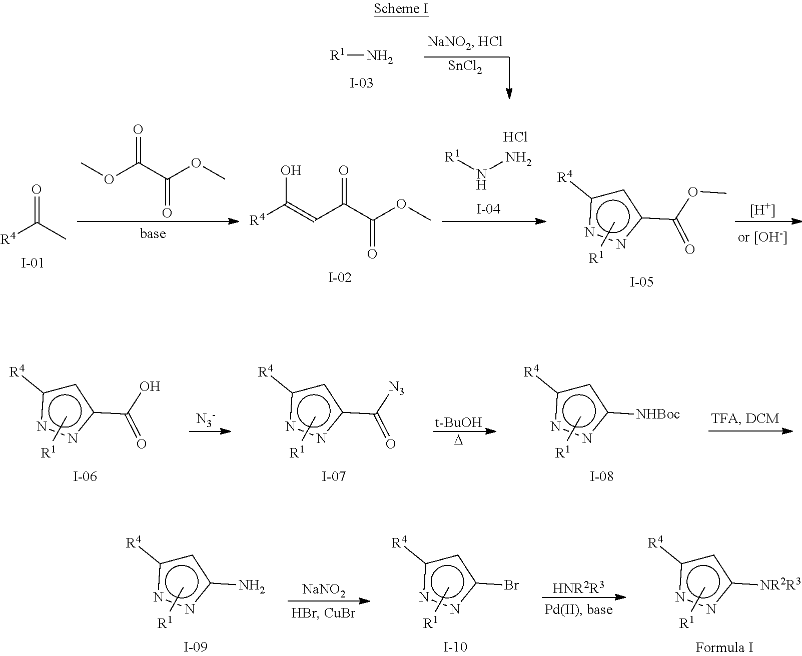

Trisubstituted pyrazoles of Formula I can be prepared as set forth in Scheme I. A Knorr pyrazole synthesis followed by ester hydrolysis affords the key carboxylic acid intermediate I-06 containing the R¹ and R⁴ groups. This compound is transformed to an acyl azide, then a Curtius rearrangement performed in t-butanol followed by a Boc deprotection affords the aminopyrazole intermediate I-09. The amine is replaced with a bromine and subsequently cross-coupled with an amine containing the R² and R³ groups to give a compound of Formula I.

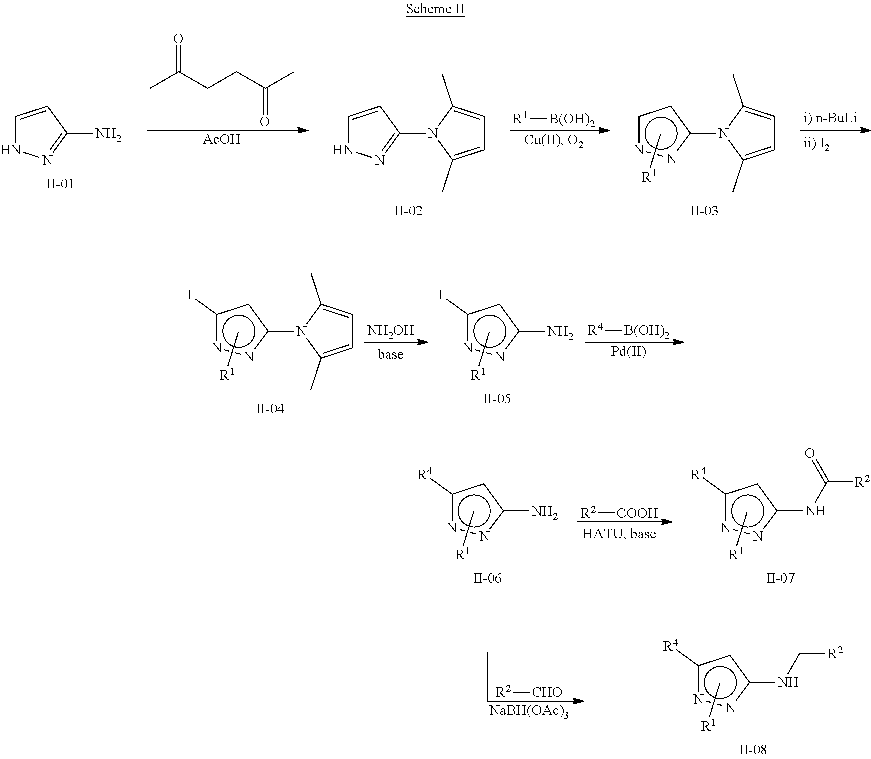

Trisubstituted pyrazoles of general Formulas II-07 and II-08 can be prepared as set forth in Scheme II. Introduction of R¹ and R⁴ groups into 2-aminopyrazole affords key intermediate II-06. This compound can be coupled with a carboxylic acid to give amide II-07, or alternatively reacted with an aldehyde under reductive amination conditions to give substituted amine II-08.

The following disclosure is further illustrated by the following Examples.

In the Examples below and throughout the disclosure, the following abbreviations may be used: RT=Room Temperature; SM=Starting Material; MeCN or ACN=acetonitrile; acac=acetylacetonate anion; AcOH=acetic acid; B₂Pin₂=bis(pinacolato)diboron=4,4,4′,4′,5,5,5′,5′-octamethyl-2,2′-bi-1,3,2-dioxaborolane; BINAP= (2,2′-bis(diphenylphosphino)-1,1′-binaphthyl); DCE=dichloroethane; DCM=dichloromethane; DIEA or DIPEA=N,N-Diisopropylethylamine; DMA=dimethyl acetamide; DMAP=4-dimethylaminopyridine; DMF=dimethyl formamide; DMSO=dimethylsulfoxide; DPPA=diphenylphosphoryl azide; Et₃N or TEA=triethylamine; Et₂O=diethyl ether; EtOAc=ethyl acetate; EtOH=ethanol; H₂O=water; HATU=(1-[Bis(dimethylamino)methylene]-1H-1,2,3-triazolo[4,5-b]pyridinium 3-oxide hexafluorophosphate; IPA=propan-2-ol; MeOH=methanol; NCS=N-Chlorosuccinimide; PE=petroleum ether; t-BuOH=t-butanol; TFA=tri-fluoroacetic acid; THF=tetrahydrofuran; ¹H-NMR=Proton Nuclear magnetic Resonance; TLC=thin layer chromatography; and HPLC=High Performance Liquid Chromatography. Other abbreviations may be used and will be familiar in context to those of skill in the art.

### Intermediate X-1

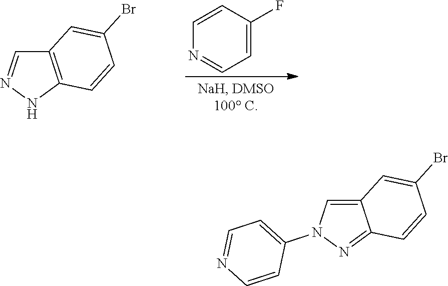 [SMILES: Brc1ccc2[nH]ncc2c1 ; Fc1ccncc1 ; Brc1ccc2nn(-c3ccncc3)cc2c1]

5-bromo-2-(pyridin-4-yl)-2H-indazole To a solution 5-bromo-1H-indazole (11.2 g, 56.6 mmol, 1.10 equiv) and 4-fluoropyridine hydrochloride (5.0 g, 51.5 mmol, 1.00 equiv) in DMSO (200 mL) was added NaH (60% wt., 6.18 g, 154 mmol, 3.00 equiv) in portions at 0° C. The resulting mixture was stirred for 4 h at 100° C. The reaction was quenched with H₂O (500 mL) at 0° C. and extracted with EtOAc (3×500 mL). The combined organic layers were washed with brine (3×700 mL), dried over anhydrous Na₂SO₄ and concentrated under vacuum. The residue was purified with silica gel chromatography using PE/EtOAc (1:1) to afford the title compound (1.5 g, 10.6%) as a white solid. MS-ESI: 274/276 (M+1).

### Intermediate X-2

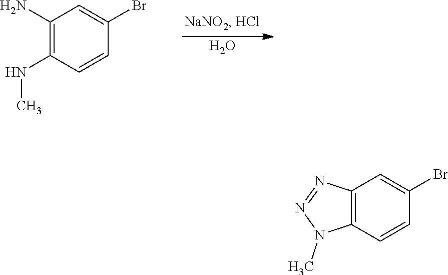

5-bromo-1-methyl-1H-benzo[d][1,2,3]triazole To a solution of 4-bromo-N¹-methylbenzene-1,2-diamine (9.9 g, 49.2 mmol, 1.00 equiv) in HCl (150 mL) was added NaNO₂ (5.1 g, 73.8 mmol, 1.50 equiv) in H₂O (9.0 mL) dropwise at 0° C. The resulting mixture was stirred for 2 h at 0° C. The mixture was adjusted to pH 7-8 with sat. Na₂CO₃ aq. and extracted with EtOAc (3×500 mL). The combined organic layers were concentrated under reduced pressure to afford the title compound (9.9 g, 82%) as a yellow solid. MS-ESI: 212/214 (M+1).

### Intermediate X-3

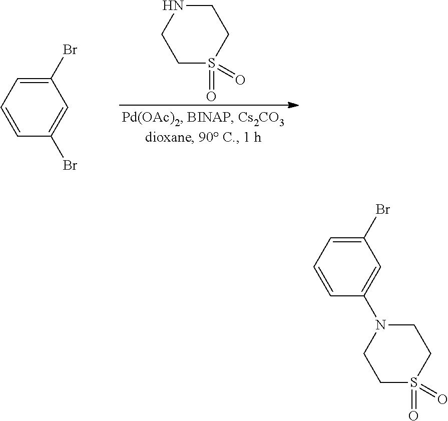 [SMILES: Brc1cccc(Br)c1 ; O=S1(=O)CCNCC1 ; O=S1(=O)CCN(c2cccc(Br)c2)CC1]

4-(3-bromophenyl)thiomorpholine 1,1-dioxide To a solution of 1,3-dibromobenzene (1.00 g, 4.24 mmol, 1.00 equiv) and thiomorpholine 1,1-dioxide (572 mg, 4.24 mmol, 1.00 equiv) in dioxane (20 mL) were added Pd(OAc)₂ (94 mg, 0.42 mmol, 0.1 equiv), BINAP (364 mg, 0.42 mmol, 0.1 equiv) and Cs₂CO₃ (2.76 g, 8.48 mmol, 2 equiv). The resulting mixture was stirred overnight at 90° C. under nitrogen. The mixture was concentrated under vacuum. The residue was purified with silica gel chromatography using PE/EtOAc (1:1) to afford the title compound (300 mg, 24%) as a white solid. MS-ESI: 290/292 (M+1).

### Intermediate X-4

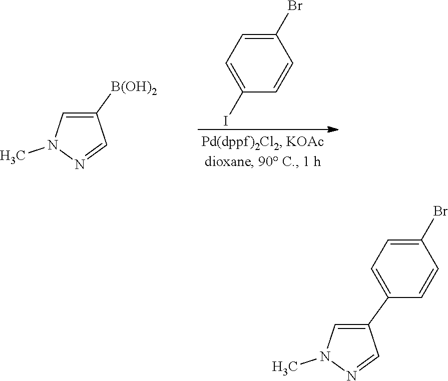

4-(4-bromophenyl)-1-methyl-1H-pyrazole To a solution of 1-bromo-4-iodobenzene (10 g, 35 mmol, 1.00 equiv) in dioxane (30 mL) and H₂O (3 mL) was added (1-methyl-1H-pyrazol-4-yl) boronic acid (2.23 g, 17.7 mmol, 0.50 equiv), Pd(dppf)Cl₂ (2.6 g, 3.54 mmol, 0.1 equiv) and K₂CO₃ (14.7 g, 106 mmol, 3.00 equiv). The resulting mixture was stirred for 2 h at 90° C. under nitrogen. The mixture was diluted with H₂O (50 mL) and extracted with EtOAc (3×50 mL). The combined organic layers were dried with anhydrous Na₂SO₄ and concentrated under vacuum. The residue was purified with silica gel chromatography using EtOAc/PE (2:1) to afford the title compound (2.2 g, 91%) as a brown solid. MS-ESI: 237/239 (M+1).

### Intermediate X-5

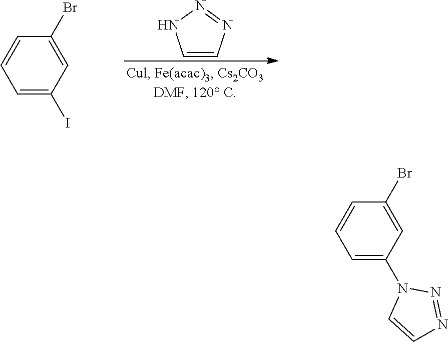 [SMILES: Brc1cccc(I)c1 ; c1c[nH]nn1 ; Brc1cccc(-n2ccnn2)c1]

1-(3-bromophenyl)-1H-1,2,3-triazole To a solution of 1-bromo-3-iodobenzene (10 g, 35 mmol, 1.00 equiv) and 1H-1,2,3-triazole (2.44 g, 35.3 mmol, 1.00 equiv) in DMF (200 mL) was added CuI (0.67 g, 3.54 mmol, 0.10 equiv), Fe(acac)₃ (3.75 g, 10.6 mmol, 0.30 equiv) and Cs₂CO₃ (23 g, 70.7 mmol, 2.00 equiv). The resulting mixture was stirred for 1 h at 120° C. under N₂. The mixture was diluted with H₂O (1000 mL) and extracted with EtOAc (3×500 mL). The combined organic layers were concentrated under vacuum. The residue was purified with silica gel chromatography using PE/EtOAc (1:1) to afford the title compound (2.0 g, 23.7%) as a white solid. MS-ESI: 224/226 (M+1).

### Intermediate X-6

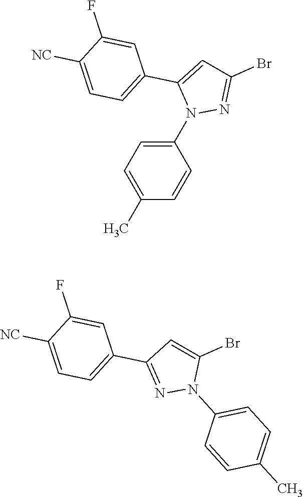 [SMILES: Cc1ccc(-n2nc(Br)cc2-c2ccc(C#N)c(F)c2)cc1 ; Cc1ccc(-n2nc(-c3ccc(C#N)c(F)c3)cc2Br)cc1]

### 4-(3-Bromo-1-(p-tolyl)-1H-pyrazol-5-yl)-2-fluorobenzonitrile and 4-(5-bromo-1-(p-tolyl)-1H-pyrazol-3-yl)-2-fluorobenzonitrile

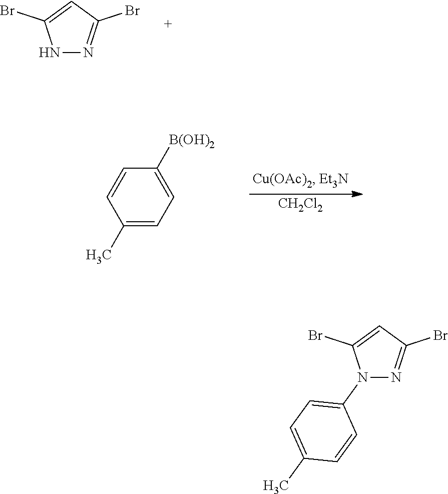

3,5-Dibromo-1-(p-tolyl)-1H-pyrazole A solution of 3,5-dibromo-1H-pyrazole (5.0 g, 22.1 mmol, 1.00 equiv), p-tolueneboronic acid (4.51 g, 33.2 mmol, 1.50 equiv), Cu(OAc)₂ (6.03 g, 33.2 mmol, 1.50 equiv), and Et₃N (6.72 g, 66.4 mmol, 3.00 equiv) in CH₂Cl₂ (200 mL) was stirred 16 h at rt, then concentrated under vacuum and purified with silica gel column chromatography, eluting with EtOAc/hexane (1:6) to afford 2 g (32%) of the title compound as an off-white solid.

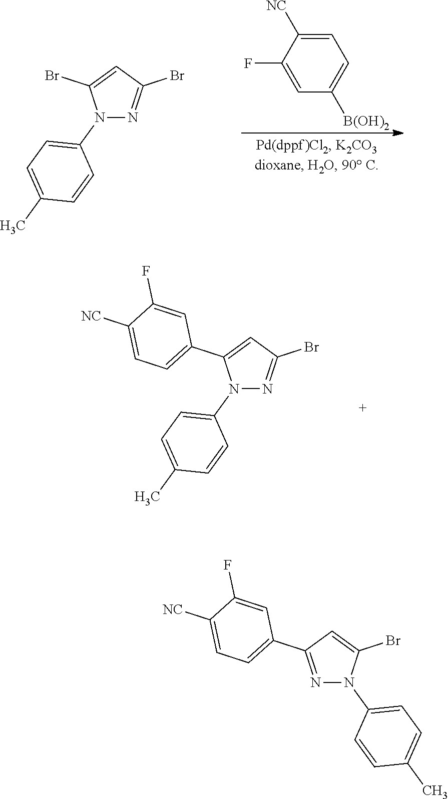 [SMILES: Cc1ccc(-n2nc(Br)cc2Br)cc1 ; N#Cc1ccc(B(O)O)cc1F ; Cc1ccc(-n2nc(Br)cc2-c2ccc(C#N)c(F)c2)cc1 ; Cc1ccc(-n2nc(-c3ccc(C#N)c(F)c3)cc2Br)cc1]

A solution of the product from the previous step (4.00 g, 12.658 mmol, 1.00 equiv), 4-cyano-3-fluorophenylboronic acid (1.04 g, 6.34 mmol, 0.50 equiv), Pd(dppf)Cl₂ (0.93 g, 1.266 mmol, 0.10 equiv) and K₂CO₃ (5.25 g, 37.9 mmol, 3.00 equiv) in dioxane (100 mL) and H₂O (10 mL) was stirred 2 h under N₂ at 90° C., then concentrated under vacuum and purified with silica gel column chromatography, eluting with EtOAc/petroleum ether (1:5), to afford 2 g (44.4%) of the title isomeric mixture as a yellow solid.

### Intermediate A-1

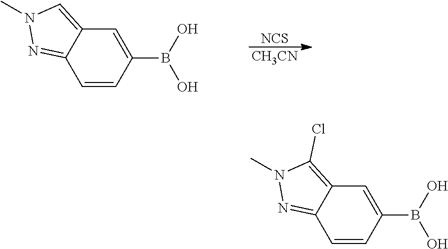

(3-Chloro-2-methyl-2H-indazol-5-yl) boronic acid To a solution of (2-methyl-2H-indazol-5-yl) boronic acid (7.0 g, 39.8 mmol, 1.00 equiv) in CH₃CN (200 mL) was added NCS (4.25 g, 31.8 mmol, 0.80 equiv). The mixture was stirred for 2 h at 70° C. The resulting mixture was concentrated under vacuum and purified with silica gel colum chromatography using with CH₂Cl₂/MeOH (50:1) to afford the title compound (5.3 g, 59.7%) as a white solid. MS-ESI: 211 (M+1).

### Intermediate A-2

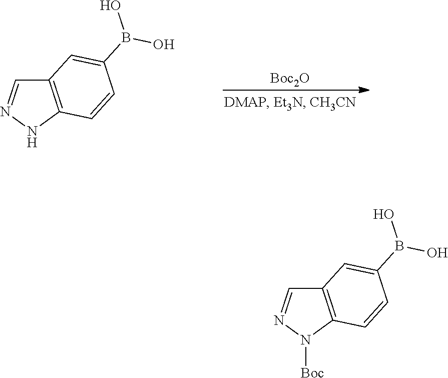 [SMILES: "Boc2O" ; OB(O)c1ccc2[nH]ncc2c1 ; CC(C)(C)OC(=O)n1ncc2cc(B(O)O)ccc21]

(1-(tert-Butoxycarbonyl)-1H-indazol-5-yl) boronic acid To a solution of (1H-indazol-5-yl) boronic acid (10 g, 61.7 mmol, 1.00 equiv) and Boc₂O (14.1 g, 64.8 mmol, 1.05 equiv) in CH₃CN (100 mL) was added DMAP (377 mg, 3.09 mmol, 0.05 equiv) and Et₃N (18.7 g, 185 mmol, 3.00 equiv). The resulting mixture was stirred overnight at room temperature, then concentrated under vacuum. The resulting solid was washed with EtOAc (10 mL) to afford the title compound (13 g, 80%) as a yellow solid. MS-ESI: 262 (M−1).

### Intermediate A-3

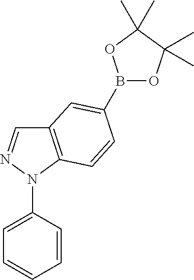 [SMILES: CC1(C)OB(c2ccc3c(cnn3-c3ccccc3)c2)OC1(C)C]

### 1-phenyl-5-(4,4,5,5-tetramethyl-1,3,2-dioxaborolan-2-yl)-1H-indazole

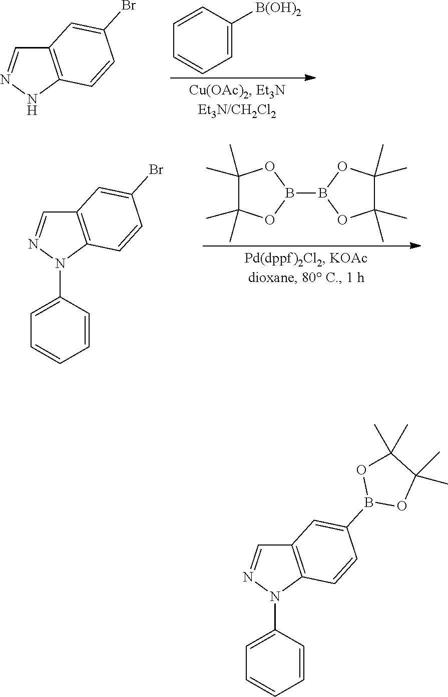

5-bromo-1-phenyl-1H-indazole To a solution of 5-bromo-1H-indazole (20 g, 101 mmol, 1.00 equiv) and phenylboronic acid (16.1 g, 132 mmol, 1.30 equiv) in CH₂Cl₂ (200 mL) was added Cu(OAc)₂ (276 mg, 1.52 mmol, 1.50 equiv) and Et₃N (30.8 g, 305 mmol, 3.00 equiv). The resulting mixture was stirred for 3 days at room temperature under O₂. The resulting mixture was concentrated under vacuum. The residue was purified with silica gel chromatography using PE/EtOAc (10:1) to afford the title compound (7.0 g, 25%) as a yellow solid. MS-ESI: 273/275 (M+1).

1-phenyl-5-(4,4,5,5-tetramethyl-1,3,2-dioxaborolan-2-yl)-1H-indazole To a solution of the product from the previous step (2.0 g, 7.32 mmol, 1.00 equiv) and B₂Pin₂ (2.23 g, 8.79 mmol, 1.20 equiv) in 1,4-dioxane (20 mL) was added Pd(dppf)Cl₂ (0.16 g, 0.22 mmol, 0.03 equiv) and KOAc (2.16 g, 22 mmol, 3.00 equiv). The resulting mixture was stirred for 2 h at 80° C. under N₂. The residue was purified with silica gel chromatography using PE/EtOAc (10:1) to afford the title compound (2.19 g, 93.4%) as a white solid. MS-ESI: 321 (M+1).

The following boronate esters were obtained similarly.

TABLE 1
Boronate esters from aryl halides.

| Aryl halide | Boronate ester | IUPAC Name | Int. No. |
|---|---|---|---|
| X-1 | 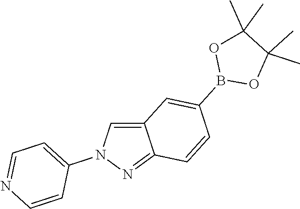 [SMILES: CC1(C)OB(c2ccc3nn(-c4ccncc4)cc3c2)OC1(C)C] | 2-(pyridin-4-yl)- 5-(4,4,5,5-tetra- methyl-1,3,2- dioxaborolan-2- yl)-2H-indazole | A-4 |

### Intermediate A-5

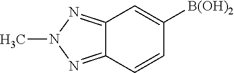

### (2-methyl-2H-benzo[d][1,2,3]triazol-5-yl) boronic acid

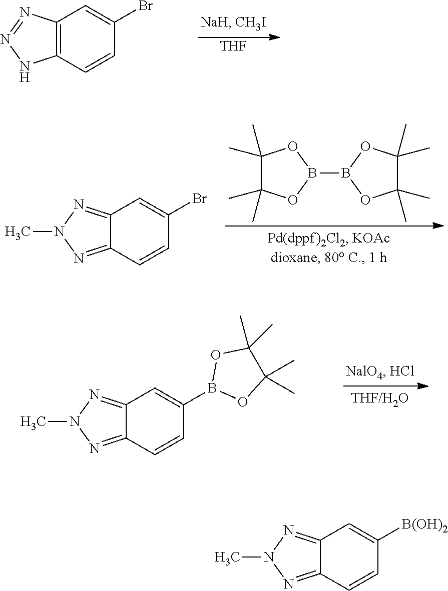

5-bromo-2-methyl-2H-benzo[d][1,2,3]triazole To a solution of 5-bromo-1H-benzo[d][1,2,3]triazole (10 g, 50.5 mmol, 1.00 equiv) in THF (200 mL) was added NaH (60% wt., 4.04 g, 101 mmol, 2.00 equiv) in portions at 0° C. The mixture was stirred for 5 min, then CH₃I (14.3 g, 101 mmol, 2.00 equiv) was added dropwise at 0° C. The mixture was stirred overnight at room temperature. The reaction mixture was quenched with H₂O (200 mL) and extracted with CH₂Cl₂ (3×150 mL). The combined organic layers were dried with anhydrous Na₂SO₄ and concentrated under vacuum. The residue was purified with silica gel chromatography using PE/EtOAc (5:1) to afford the title compound (1.2 g, 11.2%) as a white solid. MS-ESI: 212/214 (M+1).

2-methyl-5-(4,4,5,5-tetramethyl-1,3,2-dioxaborolan-2-yl)-2H-benzo[d][1,2,3]triazole To a solution of the product from the previous step (1.2 g, 5.66 mmol, 1.00 equiv) and B₂Pin₂ (1.87 g, 7.36 mmol, 1.30 equiv) in dioxane (20 mL) was added KOAc (1.66 g, 17 mmol, 3.00 equiv) and Pd(dppf)Cl₂ (420 mg, 0.57 mmol, 0.10 equiv). The resulting mixture was stirred for 4 h at 80° C. under nitrogen. The resulting mixture was concentrated under vacuum. The residue was purified with silica gel chromatography using PE/EtOAc (1:1) to afford the title compound (1.2 g, 81%) as a yellow solid. MS-ESI: 260 (M+1).

(2-methyl-2H-benzo[d][1,2,3]triazol-5-yl) boronic acid To a solution of the product from the previous step (1.2 g, 4.62 mmol, 1.00 equiv) in THF (20 mL) and H₂O (5 mL) was added NaIO₄ (2.95 g, 13.8 mmol, 3.00 equiv) in portions. The resulting mixture was stirred for 30 min at room temperature, then HCl (2 M, 4 mL) was added dropwise over 2 min at room temperature. The resulting mixture was stirred for additional 5 h at room temperature. The resulting mixture was diluted with water (100 mL). The aqueous layer was extracted with EtOAc (3×50 mL), and the combined organic layers were concentrated under vacuum to afford the title compound (378 mg, 46%) as a yellow solid. MS-ESI: 178 (M+1).

The following boronic acids were obtained similarly.

TABLE 2
Boronic acids from aryl halides.

| Aryl | | | |
|---|---|---|---|
| halide | Boronic acid | IUPAC Name | Int. No. |
| X-2 | 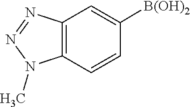 | (1-methyl-1H- benzo[d][1,2,3] triazol-5-yl) boronic acid | A-6 |
| X-3 | 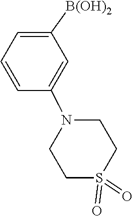 [SMILES: O=S1(=O)CCN(c2cccc(B(O)O)c2)CC1] | (3-(1,1-dioxido- thiomorpholino)- phenyl)boronic acid | A-7 |
| X-4 | 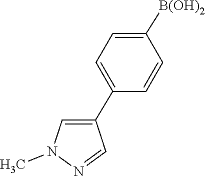 | (4-(1-methyl-1H- pyrazol-4-yl)- phenyl)boronic acid | A-8 |
| X-5 | 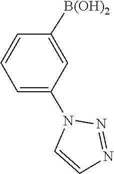 [SMILES: OB(O)c1cccc(-n2ccnn2)c1] | (3-(1H-1,2,3- triazol-1- yl)phenyl) boronic acid | A-9 |

### Intermediate C-1

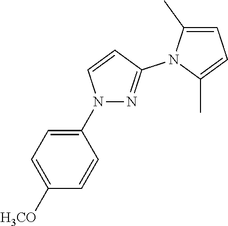 [SMILES: COc1ccc(-n2ccc(-n3c(C)ccc3C)n2)cc1]

### 3-(2,5-dimethyl-1H-pyrrol-1-yl)-1-(4-methoxyphenyl)-1H-pyrazole

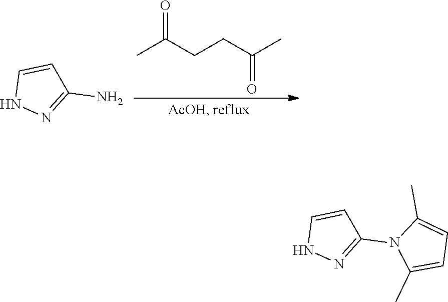

3-(2,5-dimethyl-1H-pyrrol-1-yl)-1H-pyrazole In a 500 mL round-bottom flask were combined 1H-pyrazol-3-amine (20.00 g, 240.69 mmol, 1.00 equiv), in AcOH (200 mL) and hexane-2,5-dione (20 mL) at rt. The resulting mixture was stirred 12 h at 120° C. The mixture was allowed to cool to rt, then extracted with EtOAc (2×250 mL). The combined organic layers were washed with sat aq NaHCO₃ (8×400 mL), dried over anhydrous Na₂SO₄. After filtration, the filtrate was concentrated under reduced pressure. The residue was purified with silica gel column chromatography, eluting with petroleum ether/EtOAc (7:1) to afford the title compound (20 g, 51.5%) as a yellow solid.

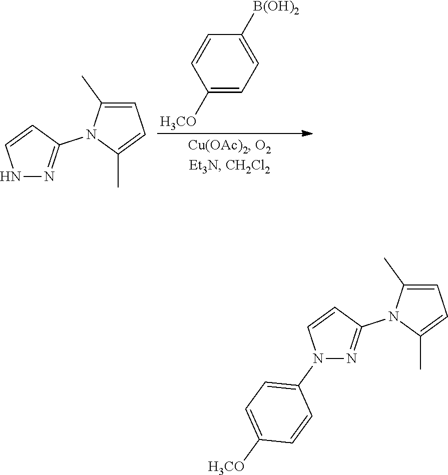 [SMILES: Cc1ccc(C)n1-c1cc[nH]n1 ; COc1ccc(B(O)O)cc1 ; COc1ccc(-n2ccc(-n3c(C)ccc3C)n2)cc1]

A solution of 3-(2,5-dimethyl-1H-pyrrol-1-yl)-1H-pyrazole (20 g, 124 mmol, 1.00 equiv), 4-methoxyphenylboronic acid (22.62 g, 148 mmol, 1.20 equiv), Cu(OAc)₂ (33.80 g, 186 mmol, 1.50 equiv), and Et₃N (37.66 g, 372 mmol, 3.00 equiv) CH₂Cl₂ (500 mL) was stirred 14 h at rt under O₂, then concentrated under reduced pressure and purified with silica gel column chromatography, eluting with petroleum ether/EtOAc (9:1), to afford the title compound (11.5 g) as a yellow crude solid. The residue was purified with reverse phase flash chromatography with the following conditions: C18 silica gel; mobile phase, CH₃CN in water, 50% to 80% gradient in 30 min, 1=254 nm, to afford the title compound (7.4 g, 22.3%) as a yellow solid.

### Intermediate C-2

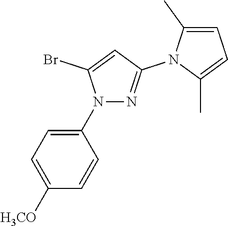 [SMILES: COc1ccc(-n2nc(-n3c(C)ccc3C)cc2Br)cc1]

### 5-bromo-3-(2,5-dimethyl-1H-pyrrol-1-yl)-1-(4-methoxyphenyl)-1H-pyrazole

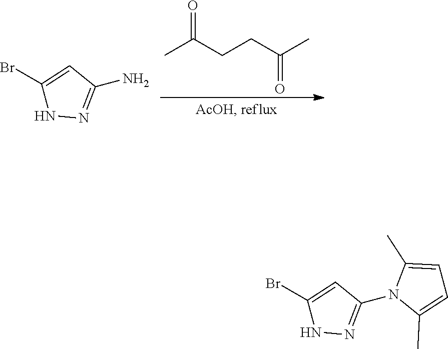

5-bromo-3-(2,5-dimethyl-1H-pyrrol-1-yl)-1H-pyrazole To a solution of 5-bromo-1H-pyrazol-3-amine (30 g, 185 mmol, 1.00 equiv) in AcOH (300 mL) was added hexane-2,5-dione (36 g, 315 mmol, 1.70 equiv) dropwise at room temperature under nitrogen. The resulting mixture was stirred overnight at 120° C. under nitrogen. The mixture was adjusted to pH 7-8 with saturated aq. NaHCO₃ and extracted with EtOAc (6×400 mL). The combined organic layers were dried with anhydrous Na₂SO₄ and concentrated under vacuum to afford the title compound (42 g, 94%) as a brown solid. MS-ESI: 240/242 (M+1).

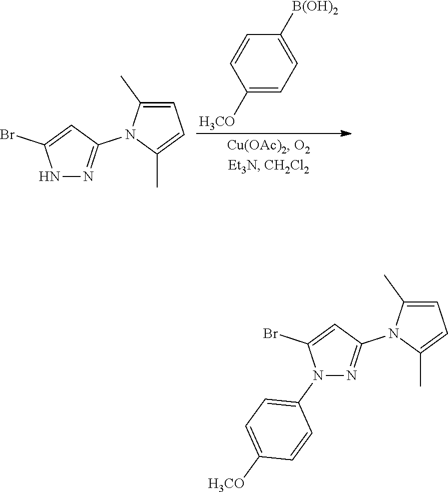 [SMILES: Cc1ccc(C)n1-c1cc(Br)[nH]n1 ; COc1ccc(B(O)O)cc1 ; COc1ccc(-n2nc(-n3c(C)ccc3C)cc2Br)cc1]

### 5-Bromo-3-(2,5-dimethyl-1H-pyrrol-1-yl)-1-(4-methoxyphenyl)-1H-pyrazole

To a solution of the product from the previous step (20 g, 83 mmol, 1.00 equiv) and (4-methoxyphenyl) boronic acid (16.5 g, 108 mmol, 1.30 equiv) in CH₂Cl₂ (300 mL) were added Cu(OAc)₂ (22.7 g, 125 mmol, 1.50 equiv) and Et₃N (25.3 g, 250 mmol, 3.00 equiv). The resulting mixture was stirred for 2 days at room temperature under O₂, then concentrated under vacuum. The residue was purified with silica gel chromatography using PE/EtOAc (100:3) to afford the title compound (5.9 g, 20%) as a white solid. MS-ESI: 346/348 (M+1).

The following compounds were obtained from reaction of 5-bromo-3-(2,5-dimethyl-1H-pyrrol-1-yl)-1H-pyrazole with boronic acids.

TABLE 3
Arylpyrazoles from boronic acids.

| | | | Int. |
|---|---|---|---|
| Boronic acid | Product | IUPAC Name | No. |
| 2-methylindazol-5- ylboronic acid | 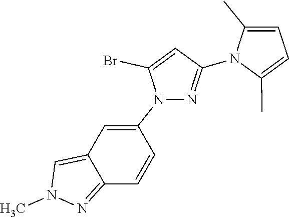 [SMILES: Cc1ccc(C)n1-c1cc(Br)n(-c2ccc3nn(C)cc3c2)n1] | 5-(5-bromo-3-(2,5- dimethyl-1H-pyrrol-1- yl)-1H-pyrazol-1-yl)-2- methyl-2H-indazole | C-3 |
| p-tolylboronic acid | 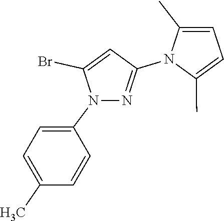 [SMILES: Cc1ccc(-n2nc(-n3c(C)ccc3C)cc2Br)cc1] | 5-bromo-3-(2,5- dimethyl-1H-pyrrol-1- yl)-1-(p-tolyl)-1H- pyrazole | C-4 |
| A-1 | 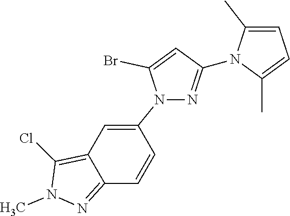 [SMILES: Cc1ccc(C)n1-c1cc(Br)n(-c2ccc3nn(C)c(Cl)c3c2)n1] | 5-(5-bromo-3-(2,5- dimethyl-1H-pyrrol-1- yl)-1H-pyrazol-1-yl)-3- chloro-2-methyl-2H- indazole | C-5 |
| A-2 | 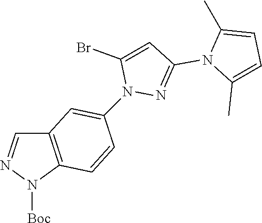 [SMILES: Cc1ccc(C)n1-c1cc(Br)n(-c2ccc3c(cnn3C(=O)OC(C)(C)C)c2)n1] | tert-Butyl 5-(5-bromo-3- (2,5-dimethyl-1H-pyrrol- 1-yl)-1H-pyrazol-1-yl)- 1H-indazole-1- carboxylate | C-6 |
| A-3 | 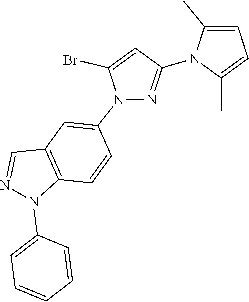 [SMILES: Cc1ccc(C)n1-c1cc(Br)n(-c2ccc3c(cnn3-c3ccccc3)c2)n1] | 5-(5-bromo-3-(2,5- dimethyl-1H-pyrrol-1- yl)-1H-pyrazol-1-yl)-1- phenyl-1H-indazole | C-7 |
| A-4 | 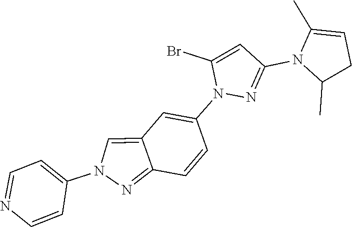 [SMILES: CC1=CCC(C)N1c1cc(Br)n(-c2ccc3nn(-c4ccncc4)cc3c2)n1] | 5-(5-bromo-3-(2,5- dimethyl-2,3-dihydro- 1H-pyrrol-1-yl)-1H- pyrazol-1-yl)-2-(pyridin- 4-yl)-2H-indazole | C-8 |
| A-5 | 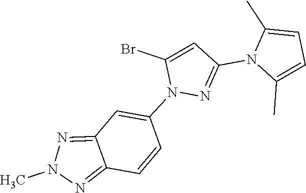 [SMILES: Cc1ccc(C)n1-c1cc(Br)n(-c2ccc3nn(C)nc3c2)n1] | 5-(5-bromo-3-(2,5- dimethyl-1H-pyrrol-1- yl)-1H-pyrazol-1-yl)-2- methyl-2H- benzo[d][1,2,3]triazole | C-9 |
| A-6 | 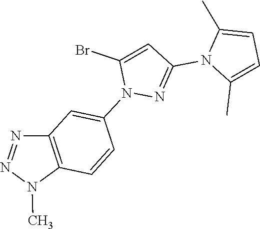 [SMILES: Cc1ccc(C)n1-c1cc(Br)n(-c2ccc3c(c2)nnn3C)n1] | 5-(5-bromo-3-(2,5- dimethyl-1H-pyrrol-1- yl)-1H-pyrazol-1-yl)-1- methyl-1H- benzo[d][1,2,3]triazole | C-10 |
| A-7 | 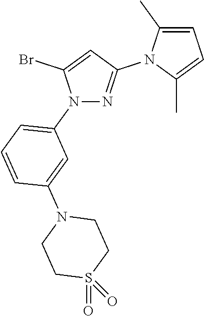 [SMILES: Cc1ccc(C)n1-c1cc(Br)n(-c2cccc(N3CCS(=O)(=O)CC3)c2)n1] | 4-(3-(5-bromo-3-(2,5- dimethyl-1H-pyrrol-1- yl)-1H-pyrazol-1- yl)phenyl)- thiomorpholine 1,1- dioxide | C-11 |
| A-8 | 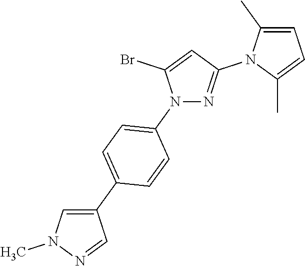 [SMILES: Cc1ccc(C)n1-c1cc(Br)n(-c2ccc(-c3cnn(C)c3)cc2)n1] | 5-bromo-3-(2,5- dimethyl-1H-pyrrol-1- yl)-1-(4-(1-methyl-1H- pyrazol-4-yl)phenyl)-1H- pyrazole | C-12 |
| A-9 | 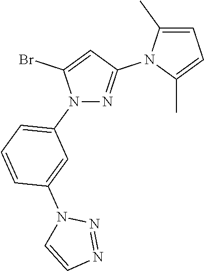 [SMILES: Cc1ccc(C)n1-c1cc(Br)n(-c2cccc(-n3ccnn3)c2)n1] | 1-(3-(5-bromo-3-(2,5- dimethyl-1H-pyrrol-1- yl)-1H-pyrazol-1- yl)phenyl)-1H-1,2,3- triazole | C-13 |
| (1H-indol-6-yl)- boronic acid | 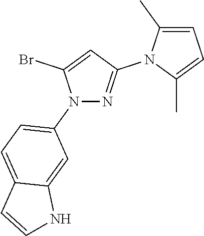 [SMILES: Cc1ccc(C)n1-c1cc(Br)n(-c2ccc3cc[nH]c3c2)n1] | 6-(5-bromo-3-(2,5- dimethyl-1H-pyrrol-1- yl)-1H-pyrazol-1-yl)-1H- indole | C-14 |
| (4-(4-(tert- butoxycarbonyl)- piperazin-1- yl)phenyl)boronic acid | 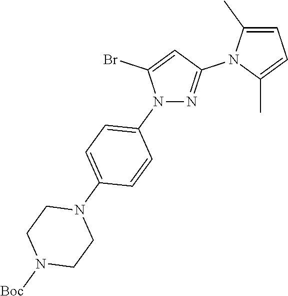 [SMILES: Cc1ccc(C)n1-c1cc(Br)n(-c2ccc(N3CCN(C(=O)OC(C)(C)C)CC3)cc2)n1] | tert-Butyl 4-(4-(5-bromo- 3-(2,5-dimethyl-1H- pyrrol-1-yl)-1H-pyrazol- 1-yl)phenyl)piperazine- 1-carboxylate | C-15 |
| (3-(4-(tert- butoxycarbonyl)- piperazin-1- yl)phenyl)boronic acid | 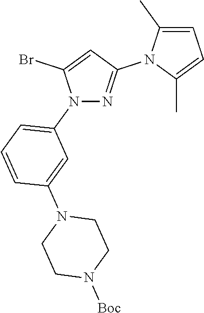 [SMILES: Cc1ccc(C)n1-c1cc(Br)n(-c2cccc(N3CCN(C(=O)OC(C)(C)C)CC3)c2)n1] | tert-Butyl 4-(3-(5-bromo- 3-(2,5-dimethyl-1H- pyrrol-1-yl)-1H-pyrazol- 1-yl)phenyl)piperazine- 1-carboxylate | C-16 |
| (4- (methoxycarbonyl)- phenyl)boronic acid | 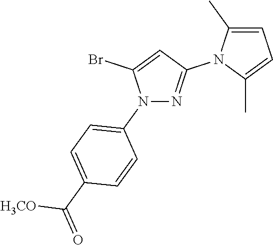 [SMILES: COC(=O)c1ccc(-n2nc(-n3c(C)ccc3C)cc2Br)cc1] | Methyl 4-(5-bromo-3- (2,5-dimethyl-1H-pyrrol- 1-yl)-1H-pyrazol-1- yl)benzoate | C-17 |

### Intermediate C-18

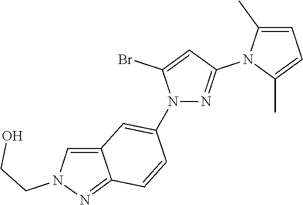 [SMILES: Cc1ccc(C)n1-c1cc(Br)n(-c2ccc3nn(CCO)cc3c2)n1]

### 2-(5-(5-bromo-3-(2,5-dimethyl-1H-pyrrol-1-yl)-1H-pyrazol-1-yl)-2H-indazol-2-yl) ethan-1-ol

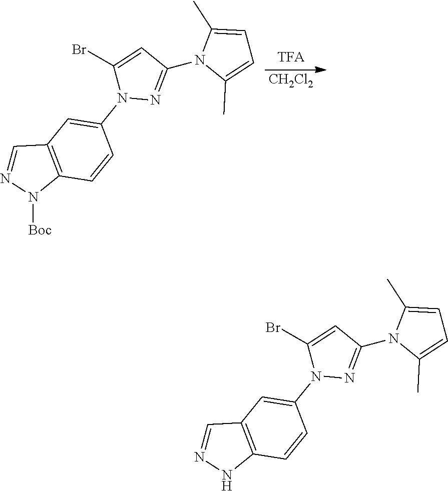

5-(5-bromo-3-(2,5-dimethyl-1H-pyrrol-1-yl)-1H-pyrazol-1-yl)-1H-indazole To a solution of Intermediate C-6 (640 mg, 1.4 mmol, 1.00 equiv) in CH₂Cl₂ (5 mL) was added TFA (1 mL). The stirred solution was stirred for 1 h at room temperature. The resulting mixture was adjusted pH to 7-8 with sat. Na₂CO₃ aq. and extracted with CH₂Cl₂ (3×50 mL). The combined organic layers were dried with anhydrous Na₂SO₄ and concentrated under vacuum. The residue purified with silica gel chromatography using PE/EtOAc (1:1) to afford the title compound (300 mg, 60%) as a white solid. MS-ESI: 356/358 (M+1).

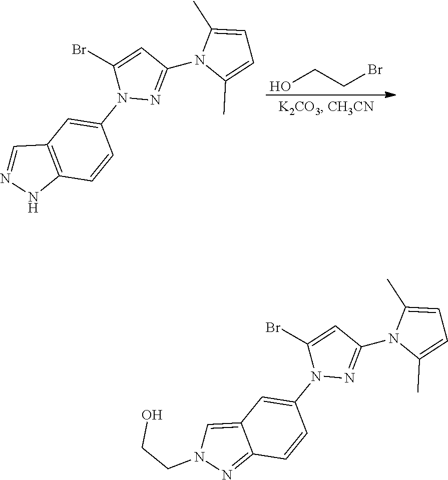

2-(5-(5-bromo-3-(2,5-dimethyl-1H-pyrrol-1-yl)-1H-pyrazol-1-yl)-2H-indazol-2-yl) ethan-1-ol To a solution of the product from the previous step (300 mg, 0.84 mmol, 1.00 equiv) in CH₃CN (10 mL) was added 2-bromoethanol (158 mg, 1.26 mmol, 1.5 equiv) and K₂CO₃ (348 mg, 2.52 mmol, 3.00 equiv). The mixture was stirred for 16 h at 80° C. The mixture was concentrated under vacuum and purified by Prep-HPLC with the following conditions: XBridge Prep OBD C18 Column, 30*150 mm, 5 um; Mobile Phase A: water (10 mM NH₄HCO₃), Mobile Phase B: ACN; Flow rate: 60 mL/min; Gradient: 35% ACN to 60% ACN in 8 min; Detector UV: 220 nm; RT1(min): 7.83 to afford the title compound (65 mg, 41%). MS-ESI: 400/402 (M+1).

### Intermediate C-19

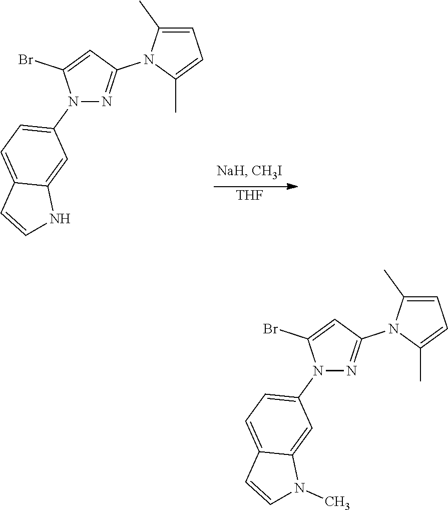 [SMILES: Cc1ccc(C)n1-c1cc(Br)n(-c2ccc3cc[nH]c3c2)n1 ; Cc1ccc(C)n1-c1cc(Br)n(-c2ccc3ccn(C)c3c2)n1 ; "THF"]

6-(5-bromo-3-(2,5-dimethyl-1H-pyrrol-1-yl)-1H-pyrazol-1-yl)-1-methyl-1H-indole To a solution of Intermediate C-14 (2.0 g, 5.63 mmol, 1.00 equiv) in THF (20 mL) was added NaH (60% wt., 450 mg, 11.3 mmol, 2.0 equiv) and CH₃I (879 mg, 6.19 mmol, 1.1 equiv). The mixture was stirred overnight at room temperature. The mixture was quenched with H₂O (50 mL) and extracted with EtOAc (3×100 mL). The combined organic layers were dried with anhydrous Na₂SO₄ and concentrated under vacuum. The residue was purified with silica gel chromatography using PE/EtOAc (3:1) to afford the title compound (200 mg, 40.5%) as a yellow solid. MS-ESI: 369/371 (M+1).

### Intermediate C-20

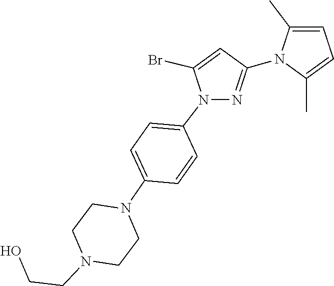 [SMILES: Cc1ccc(C)n1-c1cc(Br)n(-c2ccc(N3CCN(CCO)CC3)cc2)n1]

### 2-(4-(4-(5-bromo-3-(2,5-dimethyl-1H-pyrrol-1-yl)-1H-pyrazol-1-yl)phenyl) piperazin-1-yl) ethan-1-ol

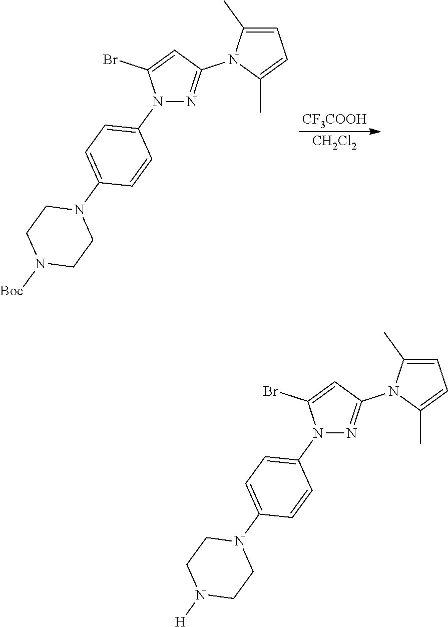

1-(4-(5-bromo-3-(2,5-dimethyl-1H-pyrrol-1-yl)-1H-pyrazol-1-yl)phenyl)-piperazine (Intermediate C-21) To a solution of Intermediate C-15 (3.0 g, 6.0 mmol, 1.00 equiv) in CH₂Cl₂ (40 mL) was added CF₃COOH (8 mL). The resulting mixture was stirred for 2 h at room temperature. The mixture was adjusted to pH 7-8 with sat. aq Na₂CO₃ and extracted with CH₂Cl₂: MeOH (10:1, 3×40 mL). The combined organic layers were dried with anhydrous Na₂SO₄ and concentrated under vacuum to afford the title compound (2.5 g, 62.5%) as a yellow solid. MS-ESI: 400/402 (M+1).

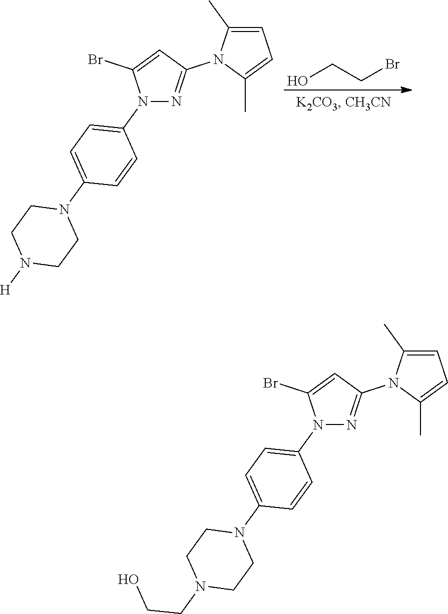

2-(4-(4-(5-bromo-3-(2,5-dimethyl-1H-pyrrol-1-yl)-1H-pyrazol-1-yl)phenyl)-piperazin-1-yl) ethan-1-ol To a solution of 1-(4-(5-bromo-3-(2,5-dimethyl-1H-pyrrol-1-yl)-1H-pyrazol-1-yl)phenyl) piperazine (600 mg, 1.5 mmol, 1.00 equiv) in CH₃CN (20 mL) were added K₂CO₃ (621 mg, 4.5 mmol, 3.00 equiv) and 2-bromoethanol (374 mg, 3.0 mmol, 2.00 equiv). The resulting mixture was stirred 24 h at 80° C. The reaction mixture was quenched with H₂O (20 mL) and extracted with CH₂Cl₂ (3×50 mL). The combined organic layers were dried with anhydrous Na₂SO₄ and concentrated under vacuum to afford the title compound (650 mg, crude) as brown oil. MS-ESI: 444/446 (M+1).

### Intermediate C-22

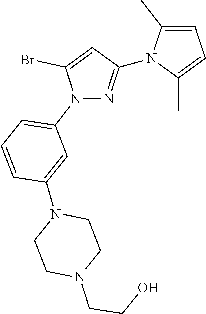 [SMILES: Cc1ccc(C)n1-c1cc(Br)n(-c2cccc(N3CCN(CCO)CC3)c2)n1]

### 2-(4-(3-(5-bromo-3-(2,5-dimethyl-1H-pyrrol-1-yl)-1H-pyrazol-1-yl)phenyl) piperazin-1-yl) ethan-1-ol

The above compound was prepared similarly to Intermediate C-20, from Intermediate C-16.

### Intermediate C-23

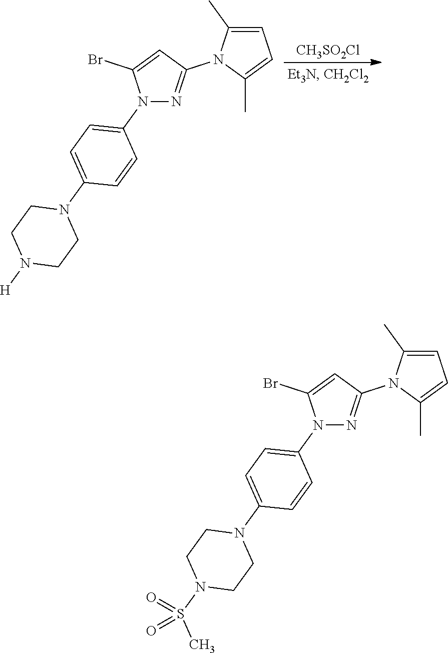

1-(4-(5-bromo-3-(2,5-dimethyl-1H-pyrrol-1-yl)-1H-pyrazol-1-yl)phenyl)-4-(methylsulfonyl) piperazine To a solution of Intermediate C-21 1-(4-(5-bromo-3-(2,5-dimethyl-1H-pyrrol-1-yl)-1H-pyrazol-1-yl)phenyl) piperazine (100 mg, 0.25 mmol, 1.00 equiv) and CH₃SO₂Cl (28.6 mg, 0.25 mmol, 1 equiv) in DCM (3 mL) was added TEA (75.8 mg, 0.75 mmol, 3 equiv). The resulting mixture was stirred for 1 h at room temperature. The mixture was concentrated under vacuum and purified with silica gel chromatography using PE/EtOAc (1:1) to afford the title compound (60 mg, 50%) as a yellow solid. MS-ESI: 478/480 (M+1).

### Intermediate C-24

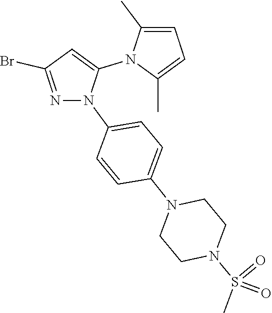 [SMILES: Cc1ccc(C)n1-c1cc(Br)nn1-c1ccc(N2CCN(S(C)(=O)=O)CC2)cc1]

### 1-(4-(3-bromo-5-(2,5-dimethyl-1H-pyrrol-1-yl)-1H-pyrazol-1-yl)phenyl)-4-(methylsulfonyl) piperazine

The above compound was prepared similarly to Intermediate C-23, from Intermediate C-16.

### Intermediate C-25

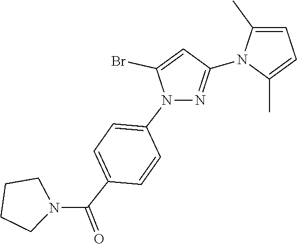 [SMILES: Cc1ccc(C)n1-c1cc(Br)n(-c2ccc(C(=O)N3CCCC3)cc2)n1]

### (4-(5-Bromo-3-(2,5-dimethyl-1H-pyrrol-1-yl)-1H-pyrazol-1-yl)phenyl) (pyrrolidin-1-yl) methanone

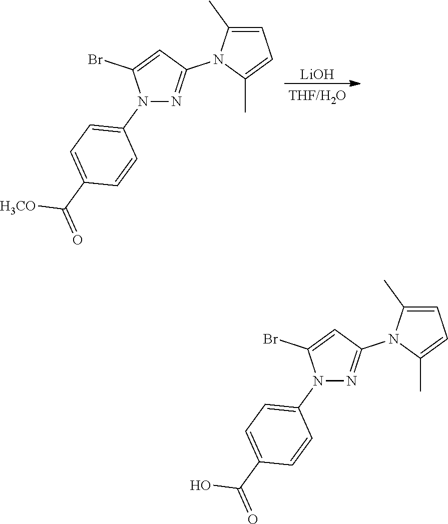

### 4-(5-bromo-3-(2,5-dimethyl-1H-pyrrol-1-yl)-1H-pyrazol-1-yl)benzoic acid

To a solution of Intermediate C-17 (1.79 g, 4.78 mmol, 1.00 equiv) in THF (50 mL) and H₂O (20 mL) was added LiOH (0.23 g, 9.56 mmol, 2.00 equiv). The mixture was stirred for 1 h at room temperature. The mixture was adjusted to pH 3-4 with HCl (2 M) and extracted with CH₂Cl₂ (3×70 mL). The combined organic layers were washed with water (3×150 mL), dried over anhydrous Na₂SO₄ and concentrated under vacuum to afford the title compound (1.7 g, 98%) as a white solid. MS-ESI: 360/362 (M+1).

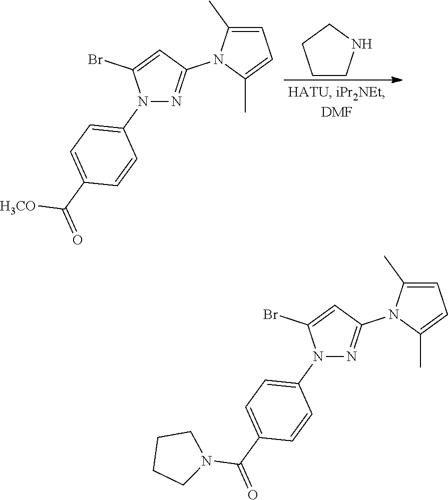

(4-(5-Bromo-3-(2,5-dimethyl-1H-pyrrol-1-yl)-1H-pyrazol-1-yl)phenyl)-(pyrrolidin-1-yl) methanone To a solution of the product from the previous step (1.6 g, 4.5 mmol, 1 equiv) in DMF (15 mL) was added HATU (3.43 g, 9.03 mmol, 2.00 equiv). The mixture was stirred for 10 min at room temperature. To the above mixture were added pyrrolidine (0.32 g, 4.52 mmol, 1.00 equiv) and DIEA (1.75 g, 13.6 mmol, 3.00 equiv). The resulting mixture was stirred for additional 2 h at room temperature. The mixture was diluted with H₂O (50 mL) and extracted with EtOAc (3×100 mL). The combined organic layers were concentrated under vacuum. The residue was purified with silica gel chromatography using PE/EtOAc (1:1) to afford the title compound (1.2 g, 64%) as a pink solid. MS-ESI: 413/415 (M+1).

### Intermediate D-1

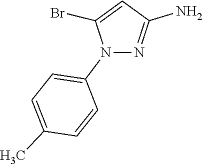

### 5-bromo-1-(p-tolyl)-1H-pyrazol-3-amine

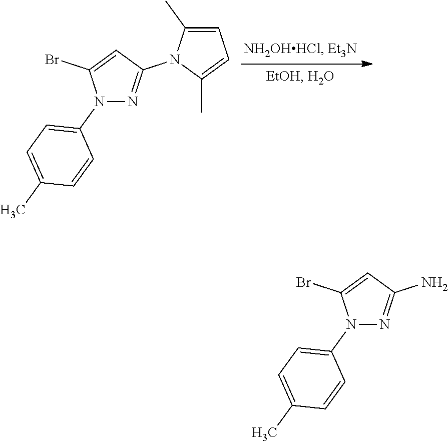 [SMILES: Cc1ccc(-n2nc(-n3c(C)ccc3C)cc2Br)cc1 ; Cc1ccc(-n2nc(N)cc2Br)cc1]

To a stirred mixture of Intermediate C-4 (1.1 g, 3.36 mmol, 1.00 equiv) in EtOH (20 mL) and H₂O (10 mL) were added Et₃N (3.35 g, 33.6 mmol, 10 equiv) and NH₂OH·HCl (9.42 g, 134.40 mmol, 40 equiv) in portions at 90° C. The resulting mixture was stirred overnight at 90° C., then concentrated under reduced pressure. The aqueous layer was extracted with EtOAc (3×100 mL). The combined organic layers were concentrated under reduced pressure to afford the title compound (370 mg, 11.3%) as a brown yellow oil.

The following aminopyrazoles were obtained from reaction of (2,5-dimethyl-1H-pyrrol-1-yl)-1H-pyrazoles with hydroxylamine.

TABLE 4
Aminopyrazoles from pyrrolyl pyrazoles.

| Pyrrolyl | | | Int. |
|---|---|---|---|
| pyrazole | Aminopyrazole | IUPAC Name | No. |
| C-2 | 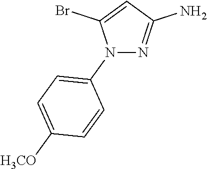 [SMILES: COc1ccc(-n2nc(N)cc2Br)cc1] | 5-bromo-1-(4- methoxyphenyl)-1H- pyrazol-3-amine | D-2 |
| C-3 | 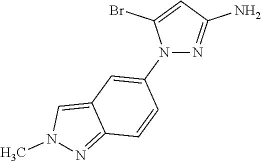 [SMILES: Cn1cc2cc(-n3nc(N)cc3Br)ccc2n1] | 5-bromo-1-(2-methyl- 2H-indazol-5-yl)-1H- pyrazol-3-amine | D-3 |
| C-5 | 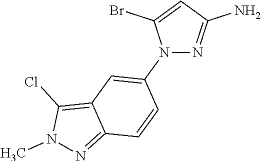 [SMILES: Cn1nc2ccc(-n3nc(N)cc3Br)cc2c1Cl] | 5-bromo-1-(3-chloro-2- methyl-2H-indazol-5-yl)- 1H-pyrazol-3-amine | \|D-4 |
| C-6 | 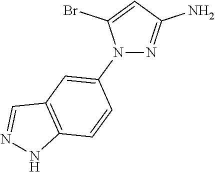 [SMILES: Nc1cc(Br)n(-c2ccc3[nH]ncc3c2)n1] | 5-bromo-1-(1H-indazol- 5-yl)-1H-pyrazol-3- amine | D-5 |
| C-18 | 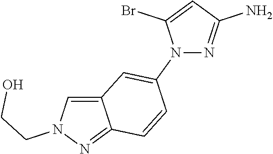 [SMILES: Nc1cc(Br)n(-c2ccc3nn(CCO)cc3c2)n1] | 2-(5-(3-amino-5-bromo- 1H-pyrazol-1-yl)-2H - indazol-2-yl)ethan-1-ol | D-6 |
| C-19 | 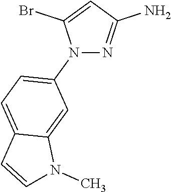 [SMILES: Cn1ccc2ccc(-n3nc(N)cc3Br)cc21] | 5-bromo-1-(1-methyl- 1H-indol-6-yl)-1H- pyrazol-3-amine | D-7 |
| C-7 | 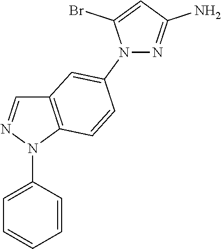 [SMILES: Nc1cc(Br)n(-c2ccc3c(cnn3-c3ccccc3)c2)n1] | 5-bromo-1-(1-phenyl- 1H-indazol-5-yl)-1H- pyrazol-3-amine | D-8 |
| | 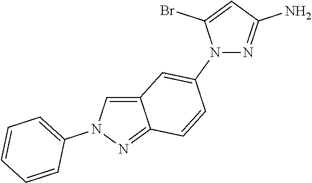 [SMILES: Nc1cc(Br)n(-c2ccc3nn(-c4ccccc4)cc3c2)n1] | 5-bromo-1-(2-phenyl- 2H-indazol-5-yl)-1H- pyrazol-3-amine | D-9 |
| C-8 | 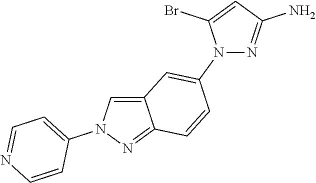 [SMILES: Nc1cc(Br)n(-c2ccc3nn(-c4ccncc4)cc3c2)n1] | 5-bromo-1-(2-(pyridin-4- yl)-2H-indazol-5-yl)-1H- pyrazol-3-amine | D-10 |
| | 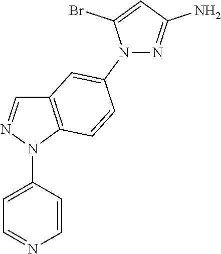 [SMILES: Nc1cc(Br)n(-c2ccc3c(cnn3-c3ccncc3)c2)n1] | 5-bromo-1-(1-(pyridin-4- yl)-1H-indazol-5-yl)-1H- pyrazol-3-amine | D-11 |
| C-9 | 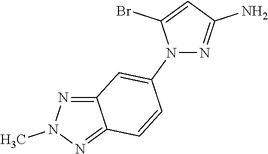 [SMILES: Cn1nc2ccc(-n3nc(N)cc3Br)cc2n1] | 5-bromo-1-(2-methyl- 2H- benzo[d][1,2,3]triazol-5- yl)-1H-pyrazol-3-amine | D-12 |
| | 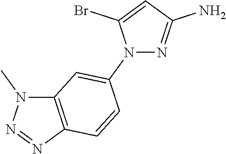 [SMILES: Cn1nnc2ccc(-n3nc(N)cc3Br)cc21] | 5-bromo-1-(1-methyl- 1H- benzo[d][1,2,3]triazol-6- yl)-1H-pyrazol-3-amine | D-13 |
| C-10 | 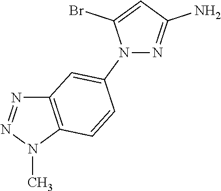 [SMILES: Cn1nnc2cc(-n3nc(N)cc3Br)ccc21] | 5-bromo-1-(1-methyl- 1H- benzo[d][1,2,3]triazol-5- yl)-1H-pyrazol-3-amine | D-14 |
| C-11 | 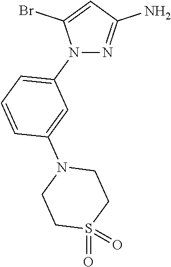 [SMILES: Nc1cc(Br)n(-c2cccc(N3CCS(=O)(=O)CC3)c2)n1] | 4-(3-(3-amino-5-bromo- 1H-pyrazol-1- yl)phenyl)thiomorpholine 1,1-dioxide | D-15 |
| | 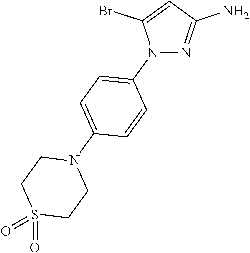 [SMILES: Nc1cc(Br)n(-c2ccc(N3CCS(=O)(=O)CC3)cc2)n1] | 4-(4-(3-amino-5-bromo- 1H-pyrazol-1- yl)phenyl)thiomorpholine 1,1-dioxide | D-16 |
| C-12 | 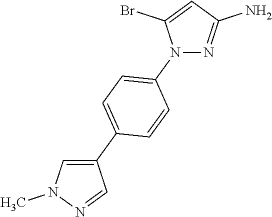 [SMILES: Cn1cc(-c2ccc(-n3nc(N)cc3Br)cc2)cn1] | 5-bromo-1-(4-(1-methyl- 1H-pyrazol-4-yl)phenyl)- 1H-pyrazol-3-amine | D-17 |
| | 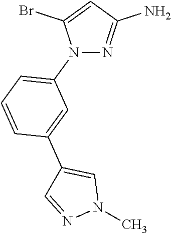 [SMILES: Cn1cc(-c2cccc(-n3nc(N)cc3Br)c2)cn1] | 5-bromo-1-(3-(1-methyl- 1H-pyrazol-4-yl)phenyl)- 1H-pyrazol-3-amine | D-18 |
| | 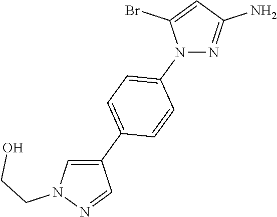 [SMILES: Nc1cc(Br)n(-c2ccc(-c3cnn(CCO)c3)cc2)n1] | 2-(4-(4-(3-amino-5- bromo-1H-pyrazol-1- yl)phenyl)-1H-pyrazol-1- yl)ethan-1-ol | D-19 |
| | 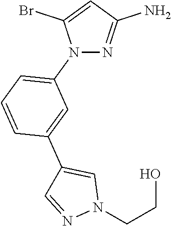 [SMILES: Nc1cc(Br)n(-c2cccc(-c3cnn(CCO)c3)c2)n1] | 2-(4-(3-(3-amino-5- bromo-1H-pyrazol-1- yl)phenyl)-1H-pyrazol-1- yl)ethan-1-ol | D-20 |
| C-13 | 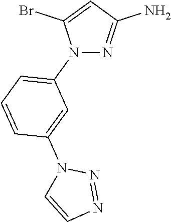 [SMILES: Nc1cc(Br)n(-c2cccc(-n3ccnn3)c2)n1] | 1-(3-(1H-1,2,3-triazol-1- yl)phenyl)-5-bromo-1H- pyrazol-3-amine | D-21 |
| | 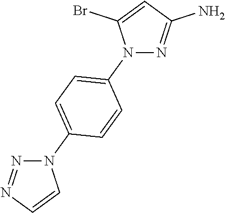 [SMILES: Nc1cc(Br)n(-c2ccc(-n3ccnn3)cc2)n1] | 1-(4-(1H-1,2,3-triazol-1- yl)phenyl)-5-bromo-1H- pyrazol-3-amine | D-22 |
| C-20 | 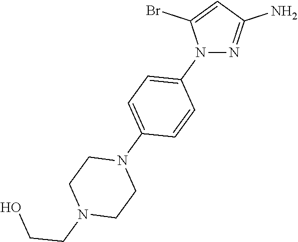 [SMILES: Nc1cc(Br)n(-c2ccc(N3CCN(CCO)CC3)cc2)n1] | 2-(4-(4-(3-amino-5- bromo-1H-pyrazol-1- yl)phenyl)piperazin-1- yl)ethan-1-ol | D-23 |
| C-22 | 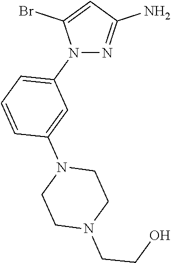 [SMILES: Nc1cc(Br)n(-c2cccc(N3CCN(CCO)CC3)c2)n1] | 2-(4-(3-(3-amino-5- bromo-1H-pyrazol-1-yl)- phenyl)piperazin-1-yl)- ethan-1-ol | D-24 |
| C-23 | 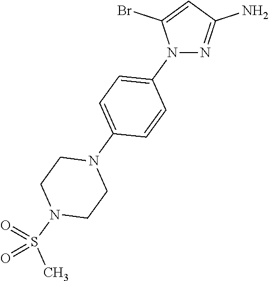 [SMILES: CS(=O)(=O)N1CCN(c2ccc(-n3nc(N)cc3Br)cc2)CC1] | 5-bromo-1-(4-(4- (methylsulfonyl)piperazin- 1-yl)phenyl)-1H- pyrazol-3-amine | D-25 |
| C-24 | 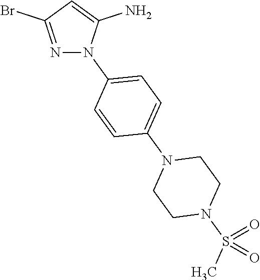 [SMILES: CS(=O)(=O)N1CCN(c2ccc(-n3nc(Br)cc3N)cc2)CC1] | 3-bromo-1-(4-(4- (methylsulfonyl)piperazin- 1-yl)phenyl)-1H- pyrazol-5-amine | D-26 |
| C-25 | 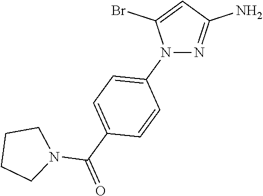 [SMILES: Nc1cc(Br)n(-c2ccc(C(=O)N3CCCC3)cc2)n1] | (4-(3-amino-5-bromo- 1H-pyrazol-1-yl)phenyl)- (pyrrolidin-1- yl)methanone | D-27 |

### Intermediate B-1=EXAMPLE 1

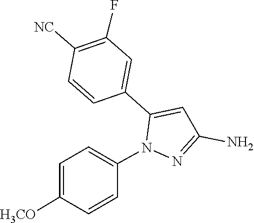 [SMILES: COc1ccc(-n2nc(N)cc2-c2ccc(C#N)c(F)c2)cc1]

### 4-(3-Amino-1-(4-methoxyphenyl)-1H-pyrazol-5-yl)-2-fluorobenzonitrile

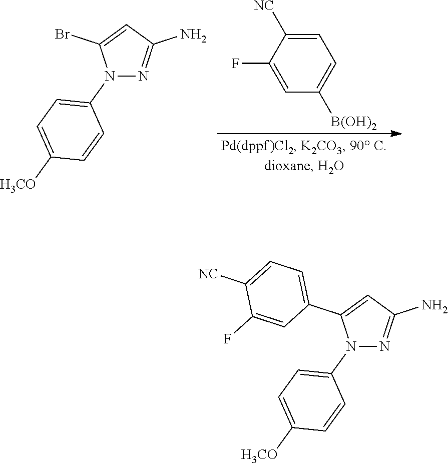 [SMILES: COc1ccc(-n2nc(N)cc2Br)cc1 ; N#Cc1ccc(B(O)O)cc1F ; COc1ccc(-n2nc(N)cc2-c2ccc(C#N)c(F)c2)cc1]

4-(3-Amino-1-(4-methoxyphenyl)-1H-pyrazol-5-yl)-2-fluorobenzonitrile To a solution of Intermediate D-2 (3.0 g, 11.2 mmol, 1.00 equiv) and (4-cyano-3-fluorophenyl)-boronic acid (1.85 g, 11.2 mmol, 1 equiv) in dioxane (30 mL) and H₂O (3 mL) was added Pd(dppf)Cl₂ (0.82 g, 1.12 mmol, 0.1 equiv) and K₂CO₃ (4.64 g, 33.5 mmol, 3.00 equiv). The resulting mixture was stirred overnight at 90° C. under N₂. The resulting mixture was quenched with H₂O (30 mL) and extracted with EtOAc (3×100 mL). The combined organic layers were dried over anhydrous Na₂SO₄ and concentrated under vacuum. The residue was purified with silica gel chromatography using PE/EtOAc (1:1) to afford the title compound (2.4 g, 69%) as a yellow solid. MS-ESI: 309 (M+1).

### Intermediate B-2

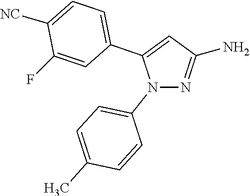 [SMILES: Cc1ccc(-n2nc(N)cc2-c2ccc(C#N)c(F)c2)cc1]

### 4-(3-Amino-1-(p-tolyl)-1H-pyrazol-5-yl)-2-fluorobenzonitrile

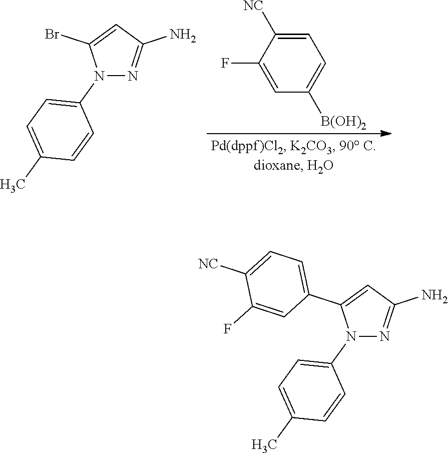 [SMILES: Cc1ccc(-n2nc(N)cc2Br)cc1 ; N#Cc1ccc(B(O)O)cc1F ; Cc1ccc(-n2nc(N)cc2-c2ccc(C#N)c(F)c2)cc1]

To a stirred mixture of Intermediate D-1 (350 mg, 1.388 mmol, 1 equiv) in dioxane (10 mL), (1 mL) were added (4-cyano-3-fluorophenyl) boronic acid (297.65 mg, 1.805 mmol, 1.3 equiv), Pd(dppf)Cl₂ (101.58 mg, 0.139 mmol, 0.10 equiv) and K₂CO₃ (575.59 mg, 4.165 mmol, 3 equiv) in portions at 90° C. under N₂. The resulting mixture was stirred overnight at 90° C. under N₂. The resulting mixture was filtered, and the filter cake was washed with EtOAc (3×10 mL). The filtrate was concentrated under reduced pressure and purified with Prep-TLC (petroleum ether/EtOAc 1:1) to afford the title compound (270 mg, 71.87%) as a yellow solid.

### Intermediate B-3

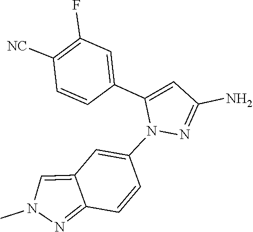 [SMILES: Cn1cc2cc(-n3nc(N)cc3-c3ccc(C#N)c(F)c3)ccc2n1]

### 4-(3-Amino-1-(2-methyl-2H-indazol-5-yl)-1H-pyrazol-5-yl)-2-fluorobenzonitrile

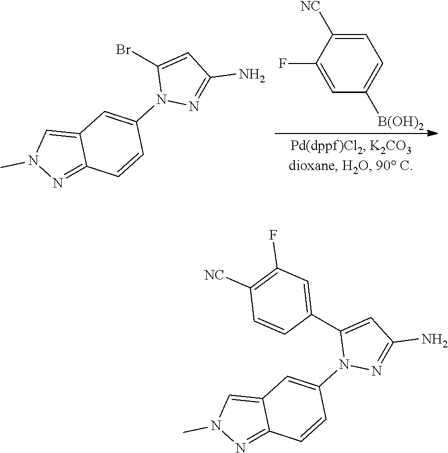

4-(3-Amino-1-(2-methyl-2H-indazol-5-yl)-1H-pyrazol-5-yl)-2-fluorobenzonitrile A solution of Intermediate D-3 (1.17 g, 4.01 mmol, 1.00 equiv), 4-cyano-3-fluorophenylboronic acid (0.79 g, 4.8 mmol, 1.2 equiv), K₂CO₃ (1.66 g, 12 mmol, 3.00 equiv) and Pd(dppf)Cl₂ (283 mg, 0.40 mmol, 0.10 equiv) in dioxane (50 mL) and H₂O (5 mL) was stirred 12 h at 90° C. under N₂. The mixture was quenched with H₂O (100 mL) and extracted with EtOAc (4×100 mL). The combined organic layers were dried over anhydrous Na₂SO₄, concentrated under vacuum, and purified with silica gel chromatography, eluting with CH₂Cl₂/CH₃OH (40:1), to afford 1.2 g (90%) of the title compound as a tan solid. MS-ESI: 333 (M+1).

The following compounds were obtained with methods similar to those used to obtain Intermediate B-1.

TABLE 5
Pyrazol-3-amines.

| | | | MWt/ |
|---|---|---|---|
| Structure | IUPAC Name | Int. No. | M + H⁺ |
| 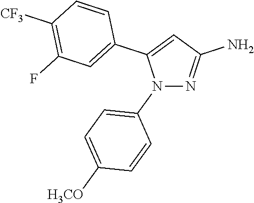 [SMILES: COc1ccc(-n2nc(N)cc2-c2ccc(C(F)(F)F)c(F)c2)cc1] | 5-(3-fluoro-4- (trifluoromethyl)phenyl)- 1-(4-methoxyphenyl)- 1H-pyrazol-3-amine | B-4 | 351/352 |
| 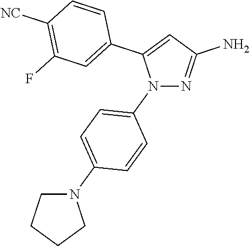 [SMILES: N#Cc1ccc(-c2cc(N)nn2-c2ccc(N3CCCC3)cc2)cc1F] | 4-(3-amino-1-(4- (pyrrolidin-1- yl)phenyl)-1H-pyrazol- 5-yl)-2- fluorobenzonitrile | B-5 | 347/348 |
| 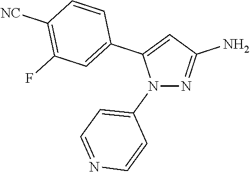 [SMILES: N#Cc1ccc(-c2cc(N)nn2-c2ccncc2)cc1F] | 4-(3-amino-1-(pyridin- 4-yl)-1H-pyrazol-5-yl)- 2-fluorobenzonitrile | B-6 | 279/280 |
| 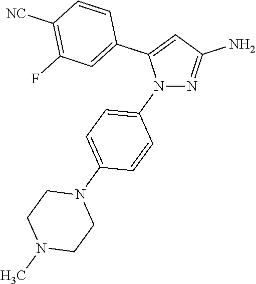 [SMILES: CN1CCN(c2ccc(-n3nc(N)cc3-c3ccc(C#N)c(F)c3)cc2)CC1] | 4-(3-amino-1-(4-(4- methylpiperazin-1- yl)phenyl)-1H-pyrazol- 5-yl)-2- fluorobenzonitrile | B-7 | 376/377 |
| 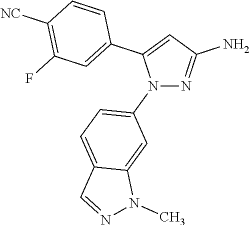 [SMILES: Cn1ncc2ccc(-n3nc(N)cc3-c3ccc(C#N)c(F)c3)cc21] | 4-(3-amino-1-(1- methyl-1H-indazol-6- yl)-1H-pyrazol-5-yl)-2- fluorobenzonitrile | B-8 | 332/333 |
| 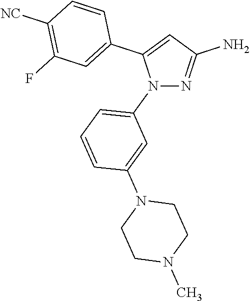 [SMILES: CN1CCN(c2cccc(-n3nc(N)cc3-c3ccc(C#N)c(F)c3)c2)CC1] | 4-(3-amino-1-(3-(4- methylpiperazin-1- yl)phenyl)-1H-pyrazol- 5-yl)-2- fluorobenzonitrile | B-9 | 376/377 |
| 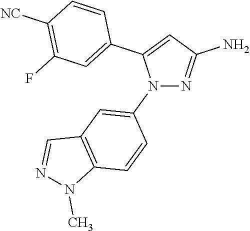 [SMILES: Cn1ncc2cc(-n3nc(N)cc3-c3ccc(C#N)c(F)c3)ccc21] | 4-(3-amino-1-(1- methyl-1H-indazol-5- yl)-1H-pyrazol-5-yl)-2- fluorobenzonitrile | B-10 | 332/333 |

The following compounds were obtained with methods similar to those used to obtain Intermediate B-1, using the 3-amino-5-bromo pyrazole indicated.

TABLE 6
Pyrazol-3-amines from 3-amino-5-bromo pyrazole.

| | | | Int. | MWt/ |
|---|---|---|---|---|
| Pyrazole | Product | IUPAC Name | No. | M + H⁺ |
| D-4 | 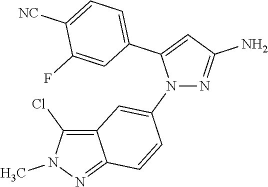 [SMILES: Cn1nc2ccc(-n3nc(N)cc3-c3ccc(C#N)c(F)c3)cc2c1Cl] | 4-(3-amino-1-(3- chloro-2-methyl- 2H-indazol-5-yl)- 1H-pyrazol-5-yl)- 2- fluorobenzonitrile | B-11 | 366/ 367 |
| D-5 | 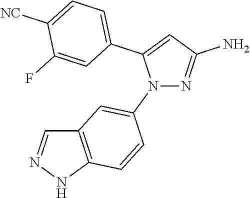 [SMILES: N#Cc1ccc(-c2cc(N)nn2-c2ccc3[nH]ncc3c2)cc1F] | 4-(3-amino-1-(1H- indazol-5-yl)-1H- pyrazol-5-yl)-2- fluorobenzonitrile | B-12 | 318/ 319 (a) |
| D-6 | 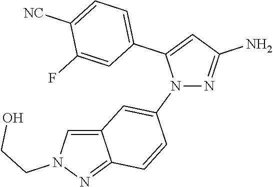 [SMILES: N#Cc1ccc(-c2cc(N)nn2-c2ccc3nn(CCO)cc3c2)cc1F] | 4-(3-amino-1-(2- (2-hydroxyethyl)- 2H-indazol-5-yl)- 1H-pyrazol-5-yl)- 2- fluorobenzonitrile | B-13 | 362/ 363 |

The following compounds were prepared using procedures similar to that for obtaining Intermediate B-13.

TABLE 7
Pyrazol-3-amines.

| | | | MWt/ |
|---|---|---|---|
| Structure | IUPAC Name | Int. No. | M + H⁺ |
| 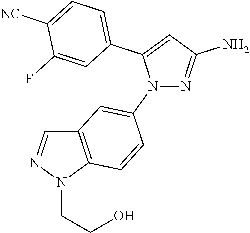 [SMILES: N#Cc1ccc(-c2cc(N)nn2-c2ccc3c(cnn3CCO)c2)cc1F] | 4-(3-amino-1-(1-(2- hydroxyethyl)-1H- indazol-5-yl)-1H- pyrazol-5-yl)-2- fluorobenzonitrile | B-14 | 362/363 |
| 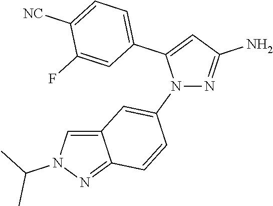 [SMILES: CC(C)n1cc2cc(-n3nc(N)cc3-c3ccc(C#N)c(F)c3)ccc2n1] | 4-(3-amino-1-(2- isopropyl-2H-indazol- 5-yl)-1H-pyrazol-5-yl)- 2-fluorobenzonitrile | B-15 | 360/361 |
| 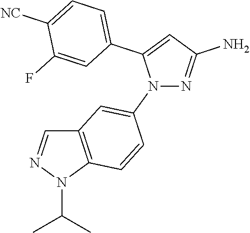 [SMILES: CC(C)n1ncc2cc(-n3nc(N)cc3-c3ccc(C#N)c(F)c3)ccc21] | 4-(3-amino-1-(1- isopropyl-1H-indazol- 5-yl)-1H-pyrazol-5-yl)- 2-fluorobenzonitrile | B-16 | 360/361 |

The following compounds were obtained with methods similar to those used to obtain Intermediate B-1, using the bromo pyrazolamine indicated.

TABLE 8
Pyrazol-3-amines from boronic acids and boronic esters.

| | | | | MWt/ |
|---|---|---|---|---|
| Acid/Ester | Product | IUPAC Name | Int. No | M + H⁺ |
| D-7 | 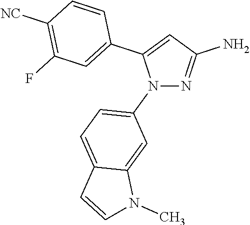 [SMILES: Cn1ccc2ccc(-n3nc(N)cc3-c3ccc(C#N)c(F)c3)cc21] | 4-(3-amino-1-(1- methyl-1H-indol-6- yl)-1H-pyrazol-5- yl)-2- fluorobenzonitrile | B-17 | 331/ 332 |
| D-8 | 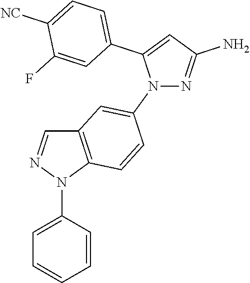 [SMILES: N#Cc1ccc(-c2cc(N)nn2-c2ccc3c(cnn3-c3ccccc3)c2)cc1F] | 4-(3-amino-1-(1- phenyl-1H-indazol- 5-yl)-1H-pyrazol- 5-yl)-2- fluorobenzonitrile | B-18 | 394/ 395 |
| D-9 | 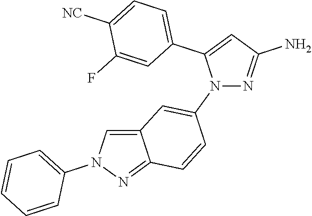 [SMILES: N#Cc1ccc(-c2cc(N)nn2-c2ccc3nn(-c4ccccc4)cc3c2)cc1F] | 4-(3-amino-1-(2- phenyl-2H-indazol- 5-yl)-1H-pyrazol- 5-yl)-2- fluorobenzonitrile | B-19 | 394/ 395 |
| D-10 | 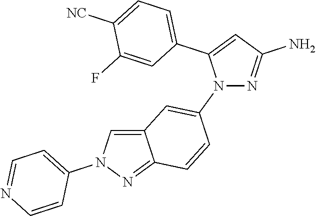 [SMILES: N#Cc1ccc(-c2cc(N)nn2-c2ccc3nn(-c4ccncc4)cc3c2)cc1F] | 4-(3-amino-1-(2- (pyridin-4-yl)-2H- indazol-5-yl)-1H- pyrazol-5-yl)-2- fluorobenzonitrile | B-20 | 395/ 396 |
| D-11 | 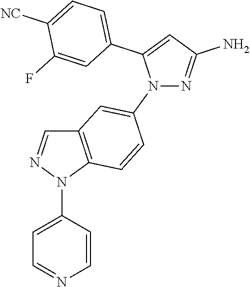 [SMILES: N#Cc1ccc(-c2cc(N)nn2-c2ccc3c(cnn3-c3ccncc3)c2)cc1F] | 4-(3-amino-1-(1- (pyridin-4-yl)-1H- indazol-5-yl)-1H- pyrazol-5-yl)-2- fluorobenzonitrile | B-21 | 395/ 396 |
| D-12 | 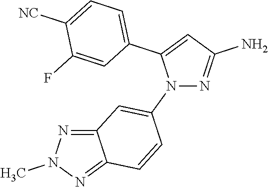 [SMILES: Cn1nc2ccc(-n3nc(N)cc3-c3ccc(C#N)c(F)c3)cc2n1] | 4-(3-amino-1-(2- methyl-2H- benzo[d][1,2,3] triazol-5-yl)-1H- pyrazol-5-yl)-2- fluorobenzonitrile | B-22 | 333/ 334 |
| D-13 | 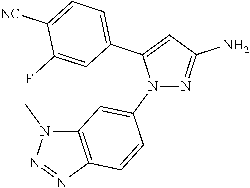 [SMILES: Cn1nnc2ccc(-n3nc(N)cc3-c3ccc(C#N)c(F)c3)cc21] | 4-(3-amino-1-(1- methyl-1H- benzo[d][1,2,3] triazol-6-yl)-1H- pyrazol-5-yl)-2- fluorobenzonitrile | B-23 | 333/ 334 |
| D-14 | 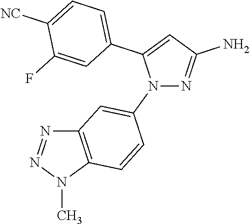 [SMILES: Cn1nnc2cc(-n3nc(N)cc3-c3ccc(C#N)c(F)c3)ccc21] | 4-(3-amino-1-(1- methyl-3a,7a- dihydro-1H- benzo[d]- [1,2,3]triazol-5-yl)- 1H-pyrazol-5-yl)- 2- fluorobenzonitrile | B-24 | 333/ 334 |
| D-15 | 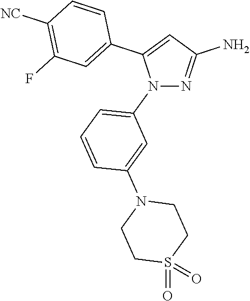 [SMILES: N#Cc1ccc(-c2cc(N)nn2-c2cccc(N3CCS(=O)(=O)CC3)c2)cc1F] | 4-(3-amino-1-(3- (1,1- dioxidothio- morpholino) phenyl)-1H- pyrazol-5-yl)-2- fluorobenzonitrile | B-25 | 411/ 412 |
| D-16 | 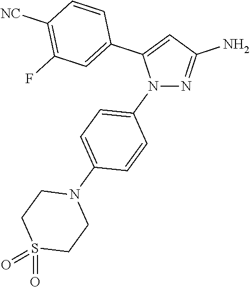 [SMILES: N#Cc1ccc(-c2cc(N)nn2-c2ccc(N3CCS(=O)(=O)CC3)cc2)cc1F] | 4-(3-amino-1-(4- (1,1- dioxidothio- morpholino) phenyl)-1H- pyrazol-5-yl)-2- fluorobenzonitrile | B-26 | 411/ 412 |
| D-17 | 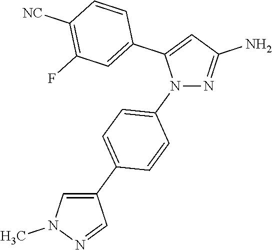 [SMILES: Cn1cc(-c2ccc(-n3nc(N)cc3-c3ccc(C#N)c(F)c3)cc2)cn1] | 4-(3-amino-1-(4- (1-methyl-1H- pyrazol-4- yl)phenyl)-1H- pyrazol-5-yl)-2- fluorobenzonitrile | B-27 | 358/ 359 |
| D-18 | 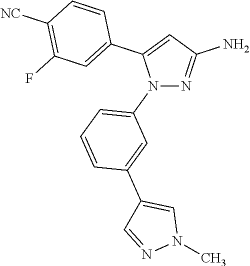 [SMILES: Cn1cc(-c2cccc(-n3nc(N)cc3-c3ccc(C#N)c(F)c3)c2)cn1] | 4-(3-amino-1-(3- (1-methyl-1H- pyrazol-4- yl)phenyl)-1H- pyrazol-5-yl)-2- fluorobenzonitrile | B-28 | 358/ 359 |
| D-19 | 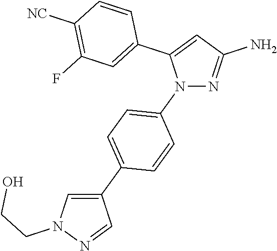 [SMILES: N#Cc1ccc(-c2cc(N)nn2-c2ccc(-c3cnn(CCO)c3)cc2)cc1F] | 4-(3-amino-1-(4- (1-(2- hydroxyethyl)-1H- pyrazol-4- yl)phenyl)-1H- pyrazol-5-yl)-2- fluorobenzonitrile | B-29 | 388/ 389 |
| D-20 | 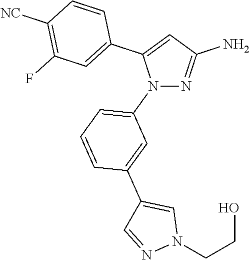 [SMILES: N#Cc1ccc(-c2cc(N)nn2-c2cccc(-c3cnn(CCO)c3)c2)cc1F] | 4-(3-amino-1-(3- (1-(2- hydroxyethyl)-1H- pyrazol-4- yl)phenyl)-1H- pyrazol-5-yl)-2- fluorobenzonitrile | B-30 | 388/ 389 |
| D-21 | 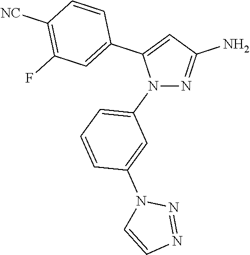 [SMILES: N#Cc1ccc(-c2cc(N)nn2-c2cccc(-n3ccnn3)c2)cc1F] | 4-(1-(3-(1H-1,2,3- triazol-1- yl)phenyl)-3- amino-1H-pyrazol- 5-yl)-2- fluorobenzonitrile | B-31 | 345/ 346 |
| D-22 | 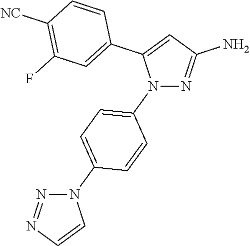 [SMILES: N#Cc1ccc(-c2cc(N)nn2-c2ccc(-n3ccnn3)cc2)cc1F] | 4-(1-(4-(1H-1,2,3- triazol-1- yl)phenyl)-3- amino-1H-pyrazol- 5-yl)-2- fluorobenzonitrile | B-32 | 345/ 346 |
| D-23 | 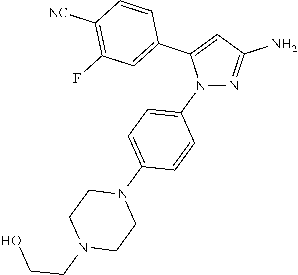 [SMILES: N#Cc1ccc(-c2cc(N)nn2-c2ccc(N3CCN(CCO)CC3)cc2)cc1F] | 4-(3-amino-1-(4- (4-(2- hydroxyethyl) piperazin- 1-yl)phenyl)- 1H-pyrazol-5-yl)- 2- fluorobenzonitrile | B-33 | 406/ 407 |
| D-24 | 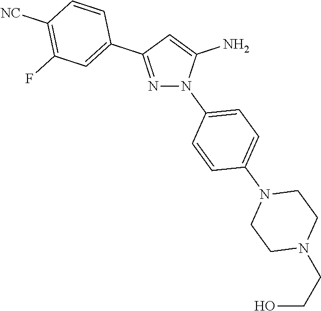 [SMILES: N#Cc1ccc(-c2cc(N)n(-c3ccc(N4CCN(CCO)CC4)cc3)n2)cc1F] | 4-(5-amino-1-(4- (4-(2- hydroxyethyl) piperazin- 1-yl)phenyl)- 1H-pyrazol-3-yl)- 2- fluorobenzonitrile | B-34 | 406/ 407 |
| D-25 | 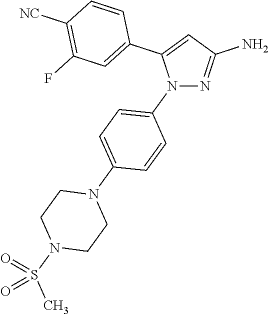 [SMILES: CS(=O)(=O)N1CCN(c2ccc(-n3nc(N)cc3-c3ccc(C#N)c(F)c3)cc2)CC1] | 4-(3-amino-1-(4- (4- (methylsulfonyl)- piperazin-1- yl)phenyl)-1H- pyrazol-5-yl)-2- fluorobenzonitrile | B-35 | 440/ 441 |
| D-26 | 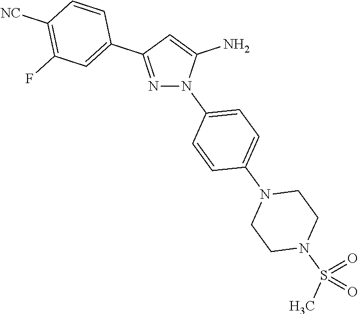 [SMILES: CS(=O)(=O)N1CCN(c2ccc(-n3nc(-c4ccc(C#N)c(F)c4)cc3N)cc2)CC1] | 4-(5-amino-1-(4- (4- (methylsulfonyl) piperazin-1- yl)phenyl)-1H- pyrazol-3-yl)-2- fluorobenzonitrile | B-36 | 440/ 441 |
| D-27 | 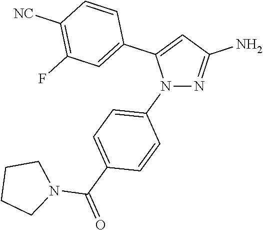 [SMILES: N#Cc1ccc(-c2cc(N)nn2-c2ccc(C(=O)N3CCCC3)cc2)cc1F] | 4-(3-amino-1-(4- (pyrrolidine-1- carbonyl)phenyl)- 1H-pyrazol-5-yl)- 2- fluorobenzonitrile | B-37 | 375/ 376 |

### Intermediate B-38

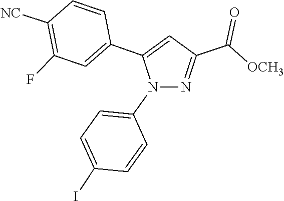 [SMILES: COC(=O)c1cc(-c2ccc(C#N)c(F)c2)n(-c2ccc(I)cc2)n1]

### Methyl 5-(4-cyano-3-fluorophenyl)-1-(4-iodophenyl)-1H-pyrazole-3-carboxylate

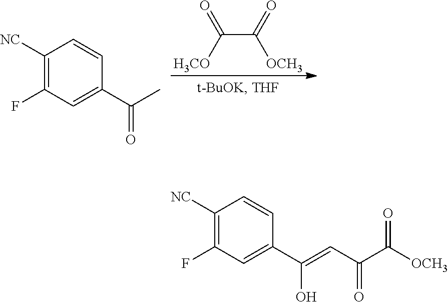 [SMILES: CC(=O)c1ccc(C#N)c(F)c1 ; COC(=O)C(=O)OC ; COC(=O)C(=O)/C=C(\O)c1ccc(C#N)c(F)c1]

Methyl (Z)-4-(4-cyano-3-fluorophenyl)-4-hydroxy-2-oxobut-3-enoate To a solution of t-BuOK (8.05 g, 71.7 mmol, 1.30 equiv) in THF (200 mL) was added 4-acetyl-2-fluorobenzonitrile (9.0 g, 55.2 mmol, 1.00 equiv) dropwise at 0° C. under N₂. The resulting mixture was stirred for 1 h at 0° C. under nitrogen. To above mixture was added dimethyl oxalate (7.82 g, 66.2 mmol, 1.20 equiv) dropwise over 10 min at 0° C. The resulting mixture was stirred overnight at room temperature. The precipitated solids were collected by filtration and washed with THF (10 mL). The residue was dissolved in water (100 mL) and adjusted to pH 3-4 with HCl (2 M). The precipitated solids were collected by filtration and washed with water (10 mL) to afford the title compound (8.0 g, 49%) as a yellow solid. MS-ESI: 250 (M+1).

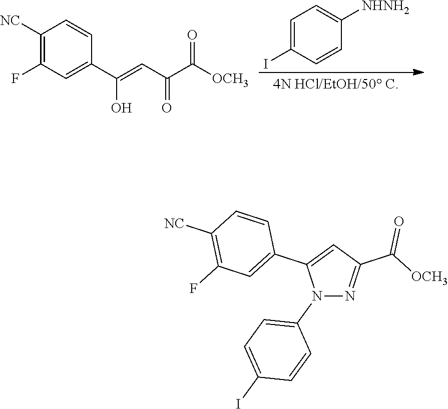 [SMILES: COC(=O)C(=O)/C=C(\O)c1ccc(C#N)c(F)c1 ; NNc1ccc(I)cc1 ; COC(=O)c1cc(-c2ccc(C#N)c(F)c2)n(-c2ccc(I)cc2)n1]

Methyl 5-(4-cyano-3-fluorophenyl)-1-(4-iodophenyl)-1H-pyrazole-3-carboxylate To a solution of the product from the previous step (5.0 g, 20 mmol, 1.00 equiv) in EtOH (100 mL) were added (4-iodophenyl) hydrazine (4.7 g, 20 mmol, 1.00 equiv) and HCl (gas) in 1,4-dioxane (4 M, 5.02 mL, 20 mmol, 1.00 equiv) dropwise. The resulting mixture was stirred overnight at 50° C., then concentrated under vacuum. The residue was purified with silica gel chromatography using PE/EtOAc (5:1) to afford the title compound (900 mg, 8.42%) as a yellow solid. MS-ESI: 448 (M+1).

### Intermediate B-39

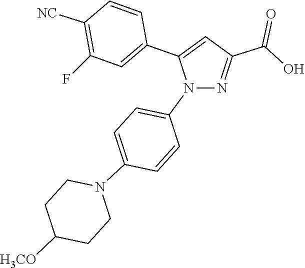 [SMILES: COC1CCN(c2ccc(-n3nc(C(=O)O)cc3-c3ccc(C#N)c(F)c3)cc2)CC1]

### 5-(4-Cyano-3-fluorophenyl)-1-(4-(4-methoxypiperidin-1-yl)phenyl)-1H-pyrazole-3-carboxylic acid

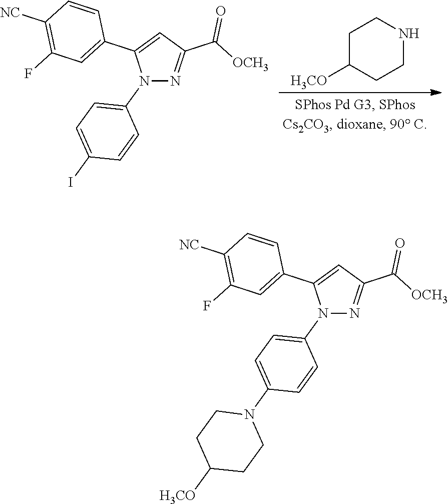 [SMILES: COC(=O)c1cc(-c2ccc(C#N)c(F)c2)n(-c2ccc(I)cc2)n1 ; COC1CCNCC1 ; COC(=O)c1cc(-c2ccc(C#N)c(F)c2)n(-c2ccc(N3CCC(OC)CC3)cc2)n1]

Methyl 5-(4-cyano-3-fluorophenyl)-1-(4-(4-methoxypiperidin-1-yl)phenyl)-1H-pyrazole-3-carboxylate To a solution of Intermediate B-38 (850 mg, 1.84 mmol, 1.00 equiv) and 4-methoxypiperidine (425 mg, 3.67 mmol, 2.00 equiv) in dioxane (15 mL) were added SPhos Pd Gen.3 (144 mg, 0.184 mmol, 0.10 equiv), SPhos (75.7 mg, 0.184 mmol, 0.10 equiv) and Cs₂CO₃ (1.20 g, 3.67 mmol, 2.00 equiv). The resulting mixture was stirred for 16 h at 90° C. under N₂. The resulting mixture was concentrated under vacuum. The residue was purified with silica gel chromatography using PE/EtOAc (1:1) to afford the title compound (670 mg, 64%) as a yellow solid. MS-ESI: 435 (M+1).

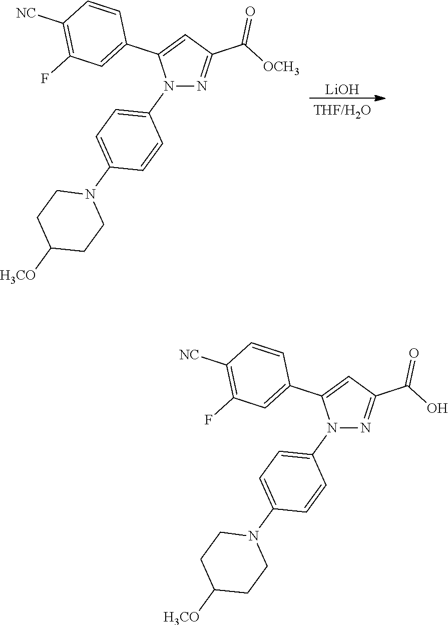 [SMILES: COC(=O)c1cc(-c2ccc(C#N)c(F)c2)n(-c2ccc(N3CCC(OC)CC3)cc2)n1 ; "LiOH" ; COC1CCN(c2ccc(-n3nc(C(=O)O)cc3-c3ccc(C#N)c(F)c3)cc2)CC1]

5-(4-Cyano-3-fluorophenyl)-1-(4-(4-methoxypiperidin-1-yl)phenyl)-1H-pyrazole-3-carboxylic acid To a solution of the product from the previous step (650 mg, 1.45 mmol, 1.00 equiv) in THF (16 mL) and H₂O (8.00 mL) were added LiOH (52 mg, 2.17 mmol, 1.50 equiv) in portions. The resulting mixture was stirred for 1 h at room temperature. The mixture was adjusted to pH 3-4 with HCl (2 M) and extracted with EtOAc (3×30 mL). The combined organic layers were dried with anhydrous Na₂SO₄ and concentrated under vacuum to afford the title compound (620 mg, 81%) as a yellow solid. MS-ESI: 421 (M+1).

The following carboxylic acids were obtained using similar methods.

TABLE 9
Pyrazol-3-carboxylic acids.

| | | | MWt/ |
|---|---|---|---|
| Product | IUPAC Name | Int. No | M + H⁺ |
| 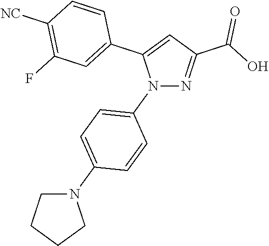 [SMILES: N#Cc1ccc(-c2cc(C(=O)O)nn2-c2ccc(N3CCCC3)cc2)cc1F] | 5-(4-cyano-3- fluorophenyl)-1-(4- (pyrrolidin-1- yl)phenyl)-1H- pyrazole-3- carboxylic acid | B-40 | 376/ 377 |
| 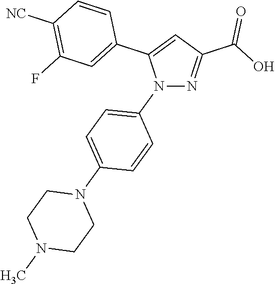 [SMILES: CN1CCN(c2ccc(-n3nc(C(=O)O)cc3-c3ccc(C#N)c(F)c3)cc2)CC1] | 5-(4-cyano-3- fluorophenyl)-1-(4- (4-methylpiperazin- 1-yl)phenyl)-1H- pyrazole-3- carboxylic acid | B-41 | 405/ 406 |

### Intermediate B-42

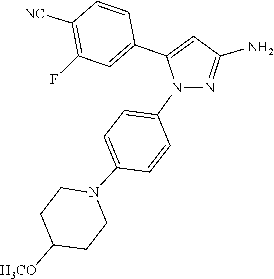 [SMILES: COC1CCN(c2ccc(-n3nc(N)cc3-c3ccc(C#N)c(F)c3)cc2)CC1]

### 4-(3-Amino-1-(4-(4-methoxypiperidin-1-yl)phenyl)-1H-pyrazol-5-yl)-2-fluorobenzonitrile

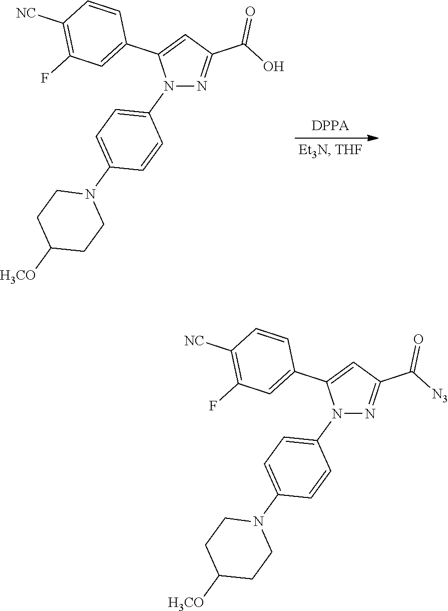 [SMILES: COC1CCN(c2ccc(-n3nc(C(=O)O)cc3-c3ccc(C#N)c(F)c3)cc2)CC1 ; COC1CCN(c2ccc(-n3nc(C(=O)N=[N+]=[N-])cc3-c3ccc(C#N)c(F)c3)cc2)CC1]

5-(4-Cyano-3-fluorophenyl)-1-(4-(4-methoxypiperidin-1-yl)phenyl)-1H-pyrazole-3-carbonyl azide To a solution of 5-(4-cyano-3-fluorophenyl)-1-(4-(4-methoxypiperidin-1-yl)phenyl)-1H-pyrazole-3-carboxylic acid (600 mg, 1.43 mmol, 1.00 equiv) and DPPA (1.57 g, 5.71 mmol, 4.00 equiv) in THF (10 mL) was added TEA (577 mg, 5.7 mmol, 4.00 equiv) dropwise. The resulting mixture was stirred for 24 h at room temperature, then concentrated under vacuum to afford the title compound (850 mg, crude) as a yellow oil. MS-ESI: 446 (M+1).

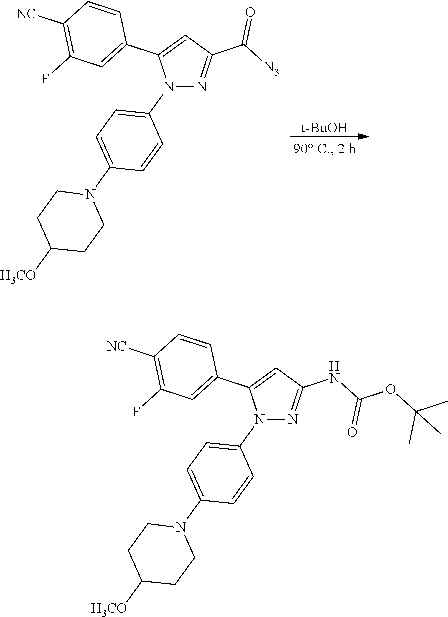 [SMILES: "t-BuOH" ; COC1CCN(c2ccc(-n3nc(C(=O)N=[N+]=[N-])cc3-c3ccc(C#N)c(F)c3)cc2)CC1 ; COC1CCN(c2ccc(-n3nc(NC(=O)OC(C)(C)C)cc3-c3ccc(C#N)c(F)c3)cc2)CC1]

tert-Butyl (5-(4-cyano-3-fluorophenyl)-1-(4-(4-methoxypiperidin-1-yl)phenyl)-1H-pyrazol-3-yl) carbamate A solution of the product from the previous step (850 mg, crude) in t-BuOH (10 mL) was stirred for 2 h at 90° C. The resulting mixture was concentrated under vacuum. The residue was purified with silica gel using PE/EtOAc (2:1) to afford the title compound (300 mg, 32%) as a yellow solid. MS-ESI: 492 (M+1).

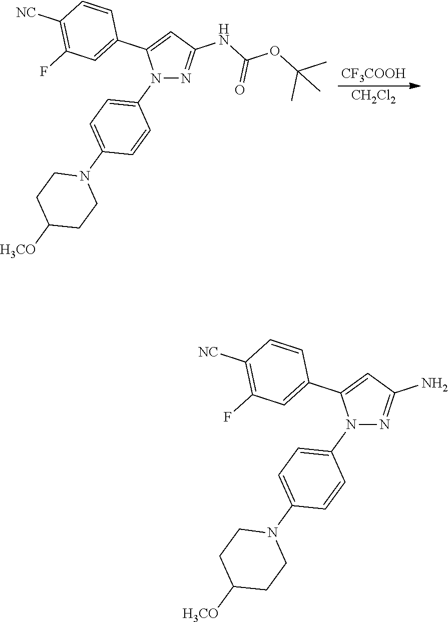

4-(3-Amino-1-(4-(4-methoxypiperidin-1-yl)phenyl)-1H-pyrazol-5-yl)-2-fluorobenzonitrile To a solution of the product from the previous step (280 mg, 0.57 mmol, 1.00 equiv) in CH₂Cl₂ (10 mL) was added CF₃COOH (2 mL) dropwise. The resulting mixture was stirred for 2 h at room temperature. The mixture was adjusted to pH 7-8 with sat. Na₂CO₃ (aq.). The resulting mixture was diluted with water (20 mL) and extracted with DCM (3×20 mL). The combined organic layers were concentrated under vacuum to afford the title compound (200 mg, 69%) as a yellow solid. MS-ESI: 392 (M+1).

The following amine was obtained using similar methods.

TABLE 10
Pyrazol-3-amines.

| | | | MWt/ |
|---|---|---|---|
| Product | IUPAC Name | Int. No | M + H⁺ |
| 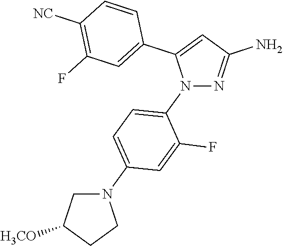 [SMILES: CO[C@H]1CCN(c2ccc(-n3nc(N)cc3-c3ccc(C#N)c(F)c3)c(F)c2)C1] | (S)-4-(3-amino-1-(2- fluoro-4-(3- methoxypyrrolidin- 1-yl)phenyl)-1H- pyrazol-5-yl)-2- fluorobenzonitrile | B-43 | 395/ 396 |

### Intermediate B-44

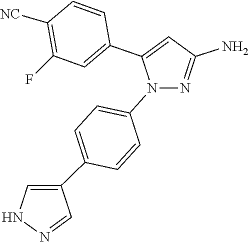 [SMILES: N#Cc1ccc(-c2cc(N)nn2-c2ccc(-c3cn[nH]c3)cc2)cc1F]

### 4-(1-(4-(1H-pyrazol-4-yl)phenyl)-3-amino-1H-pyrazol-5-yl)-2-fluorobenzonitrile

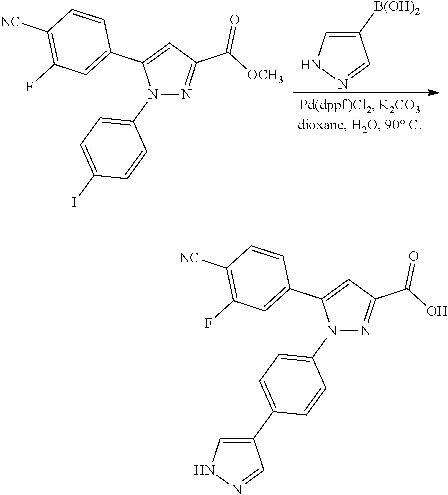 [SMILES: COC(=O)c1cc(-c2ccc(C#N)c(F)c2)n(-c2ccc(I)cc2)n1 ; OB(O)c1cn[nH]c1 ; N#Cc1ccc(-c2cc(C(=O)O)nn2-c2ccc(-c3cn[nH]c3)cc2)cc1F]

1-(4-(1H-pyrazol-4-yl)phenyl)-5-(4-cyano-3-fluorophenyl)-1H-pyrazole-3-carboxylic acid To a solution of Intermediate B-38 (500 mg, 1.1 mmol, 1.0 equiv) and (1H-pyrazol-4-yl) boronic acid (250 mg, 2.2 mmol, 2.0 equiv) in dioxane (10 mL) and H₂O (1 mL) were added Pd(dppf)Cl₂ (82 mg, 0.11 mmol, 0.1 equiv) and K₂CO₃ (464 mg, 3.4 mmol, 3.0 equiv) at room temperature under N₂. The resulting mixture was stirred overnight at 90° C. under N₂. The mixture was adjusted to pH 3 with HCl (2 M). The mixture was diluted with H₂O (10 mL) and extracted with EtOAc (3×20 mL). The combined organic layers were dried with anhydrous Na₂SO₄ and concentrated under vacuum to afford the title compound (350 mg, crude) as a light yellow solid. MS-ESI: 374 (M+1).

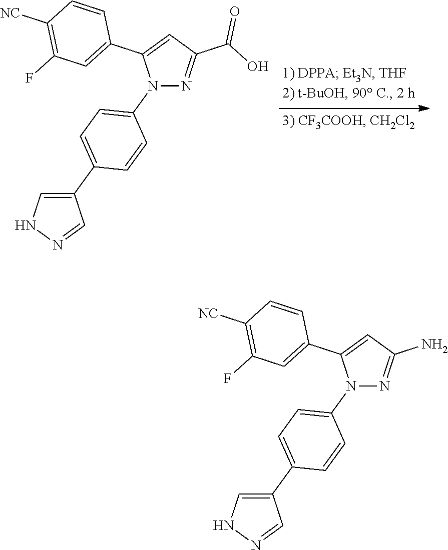 [SMILES: N#Cc1ccc(-c2cc(C(=O)O)nn2-c2ccc(-c3cn[nH]c3)cc2)cc1F ; N#Cc1ccc(-c2cc(N)nn2-c2ccc(-c3cn[nH]c3)cc2)cc1F]

4-(1-(4-(1H-pyrazol-4-yl)phenyl)-3-amino-1H-pyrazol-5-yl)-2-fluorobenzonitrile Synthesis was accomplished using analogous steps to those used to obtain B-42. MS-ESI: 345 (M+1).

The following amine was obtained using similar methods.

TABLE 11
Pyrazol-3-amines.

| | | | MWt/ |
|---|---|---|---|
| Product | IUPAC Name | Int. No | M + H⁺ |
| 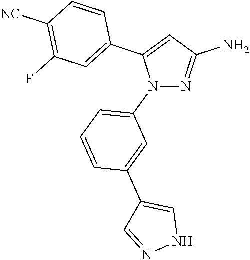 [SMILES: N#Cc1ccc(-c2cc(N)nn2-c2cccc(-c3cn[nH]c3)c2)cc1F] | 4-(1-(3-(1H-pyrazol- 4-yl)phenyl)-3- amino-1H-pyrazol- 5-yl)-2- fluorobenzonitrile | B-45 | 344/ 345 |

### Intermediate B-46

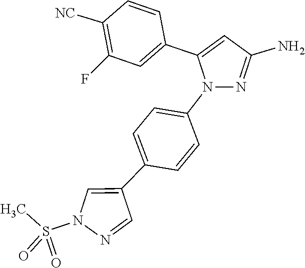 [SMILES: CS(=O)(=O)n1cc(-c2ccc(-n3nc(N)cc3-c3ccc(C#N)c(F)c3)cc2)cn1]

### 4-(3-amino-1-(4-(1-(methylsulfonyl)-1H-pyrazol-4-yl)phenyl)-1H-pyrazol-5-yl)-2-fluorobenzonitrile

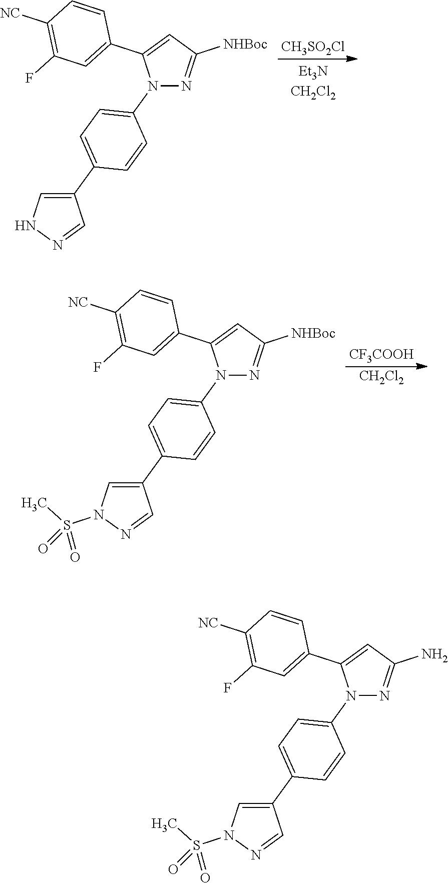

tert-Butyl (5-(4-cyano-3-fluorophenyl)-1-(4-(1-(methylsulfonyl)-1H-pyrazol-4-yl)phenyl)-1H-pyrazol-3-yl) carbamate To a solution of tert-butyl (1-(4-(1H-pyrazol-4-yl)phenyl)-5-(4-cyano-3-fluorophenyl)-1H-pyrazol-3-yl) carbamate (150 mg, 0.34 mmol, 1.0 equiv) in CH₂Cl₂ (3 mL) was added CH₃SO₂Cl (46 mg, 0.41 mmol, 1.2 equiv) and Et₃N (44 mg, 0.44 mmol, 1.3 equiv) dropwise at 0° C. under N₂. The resulting mixture was stirred for 2 h at room temperature. The mixture was concentrated under vacuum and purified with silica gel chromatography using PE/EtOAc (1:1) to afford the title compound (90 mg, 51%) as a white solid. MS-ESI: 523 (M+1).

4-(3-amino-1-(4-(1-(methylsulfonyl)-1H-pyrazol-4-yl)phenyl)-1H-pyrazol-5-yl)-2-fluorobenzonitrile Synthesis was accomplished using a procedure similar to that used to obtain B-42. MS-ESI: 423 (M+1).

The following amine was obtained using similar methods.

TABLE 12
Pyrazol-3-amines.

| | | | MWt/ |
|---|---|---|---|
| Product | IUPAC Name | Int. No | M + H⁺ |
| 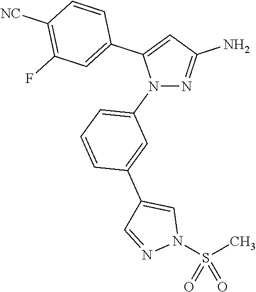 [SMILES: CS(=O)(=O)n1cc(-c2cccc(-n3nc(N)cc3-c3ccc(C#N)c(F)c3)c2)cn1] | 4-(3-amino-1-(3-(1- (methylsulfonyl)- 1H-pyrazol-4- yl)phenyl)-1H- pyrazol-5-yl)-2- fluorobenzonitrile | B-47 | 422/ 423 |

The following compounds were obtained with methods similar to those used to obtain Intermediate B-1.

| | | | MWt/ |
|---|---|---|---|
| Structure | IUPAC Name | Int. No. | M + H⁺ |
| 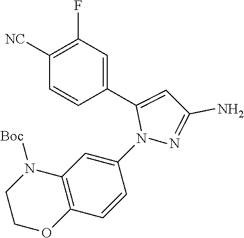 [SMILES: CC(C)(C)OC(=O)N1CCOc2ccc(-n3nc(N)cc3-c3ccc(C#N)c(F)c3)cc21] | Tert-butyl 6-(3-amino- 5-(4-cyano-3- fluorophenyl)-1H- pyrazol-1-yl)-2,3- dihydro-4H- benzo[b][1,4]oxazine- 4-carboxylate | B-48 | 436 |

### Example 2

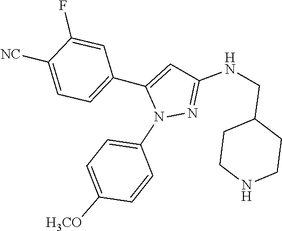 [SMILES: COc1ccc(-n2nc(NCC3CCNCC3)cc2-c2ccc(C#N)c(F)c2)cc1]

### 2-Fluoro-4-(1-(4-methoxyphenyl)-3-((piperidin-4-ylmethyl)amino)-1H-pyrazol-5-yl)benzonitrile

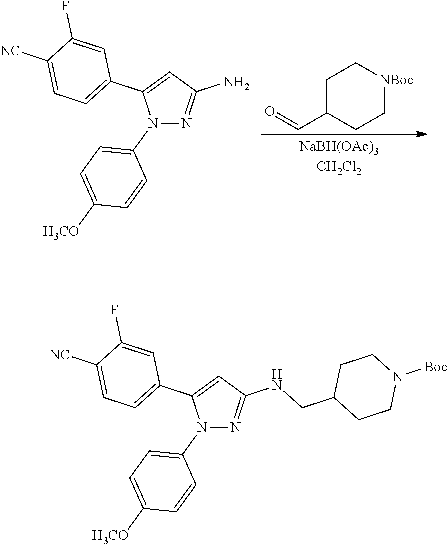 [SMILES: COc1ccc(-n2nc(N)cc2-c2ccc(C#N)c(F)c2)cc1 ; CC(C)(C)OC(=O)N1CCC(C=O)CC1 ; COc1ccc(-n2nc(NCC3CCN(C(=O)OC(C)(C)C)CC3)cc2-c2ccc(C#N)c(F)c2)cc1]

tert-Butyl 4-(((5-(4-cyano-3-fluorophenyl)-1-(4-methoxyphenyl)-1H-pyrazol-3-yl)amino)methyl) piperidine-1-carboxylate To a stirred solution of Intermediate B-1 (200 mg, 0.65 mmol, 1.00 equiv) in CH₂Cl₂ (20 mL) were added tert-butyl 4-formylpiperidine-1-carboxylate (208 mg, 0.97 mmol, 1.50 equiv) and NaBH(OAc)₃ (412 mg, 1.95 mmol, 3.00 equiv) dropwise at rt. The resulting mixture was stirred 1 h at rt. The reaction was quenched with MeOH at rt. The resulting mixture was concentrated under reduced pressure and purified with Prep-TLC (petroleum ether/EtOAc 1:1) to afford the title compound (140 mg, 42.6%) as a yellow solid.

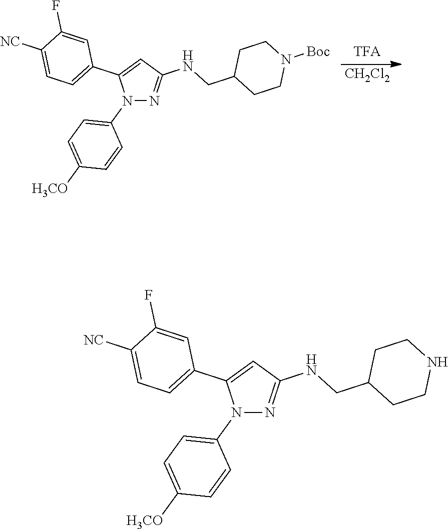

2-Fluoro-4-(1-(4-methoxyphenyl)-3-((piperidin-4-ylmethyl)amino)-1H-pyrazol-5-yl)benzonitrile To a solution of the product from the previous step (140 mg, 0.28 mmol, 1.00 equiv) in CH₂Cl₂ (30 mL) was added TFA (3 mL) at rt. The above solution was stirred 1 h at rt, then concentrated under reduced pressure. The crude product was purified with Prep-HPLC with the following conditions: Sunfire prep C18 column, 30×150, 5 um; mobile phase A: H₂O (0.05% TFA), mobile phase B: CH₃CN; flow rate: 60 mL/min; gradient: 20% B to 45% B in 7 min; 1=254/210 nm; to afford the trifluoroacetate salt of the title compound (60 mg, 35.6%) as a yellow solid. LC-MS: (ES, m/z): 406 [M+H]⁺. ¹H NMR (400 MHZ, CD₃OD) δ ppm: 7.68-7.64 (t, J=7.4 Hz, 1H), 7.21-7.14 (m, 4H), 6.97-6.93 (m, 2H), 6.08 (s, 1H), 3.82 (s, 3H), 3.43-3.40 (m, 2H), 3.16-3.14 (d, J=6.8 Hz, 2H), 3.02-2.95 (m, 2H), 2.08-1.95 (m, 3H), 1.48-1.37 (m, 2H).

### Example 3

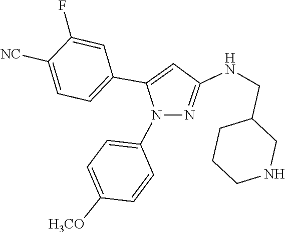 [SMILES: COc1ccc(-n2nc(NCC3CCCNC3)cc2-c2ccc(C#N)c(F)c2)cc1]

### 2-fluoro-4-(1-(4-methoxyphenyl)-3-((piperidin-3-ylmethyl)amino)-1H-pyrazol-5-yl)benzonitrile

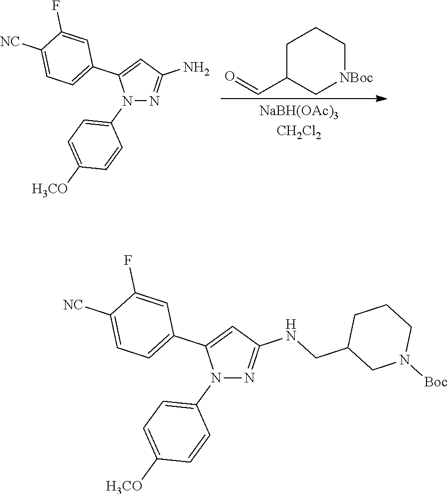

tert-Butyl 3-(((5-(4-cyano-3-fluorophenyl)-1-(4-methoxyphenyl)-1H-pyrazol-3-yl)amino)methyl) piperidine-1-carboxylate To a solution of Intermediate B-1 (300 mg, 0.97 mmol, 1.00 equiv) in CH₂Cl₂ (10 mL) was added tert-butyl 3-formylpiperidine-1-carboxylate (311 mg, 1.46 mmol, 1.50 equiv). The mixture was stirred 20 min at rt, then NaBH(OAc)₃ (619 mg, 2.92 mmol, 3.00 equiv) was added dropwise at rt. The resulting mixture was stirred an additional 1 h at rt, then concentrated under reduced pressure and purified with Prep-TLC (petroleum ether/EtOAc 1:1) to afford the title compound (220 mg, 44.7%) as a white solid.

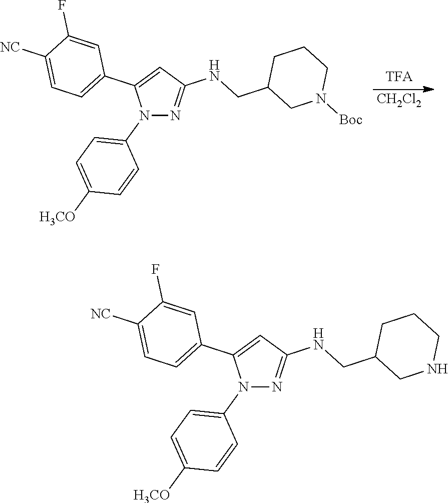

2-Fluoro-4-(1-(4-methoxyphenyl)-3-((piperidin-3-ylmethyl)amino)-1H-pyrazol-5-yl)benzonitrile To a solution of the product from the previous step (200 mg, 0.40 mmol, 1.00 equiv) in CH₂Cl₂ (20 mL) was added TFA (4 mL) at rt. The resulting mixture was stirred 1 h at rt, then concentrated under reduced pressure and purified with Prep-HPLC with the following conditions: XSelect CSH Prep C18 OBD Column, 5 μm, 19×150 mm; mobile phase A: H₂O (0.05% TFA), mobile phase B: CH₃CN; flow rate: 25 mL/min; gradient: 20% B to 45% B in 7 min; 1=254/210 nm; RT1:5.08 to afford the trifluoroacetate salt of the title compound (73 mg, 35.5%) as a yellow solid. LC-MS: (ES, m/z): 406 [M+H]⁺. ¹H NMR (300 MHz, CD₃OD) δ ppm: 7.70-7.65 (m, 1H), 7.23-7.16 (m, 4H), 6.99-6.96 (m, 2H), 6.12 (s, 1H), 3.84 (s, 3H), 3.50-3.32 (m, 2H), 3.28-3.14 (m, 2H), 2.94-2.91 (m, 1H), 2.77-2.73 (m, 1H), 2.19-2.16 (m, 1H), 2.01-1.95 (m, 2H), 1.84-1.68 (m, 1H), 1.41-1.26 (m, 1H).

The following achiral/racemic compounds were obtained using similar procedures to that used for Example 3, using achiral/racemic aldehydes.

TABLE 13
Aminomethyl pyrazoles from achiral/racemic aldehydes.

| Ex. | | | | |
|---|---|---|---|---|
| No. | Amine | Aldehyde | Deprotected Product | IUPAC Name |
| 4 | B-1 | 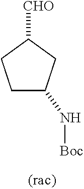 [SMILES: CC(C)(C)OC(=O)N[C@@H]1CC[C@H](C=O)C1] | 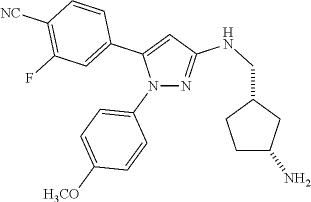 | rac-4-(3-((((1S,3R)-3- aminocyclopentyl) methyl)amino)-1-(4- methoxyphenyl)-1H- pyrazol-5-yl)-2- fluorobenzonitrile |
| 5 | B-1 | 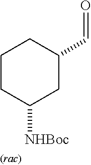 [SMILES: CC(C)(C)OC(=O)N[C@@H]1CCC[C@H](C=O)C1] | 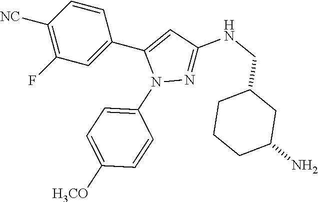 [SMILES: COc1ccc(-n2nc(NC[C@H]3CCC[C@@H](N)C3)cc2-c2ccc(C#N)c(F)c2)cc1] | rac-4-(3-((((15,3R)-3- aminocyclohexyl) methyl)amino)-1-(4- methoxyphenyl)-1H- pyrazol-5-yl)-2- fluorobenzonitrile |
| 6 | B-1 | 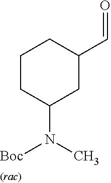 [SMILES: CN(C(=O)OC(C)(C)C)C1CCCC(C=O)C1] | 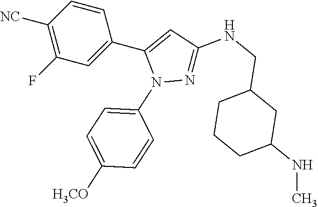 [SMILES: CNC1CCCC(CNc2cc(-c3ccc(C#N)c(F)c3)n(-c3ccc(OC)cc3)n2)C1] | rac-2-fluoro-4-(1-(4- methoxyphenyl)-3- (((-3-(methylamino)- cyclohexyl)methyl)- amino)-1H-pyrazol-5- yl)benzonitrile |
| 7 | B-10 | 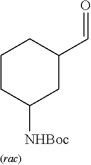 [SMILES: CC(C)(C)OC(=O)NC1CCCC(C=O)C1] | 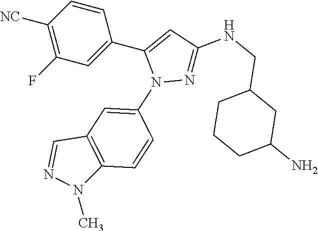 [SMILES: Cn1ncc2cc(-n3nc(NCC4CCCC(N)C4)cc3-c3ccc(C#N)c(F)c3)ccc21] | rac-4-(3-(((3- aminocyclohexyl) methyl)amino)-1-(1- methyl-1H-indazol-5- yl)-1H-pyrazol-5-yl)- 2-fluorobenzonitrile |
| 8 | B-1 | 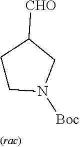 [SMILES: CC(C)(C)OC(=O)N1CCC(C=O)C1] | 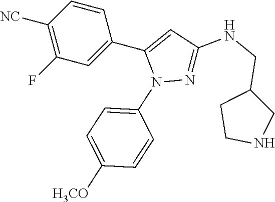 [SMILES: COc1ccc(-n2nc(NCC3CCNC3)cc2-c2ccc(C#N)c(F)c2)cc1] | 2-fluoro-4-(1-(4- methoxyphenyl)-3- ((pyrrolidin-3- ylmethyl)amino)-1H- pyrazol-5- yl)benzonitrile |
| 9 | B-3 | 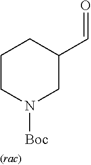 [SMILES: CC(C)(C)OC(=O)N1CCCC(C=O)C1] | 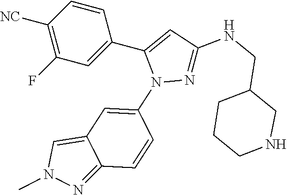 [SMILES: Cn1cc2cc(-n3nc(NCC4CCCNC4)cc3-c3ccc(C#N)c(F)c3)ccc2n1] | 2-Fluoro-4-(1-(2- methyl-2H-indazol-5- yl)-3-((piperidin-3- ylmethyl)amino)-1H- pyrazol-5- yl)benzonitrile |
| 10 | B-3 | 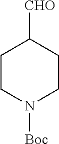 [SMILES: CC(C)(C)OC(=O)N1CCC(C=O)CC1] | 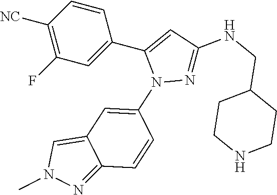 [SMILES: Cn1cc2cc(-n3nc(NCC4CCNCC4)cc3-c3ccc(C#N)c(F)c3)ccc2n1] | 2-Fluoro-4-(1-(2- methyl-2H-indazol-5- yl)-3-((piperidin-4- ylmethyl)amino)-1H- pyrazol-5- yl)benzonitrile |

### Example 11

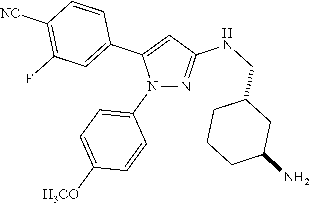

### 4-(3-((((1S,3S)-3-aminocyclohexyl)methyl)amino)-1-(4-methoxyphenyl)-1H-pyrazol-5-yl)-2-fluorobenzonitrile

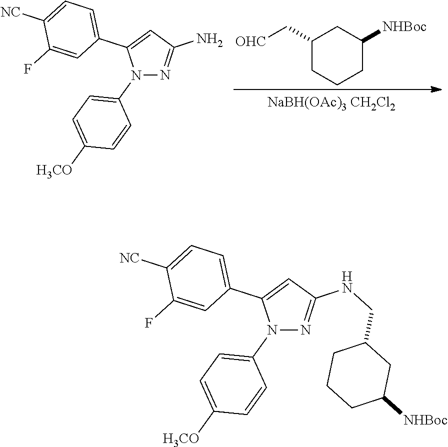 [SMILES: COc1ccc(-n2nc(N)cc2-c2ccc(C#N)c(F)c2)cc1 ; CC(C)(C)OC(=O)N[C@H]1CCC[C@H](CC=O)C1 ; COc1ccc(-n2nc(NC[C@H]3CCC[C@H](NC(=O)OC(C)(C)C)C3)cc2-c2ccc(C#N)c(F)c2)cc1]

tert-Butyl ((1S,3S)-3-(((5-(4-cyano-3-fluorophenyl)-1-(4-methoxyphenyl)-1H-pyrazol-3-yl)amino)methyl)cyclohexyl) carbamate A solution of Intermediate B-1 (2.00 g, 6.49 mmol, 1.00 equiv) in CH₂Cl₂ (40 mL) was added tert-butyl ((1S,3S)-3-formyl-cyclohexyl) carbamate (2.21 g, 9.73 mmol, 1.50 equiv). The mixture was stirred for 30 min at room temperature, then NaBH(OAc)₃ (4.12 g, 19.5 mmol, 3.00 equiv) was added in portions at room temperature. The mixture was stirred for additional 30 min at room temperature, then concentrated under vacuum. The residue was purified with silica gel using PE/EtOAc (1:1) to afford the title compound (1.6 g, 47.5%) as a yellow solid. MS-ESI: 520 (M+1).

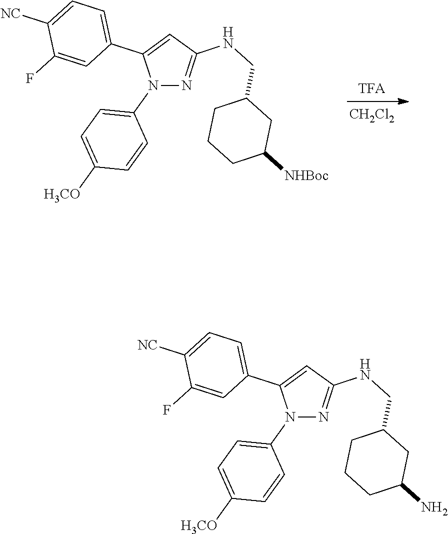 [SMILES: COc1ccc(-n2nc(NC[C@H]3CCC[C@H](NC(=O)OC(C)(C)C)C3)cc2-c2ccc(C#N)c(F)c2)cc1 ; COc1ccc(-n2nc(NC[C@H]3CCC[C@H](N)C3)cc2-c2ccc(C#N)c(F)c2)cc1]

4-(3-((((1S,3S)-3-aminocyclohexyl)methyl)amino)-1-(4-methoxyphenyl)-1H-pyrazol-5-yl)-2-fluorobenzonitrile To a solution of the product from the previous step (1.6 g, 3.08 mmol, 1.00 equiv) in CH₂Cl₂ (40 mL) was added CF₃COOH (5 mL) dropwise at room temperature. The resulting mixture was stirred for 2 h at room temperature, then concentrated under vacuum. The crude product was purified with Prep-HPLC with the following conditions: Xselect CSH OBD Column, 30*150 mm 5 um; Mobile Phase A: water (0.05% TFA), Mobile Phase B: ACN; Flow rate: 60 mL/min; Gradient: 17% ACN to 39% ACN in 7 min; Detector UV: 210/254 nm; RT1(min): 6.23 to afford the title compound (890 mg, 54%) as a yellow solid.

MS-ESI: 420 (M+1).

¹H NMR (400 MHZ, MeOH-d₄) 7.69 (t, J=8.0 Hz, 1H), 7.25-7.15 (m, 4H), 7.02-6.95 (m, 2H), 6.11 (s, 1H), 3.84 (s, 3H), 3.54-3.43 (m, 1H), 3.27-3.12 (m, 2H), 2.15-2.07 (m, 1H), 1.95-1.88 (m, 1H), 1.87-1.79 (m, 2H), 1.78-1.55 (m, 4H), 1.53-1.41 (m, 1H).

The following enantiopure compounds were obtained using similar procedures to that used for Example 3, using enantiopure aldehydes.

TABLE 14
Aminomethyl pyrazoles from enantiopure aldehydes.

| Ex. | | | | |
|---|---|---|---|---|
| No. | Amine | Aldehyde | Deprotected Product | IUPAC Name |
| 3R | B-1 | 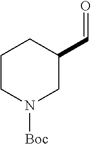 [SMILES: CC(C)(C)OC(=O)N1CCC[C@@H](C=O)C1] | 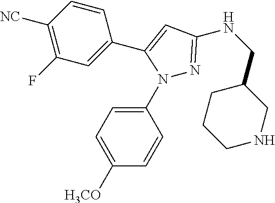 | (R)-2-fluoro-4-(1-(4- methoxyphenyl)-3- ((piperidin-3- ylmethyl)amino)-1H- pyrazol-5- yl)benzonitrile |
| 3S | B-1 | 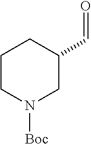 [SMILES: CC(C)(C)OC(=O)N1CCC[C@H](C=O)C1] | 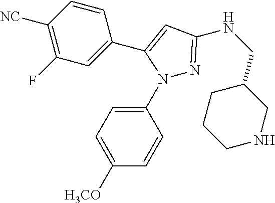 | (S)-2-fluoro-4-(1-(4- methoxyphenyl)-3- ((piperidin-3- ylmethyl)amino)-1H- pyrazol-5- yl)benzonitrile |
| 11RR | B-1 | 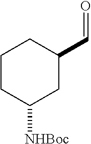 [SMILES: CC(C)(C)OC(=O)N[C@@H]1CCC[C@@H](C=O)C1] | 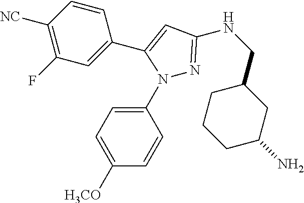 | 4-(3-((((1R,3R)-3- aminocyclohexyl) methyl)amino)-1-(4- methoxyphenyl)-1H- pyrazol-5-yl)-2- fluorobenzonitrile |
| 11SS | B-1 | 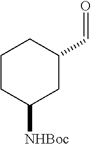 [SMILES: CC(C)(C)OC(=O)N[C@H]1CCC[C@H](C=O)C1] | 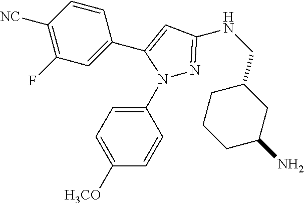 | 4-(3-((((1S,3S)-3- aminocyclohexyl) methyl)amino)-1-(4- methoxyphenyl)-1H- pyrazol-5-yl)-2- fluorobenzonitrile |
| 12RR | B-2 | 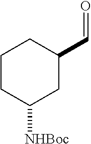 [SMILES: CC(C)(C)OC(=O)N[C@@H]1CCC[C@@H](C=O)C1] | 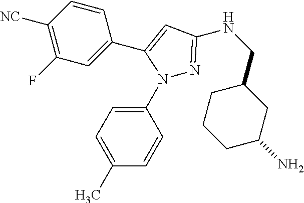 | 4-(3-((((1R,3R)-3- aminocyclohexyl) methyl)amino)-1-(p- tolyl)-1H-pyrazol-5- yl)-2- fluorobenzonitrile |
| 12SS | B-1 | 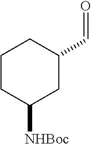 [SMILES: CC(C)(C)OC(=O)N[C@H]1CCC[C@H](C=O)C1] | 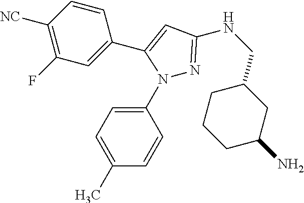 | 4-(3-((((1S,3S)-3- aminocyclohexyl) methyl)amino)-1-(p- tolyl)-1H-pyrazol-5- yl)-2- fluorobenzonitrile |
| 13RR | | 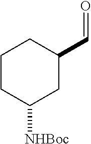 [SMILES: CC(C)(C)OC(=O)N[C@@H]1CCC[C@@H](C=O)C1] | 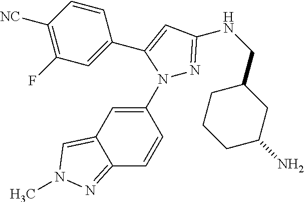 | 4-(3-((((1R,3R)-3- aminocyclohexyl) methyl)amino)-1-(2- methyl-2H-indazol-5- yl)-1H-pyrazol-5-yl)- 2-fluorobenzonitrile |
| 13SS | | 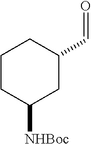 [SMILES: CC(C)(C)OC(=O)N[C@H]1CCC[C@H](C=O)C1] | 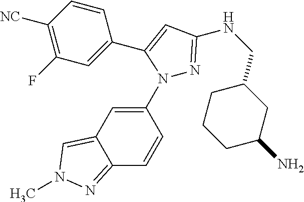 | 4-(3-((((1S,3S)-3- aminocyclohexyl) methyl)amino)-1-(2- methyl-2H-indazol-5- yl)-1H-pyrazol-5-yl)- 2-fluorobenzonitrile |
| 14RR | | 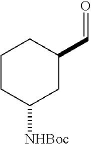 [SMILES: CC(C)(C)OC(=O)N[C@@H]1CCC[C@@H](C=O)C1] | 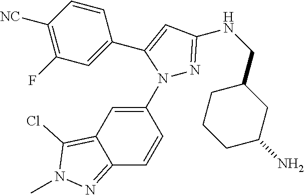 | 4-(3-((((1R,3R)-3- aminocyclohexyl) methyl)amino)-1-(3- chloro-2-methyl-2H- indazol-5-yl)-1H- pyrazol-5-yl)-2- fluorobenzonitrile |
| 14SS | | 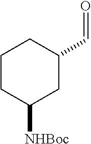 [SMILES: CC(C)(C)OC(=O)N[C@H]1CCC[C@H](C=O)C1] | 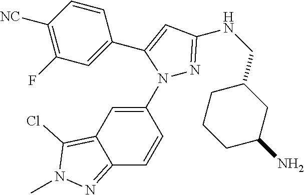 | 4-(3-((((1S,3S)-3- aminocyclohexyl) methyl)amino)-1-(3- chloro-2-methyl-2H- indazol-5-yl)-1H- pyrazol-5-yl)-2- fluorobenzonitrile |
| 15 | | 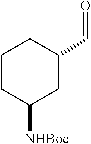 [SMILES: CC(C)(C)OC(=O)N[C@H]1CCC[C@H](C=O)C1] | 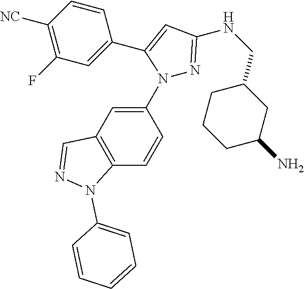 | 4-(3-((((1S,3S)-3- aminocyclohexyl) methyl)amino)-1-(1- phenyl-1H-indazol-5- yl)-1H-pyrazol-5-yl)- 2-fluorobenzonitrile |
| 16 | | 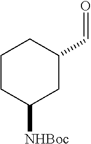 [SMILES: CC(C)(C)OC(=O)N[C@H]1CCC[C@H](C=O)C1] | 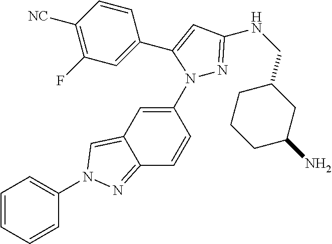 | 4-(3-((((1S,3S)-3- aminocyclohexyl) methyl)amino)-1-(2- phenyl-2H-indazol-5- yl)-1H-pyrazol-5-yl)- 2-fluorobenzonitrile |
| 17 | | 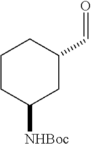 [SMILES: CC(C)(C)OC(=O)N[C@H]1CCC[C@H](C=O)C1] | 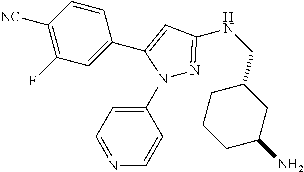 | 4-(3-((((1S,3S)-3- aminocyclohexyl) methyl)amino)-1- (pyridin-4-yl)-1H- pyrazol-5-yl)-2- fluorobenzonitrile |
| 18 | B-4 | 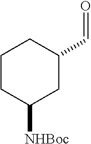 [SMILES: CC(C)(C)OC(=O)N[C@H]1CCC[C@H](C=O)C1] | 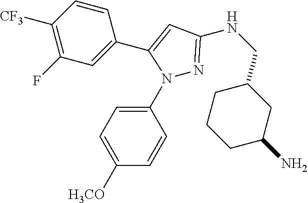 | N-(((1S,3S)-3- aminocyclohexyl) methyl)-5-(3-fluoro-4- (trifluoromethyl) phenyl)-1-(4- methoxyphenyl)-1H- pyrazol-3-amine |
| 19 | | 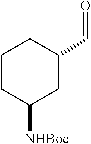 [SMILES: CC(C)(C)OC(=O)N[C@H]1CCC[C@H](C=O)C1] | 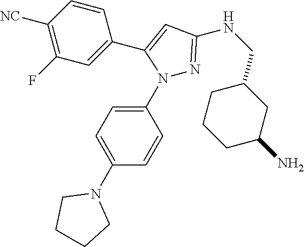 | 4-(3-((((1S,3S)-3- aminocyclohexyl) methyl)amino)-1-(4- (pyrrolidin-1- yl)phenyl)-1H- pyrazol-5-yl)-2- fluorobenzonitrile |
| 20 | | 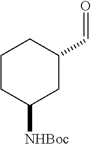 [SMILES: CC(C)(C)OC(=O)N[C@H]1CCC[C@H](C=O)C1] | 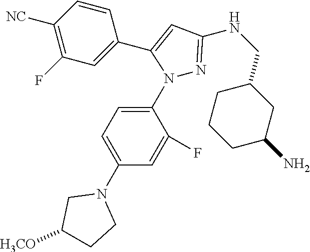 | 4-(3-((((1S,3S)-3- aminocyclohexyl) methyl)amino)-1-(2- fluoro-4-((S)-3- methoxypyrrolidin-1- yl)phenyl)-1H- pyrazol-5-yl)-2- fluorobenzonitrile |
| 21R | B-3 | 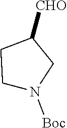 [SMILES: CC(C)(C)OC(=O)N1CC[C@@H](C=O)C1] | 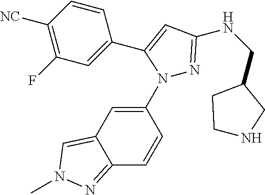 | (R)-2-fluoro-4-(1-(2- methyl-2H-indazol-5- yl)-3-((pyrrolidin-3- ylmethyl)amino)-1H- pyrazol-5- yl)benzonitrile |
| 21S | B-3 | 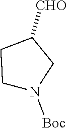 [SMILES: CC(C)(C)OC(=O)N1CC[C@H](C=O)C1] | 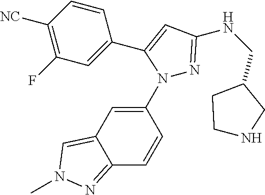 | (S)-2-fluoro-4-(1-(2- methyl-2H-indazol-5- yl)-3-((pyrrolidin-3- ylmethyl)amino)-1H- pyrazol-5- yl)benzonitrile |

### Example 22

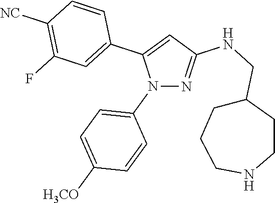 [SMILES: COc1ccc(-n2nc(NCC3CCCNCC3)cc2-c2ccc(C#N)c(F)c2)cc1]

### 4-(3-((azepan-4-ylmethyl)amino)-1-(4-methoxyphenyl)-1H-pyrazol-5-yl)-2-fluorobenzonitrile

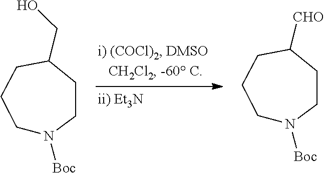 [SMILES: CC(C)(C)OC(=O)N1CCCC(CO)CC1 ; CC(C)(C)OC(=O)N1CCCC(C=O)CC1]

tert-Butyl 4-formylazepane-1-carboxylate To a solution of DMSO (503 mg, 6.54 mmol, 3.00 equiv) in CH₂Cl₂ (20 mL) was added (COCl)₂ (830 mg, 6.54 mmol, 3.00 equiv) dropwise at −60° C. under N₂. The resulting solution was stirred for 30 min at −60° C., then tert-butyl 4-(hydroxymethyl) azepane-1-carboxylate (500 mg, 2.18 mmol, 1.00 equiv) was added. The resulting mixture was stirred for 30 min at −60° C., then Et₃N (1.11 g, 10.9 mmol, 5.00 equiv) was added at −60° C. The resulting mixture was stirred for 10 min at room temperature. The mixture was diluted with H₂O (20 mL) and extracted with CH₂Cl₂ (3×20 mL). The combined organic layers were dried over anhydrous Na₂SO₄ and concentrated under vacuum to afford the title compound (400 mg, crude) as yellow oil. MS-ESI: 228 (M+1).

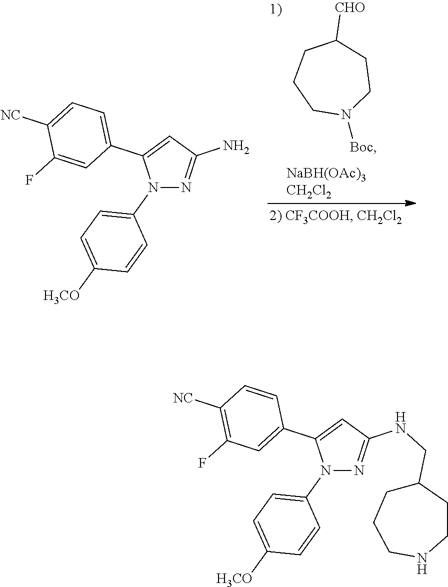 [SMILES: COc1ccc(-n2nc(N)cc2-c2ccc(C#N)c(F)c2)cc1 ; CC(C)(C)OC(=O)N1CCCC(C=O)CC1 ; COc1ccc(-n2nc(NCC3CCCNCC3)cc2-c2ccc(C#N)c(F)c2)cc1]

4-(3-((azepan-4-ylmethyl)amino)-1-(4-methoxyphenyl)-1H-pyrazol-5-yl)-2-fluorobenzonitrile The title compound was obtained using procedures similar to those used for Example Compound 11.

MS-ESI: 420 (M+1).

¹H-NMR (400 MHZ, MeOD-d₄) δ ppm: 7.66 (t, J=7.2 Hz, 1H), 7.23-7.14 (m, 4H), 7.00-6.93 (m, 2H), 6.09 (s, 1H), 3.83 (s, 3H), 3.41-3.36 (m, 1H) 3.30-3.28 (m, 1H), 3.22-3.05 (m, 4H), 2.23-1.93 (m, 4H), 1.90-1.79 (m, 1H), 1.71-1.61 (m, 1H), 1.42-1.38 (m, 1H).

The following compounds were obtained using procedures similar to those used for Example Compound 22.

TABLE 15
Example Compounds.

| Ex. | | | MWt/ |
|---|---|---|---|
| No. | Structure | IUPAC Name | M + H⁺ |
| 23 | 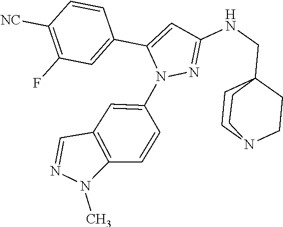 [SMILES: Cn1ncc2cc(-n3nc(NCC45CCN(CC4)CC5)cc3-c3ccc(C#N)c(F)c3)ccc21] | 2-fluoro-4-(1-(1-methyl- 1H-indazol-5-yl)-3- ((quinuclidin-4- ylmethyl)amino)-1H- pyrazol-5-yl)benzonitrile | |
| 24 | 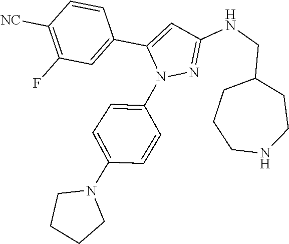 [SMILES: N#Cc1ccc(-c2cc(NCC3CCCNCC3)nn2-c2ccc(N3CCCC3)cc2)cc1F] | 4-(3-((azepan-4- ylmethyl)amino)-1-(4- (pyrrolidin-1-yl)phenyl)- 1H-pyrazol-5-yl)-2- fluorobenzonitrile | |
| 25 | 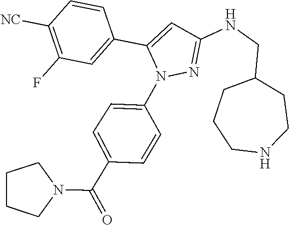 [SMILES: N#Cc1ccc(-c2cc(NCC3CCCNCC3)nn2-c2ccc(C(=O)N3CCCC3)cc2)cc1F] | 4-(3-((azepan-4- ylmethyl)amino)-1-(4- (pyrrolidine-1- carbonyl)phenyl)-1H- pyrazol-5-yl)-2- fluorobenzonitrile | |
| 26 | 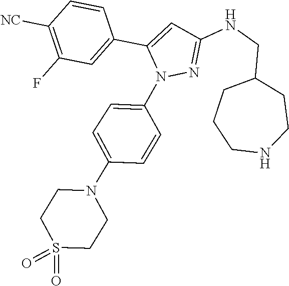 [SMILES: N#Cc1ccc(-c2cc(NCC3CCCNCC3)nn2-c2ccc(N3CCS(=O)(=O)CC3)cc2)cc1F] | 4-(3-((azepan-4- ylmethyl)amino)-1-(4- (1,1- dioxidothiomorpholino)- phenyl)-1H-pyrazol-5- yl)-2-fluorobenzonitrile | |
| 27 | 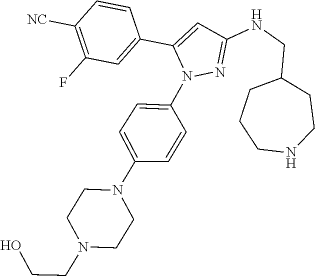 [SMILES: N#Cc1ccc(-c2cc(NCC3CCCNCC3)nn2-c2ccc(N3CCN(CCO)CC3)cc2)cc1F] | 4-(3-((azepan-4- ylmethyl)amino)-1-(4-(4- (2- hydroxyethyl)piperazin- 1-yl)phenyl)-1H-pyrazol- 5-yl)-2- fluorobenzonitrile | |

### Example 28

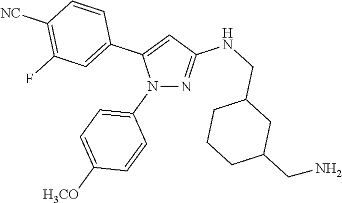 [SMILES: COc1ccc(-n2nc(NCC3CCCC(CN)C3)cc2-c2ccc(C#N)c(F)c2)cc1]

### 4-(3-(((-3-(aminomethyl)cyclohexyl)methyl)amino)-1-(4-methoxyphenyl)-1H-pyrazol-5-yl)-2-fluorobenzonitrile

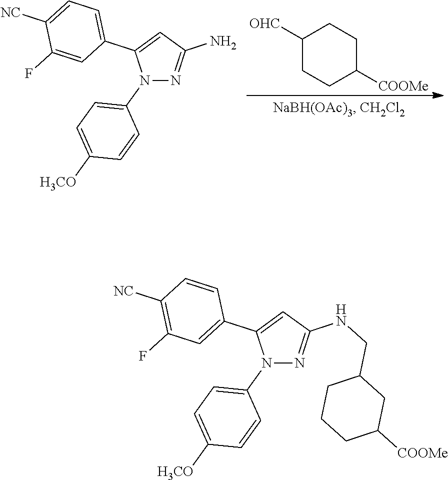 [SMILES: COc1ccc(-n2nc(N)cc2-c2ccc(C#N)c(F)c2)cc1 ; COC(=O)C1CCC(C=O)CC1 ; COC(=O)C1CCCC(CNc2cc(-c3ccc(C#N)c(F)c3)n(-c3ccc(OC)cc3)n2)C1]

Methyl 3-(((5-(4-cyano-3-fluorophenyl)-1-(4-methoxyphenyl)-1H-pyrazol-3-yl)amino)methyl)cyclohexane-1-carboxylate To a solution of 4-(3-amino-1-(4-methoxyphenyl)-1H-pyrazol-5-yl)-2-fluorobenzonitrile (1.00 g, 3.24 mmol, 1.00 equiv) in CH₂Cl₂ (30 mL) was added methyl 3-formylcyclohexane-1-carboxylate (662 mg, 3.89 mmol, 1.20 equiv). The mixture was stirred for 30 min at room temperature, then NaBH(OAc)₃ (2.06 g, 9.72 mmol, 3.00 equiv) was added in portions at room temperature. The resulting mixture was stirred for additional 40 min at room temperature, then quenched with H₂O (20 mL) and extracted with CH₂Cl₂ (3×50 mL). The combined organic layers were dried with anhydrous Na₂SO₄ and concentrated under vacuum. The residue was purified by Prep-TLC (PE/EtOAc 1:2) to afford the title compound (1.1 g, 73%) as a yellow solid. MS-ESI: 463 (M+1).

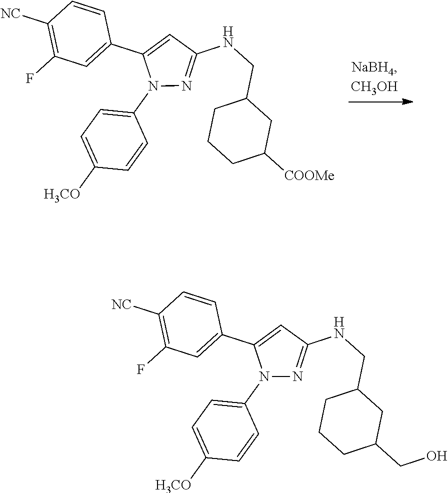 [SMILES: COC(=O)C1CCCC(CNc2cc(-c3ccc(C#N)c(F)c3)n(-c3ccc(OC)cc3)n2)C1 ; COc1ccc(-n2nc(NCC3CCCC(CO)C3)cc2-c2ccc(C#N)c(F)c2)cc1]

2-fluoro-4-(3-(((3-(hydroxymethyl)cyclohexyl)methyl)amino)-1-(4-methoxyphenyl)-1H-pyrazol-5-yl)benzonitrile To a solution of the product from the previous step (1.0 g, 2.16 mmol, 1.00 equiv) in MeOH (30 mL) was added NaBH₄ (245 mg, 6.49 mmol, 3.00 equiv) in portions at room temperature. The resulting mixture was stirred for 10 h at room temperature, then quenched with H₂O (50 mL) and extracted with CH₂Cl₂ (3×30 mL). The combined organic layers were dried with anhydrous Na₂SO₄ and concentrated under vacuum. The residue was purified by Prep-TLC (PE/EtOAc 1:2) to afford the title compound (800 mg, 85%) as a yellow solid. MS-ESI: 435 (M+1).

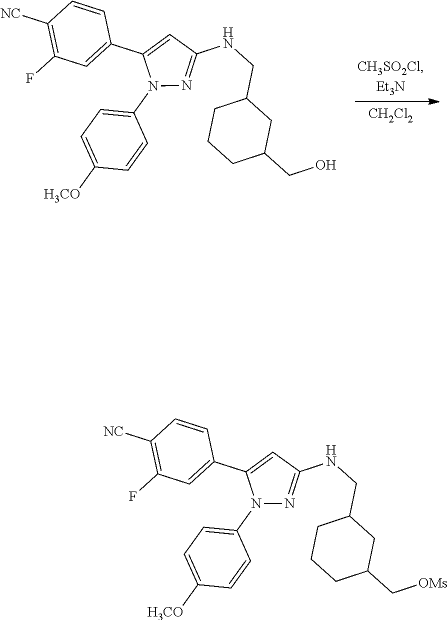 [SMILES: COc1ccc(-n2nc(NCC3CCCC(CO)C3)cc2-c2ccc(C#N)c(F)c2)cc1 ; COc1ccc(-n2nc(NCC3CCCC(COS(C)(=O)=O)C3)cc2-c2ccc(C#N)c(F)c2)cc1]

(3-(((5-(4-cyano-3-fluorophenyl)-1-(4-methoxyphenyl)-1H-pyrazol-3-yl)amino)methyl)cyclohexyl)methyl methanesulfonate To a solution of the product from the previous step (500 mg, 1.15 mmol, 1.00 equiv) in CH₂Cl₂ (30 mL) was added Et₃N (349 mg, 3.45 mmol, 3.00 equiv) and CH₃SO₂Cl (197 mg, 1.73 mmol, 1.50 equiv) dropwise at room temperature. The resulting mixture was stirred for 1 h at room temperature, then quenched with sat. NH₄Cl (aq.) and extracted with CH₂Cl₂ (3×50 mL). The combined organic layers were dried with anhydrous Na₂SO₄ and concentrated under vacuum to afford the title compound (550 mg, 93%) as a yellow solid. MS-ESI: 513 (M+1).

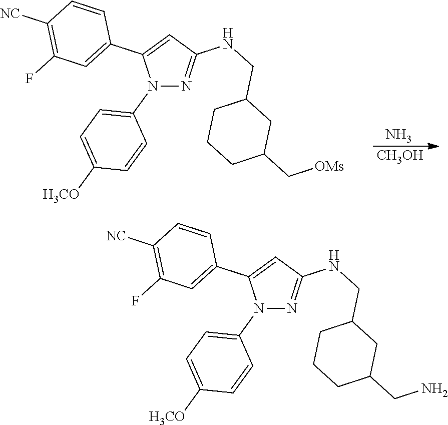

4-(3-(((3-(aminomethyl)cyclohexyl)methyl)amino)-1-(4-methoxyphenyl)-1H-pyrazol-5-yl)-2-fluorobenzonitrile A solution of the product from the previous step (500 mg, 0.97 mmol, 1.00 equiv) in NH₃ (g) in MeOH (7 M, 20 mL) was stirred overnight at 70° C. The resulting mixture was concentrated under vacuum. The crude product was purified by Prep-HPLC with the following conditions: Sunfire prep C18 Column, 30*150 mm, 5 um; Mobile Phase A: water (0.05% TFA), Mobile Phase B: ACN; Flow rate: 60 mL/min; Gradient: 25% B to 38% B in 8 min, Detector UV: 254 nm; RT1(min): 7.32 to afford the title compound (240 mg, 44%) as a yellow solid.

MS-ESI: 434 (M+1).

¹H NMR (400 MHZ, MeOD-d₄): δ 7.68 (t, J=7.4 Hz, 1H), 7.24-7.13 (m, 4H), 7.01-7.69 (m, 2H), 6.10 (s, 1H), 3.84 (s, 3H), 3.10 (dd, J=2.4 Hz, 6.4 Hz, 2H), 2.82 (d, J=6.8 Hz, 2H), 2.05-1.86 (m, 4H), 1.74-1.60 (m, 2H), 1.46-1.31 (m, 1H), 1.07-0.99 (m, 2H), 0.77 (q, J=12.2 Hz, 1H).

### Example 29

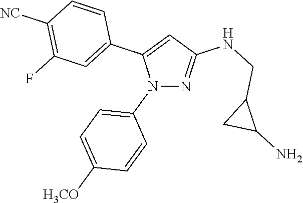 [SMILES: COc1ccc(-n2nc(NCC3CC3N)cc2-c2ccc(C#N)c(F)c2)cc1]

### (±)-(trans)-4-(3-((azepan-4-ylmethyl)amino)-1-(4-methoxyphenyl)-1H-pyrazol-5-yl)-2-fluorobenzonitrile

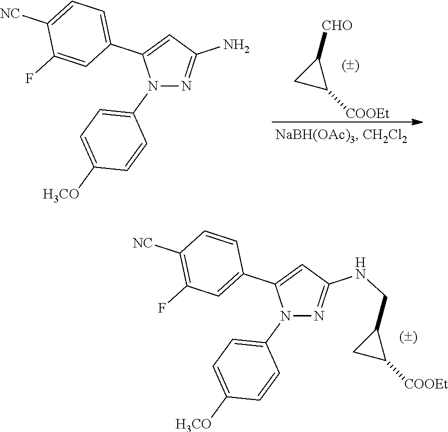 [SMILES: COc1ccc(-n2nc(N)cc2-c2ccc(C#N)c(F)c2)cc1 ; CCOC(=O)[C@@H]1C[C@H]1C=O ; CCOC(=O)[C@@H]1C[C@H]1CNc1cc(-c2ccc(C#N)c(F)c2)n(-c2ccc(OC)cc2)n1]

(±)-trans-Ethyl 2-(((5-(4-cyano-3-fluorophenyl)-1-(4-methoxyphenyl)-1H-pyrazol-3-yl)amino)methyl)cyclopropane-1-carboxylate A solution of 4-(3-amino-1-(4-methoxyphenyl)-1H-pyrazol-5-yl)-2-fluorobenzonitrile (4.0 g, 12.98 mmol, 1.00 equiv) in CH₂Cl₂ (150 mL) and ethyl 2-formylcyclopropane-1-carboxylate (2.77 g, 19.5 mmol, 1.50 equiv) was stirred for 30 min, then NaBH(OAc)₃ (8.25 g, 38.9 mmol, 3.00 equiv) was added in portions. The reaction mixture was stirred for 16 h at room temperature, then concentrated under vacuum. The residue was purified with silica gel chromatography using EtOAc/PE (1:1) to afford the title compound (2.4 g, 42%) as brown yellow oil. MS-ESI: 435 (M+1).

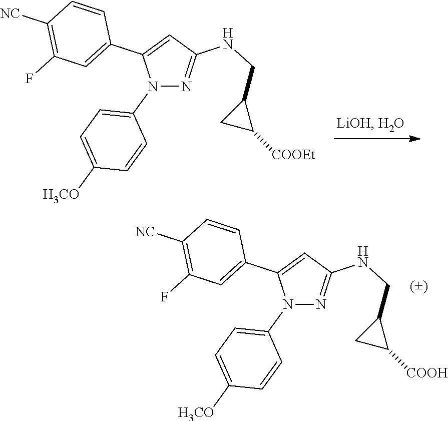 [SMILES: CCOC(=O)[C@@H]1C[C@H]1CNc1cc(-c2ccc(C#N)c(F)c2)n(-c2ccc(OC)cc2)n1 ; COc1ccc(-n2nc(NC[C@@H]3C[C@H]3C(=O)O)cc2-c2ccc(C#N)c(F)c2)cc1]

(±)-trans-2-(((5-(4-Cyano-3-fluorophenyl)-1-(4-methoxyphenyl)-1H-pyrazol-3-yl)amino)methyl)cyclopropane-1-carboxylic acid To a solution of the product from the previous step (2.4 g, 5.52 mmol, 1.00 equiv) in THF (100 mL) and H₂O (20 mL) was added LiOH (265 mg, 11 mmol, 2.00 equiv). The resulting mixture was stirred for 16 h at room temperature. The reaction mixture was diluted with H₂O (100 mL). The mixture was adjusted pH to 3-4 with HCl (2 M) and extracted with EtOAc (3×200 mL). The combined organic layers were dried with anhydrous Na₂SO₄ and concentrated under reduced pressure to afford the title compound (1.6 g, crude) as a yellow solid. MS-ESI: 407 (M+1).

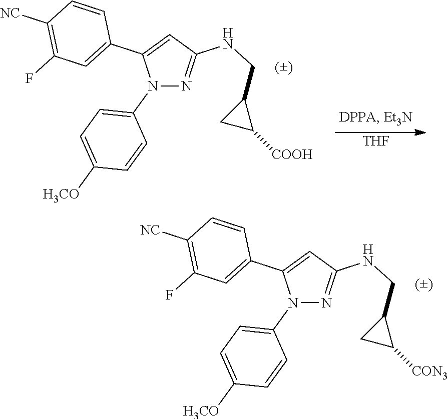

(±)-trans-2-(((5-(4-cyano-3-fluorophenyl)-1-(4-methoxyphenyl)-1H-pyrazol-3-yl)amino)methyl)cyclopropane-1-carbonyl azide To a solution of the product from the previous step (1.6 g, crude) in THF (100 mL) was added DPPA (1.63 g, 5.9 mmol, 1.50 equiv) and Et₃N (598 mg, 5.9 mmol, 1.50 equiv). The resulting mixture was stirred for 6 h at room temperature, then concentrated under vacuum. The residue was purified with silica gel using EtOAc/PE (1:1) to afford the title compound (1.12 g, 47%, over two steps) as a yellow solid. MS-ESI: 432 (M+1).

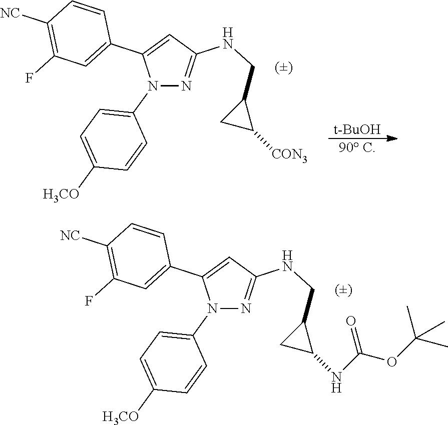

(±)-trans-tert-Butyl (2-(((5-(4-cyano-3-fluorophenyl)-1-(4-methoxyphenyl)-1H-pyrazol-3-yl)amino)methyl)cyclopropyl) carbamate A solution of the product from the previous step (1.12 g, 2.59 mmol, 1.00 equiv) in t-BuOH (20 mL) was stirred for 4 h at 70° C. The resulting mixture was concentrated under vacuum, and purified with silica gel chromatography using EtOAc/PE (1:1) to afford the title compound (272 mg, 22%) as a yellow oil. MS-ESI: 478 (M+1).

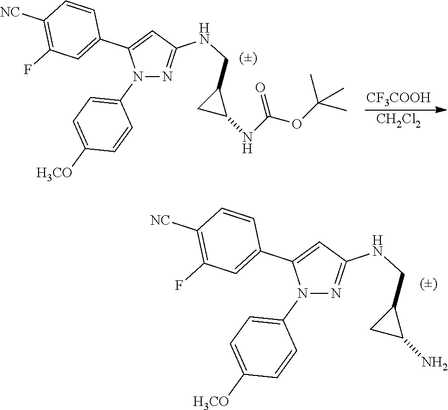

(±)-trans-4-(3-(((2-aminocyclopropyl)methyl)amino)-1-(4-methoxyphenyl)-1H-pyrazol-5-yl)-2-fluorobenzonitrile To a solution of the product from the previous step (272 mg, 0.57 mmol, 1.00 equiv) in CH₂Cl₂ (5 mL) was added CF₃COOH (1 mL). The solution was stirred for 16 h at room temperature. The resulting mixture was concentrated under vacuum. The crude product (300 mg) was purified by Prep-HPLC with the following conditions: Sunfire prep C18 Column, 30*150 mm, 5 μm; Mobile Phase A:

water (0.05% TFA), Mobile Phase B: ACN; Flow rate: 60 mL/min; Gradient: 20% B to 40% B in 7 min, Detector UV: 220 nm; RT1(min): 7.02, to afford 135 mg (49%) of the title compound as a dark yellow solid.

MS-ESI: 378 (M+1).

¹H NMR (400 MHZ, MeOD-d₄) δ 7.68 (t, J=8.0 Hz, 1H), 7.25-7.17 (m, 4H), 7.06-6.97 (m, 2H), 6.12 (s, 1H), 3.85 (s, 3H), 3.23 (d, J=6.8 Hz, 2H), 2.64-2.60 (m, 1H), 1.62-1.55 (m, 1H), 1.02-0.91 (m, 2H).

### Example 22A/b

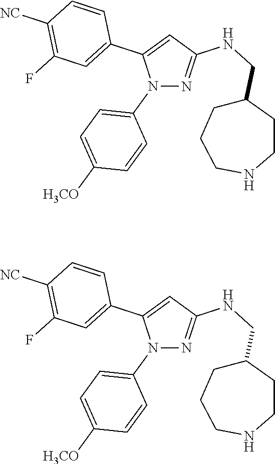

### (R)-4-(3-((azepan-4-ylmethyl)amino)-1-(4-methoxyphenyl)-1H-pyrazol-5-yl)-2-fluorobenzonitrile and

### (S)-4-(3-((azepan-4-ylmethyl)amino)-1-(4-methoxyphenyl)-1H-pyrazol-5-yl)-2-fluorobenzonitrile

Example Compound 22 was separated into enantiomers of undetermined absolute configuration by Chiral-Prep-HPLC with following conditions: CHIRALPAK IH Column, 2*25 cm, 5 um; Mobile Phase A: Hex (0.2% DEA), Mobile Phase B: EtOH: DCM=1: 1; Flow rate: 20 mL/min; Gradient: 50% B to 50% B in 11 min; Detector UV: 220/254 nm; RT1(min): 3.59, to afford 22a (17.7 mg, 17.70%) and 22b (17.4 mg, 17.40%) and as yellow solids. MS-ESI: 420 (M+1).

Example Compound 22a: ¹H-NMR (400 MHZ, MeOD-d₄) δ 7.68 (t, J=8.0 Hz, 1H), 7.23-7.16 (m, 4H), 7.00-6.96 (m, 2H), 6.09 (s, 1H), 3.83 (s, 3H), 3.32-3.23 (m, 1H) 3.21-3.18 (m, 1H), 3.14-3.06 (m, 4H), 2.13-1.94 (m, 4H), 1.83-1.76 (m, 1H), 1.69-1.55 (m, 1H), 1.41-1.37 (m, 1H).

Example Compound 22b: ¹H-NMR (400 MHZ, MeOD-d₄) δ 7.66 (t, J=7.2 Hz, 1H), 7.23-7.16 (m, 4H), 7.00-6.97 (m, 2H), 6.09 (s, 1H), 3.83 (s, 3H), 3.41-3.36 (m, 1H) 3.30-3.28 (m, 1H), 3.22-3.12 (m, 4H), 2.20-1.98 (m, 4H), 1.89-1.78 (m, 1H), 1.70-1.61 (m, 1H), 1.41-1.38 (m, 1H).

### Example 28a/b/c/d

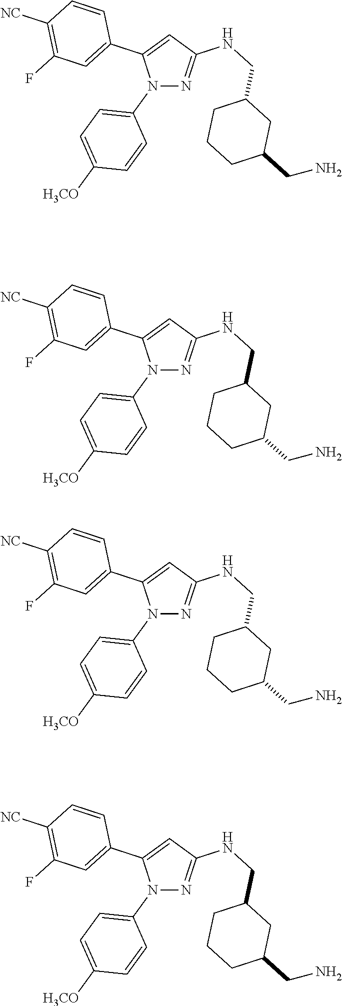

### 4-(3-((((1S,3S)-3-(aminomethyl)cyclohexyl)methyl)amino)-1-(4-methoxyphenyl)-1H-pyrazol-5-yl)-2-fluorobenzonitrile and

### 4-(3-((((1R,3R)-3-(aminomethyl)cyclohexyl)methyl)amino)-1-(4-methoxyphenyl)-1H-pyrazol-5-yl)-2-fluorobenzonitrile and

### 4-(3-((((1S,3R)-3-(aminomethyl)cyclohexyl)methyl)amino)-1-(4-methoxyphenyl)-1H-pyrazol-5-yl)-2-fluorobenzonitrile and

### 4-(3-((((1R,3S)-3-(aminomethyl)cyclohexyl)methyl)amino)-1-(4-methoxyphenyl)-1H-pyrazol-5-yl)-2-fluorobenzonitrile

Example Compound 28 was separated into enantiopure isomers of undetermined absolute configuration by Chiral-Prep-HPLC with following conditions: CHIRALPAK IG-3 Column, 0.46*5 cm, 3 μm; Mobile Phase A: Hex (0.2% DEA), Mobile Phase B: EtOH: DCM=1:1; Flow rate: 1 mL/min afford 28a (4.4 mg), 28b (21.8 mg), 28c (3.5 mg) and 28 (26.5 mg) as a yellow solids. MS-ESI: 434 (M+1).

28a ¹H NMR (400 MHZ, MeOD-d₄): δ 7.68 (t, J=7.6 Hz, 1H), 7.24-7.13 (m, 4H), 7.01-7.69 (m, 2H), 6.09 (s, 1H), 3.84 (s, 3H), 3.28-3.02 (m, 2H), 2.87 (d, J=7.2 Hz, 1H), 2.78 (d, J=6.8 Hz, 1H), 2.05-1.86 (m, 4H), 1.74-1.60 (m, 2H), 1.46-1.31 (m, 1H), 1.07-0.99 (m, 2H), 0.77 (q, J=12.2 Hz, 1H).

28b ¹H NMR (400 MHZ, MeOD-d₄): δ 7.68 (t, J=7.4 Hz, 1H), 7.24-7.13 (m, 4H), 7.01-7.69 (m, 2H), 6.10 (s, 1H), 3.84 (s, 3H), 3.10 (dd, J=2.8, 6.8 Hz, 2H), 2.82 (d, J=6.8 Hz, 2H), 2.05-1.86 (m, 4H), 1.74-1.60 (m, 2H), 1.46-1.31 (m, 1H), 1.07-0.99 (m, 2H), 0.77 (q, J=12.4 Hz, 1H).

28c ¹H NMR (400 MHZ, MeOD-d₄): δ 7.68 (t, J=7.4 Hz, 1H), 7.24-7.13 (m, 4H), 7.01-7.69 (m, 2H), 6.10 (s, 1H), 3.84 (s, 3H), 3.26-3.04 (m, 2H), 2.90 (d, J=7.6 Hz, 1H), 2.81 (d, J=6.8 Hz, 1H), 2.05-1.86 (m, 4H), 1.74-1.60 (m, 2H), 1.46-1.31 (m, 1H), 1.07-0.99 (m, 2H), 0.77 (q, J=12.2 Hz, 1H).

28d ¹H NMR (400 MHZ, MeOD-d₄): δ 7.68 (t, J=7.4 Hz, 1H), 7.24-7.13 (m, 4H), 7.01-7.69 (m, 2H), 6.10 (s, 1H), 3.84 (s, 3H), 3.10 (dd, J=2.8, 6.8 Hz, 2H), 2.82 (d, J=6.8 Hz, 2H), 2.05-1.86 (m, 4H), 1.74-1.60 (m, 2H), 1.46-1.31 (m, 1H), 1.07-0.99 (m, 2H), 0.76 (q, J=12.2 Hz, 1H).

### Example 29a/b

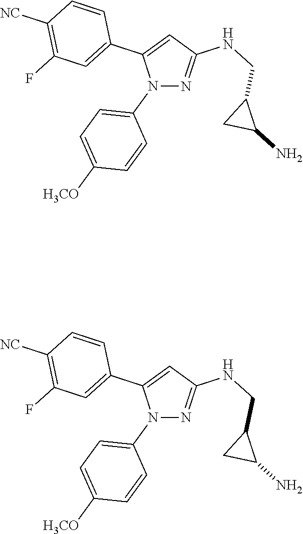

### 4-(3-((((1R,2S)-2-aminocyclopropyl)methyl)amino)-1-(4-methoxyphenyl)-1H-pyrazol-5-yl)-2-fluorobenzonitrile and

### 4-(3-((((1S,2R)-2-aminocyclopropyl)methyl)amino)-1-(4-methoxyphenyl)-1H-pyrazol-5-yl)-2-fluorobenzonitrile

Example Compound 29 was separated into enantiomers of undetermined absolute configuration by Chiral-Prep-HPLC with following conditions: CHIRALPAK IH Column, 2*25 cm, 5 μm; Mobile Phase A: Hex (0.2% DEA), Mobile Phase B: EtOH: DCM=1:1; Flow rate: 20 mL/min; Gradient: 60% B to 60% B in 5 min; Detector UV: 220/254 nm; RT1(min): 3.18, to afford 17.6 mg (17.6%) of 29a and 17.9 mg (17.9%) of 29b as yellow solids. MS-ESI: 378 (M+1).

29a: ¹H-NMR (400 MHZ, MeOD-d₄) δ 7.69 (t, J=7.6 Hz, 1H), 7.23-7.16 (m, 4H), 7.01-6.96 (m, 2H), 6.12 (s, 1H), 3.83 (s, 3H), 3.23 (d, J=6.4 Hz, 2H), 2.64-2.60 (m, 1H), 1.62-1.56 (m, 1H), 1.02-0.91 (m, 2H).

29b: ¹H-NMR (400 MHZ, MeOD-d₄) δ 7.69 (t, J=7.6 Hz, 1H), 7.23-7.16 (m, 4H), 7.01-6.96 (m, 2H), 6.12 (s, 1H), 3.83 (s, 3H), 3.23 (d, J=6.4 Hz, 2H), 2.64-2.60 (m, 1H), 1.62-1.56 (m, 1H), 1.02-0.91 (m, 2H).

### Example 30a/b

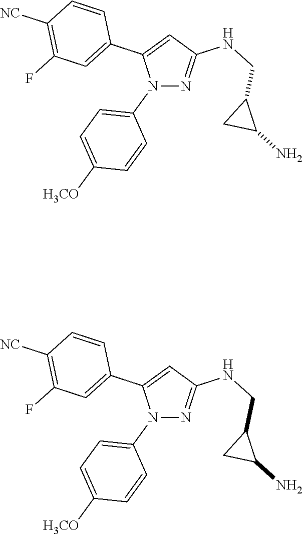

### 4-(3-((((1R,2R)-2-aminocyclopropyl)methyl)amino)-1-(4-methoxyphenyl)-1H-pyrazol-5-yl)-2-fluorobenzonitrile and

### 4-(3-((((1S,2S)-2-aminocyclopropyl)methyl)amino)-1-(4-methoxyphenyl)-1H-pyrazol-5-yl)-2-fluorobenzonitrile

Enantiomeric pair 30a and 30b of undetermined absolute configuration were obtained similarly.

The following racemic mixtures were resolved by chiral chromatography into enantiomeric pairs, of undetermined absolute stereochemistry.

TABLE 16
Resolved aminomethyl pyrazoles.

| Ex. | | | |
|---|---|---|---|
| No. | Racemate | Deprotected Product | IUPAC Name |
| 4a and 4b | 4 | 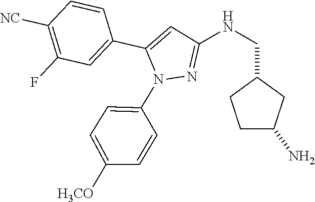 [SMILES: COc1ccc(-n2nc(NC[C@H]3CC[C@@H](N)C3)cc2-c2ccc(C#N)c(F)c2)cc1] | 4-(3-((((1S,3R)-3-amino- cyclopentyl)methyl)amino)- 1-(4-methoxyphenyl)-1H- pyrazol-5-yl)-2- fluorobenzonitrile |
| | | / | |
| | | 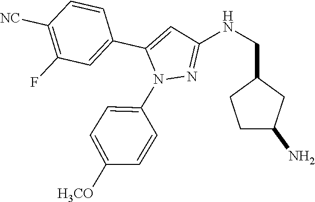 [SMILES: COc1ccc(-n2nc(NC[C@@H]3CC[C@H](N)C3)cc2-c2ccc(C#N)c(F)c2)cc1] | 4-(3-((((1R,3S)-3-amino- cyclopentyl)methyl)amino)- 1-(4-methoxyphenyl)-1H- pyrazol-5-yl)-2- fluorobenzonitrile |
| 5a and 5b | 5 | 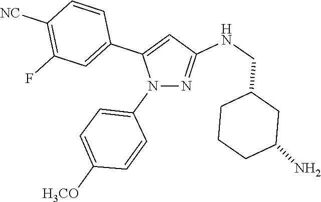 [SMILES: COc1ccc(-n2nc(NC[C@H]3CCC[C@@H](N)C3)cc2-c2ccc(C#N)c(F)c2)cc1] | 4-(3-((((1S,3R)-3-amino- cyclohexyl)methyl)amino)- 1-(4-methoxyphenyl)-1H- pyrazol-5-yl)-2- fluorobenzonitrile |
| | | / | |
| | | 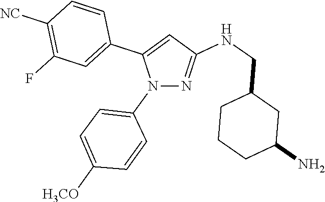 [SMILES: COc1ccc(-n2nc(NC[C@@H]3CCC[C@H](N)C3)cc2-c2ccc(C#N)c(F)c2)cc1] | 4-(3-((((1R,3S)-3-amino- cyclohexyl)methyl)amino)- 1-(4-methoxyphenyl)-1H- pyrazol-5-yl)-2- fluorobenzonitrile |
| 7a and 7b | 7 | 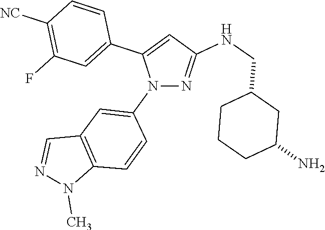 [SMILES: Cn1ncc2cc(-n3nc(NC[C@H]4CCC[C@@H](N)C4)cc3-c3ccc(C#N)c(F)c3)ccc21] | 4-(3-((((1S,3R)-3-amino- cyclohexyl)methyl)amino)- 1-(1-methyl-1H-indazol-5- yl)-1H-pyrazol-5-yl)-2- fluorobenzonitrile |
| | | / | |
| | | 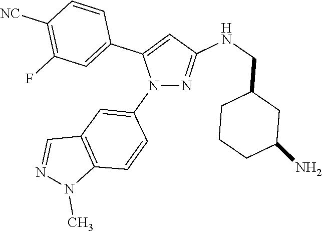 [SMILES: Cn1ncc2cc(-n3nc(NC[C@@H]4CCC[C@H](N)C4)cc3-c3ccc(C#N)c(F)c3)ccc21] | 4-(3-((((1R,3S)-3-amino- cyclohexyl)methyl)amino)- 1-(1-methyl-1H-indazol-5- yl)-1H-pyrazol-5-yl)-2- fluorobenzonitrile |
| 7c and 7d | 7 | 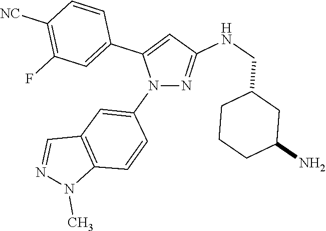 [SMILES: Cn1ncc2cc(-n3nc(NC[C@H]4CCC[C@H](N)C4)cc3-c3ccc(C#N)c(F)c3)ccc21] | 4-(3-((((1S,3S)-3-amino- cyclohexyl)methyl)amino)- 1-(1-methyl-1H-indazol-5- yl)-1H-pyrazol-5-yl)-2- fluorobenzonitrile |
| | | / | |
| | | 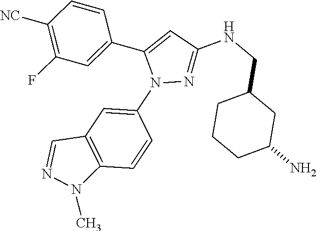 | 4-(3-((((1R,3R)-3-amino- cyclohexyl)methyl)amino)- 1-(1-methyl-1H-indazol-5- yl)-1H-pyrazol-5-yl)-2- fluorobenzonitrile |
| 9a and 9b | 9 | 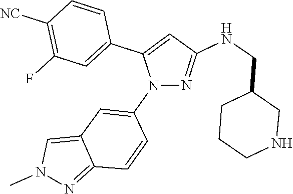 [SMILES: Cn1cc2cc(-n3nc(NC[C@@H]4CCCNC4)cc3-c3ccc(C#N)c(F)c3)ccc2n1] | (R)-2-fluoro-4-(1-(2-methyl- 2H-indazol-5-yl)-3- ((piperidin-3-ylmethyl)- amino)-1H-pyrazol-5- yl)benzonitrile |
| | | / | |
| | | 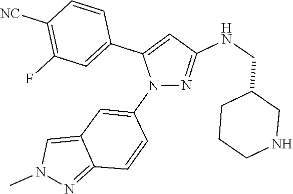 [SMILES: Cn1cc2cc(-n3nc(NC[C@H]4CCCNC4)cc3-c3ccc(C#N)c(F)c3)ccc2n1] | (S)-2-fluoro-4-(1-(2-methyl- 2H-indazol-5-yl)-3- ((piperidin-3-ylmethyl)- amino)-1H-pyrazol-5- yl)benzonitrile |
| 24a and 24b | 24 | 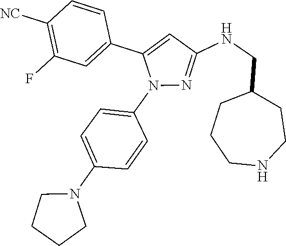 [SMILES: N#Cc1ccc(-c2cc(NC[C@@H]3CCCNCC3)nn2-c2ccc(N3CCCC3)cc2)cc1F] | (R)-4-(3-((azepan-4- ylmethyl)amino)-1-(4- (pyrrolidin-1-yl)phenyl)- 1H-pyrazol-5-yl)-2- fluorobenzonitrile |
| | | / | |
| | | 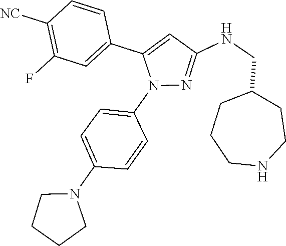 [SMILES: N#Cc1ccc(-c2cc(NC[C@H]3CCCNCC3)nn2-c2ccc(N3CCCC3)cc2)cc1F] | (S)-4-(3-((azepan-4- ylmethyl)amino)-1-(4- (pyrrolidin-1-yl)phenyl)- 1H-pyrazol-5-yl)-2- fluorobenzonitrile |
| 25a and 25b | 25 | 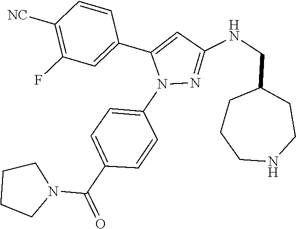 [SMILES: N#Cc1ccc(-c2cc(NC[C@@H]3CCCNCC3)nn2-c2ccc(C(=O)N3CCCC3)cc2)cc1F] | (R)-4-(3-((azepan-4- ylmethyl)amino)-1-(4- (pyrrolidine-1- carbonyl)phenyl)-1H- pyrazol-5-yl)-2- fluorobenzonitrile |
| | | / | |
| | | 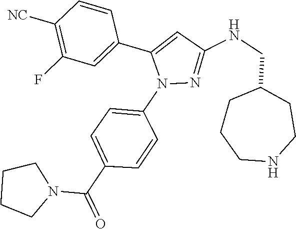 [SMILES: N#Cc1ccc(-c2cc(NC[C@H]3CCCNCC3)nn2-c2ccc(C(=O)N3CCCC3)cc2)cc1F] | (S)-4-(3-((azepan-4- ylmethyl)amino)-1-(4- (pyrrolidine-1- carbonyl)phenyl)-1H- pyrazol-5-yl)-2- fluorobenzonitrile |
| 26a and 26b | 26 | 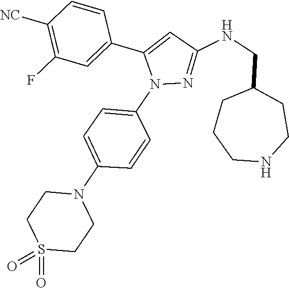 [SMILES: N#Cc1ccc(-c2cc(NC[C@@H]3CCCNCC3)nn2-c2ccc(N3CCS(=O)(=O)CC3)cc2)cc1F] | (R)-4-(3-((azepan-4- ylmethyl)amino)-1-(4-(1,1- dioxidothiomorpholino)- phenyl)-1H-pyrazol-5-yl)-2- fluorobenzonitrile/ |
| | | / | |
| | | 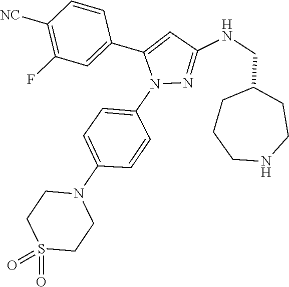 [SMILES: N#Cc1ccc(-c2cc(NC[C@H]3CCCNCC3)nn2-c2ccc(N3CCS(=O)(=O)CC3)cc2)cc1F] | (S)-4-(3-((azepan-4- ylmethyl)amino)-1-(4-(1,1- dioxidothiomorpholino)phenyl)- 1H-pyrazol-5-yl)-2- fluorobenzonitrile |
| 27a and 27b | 27 | 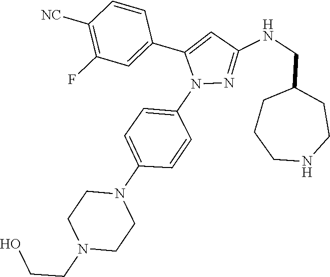 [SMILES: N#Cc1ccc(-c2cc(NC[C@@H]3CCCNCC3)nn2-c2ccc(N3CCN(CCO)CC3)cc2)cc1F] | (R)-4-(3-((azepan-4- ylmethyl)amino)-1-(4-(4-(2- hydroxyethyl)piperazin-1- yl)phenyl)-1H-pyrazol-5- yl)-2-fluorobenzonitrile |
| | | / | |
| | | 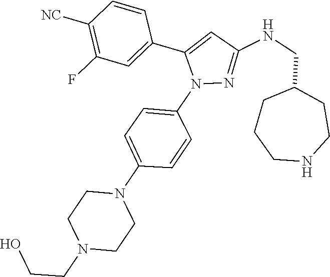 [SMILES: N#Cc1ccc(-c2cc(NC[C@H]3CCCNCC3)nn2-c2ccc(N3CCN(CCO)CC3)cc2)cc1F] | (S)-4-(3-((azepan-4- ylmethyl)amino)-1-(4-(4-(2- hydroxyethyl)piperazin-1- yl)phenyl)-1H-pyrazol-5- yl)-2-fluorobenzonitrile |
| 31a and 31b | | 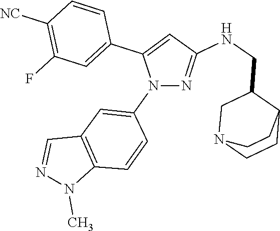 [SMILES: Cn1ncc2cc(-n3nc(NC[C@@H]4CN5CCC4CC5)cc3-c3ccc(C#N)c(F)c3)ccc21] | (R)-2-fluoro-4-(1-(1-methyl- 1H-indazol-5-yl)-3- ((quinuclidin-3-ylmethyl)- amino)-1H-pyrazol-5- yl)benzonitrile |
| | | / | |
| | | 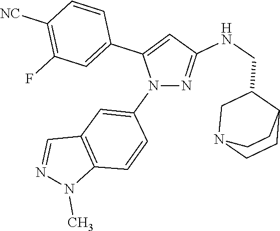 [SMILES: Cn1ncc2cc(-n3nc(NC[C@H]4CN5CCC4CC5)cc3-c3ccc(C#N)c(F)c3)ccc21] | (S)-2-fluoro-4-(1-(1-methyl- 1H-indazol-5-yl)-3- ((quinuclidin-3-ylmethyl)- amino)-1H-pyrazol-5- yl)benzonitrile |

The following pairs of enantiopure compounds, of undetermined absolute configuration, were obtained by chiral chromatography performed on the Boc-protected intermediates, followed by deprotection (TFA/CH₂Cl₂).

TABLE 17
Aminomethyl pyrazoles from resolved Boc-protected intermediates

| Ex. | | | | |
|---|---|---|---|---|
| No. | Amine | Aldehyde | Deprotected Product | IUPAC Name |
| 32a and 32b | B-1 | 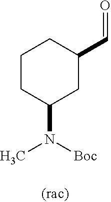 [SMILES: CN(C(=O)OC(C)(C)C)[C@H]1CCC[C@@H](C=O)C1] | 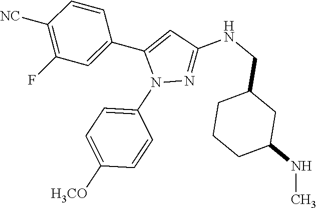 | 2-fluoro-4-(1-(4- methoxyphenyl)-3- ((((1R,3S)-3- (methylamino)- cyclohexyl)methyl)- amino)-1H-pyrazol-5- yl)benzonitrile |
| | | | / | |
| | | | 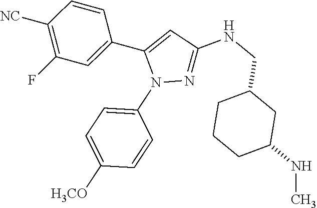 | 2-fluoro-4-(1-(4- methoxyphenyl)-3- ((((1S,3R)-3- (methylamino)- cyclohexyl)methyl)- amino)-1H-pyrazol-5- yl)benzonitrile |
| 33a and 33b | B-1 | 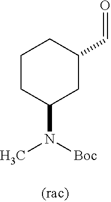 [SMILES: CN(C(=O)OC(C)(C)C)[C@H]1CCC[C@H](C=O)C1] | 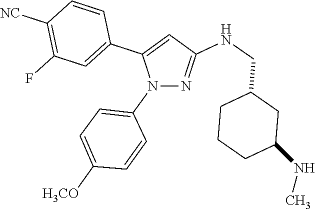 [SMILES: CN[C@H]1CCC[C@H](CNc2cc(-c3ccc(C#N)c(F)c3)n(-c3ccc(OC)cc3)n2)C1] | 2-fluoro-4-(1-(4- methoxyphenyl)-3- ((((1S,3S)-3- (methylamino)- cyclohexyl)methyl)- amino)-1H-pyrazol-5- yl)benzonitrile |
| | | | / | |
| | | | 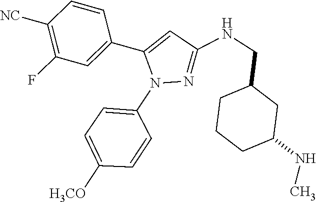 [SMILES: CN[C@@H]1CCC[C@@H](CNc2cc(-c3ccc(C#N)c(F)c3)n(-c3ccc(OC)cc3)n2)C1] | 2-fluoro-4-(1-(4- methoxyphenyl)-3- ((((1R,3R)-3- (methylamino)- cyclohexyl)methyl)- amino)-1H-pyrazol-5- yl)benzonitrile |

### Example 34

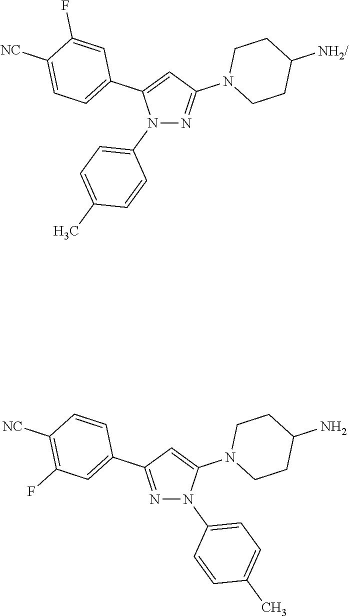

### 4-(3-(4-aminopiperidin-1-yl)-1-(p-tolyl)-1H-pyrazol-5-yl)-2-fluorobenzonitrile/4-(5-(4-aminopiperidin-1-yl)-1-(p-tolyl)-1H-pyrazol-3-yl)-2-fluorobenzonitrile

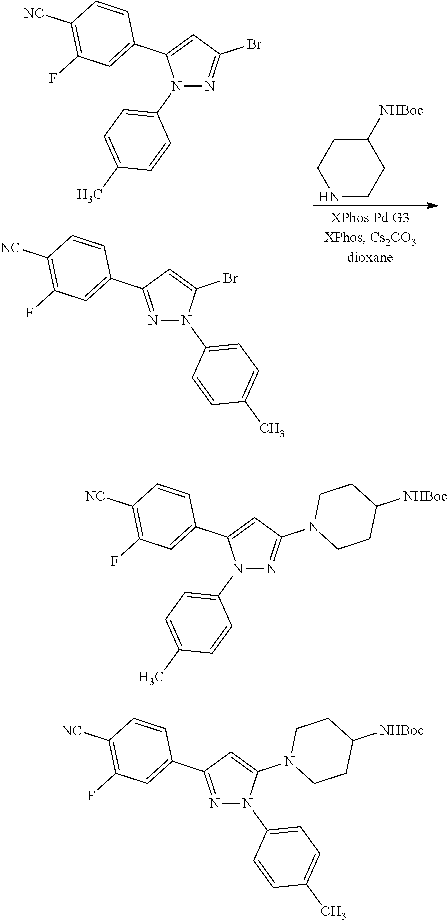 [SMILES: Cc1ccc(-n2nc(Br)cc2-c2ccc(C#N)c(F)c2)cc1 ; CC(C)(C)OC(=O)NC1CCNCC1 ; Cc1ccc(-n2nc(-c3ccc(C#N)c(F)c3)cc2Br)cc1 ; Cc1ccc(-n2nc(N3CCC(NC(=O)OC(C)(C)C)CC3)cc2-c2ccc(C#N)c(F)c2)cc1 ; Cc1ccc(-n2nc(-c3ccc(C#N)c(F)c3)cc2N2CCC(NC(=O)OC(C)(C)C)CC2)cc1]

tert-Butyl (1-(5-(4-cyano-3-fluorophenyl)-1-(p-tolyl)-1H-pyrazol-3-yl) piperidin-4-yl) carbamate and tert-butyl (1-(3-(4-cyano-3-fluorophenyl)-1-(p-tolyl)-1H-pyrazol-5-yl) piperidin-4-yl) carbamate In a 50-mL round-bottom flask purged and maintained under an atmosphere of N₂ were combined Intermediate X-6 (2.0 g, 5.62 mmol, 1.00 equiv) in dioxane (20 mL), tert-butyl piperidin-4-ylcarbamate (1.69 g, 8.42 mmol, 1.50 equiv), XPhos Pd G3 (475 mg, 0.56 mmol, 0.10 equiv), Xphos (268 mg, 0.56 mmol, 0.10 equiv), Cs₂CO₃ (5.49 g, 16.8 mmol, 3.00 equiv). The resulting solution was stirred 4 h at 90° C. The resulting mixture was concentrated under vacuum. The residue was purified with silica gel column chromatography, eluting with EtOAc/petroleum ether (1:3), to afford the title isomeric mixture (300 mg, 11.2%) as a yellow solid.

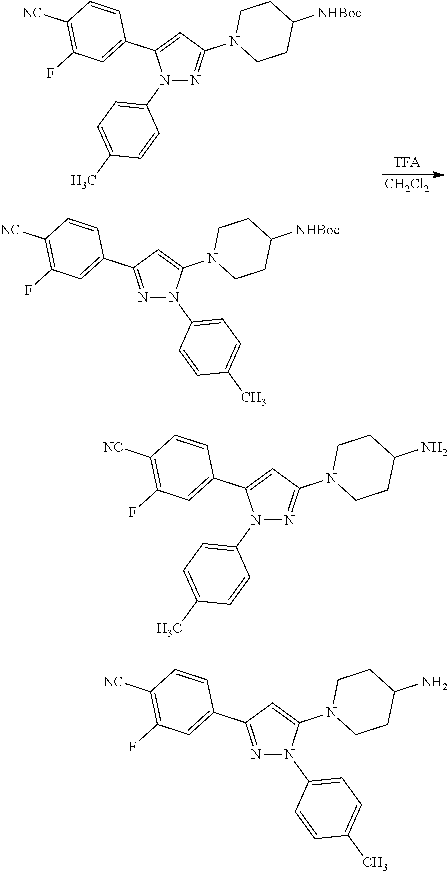

4-(3-(4-aminopiperidin-1-yl)-1-(p-tolyl)-1H-pyrazol-5-yl)-2-fluorobenzonitrile and 4-(5-(4-aminopiperidin-1-yl)-1-(p-tolyl)-1H-pyrazol-3-yl)-2-fluorobenzonitrile In a 50-mL round-bottom flask were combined the isomeric mixture from the previous step (300 mg) in CH₂Cl₂ (10 mL) and TFA (2 mL). The resulting solution was stirred 1 hr at rt. The resulting mixture was concentrated under vacuum. The crude product was purified with Flash-Prep-SFC with the following conditions (IntelFlash-1): Column: CHIRALPAK IC, 20×250 mm×5 μm; mobile phase A: CO₂, mobile phase B: MeOH (2 mM NH₃/MeOH); flow rate: 40 mL/min; gradient: isocratic 50% B; 1=220 nm; RT1(min): 4.09; RT2 (min): 5.14; Sample Solvent: MeOH—Preparative; Injection Volume: 1 mL; Number Of Runs: 13, to afford 35.2 mg of a mixture as the trifluoroacetate salts of the title compounds, as a yellow solid.

LC-MS: (ES, m/z): 376 [M+H]⁺.

¹H NMR (400 MHZ, CD₃OD) δ ppm: 7.71-7.66 (m, 1H), 7.27-7.13 (m, 6H), 6.34 (s, 1H), 3.86-3.82 (m, 2H), 2.89-2.82 (m, 3H), 2.40 (s, 3H), 1.96-1.92 (m, 2H), 1.56-1.53 (m, 2H).

### Example 35

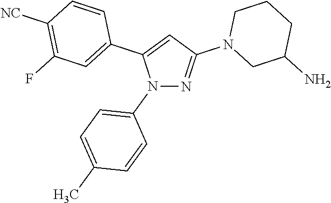 [SMILES: Cc1ccc(-n2nc(N3CCCC(N)C3)cc2-c2ccc(C#N)c(F)c2)cc1]

### 4-(3-(3-Aminopiperidin-1-yl)-1-(p-tolyl)-1H-pyrazol-5-yl)-2-fluorobenzonitrile

### Example 36

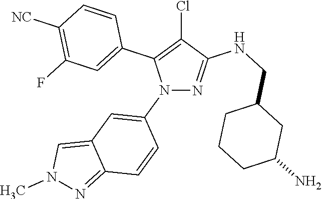

### 4-(3-((((1R,3R)-3-aminocyclohexyl)methyl)amino)-4-chloro-1-(2-methyl-2H-indazol-5-yl)-1H-pyrazol-5-yl)-2-fluorobenzonitrile

To a solution of Example Compound 13RR (50 mg, 0.11 mmol, 1.00 equiv) in CH₃CN (3 mL) was added NCS (12 mg, 0.090 mmol, 0.80 equiv). The mixture was stirred for 1 h at 70° C. The residue was purified by Prep-HPLC with the following conditions: Sunfire prep C18 Column, 30*150 mm, 5 μm; Mobile Phase A: water (0.05% TFA), Mobile Phase B: ACN; Flow rate: 60 mL/min; Gradient: 20% B to 48% B in 8 min, Detector UV: 220/254 nm; RT1(min): 7.48 to afford the title compound (26.2 mg, 41%) as a yellow solid.

MS-ESI: 576 (M+1).

¹H NMR (400 MHZ, MeOD-d₄) δ 8.21 (s, 1H), 7.73 (dd, J=6.8 Hz, 8.0 Hz, 1H), 7.64 (d, J=9.2 Hz, 1H), 7.47 (dd, J=0.8, 2.0 Hz, 1H), 7.35 (dd, J=1.6, 10.0 Hz, 1H), 7.28-7.23 (m, 2H), 4.23 (s, 3H), 3.53-3.45 (m, 1H), 3.30 (s, 2H), 2.27-2.14 (m, 1H), 1.98-1.55 (m, 7H), 1.54-1.48 (m, 1H).

Examples in the following table were prepared using similar conditions as used for Example Compound 36.

TABLE 18
Example Compounds. .

| Ex. | | | MWt/ |
|---|---|---|---|
| No. | Structure | IUPAC Name | M + H⁺ |
| 37 | 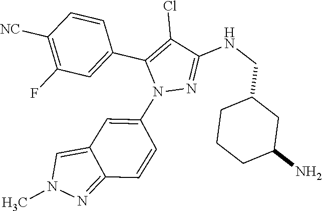 | 4-(3-((((1S,3S)-3- aminocyclohexyl)methyl) amino)-4-chloro-1-(2- methyl-2H-indazol-5-yl)- 1H-pyrazol-5-yl)-2- fluorobenzonitrile | |

### Examples 38 and 39

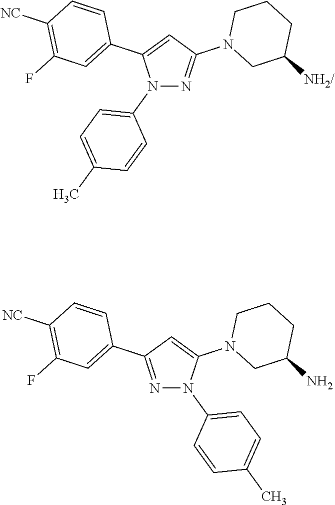

### (R)-4-(3-(3-aminopiperidin-1-yl)-1-(p-tolyl)-1H-pyrazol-5-yl)-2-fluorobenzonitrile/(R)-4-(5-(3-aminopiperidin-1-yl)-1-(p-tolyl)-1H-pyrazol-3-yl)-2-fluorobenzonitrile and

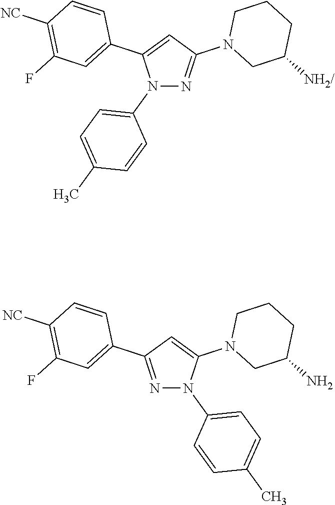

### (S)-4-(3-(3-aminopiperidin-1-yl)-1-(p-tolyl)-1H-pyrazol-5-yl)-2-fluorobenzonitrile/(S)-4-(5-(3-aminopiperidin-1-yl)-1-(p-tolyl)-1H-pyrazol-3-yl)-2-fluorobenzonitrile

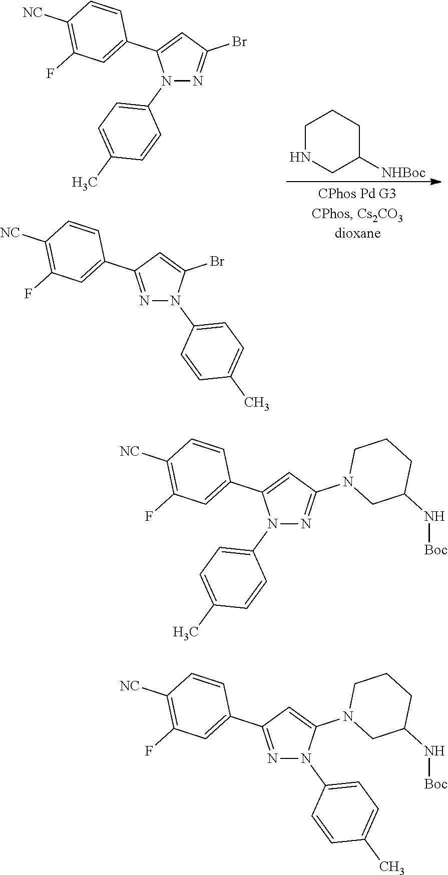

tert-Butyl (1-(5-(4-cyano-3-fluorophenyl)-1-(p-tolyl)-1H-pyrazol-3-yl) piperidin-3-yl) carbamate and tert-butyl (1-(3-(4-cyano-3-fluorophenyl)-1-(p-tolyl)-1H-pyrazol-5-yl) piperidin-3-yl) carbamate In a 100-mL round-bottom flask purged and maintained under an atmosphere of N₂ were combined Intermediate X-6 (2 g, 5.62 mmol, 1.00 equiv) in dioxane (40 mL), tert-butyl piperidin-3-ylcarbamate (1.69 g, 8.42 mmol, 1.50 equiv), CPhos Pd G3 (0.46 g, 0.562 mmol, 0.10 equiv), CPhos (0.25 g, 0.562 mmol, 0.10 equiv), Cs₂CO₃ (5.49 g, 16.8 mmol, 3.00 equiv). The resulting solution was stirred 4 h at 90° C. The resulting mixture was concentrated under vacuum. The residue was purified with silica gel column chromatography, eluting with EtOAc/petroleum ether (1:3), to afford 200 mg (7.5%) of the title isomeric mixture as a yellow solid.

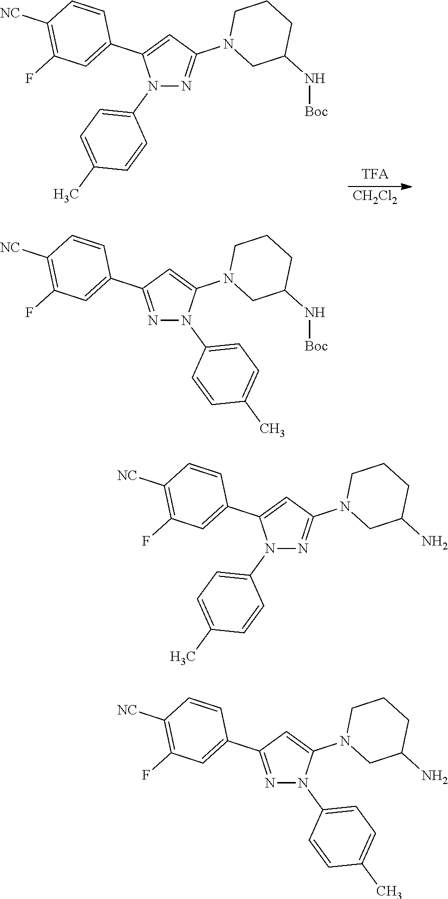

4-(3-(3-Aminopiperidin-1-yl)-1-(p-tolyl)-1H-pyrazol-5-yl)-2-fluorobenzonitrile and 4-(5-(3-aminopiperidin-1-yl)-1-(p-tolyl)-1H-pyrazol-3-yl)-2-fluorobenzonitrile In a 50-mL round-bottom flask were combined the isomeric mixture from the previous step (200 mg) in CH₂Cl₂ (10 mL) and TFA (2 mL). The resulting solution was stirred 1 h at rt. The resulting mixture was concentrated under vacuum, to afford 60 mg (38%) of title isomeric mixture, as their respective trifluoroacetate salts, as a yellow solid.

The product was purified with Chiral-Prep-HPLC with the following conditions: Column: CHIRALPAK IG, 20×250 mm×5 mm; mobile phase A: Hex (8 mM NH₃/MeOH)—HPLC, mobile phase B: IPA--HPLC; flow rate: 20 mL/min; gradient: 45 B to 45 B in 16 min; 220/254 nm; affording two fractions eluting at RT: 9.57 min and RT: 12.2 min.

EXAMPLE 38: The fraction eluting at 9.57 min was further purified with Prep-HPLC with the following conditions: Column: Sunfire prep C18 column 30×150 mm×5 mm; mobile phase A: H₂O (0.05% TFA), mobile phase B: CH₃CN; flow rate: 60 mL/min; gradient: 20% B to 44% B in 7 min; 1=254 nm; 16.3 mg, light yellow solid, RT: 6.43 min, LC-MS: (ES, m/z): 376 [M+H]⁺; ¹H NMR (400 MHZ, CD₃OD) δ ppm: 7.72-7.68 (m, 1H), 7.28-7.13 (m, 6H), 6.37 (s, 1H), 3.73-3.69 (m, 1H), 3.51-3.38 (m, 2H), 3.25-3.19 (m, 2H), 3.40 (s, 3H), 2.09-2.05 (m, 1H), 1.96-1.93 (m, 1H), 1.83-1.71 (m, 2H). The substance is tentatively assigned as a mixture of the trifluoroacetate salts of (R)-4-(3-(3-aminopiperidin-1-yl)-1-(p-tolyl)-1H-pyrazol-5-yl)-2-fluorobenzonitrile and (R)-4-(5-(3-aminopiperidin-1-yl)-1-(p-tolyl)-1H-pyrazol-3-yl)-2-fluorobenzonitrile.

EXAMPLE 39: The fraction eluting at 12.2 min was further purified with Prep-HPLC with the following conditions: Column: Sunfire prep C18 column, 30×150 mm×5 μm; mobile phase A: H₂O (0.05% TFA), mobile phase B: CH₃CN; flow rate: 60 mL/min; gradient: 20% B to 45% B in 7 min; 1=254 nm: 13.3 mg, light yellow solid, RT: 6.22 min; LC-MS: (ES, m/z): 376 [M+H]⁺; ¹H NMR (400 MHZ, CD₃OD) δ ppm: 7.72-7.68 (m, 1H), 7.28-7.13 (m, 6H), 6.37 (s, 1H), 3.74-3.69 (m, 1H), 3.51-3.38 (m, 2H), 3.25-3.19 (m, 2H), 3.40 (s, 3H), 2.09-2.05 (m, 1H), 1.96-1.94 (m, 1H), 1.83-1.71 (m, 2H). The substance is tentatively assigned as a mixture of the trifluoroacetate salts of(S)-4-(3-(3-aminopiperidin-1-yl)-1-(p-tolyl)-1H-pyrazol-5-yl)-2-fluorobenzonitrile and(S)-4-(5-(3-aminopiperidin-1-yl)-1-(p-tolyl)-1H-pyrazol-3-yl)-2-fluorobenzonitrile.

TABLE 19
Example Compounds.

| Ex. | | | MWt/ |
|---|---|---|---|
| No. | Structure | IUPAC Name | M + H⁺ |
| 40 | 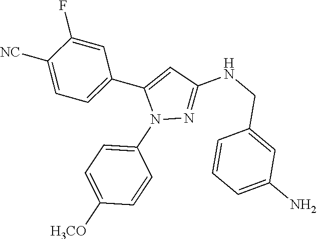 [SMILES: COc1ccc(-n2nc(NCc3cccc(N)c3)cc2-c2ccc(C#N)c(F)c2)cc1] | 4-(3-((3- aminobenzyl)amino)-1- (4-methoxyphenyl)-1H- pyrazol-5-yl)-2- fluorobenzonitrile | /414(M + 1) |
| 41 | 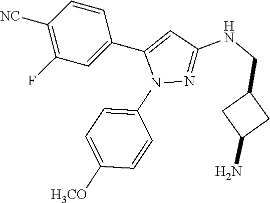 | 4-(3-((((1s,3s)-3- aminocyclobutyl)methyl) amino)-1-(4- methoxyphenyl)-1H- pyrazol-5-yl)-2- fluorobenzonitrile | /392(M + 1) |
| 42 | 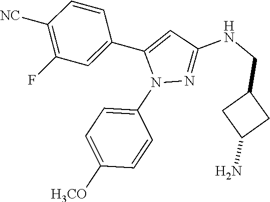 | 4-(3-((((1r,3r)-3- aminocyclobutyl)methyl) amino)-1-(4- methoxyphenyl)-1H- pyrazol-5-yl)-2- fluorobenzonitrile | /392(M + 1) |
| 43 | 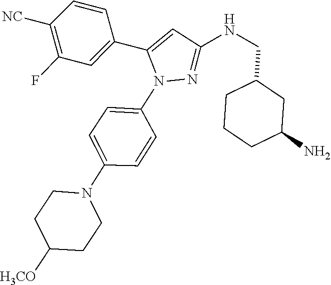 | 4-(3-((((1S,3S)-3- aminocyclohexyl)methyl) amino)-1-(4-(4- methoxypiperidin-1-yl)- phenyl)-1H-pyrazol-5- yl)-2-fluorobenzonitrile | /503(M + 1) |
| 44 | 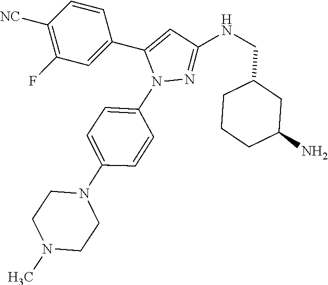 | 4-(3-((((1S,3S)-3- aminocyclohexyl)- methyl)amino)-1-(4-(4- methylpiperazin-1- yl)phenyl)-1H-pyrazol-5- yl)-2-fluorobenzonitrile | /488(M + 1) |
| 45 | 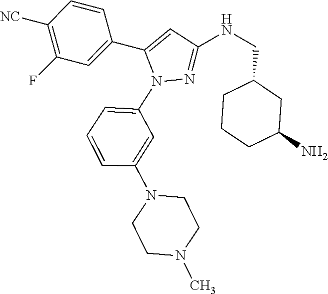 [SMILES: CN1CCN(c2cccc(-n3nc(NC[C@H]4CCC[C@H](N)C4)cc3-c3ccc(C#N)c(F)c3)c2)CC1] | 4-(3-((((1S,3S)-3- aminocyclohexyl)methyl) amino)-1-(3-(4- methylpiperazin-1- yl)phenyl)-1H-pyrazol-5- yl)-2-fluorobenzonitrile | /488(M + 1) |
| 46 | 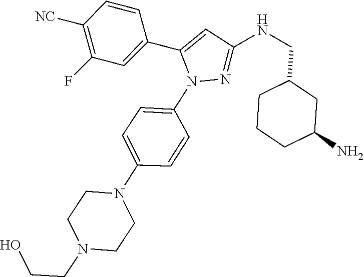 | 4-(3-((((1S,3S)-3- aminocyclohexyl)- methyl)amino)-1-(4-(4- (2- hydroxyethyl)piperazin- 1-yl)phenyl)-1H-pyrazol- 5-yl)-2- fluorobenzonitrile | /518(M + 1) |
| 47 | 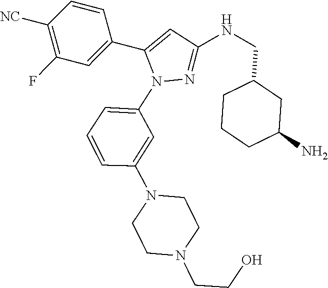 | 4-(3-((((1S,3S)-3- aminocyclohexyl)methyl) amino)-1-(3-(4-(2- hydroxyethyl)piperazin- 1-yl)phenyl)-1H-pyrazol- 5-yl)-2- fluorobenzonitrile | /518(M + 1) |
| 48 | 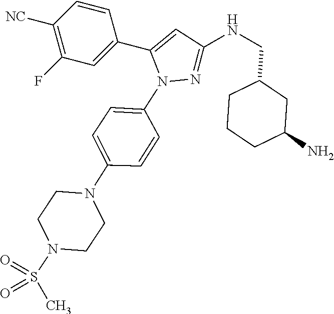 | 4-(3-((((1S,3S)-3- aminocyclohexyl)methyl) amino)-1-(4-(4- (methylsulfonyl)piperazin- 1-yl)phenyl)-1H- pyrazol-5-yl)-2- fluorobenzonitrile | /552(M + 1) |
| 49 | 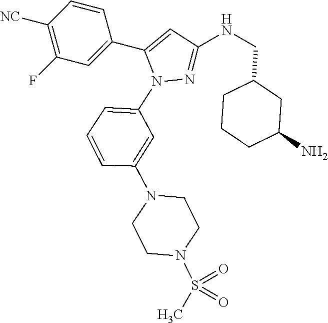 | 4-(3-((((1S,3S)-3- aminocyclohexyl)methyl) amino)-1-(3-(4- (methylsulfonyl)- piperazin-1-yl)phenyl)- 1H-pyrazol-5-yl)-2- fluorobenzonitrile | /552(M + 1) |
| 50 | 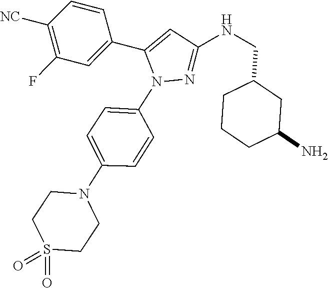 | 4-(3-((((1S,3S)-3- aminocyclohexyl)- methyl)amino)-1-(4-(1,1- dioxidothiomorpholino)- phenyl)-1H-pyrazol-5- yl)-2-fluorobenzonitrile | /523(M + 1) |
| 51 | 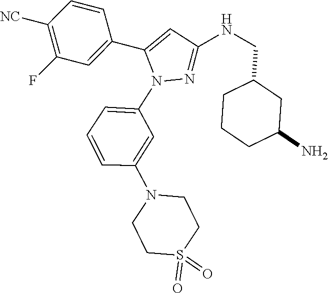 | 4-(3-((((1S,3S)-3- aminocyclohexyl)methyl) amino)-1-(3-(1,1- dioxidothiomorpholino)- phenyl)-1H-pyrazol-5- yl)-2-fluorobenzonitrile | /523(M + 1) |
| 52 | 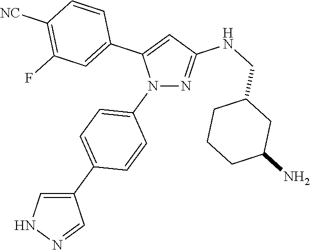 | 4-(1-(4-(1H-pyrazol-4- yl)phenyl)-3-((((1S,3S)- 3-aminocyclohexyl)- methyl)amino)-1H- pyrazol-5-yl)-2- fluorobenzonitrile | /554(M + 1) |
| 53 | 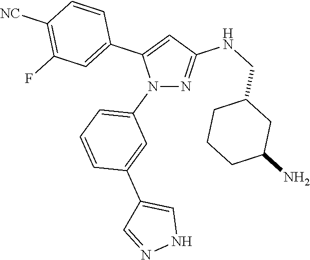 | 4-(1-(3-(1H-pyrazol-4- yl)phenyl)-3-((((1S,3S)- 3- aminocyclohexyl)methyl) amino)-1H-pyrazol-5- yl)-2-fluorobenzonitrile | /456(M + 1) |
| 54 | 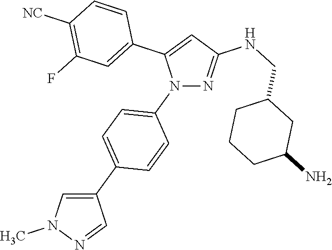 | 4-(3-((((1S,3S)-3- aminocyclohexyl)- methyl)amino)-1-(4-(1- methyl-1H-pyrazol-4- yl)phenyl)-1H-pyrazol-5- yl)-2-fluorobenzonitrile | /470(M + 1) |
| 55 | 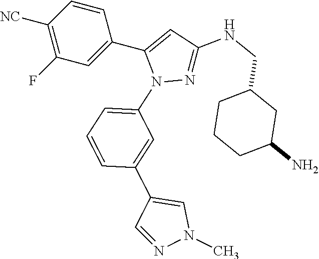 | 4-(3-((((1S,3S)-3- aminocyclohexyl)methyl) amino)-1-(3-(1-methyl- 1H-pyrazol-4-yl)phenyl)- 1H-pyrazol-5-yl)-2- fluorobenzonitrile | /470(M + 1) |
| 56 | 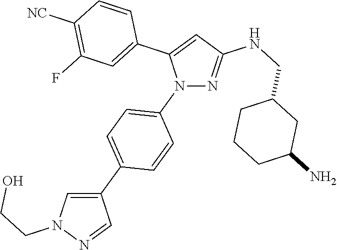 | 4-(3-((((1S,3S)-3- aminocyclohexyl)- methyl)amino)-1-(4-(1- (2-hydroxyethyl)-1H- pyrazol-4-yl)phenyl)-1H- pyrazol-5-yl)-2- fluorobenzonitrile | /500(M + 1) |
| 57 | 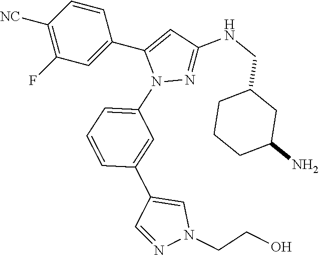 | 4-(3-((((1S,3S)-3- aminocyclohexyl)methyl) amino)-1-(3-(1-(2- hydroxyethyl)-1H- pyrazol-4-yl)phenyl)-1H- pyrazol-5-yl)-2- fluorobenzonitrile | /500(M + 1) |
| 58 | 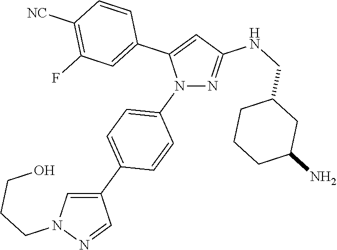 | 4-(3-((((1S,3S)-3- aminocyclohexyl)- methyl)amino)-1-(4-(1- (3-hydroxypropyl)-1H- pyrazol-4-yl)phenyl)-1H- pyrazol-5-yl)-2- fluorobenzonitrile | /514(M + 1) |
| 59 | 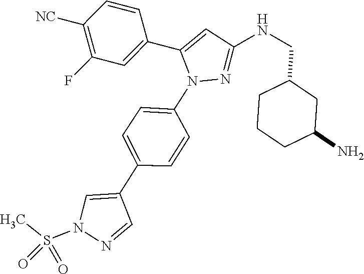 | 4-(3-((((1S,3S)-3- aminocyclohexyl)methyl) amino)-1-(4-(1- (methylsulfonyl)-1H- pyrazol-4-yl)phenyl)-1H- pyrazol-5-yl)-2- fluorobenzonitrile | /534(M + 1) |
| 60 | 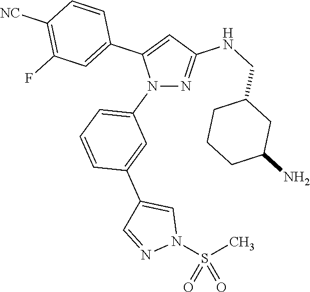 | 4-(3-((((1S,3S)-3- aminocyclohexyl)methyl) amino)-1-(3-(1- (methylsulfonyl)-1H- pyrazol-4-yl)phenyl)-1H- pyrazol-5-yl)-2- fluorobenzonitrile | /534(M + 1) |
| 61 | 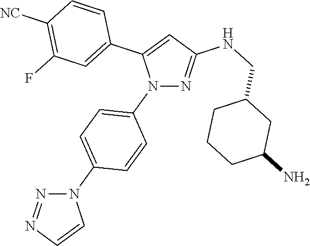 | 4-(1-(4-(1H-1,2,3-triazol- 1-yl)phenyl)-3- ((((1S,3S)-3- aminocyclohexyl)- methyl)amino)-1H- pyrazol-5-yl)-2- fluorobenzonitrile | /457(M + 1) |
| 62 | 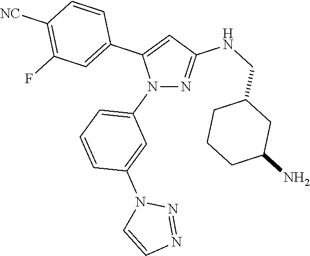 | 4-(1-(3-(1H-1,2,3-triazol- 1-yl)phenyl)-3- ((((1S,3S)-3- aminocyclohexyl)methyl) amino)-1H-pyrazol-5- yl)-2-fluorobenzonitrile | /457(M + 1) |
| 63 | 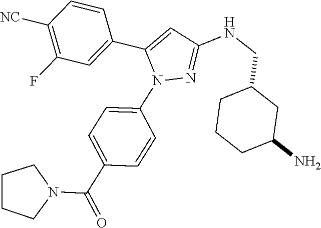 | 4-(3-((((1S,3S)-3- aminocyclohexyl)- methyl)amino)-1-(4- (pyrrolidine-1- carbonyl)phenyl)-1H- pyrazol-5-yl)-2- fluorobenzonitrile | /487(M + 1) |
| 64 | 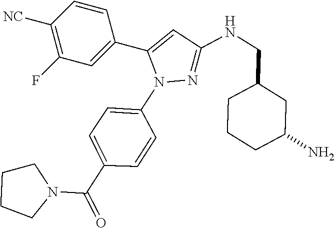 [SMILES: N#Cc1ccc(-c2cc(NC[C@@H]3CCC[C@@H](N)C3)nn2-c2ccc(C(=O)N3CCCC3)cc2)cc1F] | 4-(3-((((1R,3R)-3- aminocyclohexyl)- methyl)amino)-1-(4- (pyrrolidine-1- carbonyl)phenyl)-1H- pyrazol-5-yl)-2- fluorobenzonitrile | /487(M + 1) |
| 65 | 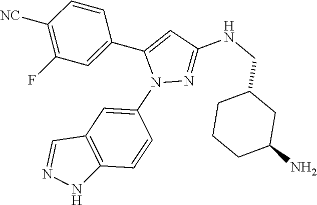 | 4-(3-((((1S,3S)-3- aminocyclohexyl)methyl) amino)-1-(1H-indazol- 5-yl)-1H-pyrazol-5-yl)-2- fluorobenzonitrile | /430(M + 1) |
| 66 | 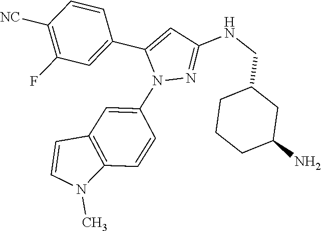 | 4-(3-((((1,3S)-3- aminocyclohexyl)methyl) amino)-1-(1-methyl-1H- indol-5-yl)-1H-pyrazol- 5-yl)-2- fluorobenzonitrile | /443(M + 1) |
| 67 | 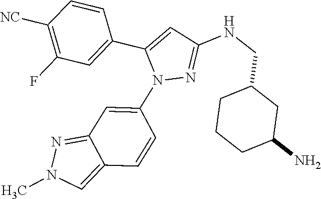 | 4-(3-((((1S,3S)-3- aminocyclohexyl)methyl) amino)-1-(2-methyl-2H- indazol-6-yl)-1H- pyrazol-5-yl)-2- fluorobenzonitrile | /444(M + 1) |
| 68 | 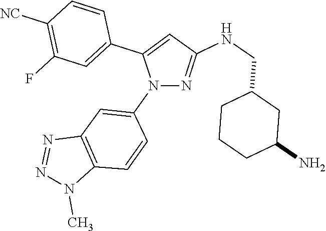 [SMILES: Cn1nnc2cc(-n3nc(NC[C@H]4CCC[C@H](N)C4)cc3-c3ccc(C#N)c(F)c3)ccc21] | 4-(3-((((1S,3S)-3- aminocyclohexyl)methyl) amino)-1-(1-methyl-1H- benzo[d][1,2,3]triazol-5- yl)-1H-pyrazol-5-yl)-2- fluorobenzonitrile | /445(M + 1) |
| 69 | 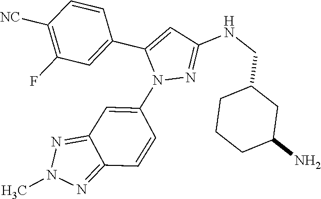 | 4-(3-((((15,3S)-3- aminocyclohexyl)methyl) amino)-1-(2-methyl-2H- benzo[d][1,2,3]triazol-5- yl)-1H-pyrazol-5-yl)-2- fluorobenzonitrile | /445(M + 1) |
| 70 | 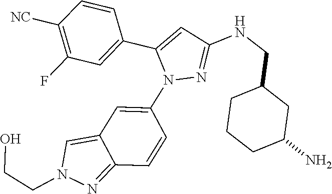 | 4-(3-((((1R,3R)-3- aminocyclohexyl)methyl) amino)-1-(2-(2- hydroxyethyl)-2H- indazol-5-yl)-1H- pyrazol-5-yl)-2- fluorobenzonitrile | /474(M + 1) |
| 71 | 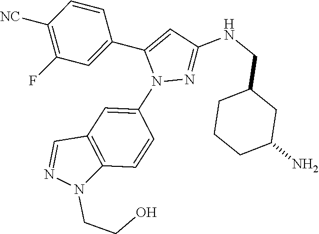 | 4-(3-((((1R,3R)-3- aminocyclohexyl)methyl) amino)-1-(1-(2- hydroxyethyl)-1H- indazol-5-yl)-1H- pyrazol-5-yl)-2- fluorobenzonitrile | /474(M + 1) |
| 72 | 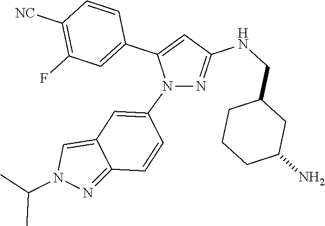 | 4-(3-((((1R,3R)-3- aminocyclohexyl)methyl) amino)-1-(2-isopropyl- 2H-indazol-5-yl)-1H- pyrazol-5-yl)-2- fluorobenzonitrile | /472(M + 1) |
| 73 | 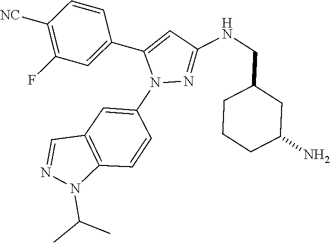 [SMILES: CC(C)n1ncc2cc(-n3nc(NC[C@@H]4CCC[C@@H](N)C4)cc3-c3ccc(C#N)c(F)c3)ccc21] | 4-(3-((((1R,3R)-3- aminocyclohexyl)methyl) amino)-1-(1-isopropyl- 1H-indazol-5-yl)-1H- pyrazol-5-yl)-2- fluorobenzonitrile | /472(M + 1) |
| 74 | 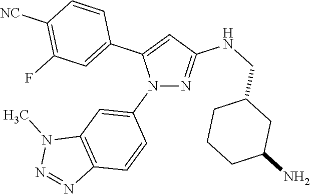 | 4-(3-((((15,3S)-3- aminocyclohexyl)methyl) amino)-1-(1-methyl-1H- benzo[d][1,2,3]triazol-6- yl)-1H-pyrazol-5-yl)-2- fluorobenzonitrile | |
| 75 | 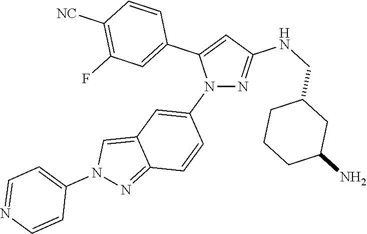 | 4-(3-((((15,3S)-3- aminocyclohexyl)methyl) amino)-1-(2-(pyridin-4- yl)-2H-indazol-5-yl)-1H- pyrazol-5-yl)-2- fluorobenzonitrile | /507(M + 1) |
| 76 | 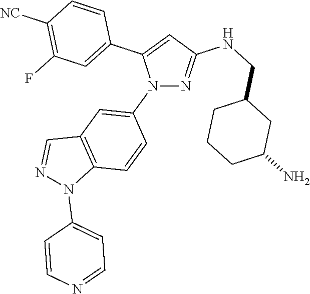 | 4-(3-((((1R,3R)-3- aminocyclohexyl)methyl) amino)-1-(1-(pyridin-4- yl)-1H-indazol-5-yl)-1H- pyrazol-5-yl)-2- fluorobenzonitrile | /507(M + 1) |
| 77 | 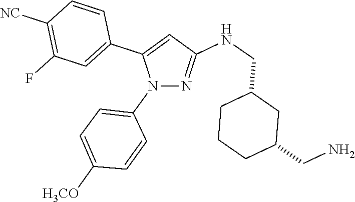 [SMILES: COc1ccc(-n2nc(NC[C@H]3CCC[C@@H](CN)C3)cc2-c2ccc(C#N)c(F)c2)cc1] | 4-(3-((((1S,3R)-3- (aminomethyl)cyclohexyl) methyl)amino)-1-(4- methoxyphenyl)-1H- pyrazol-5-yl)-2- fluorobenzonitrile | |
| 78 | 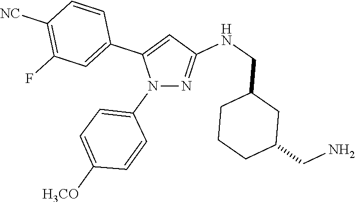 [SMILES: COc1ccc(-n2nc(NC[C@@H]3CCC[C@@H](CN)C3)cc2-c2ccc(C#N)c(F)c2)cc1] | 4-(3-((((1R,3R)-3- (aminomethyl)cyclohexyl) methyl)amino)-1-(4- methoxyphenyl)-1H- pyrazol-5-yl)-2- fluorobenzonitrile | |
| 79 | 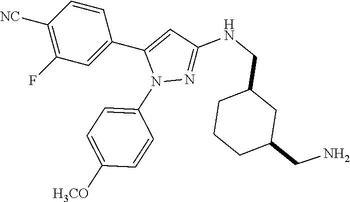 | 4-(3-((((1R,3S)-3- (aminomethyl)cyclohexyl) methyl)amino)-1-(4- methoxyphenyl)-1H- pyrazol-5-yl)-2- fluorobenzonitrile | |
| 80 | 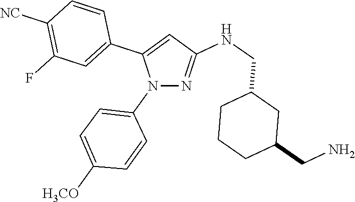 [SMILES: COc1ccc(-n2nc(NC[C@H]3CCC[C@H](CN)C3)cc2-c2ccc(C#N)c(F)c2)cc1] | 4-(3-((((15,3S)-3- (aminomethyl)cyclohexyl) methyl)amino)-1-(4- methoxyphenyl)-1H- pyrazol-5-yl)-2- fluorobenzonitrile | |
| 81 | 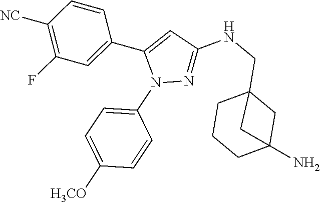 [SMILES: COc1ccc(-n2nc(NCC34CCCC(N)(C3)C4)cc2-c2ccc(C#N)c(F)c2)cc1] | 4-(3-(((5-aminobicyclo- [3.1.1]heptan-1- yl)methyl)amino)-1-(4- methoxyphenyl)-1H- pyrazol-5-yl)-2- fluorobenzonitrile | |
| 82 | 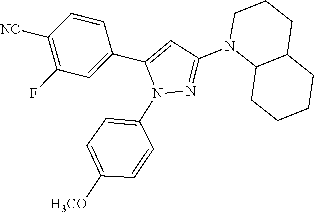 [SMILES: COc1ccc(-n2nc(N3CCCC4CCCCC43)cc2-c2ccc(C#N)c(F)c2)cc1] | 2-fluoro-4-(1-(4- methoxyphenyl)-3- (octahydroquinolin- 1(2H)-yl)-1H-pyrazol-5- yl)benzonitrile | |
| 83 | 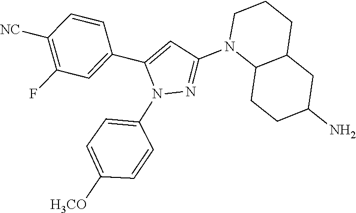 [SMILES: COc1ccc(-n2nc(N3CCCC4CC(N)CCC43)cc2-c2ccc(C#N)c(F)c2)cc1] | 4-(3-(6-amino- octahydroquinolin- 1(2H)-yl)-1-(4- methoxyphenyl)-1H- pyrazol-5-yl)-2- fluorobenzonitrile | |
| 84 | 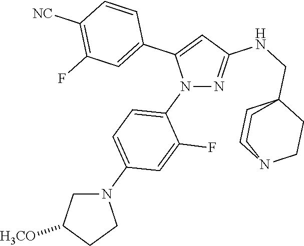 [SMILES: CO[C@H]1CCN(c2ccc(-n3nc(NCC45CCN(CC4)CC5)cc3-c3ccc(C#N)c(F)c3)c(F)c2)C1] | (S)-2-fluoro-4-(1-(2- fluoro-4-(3- methoxypyrrolidin-1- yl)phenyl)-3- ((quinuclidin-4- ylmethyl)amino)-1H- pyrazol-5-yl)benzonitrile | |
| 85 | 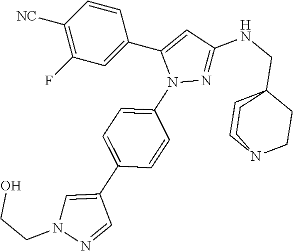 [SMILES: N#Cc1ccc(-c2cc(NCC34CCN(CC3)CC4)nn2-c2ccc(-c3cnn(CCO)c3)cc2)cc1F] | 2-fluoro-4-(1-(4-(1-(2- hydroxyethyl)-1H- pyrazol-4-yl)phenyl)-3- ((quinuclidin-4- ylmethyl)amino)-1H- pyrazol-5-yl)benzonitrile | |
| 86 | 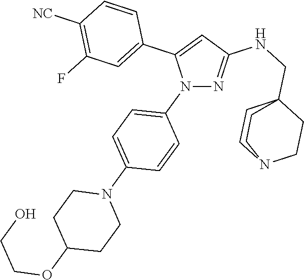 [SMILES: N#Cc1ccc(-c2cc(NCC34CCN(CC3)CC4)nn2-c2ccc(N3CCC(OCCO)CC3)cc2)cc1F] | 2-fluoro-4-(1-(4-(4-(2- hydroxyethoxy)piperidin- 1-yl)phenyl)-3- ((quinuclidin-4- ylmethyl)amino)-1H- pyrazol-5-yl)benzonitrile | |
| 87 | 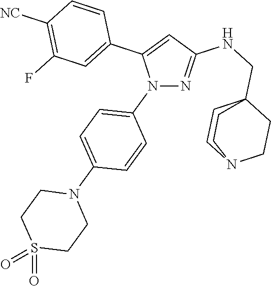 [SMILES: N#Cc1ccc(-c2cc(NCC34CCN(CC3)CC4)nn2-c2ccc(N3CCS(=O)(=O)CC3)cc2)cc1F] | 4-(1-(4-(1,1- dioxidothiomorpholino)- phenyl)-3-((quinuclidin- 4-ylmethyl)amino)-1H- pyrazol-5-yl)-2- fluorobenzonitrile | |

### Intermediate E-1=EXAMPLE 88

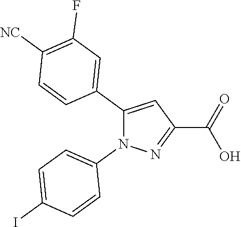 [SMILES: N#Cc1ccc(-c2cc(C(=O)O)nn2-c2ccc(I)cc2)cc1F]

5-(4-Cyano-3-fluorophenyl)-1-(4-iodophenyl)-1H-pyrazole-3-carboxylic acid To a stirred solution of Intermediate B-38 (575 mg, 1.29 mmol, 1.0 equiv) in THF (6 mL) and H₂O (3 mL) was added LiOH (46.2 mg, 1.93 mmol, 1.5 equiv). The resulting mixture was stirred for 1 h at rt. The residue was adjusted to pH 3-4 with HCl (2M). The resulting mixture was extracted with EtOAc (3×20 mL). The combined organic layers were dried with anhydrous Na₂SO₄ and concentrated under vacuum. This resulted in 560 mg (90%) of the title compound as a brown solid. MS-ESI: 434 (M+1).

The following compounds were obtained with methods similar to those used to obtain Intermediate E-1.

TABLE 20
Pyrazol-3-carboxylates.

| Structure | IUPAC Name | Int. No. | M + H⁺ |
|---|---|---|---|
| 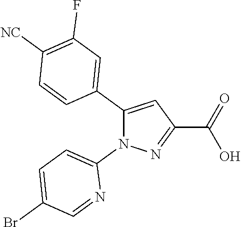 [SMILES: N#Cc1ccc(-c2cc(C(=O)O)nn2-c2ccc(Br)cn2)cc1F] | 1-(5-Bromopyridin-2- yl)-5-(4-cyano-3- fluorophenyl)-1H-pyr- azole-3-carboxylic acid | E-2 | 389 |
| 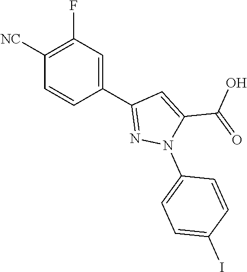 [SMILES: N#Cc1ccc(-c2cc(C(=O)O)n(-c3ccc(I)cc3)n2)cc1F] | 3-(4-Cyano-3-fluoro- phenyl)-1-(4-iodo- phenyl)-1H-pyrazole- 5-carboxylic acid | E-3 | 434 |

### Intermediate E-4

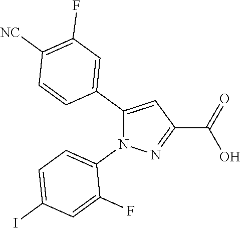 [SMILES: N#Cc1ccc(-c2cc(C(=O)O)nn2-c2ccc(I)cc2F)cc1F]

### 5-(4-Cyano-3-fluorophenyl)-1-(2-fluoro-4-iodophenyl)-1H-pyrazole-3-carboxylic acid

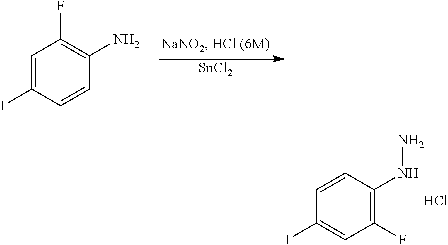 [SMILES: Nc1ccc(I)cc1F ; "SnCl2" ; NNc1ccc(I)cc1F ; Cl]

2-Fluoro-4-iodophenyl) hydrazine hydrochloride To a stirred mixture of 2-fluoro-4-iodoaniline (10 g, 42.2 mmol, 1.00 equiv) in HCl (6 M, 110 mL) was added sodium nitrite (3.2 g, 46.4 mmol, 1.1 equiv) in H₂O (5 mL) dropwise at −5° C. The resulting mixture was stirred for 1 h at −5° C. To above reaction mixture was added SnCl₂ (20.2 g, 105 mmol, 2.5 equiv) in cc.HCl (8 mL) dropwise at −10° C. The resulting mixture was stirred for 5 h at −5° C. The precipitated solids were collected by filtration and washed with DCM (3×30 mL). This resulted in 17.8 g (crude) of the title compound as a yellow solid. 289 (M+1).

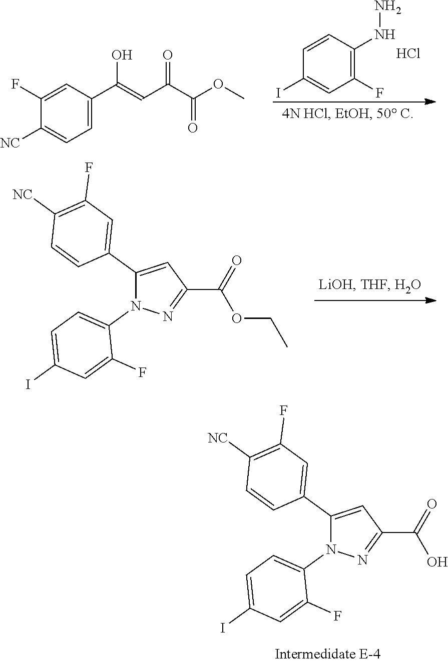

Methyl (Z)-4-(4-cyano-3-fluorophenyl)-4-hydroxy-2-oxobut-3-enoate (from Intermediate B-49) was converted to Intermediate E-4 with methods similar to those used to obtain Intermediates B-50 and E-1.

The following compounds were obtained with methods similar to those used to obtain Intermediate E-4.

TABLE 21
Pyrazol-3-carboxylates.

| Structure | IUPAC Name | Int. No. | M + H⁺ |
|---|---|---|---|
| 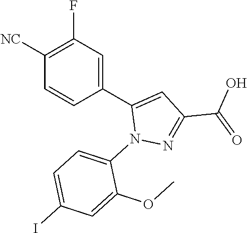 [SMILES: COc1cc(I)ccc1-n1nc(C(=O)O)cc1-c1ccc(C#N)c(F)c1] | 5-(4-Cyano-3-fluoro- phenyl)-1-(4-iodo-2- methoxyphenyl)-1H- pyrazole-3-carboxylic acid | E-5 | 464 |

### Intermediate E-6

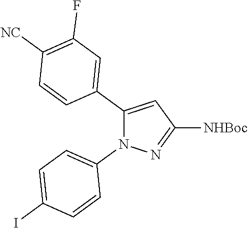 [SMILES: CC(C)(C)OC(=O)Nc1cc(-c2ccc(C#N)c(F)c2)n(-c2ccc(I)cc2)n1]

### Tert-butyl (5-(4-cyano-3-fluorophenyl)-1-(4-iodophenyl)-1H-pyrazol-3-yl) carbamate

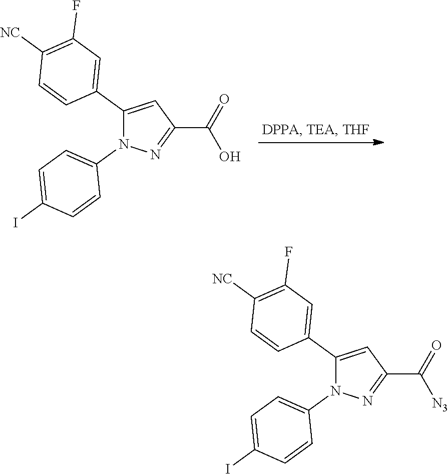 [SMILES: N#Cc1ccc(-c2cc(C(=O)O)nn2-c2ccc(I)cc2)cc1F ; N#Cc1ccc(-c2cc(C(=O)N=[N+]=[N-])nn2-c2ccc(I)cc2)cc1F]

5-(4-Cyano-3-fluorophenyl)-1-(4-iodophenyl)-1H-pyrazole-3-carbonyl azide To a stirred solution of Intermediate E-1 (11 g, 25.4 mmol, 1.0 equiv) in THF (150 mL) were added DPPA (14 g, 50.8 mmol, 2.0 equiv) and TEA (10.6 mL, 76.2 mmol, 3.0 equiv). The resulting mixture was stirred for 2 h at room temperature. The reaction mixture was diluted with water (200 mL) and extracted with EtOAc (3×500 mL). The combined organic layers were dried with anhydrous Na₂SO₄ and concentrated under vacuum. This resulted in 8.76 g (crude) of the title compound as a brown solid. MS-ESI: 459 (M+1).

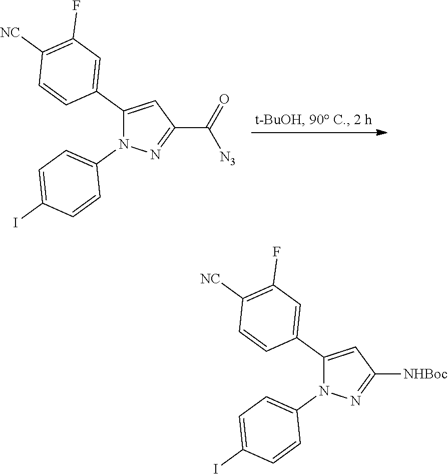 [SMILES: N#Cc1ccc(-c2cc(C(=O)N=[N+]=[N-])nn2-c2ccc(I)cc2)cc1F ; CC(C)(C)OC(=O)Nc1cc(-c2ccc(C#N)c(F)c2)n(-c2ccc(I)cc2)n1]

Tert-butyl (5-(4-cyano-3-fluorophenyl)-1-(4-iodophenyl)-1H-pyrazol-3-yl) carbamate A solution of 5-(4-cyano-3-fluorophenyl)-1-(4-iodophenyl)-1H-pyrazole-3-carbonyl azide (8.76 g, crude) in t-BuOH (80 mL) was stirred for 2 h at 90° C. The reaction mixture was diluted with water (100 mL) and extracted with EtOAc (3×200 mL). The resulting mixture was concentrated under vacuum. The residue was purified by reversed-phase flash chromatography with the following conditions: column, C18; mobile phase, MeCN in Water (10 mM NH₄HCO₃), 50% to 50% in 30 min; Detector UV, 254 nm. This resulted in 5.67 g (44.2%, over two steps) of the title compound as an off-white solid. MS-ESI: 505 (M+1). ¹H NMR (400 MHZ, DMSO-d₆) δ 10.0 (brs, s, 1H), 7.92 (t, J=7.6 Hz, 1H), 7.78 (d, J=8.4 Hz, 2H), 7.54 (dd, J=10.4, 1.2 Hz, 1H), 7.20 (dd, J=8.4, 1.6 Hz, 1H), 7.05 (d, J=8.8 Hz, 2H), 6.89 (s, 1H), 1.47 (s, 9H).

The following compounds were obtained with methods similar to those used to obtain Intermediate E-6.

TABLE 22
Pyrazol-3-carbamates.

| Structure | IUPAC Name | Int. No. | M + H⁺ |
|---|---|---|---|
| 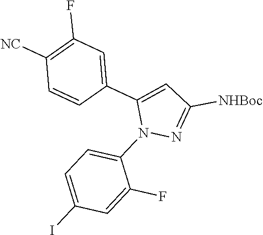 [SMILES: CC(C)(C)OC(=O)Nc1cc(-c2ccc(C#N)c(F)c2)n(-c2ccc(I)cc2F)n1] | Tert-butyl (5-(4- cyano-3-fluorophen- yl)-1-(2-fluoro-4- iodophenyl)-1H- pyrazol-3-yl)-carba- mate | E-7 | 523 |
| 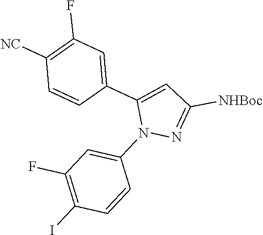 [SMILES: CC(C)(C)OC(=O)Nc1cc(-c2ccc(C#N)c(F)c2)n(-c2ccc(I)c(F)c2)n1] | Tert-butyl (5-(4- cyano-3-fluorophen- yl)-1-(3-fluoro-4- iodophenyl)-1H- pyrazol-3-yl)carba- mate | E-8 | 523 |
| 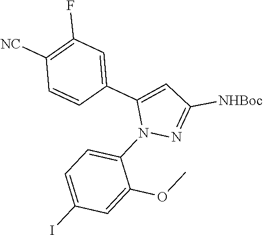 [SMILES: COc1cc(I)ccc1-n1nc(NC(=O)OC(C)(C)C)cc1-c1ccc(C#N)c(F)c1] | Tert-butyl (5-(4-cyano-3-fluoro- phenyl)-1-(4-iodo- 2-methoxyphenyl)- 1H-pyrazol-3-yl)- carbamate | E-9 | 535 |
| 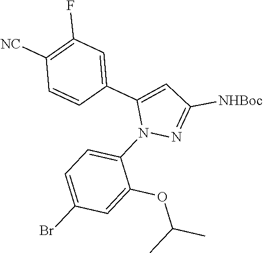 [SMILES: CC(C)Oc1cc(Br)ccc1-n1nc(NC(=O)OC(C)(C)C)cc1-c1ccc(C#N)c(F)c1] | Tert-butyl (1-(4- bromo-2-isopro- poxyphenyl)-5-(4- cyano-3-fluorophen- yl)-1H-pyrazol-3- yl)carbamate | E-10 | 515 |
| 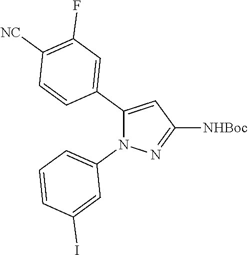 [SMILES: CC(C)(C)OC(=O)Nc1cc(-c2ccc(C#N)c(F)c2)n(-c2cccc(I)c2)n1] | Tert-butyl (5-(4-cyano-3-fluorophen- yl)-1-(3-iodophen- yl)-1H-pyrazol-3- yl)carbamate | E-11 | 505 |
| 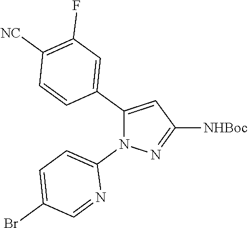 [SMILES: CC(C)(C)OC(=O)Nc1cc(-c2ccc(C#N)c(F)c2)n(-c2ccc(Br)cn2)n1] | Tert-butyl (1-(5- bromopyridin-2- yl)-5-(4-cyano-3- fluorophenyl)-1H- pyrazol-3-yl)carba- mate | E-12 | 458 |

### Example 89

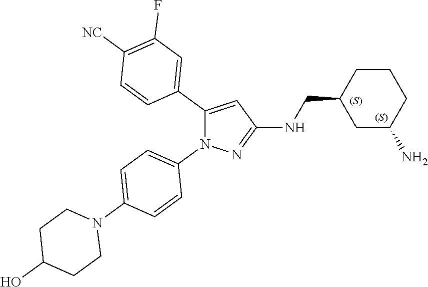

### 4-(3-((((1S,3S)-3-aminocyclohexyl)methyl)amino)-1-(4-(4-hydroxypiperidin-1-yl)phenyl)-1H-pyrazol-5-yl)-2-fluorobenzonitrile

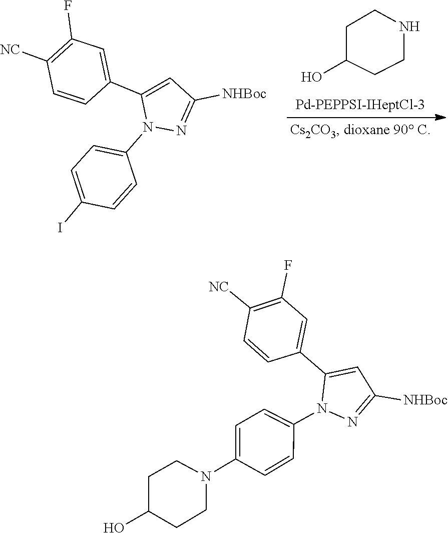 [SMILES: CC(C)(C)OC(=O)Nc1cc(-c2ccc(C#N)c(F)c2)n(-c2ccc(I)cc2)n1 ; OC1CCNCC1 ; CC(C)(C)OC(=O)Nc1cc(-c2ccc(C#N)c(F)c2)n(-c2ccc(N3CCC(O)CC3)cc2)n1]

Tert-butyl (5-(4-cyano-3-fluorophenyl)-1-(4-(4-hydroxypiperidin-1-yl)phenyl)-1H-pyrazol-3-yl) carbamate To a stirred solution of Intermediate E-6 (500 mg, 0.99 mmol, 1.0 equiv) in 1,4-dioxane (5 mL) were added piperidin-4-ol (100 mg, 0.99 mmol, 1.0 equiv), Pd-PEPPSI-IHeptCl 3-chloropyridine (97 mg, 0.099 mmol, 0.1 equiv) and Cs₂CO₃ (969 mg, 2.97 mmol, 3.0 equiv) in portions at room temperature under nitrogen atmosphere. The resulting mixture was stirred for 2 h at 100° C. under nitrogen atmosphere. The mixture was allowed to cool down to room temperature. The reaction mixture was diluted with water (10 mL) and extracted with EtOAc (3×50 mL). The combined organic layers were dried with anhydrous Na₂SO₄ and concentrated under vacuum. The residue was purified by Prep-TLC (PE/EtOAc 1:2). This resulted in 450 mg (95%) as a yellow oil. MS-ESI: 478 (M+1).

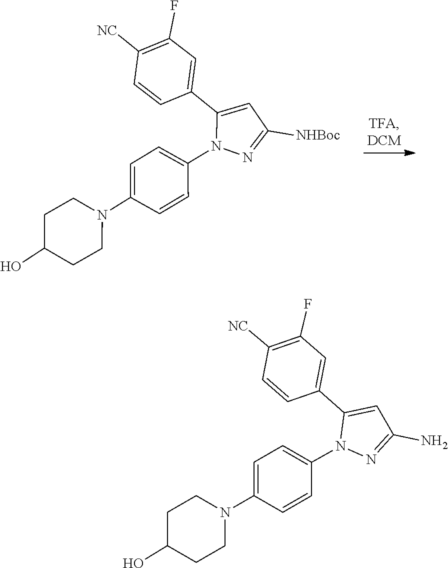 [SMILES: CC(C)(C)OC(=O)Nc1cc(-c2ccc(C#N)c(F)c2)n(-c2ccc(N3CCC(O)CC3)cc2)n1 ; N#Cc1ccc(-c2cc(N)nn2-c2ccc(N3CCC(O)CC3)cc2)cc1F]

4-(3-Amino-1-(4-(4-hydroxypiperidin-1-yl)phenyl)-1H-pyrazol-5-yl)-2-fluorobenzonitrile A solution of tert-butyl (5-(4-cyano-3-fluorophenyl)-1-(4-(4-hydroxypiperidin-1-yl)phenyl)-1H-pyrazol-3-yl) carbamate (450 mg, 0.94 mmol, 1.0 equiv) in DCM (5 mL) and TFA (1 mL) was stirred for 1 h at room temperature. The mixture was basified to pH 8 with sat. Na₂CO₃ (aq.) and extracted with DCM (3×50 mL). The combined organic layers were washed with NaCl (aq.) (3×50 mL), dried over anhydrous Na₂SO₄ and concentrated under vacuum. This resulted in 350 mg (88%) of the title compound as a yellow solid. MS-ESI: 378 (M+1).

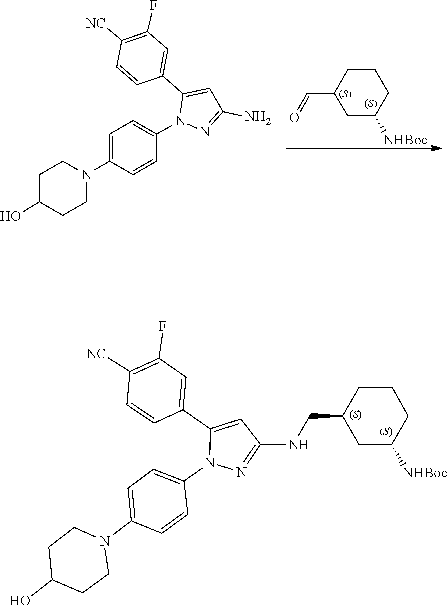

Tert-butyl ((1S,3S)-3-(((5-(4-cyano-3-fluorophenyl)-1-(4-(4-hydroxypiperidin-1-yl)phenyl)-1H-pyrazol-3-yl)amino)methyl)cyclohexyl) carbamate To a stirred solution of 4-(3-amino-1-(4-(4-hydroxypiperidin-1-yl)phenyl)-1H-pyrazol-5-yl)-2-fluorobenzonitrile (350 mg, 0.93 mmol, 1.0 equiv) in DCM (4 mL) was added tert-butyl ((1S,3S)-3-formylcyclohexyl) carbamate (211 mg, 0.93 mmol, 1.0 equiv) for 40 min at room temperature. The to above mixture was added NaBH(OAc)₃ (590 mg, 2.78 mmol, 3.0 equiv) in portions at room temperature. The resulting mixture was stirred for 2 h at room temperature. The reaction was quenched with MeOH (5 mL) at room temperature. The mixture was concentrated under vacuum. The residue was purified by Prep-TLC (DCM/MeOH 10:1). This resulted in 330 mg (60%) as a yellow solid. MS-ESI: 589 (M+1).

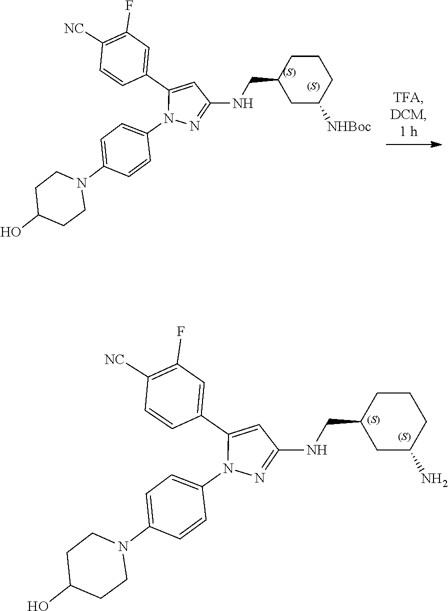

4-(3-((((1S,3S)-3-aminocyclohexyl)methyl)amino)-1-(4-(4-hydroxypiperidin-1-yl)phenyl)-1H-pyrazol-5-yl)-2-fluorobenzonitrile A solution of tert-butyl ((1S,3S)-3-(((5-(4-cyano-3-fluorophenyl)-1-(4-(4-hydroxypiperidin-1-yl)phenyl)-1H-pyrazol-3-yl)amino)methyl)cyclohexyl) carbamate (330 mg, 0.56 mmol, 1.0 equiv) in DCM (5 mL) and TFA (1 mL) was stirred for 1 h at room temperature. The resulting mixture was concentrated under vacuum. The crude product was purified by Prep-HPLC with the following conditions: Xselect CSH C18 OBD Column 30*150 mm 5 μm; Mobile Phase A: water (0.05% TFA), Mobile Phase B: ACN; Flow rate: 60 mL/min; Gradient: 14% B to 25% B in 7 min, Detector UV: 254/220 nm; RT1(min): 6.5. Then the product was separated by Prep-Chiral-HPLC with the following condition: CHIRALPAK IG, 2*25 cm, 5 μm; Mobile Phase A: Hex (0.2% DEA), Mobile Phase B: EtOH: DCM=1:1; Flow rate: 20 mL/min; Gradient: 50% B to 50% B in 22 min; Detector UV: 220/254 nm; RT1(min): 12.58; RT2(min): 15.25; Sample Solvent: EtOH: DCM=1:1; Injection Volume: 0.55 mL; Number of Runs: 4. This resulted in 40 mg (11.6%) of the title compound as a yellow solid. MS-ESI: 489 (M+1). ¹H NMR (400 MHZ, MeOD-d₄) δ 7.66 (t, J=4.4 Hz, 1H), 7.23-7.17 (m, 2H), 7.09 (d, J=9.2 Hz, 2H), 6.99 (d, J=9.2 Hz, 2H), 6.08 (s, 1H), 3.90-3.72 (m, 1H), 3.69-3.56 (m, 2H), 3.22-3.05 (m, 3H), 3.01-2.87 (m, 2H), 2.11-1.88 (m, 3H), 1.78-1.52 (m, 8H), 1.51-1.40 (m, 1H), 1.38-1.26 (m, 1H).

Examples in the following table were prepared using similar conditions as described for those of Example 89 from appropriate starting materials.

TABLE 23
Example Compounds.

| Ex. | | | |
|---|---|---|---|
| No. | Structure | IUPAC Name | Data |
| 90 | 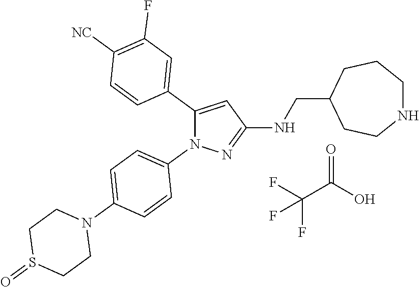 | 4-(3-((Azepan-4-yl- methyl)amino)-1-(4- (1-oxidothiomorph- olino)phenyl)-1H- pyrazol-5-yl)-2- fluorobenzonitrile 2,2,2-trifluoroacetate | MS-ESI: 507(M + 1) |
| 91 | 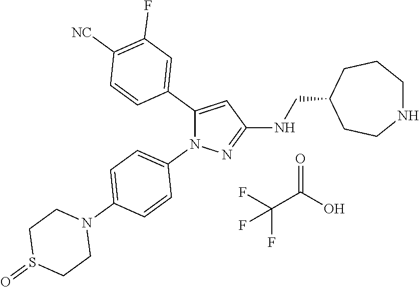 | (R)-4-(3-((azepan-4- ylmethyl)amino)-1- (4-(1-oxidothio- morpholino)phenyl)- 1H-pyrazol-5-yl)-2- fluorobenzonitrile 2,2,2-trifluoroacetate | MS-ESI: 507(M + 1) |
| 92 | 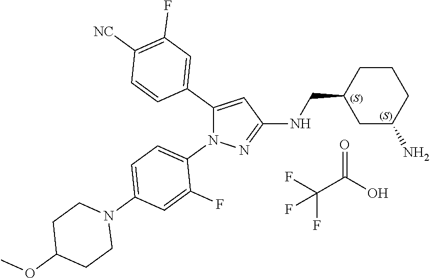 | 4-(3-((((1S,3S)-3- aminocyclohexyl)- methyl)amino)-1-(2- fluoro-4-(4-methoxy- piperidin-1-yl)phen- yl)-1H-pyrazol-5-yl)- 2-fluorobenzonitrile 2,2,2-trifluoroacetate | MS-ESI: 521(M + 1) |
| 93 | 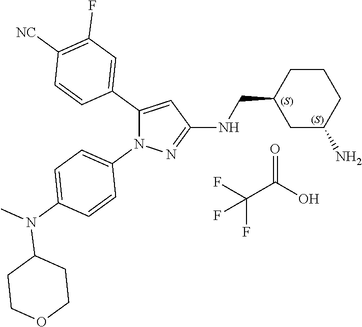 | 4-(3-((((1S,3S)-3- aminocyclohexyl)- methyl)amino)-1-(4- (methyl(tetrahydro- 2H-pyran-4-yl)- amino)phenyl)-1H- pyrazol-5-yl)-2- fluorobenzonitrile 2,2,2-trifluoroacetate | MS-ESI: 503(M + 1) |
| 94 | 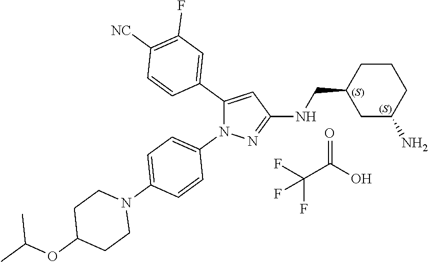 | 4-(3-((((1S,3S)-3- aminocyclohexyl)- methyl)amino)-1-(4- (4-isopropoxypiper- idin-1-yl)phenyl)-1H- pyrazol-5-yl)-2- fluorobenzonitrile 2,2,2-trifluoroacetate | MS-ESI: 531(M + 1) |
| 95 | 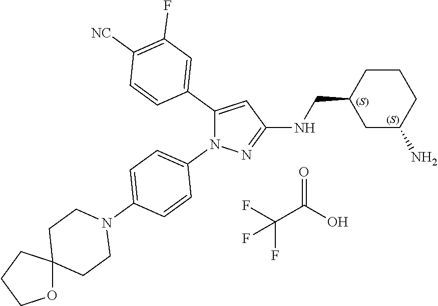 | 4-(1-(4-(1-Oxa-8- azaspiro[4.5]decan- 8-yl)phenyl)-3- ((((1S,3S)-3-amino- cyclohexyl)methyl)- amino)-1H-pyrazol- 5-yl)-2-fluorobenzo- nitrile 2,2,2-trifluoro- acetate | MS-ESI: 529(M + 1) |
| 96 | 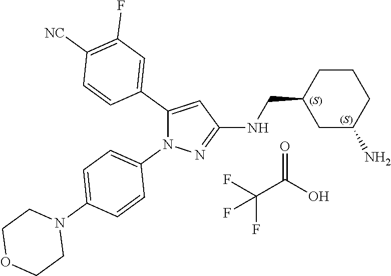 | 4-(3-((((1S,3S)-3- aminocyclohexyl)- methyl)amino)-1-(4- morpholinophenyl)- 1H-pyrazol-5-yl)-2- fluorobenzonitrile 2,2,2-trifluoroacetate | MS-ESI: 475(M + 1) |
| 97 | 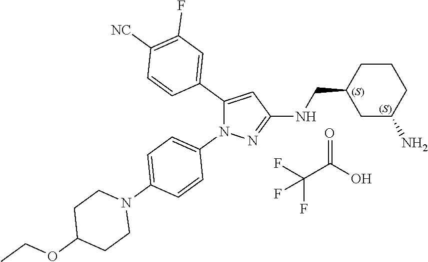 | 4-(3-((((1S,3S)-3- aminocyclohexyl)- methyl)amino)-1-(4- (4-ethoxypiperidin- 1-yl)phenyl)-1H- pyrazol-5-yl)-2- fluorobenzonitrile 2,2,2-trifluoroacetate | MS-ESI: 517(M + 1) |
| 98 | 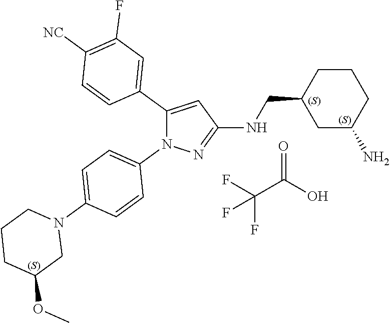 | 4-(3-((((1S,3S)-3- aminocyclohexyl)- methyl)amino)-1-(4- ((S)-3-methoxypiper- idin-1-yl)phenyl)-1H- pyrazol-5-yl)-2- fluorobenzonitrile 2,2,2-trifluoroacetate | MS-ESI: 503(M + 1) |
| 99 | 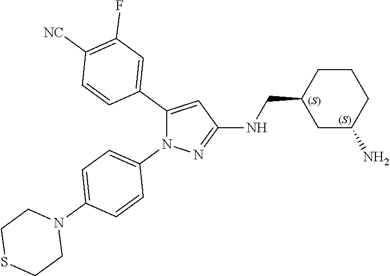 | 4-(3-((((1S,3S)-3- aminocyclohexyl)- methyl)amino)-1-(4- thiomorpholinophen- yl)-1H-pyrazol-5-yl)- 2-fluorobenzonitrile | MS-ESI: 491(M + 1) |
| 100 | 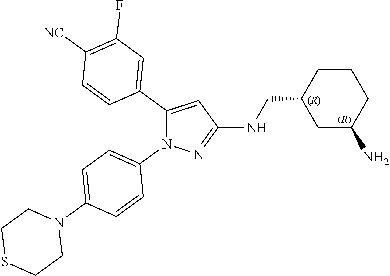 | 4-(3-((((1R,3R)-3- aminocyclohexyl)- methyl)amino)-1-(4- thiomorpholinophen- yl)-1H-pyrazol-5-yl)- 2-fluorobenzonitrile | MS-ESI: 491(M + 1) |
| 101 | 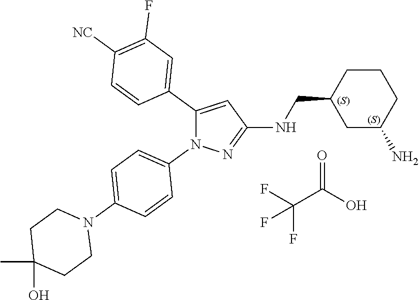 | 4-(3-((((1S,3S)-3- aminocyclohexyl)- methyl)amino)-1-(4- (4-hydroxy-4-meth- ylpiperidin-1-yl)- phenyl)-1H-pyrazol- 5-yl)-2-fluorobenzo- nitrile 2,2,2-trifluoro- acetate | MS-ESI: 503(M + 1) |
| 102 | 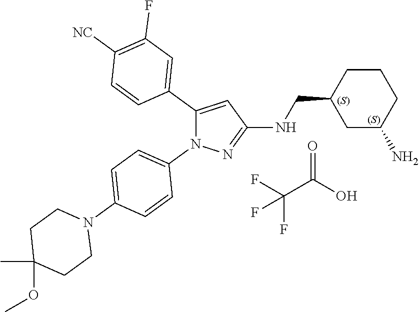 | 4-(3-((((1S,3S)-3- aminocyclohexyl)- methyl)amino)-1-(4- (4-methoxy-4-meth- ylpiperidin-1-yl)- phenyl)-1H-pyrazol- 5-yl)-2-fluorobenzo- nitrile 2,2,2-trifluoro- acetate | MS-ESI: 517(M + 1) |
| 103 | 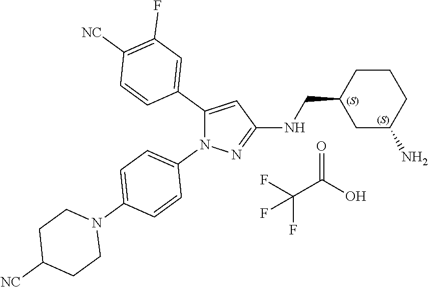 | 1-(4-(3-((((1S,3S)-3- aminocyclohexyl)- methyl)amino)-5-(4- cyano-3-fluorophen- yl)-1H-pyrazol-1- yl)phenyl)piperidine- 4-carbonitrile 2,2,2- trifluoroacetate | MS-ESI: 498(M + 1) |
| 104 | 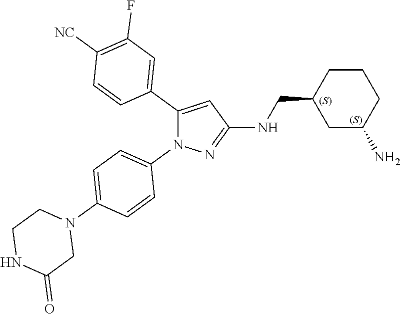 | 4-(3-((((1S,3S)-3- aminocyclohexyl)- methyl)amino)-1-(4- (3-oxopiperazin-1- yl)phenyl)-1H-pyr- azol-5-yl)-2-fluoro- benzonitrile | MS-ESI: 488(M + 1) |
| 105 | 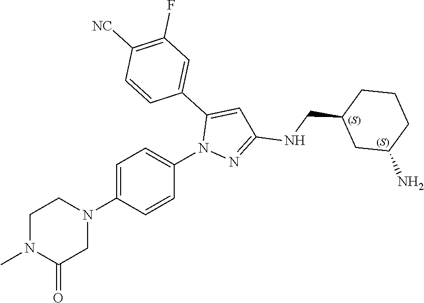 | 4-(3-((((1S,3S)-3- aminocyclohexyl)- methyl)amino)-1-(4- (4-methyl-3-oxo- piperazin-1-yl)phen- yl)-1H-pyrazol-5- yl)-2-fluorobenzo- nitrile | MS-ESI: 502(M + 1) |
| 106 | 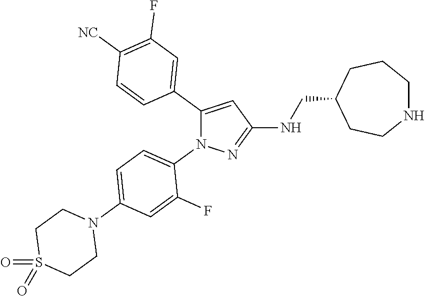 [SMILES: N#Cc1ccc(-c2cc(NC[C@@H]3CCCNCC3)nn2-c2ccc(N3CCS(=O)(=O)CC3)cc2F)cc1F] | (R)-4-(3-((azepan-4- ylmethyl)amino)-1- (4-(1,1-dioxidothio- morpholino)-2- fluorophenyl)-1H- pyrazol-5-yl)-2- fluorobenzonitrile | MS-ESI: 541(M + 1) |
| 107 | 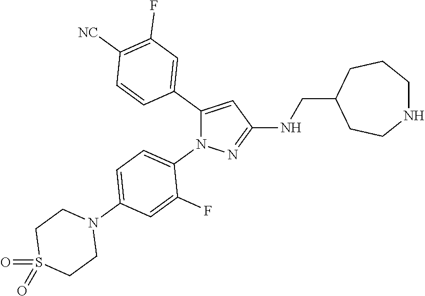 [SMILES: N#Cc1ccc(-c2cc(NCC3CCCNCC3)nn2-c2ccc(N3CCS(=O)(=O)CC3)cc2F)cc1F] | 4-(3-((azepan-4-yl- methyl)amino)-1-(4- (1,1-dioxidothiomor- pholino)-2-fluoro- phenyl)-1H-pyrazol- 5-yl)-2-fluorobenzo- nitrile | MS-ESI: 541(M + 1) |
| 108 | 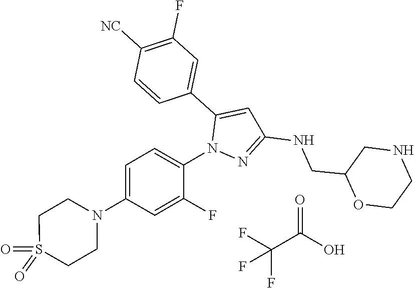 | 4-(1-(4-(1,1-dioxido- thiomorpholino)-2- fluorophenyl)-3- ((morpholin-2-yl- methyl)amino)-1H- pyrazol-5-yl)-2- fluorobenzonitrile 2,2,2-trifluoroacetate | MS-ESI: 529(M + 1) |
| 109 | 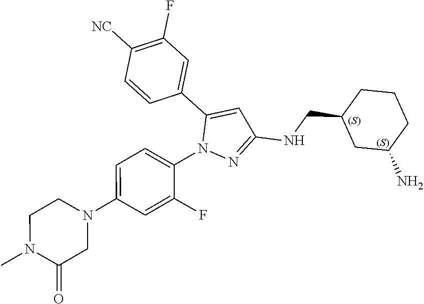 | 4-(3-((((1S,3S)-3- aminocyclohexyl)- methyl)amino)-1-(2- fluoro-4-(4-methyl-3- oxopiperazin-1-yl)- phenyl)-1H-pyrazol- 5-yl)-2-fluorobenzo- nitrile | MS-ESI: 520(M + 1) |
| 110 | 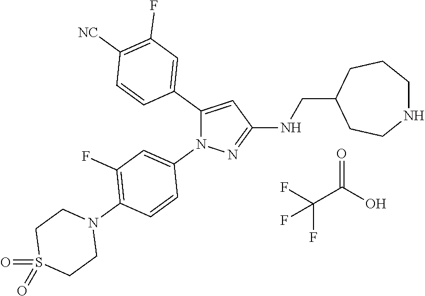 | 4-(3-((azepan-4-yl- methyl)amino)-1-(4- (1,1-dioxidothiomor- pholino)-3-fluoro- phenyl)-1H-pyrazol- 5-yl)-2-fluorobenzo- nitrile 2,2,2-trifluoro- acetate | MS-ESI: 541(M + 1) |
| 111 | 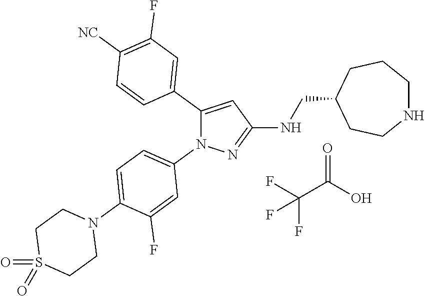 | (R)-4-(3-((azepan-4- ylmethyl)amino)-1- (4-(1,1-dioxidothio- morpholino)-3-fluorophenyl)-1H- pyrazol-5-yl)-2- fluorobenzonitrile 2,2,2-trifluoroacetate | MS-ESI: 541(M + 1) |
| 112 | 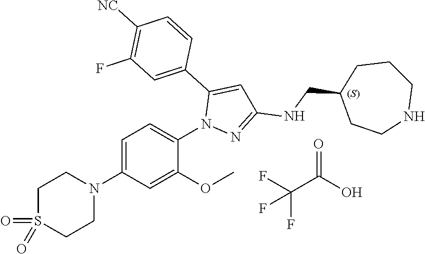 | (S)-4-(3-((azepan-4- ylmethyl)amino)-1- (4-(1,1-dioxidothio- morpholino)-2-meth- oxyphenyl)-1H-pyr- azol-5-yl)-2-fluoro- benzonitrile 2,2,2- trifluoroacetate | MS-ESI: 553(M + 1) |
| 113 | 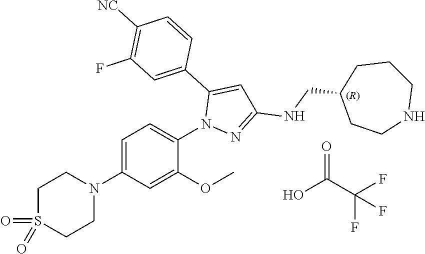 | (R)-4-(3-((azepan-4- ylmethyl)amino)-1- (4-(1,1-dioxidothio- morpholino)-2-meth- oxyphenyl)-1H-pyr- azol-5-yl)-2-fluoro- benzonitrile 2,2,2- trifluoroacetate | MS-ESI: 553(M + 1) |
| 114 | 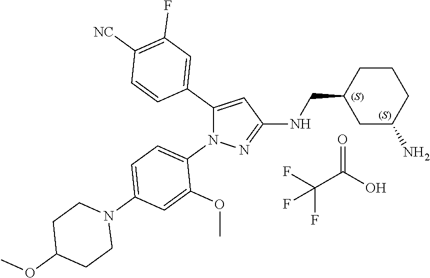 | 4-(3-((((1S,3S)-3- aminocyclohexyl)- methyl)amino)-1-(2- methoxy-4-(4-meth- oxypiperidin-1-yl)- phenyl)-1H-pyrazol- 5-yl)-2-fluorobenzo- nitrile 2,2,2-trifluoro- acetate | MS-ESI: 533(M + 1) |
| 115 | 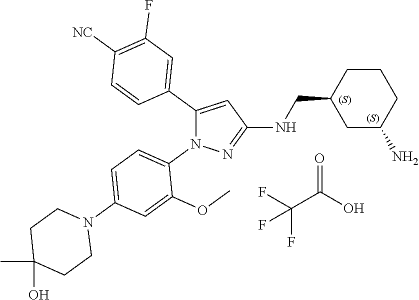 | 4-(3-((((1S,3S)-3- aminocyclohexyl)- methyl)amino)-1-(4- (4-hydroxy-4-meth- ylpiperidin-1-yl)-2- methoxyphenyl)-1H- pyrazol-5-yl)-2- fluorobenzonitrile 2,2,2-trifluoroacetate | MS-ESI: 553(M + 1) |
| 116 | 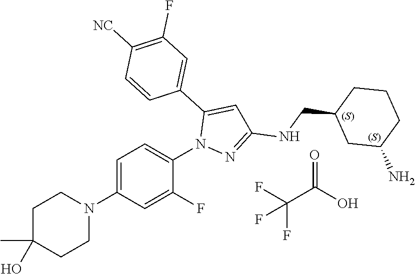 | 4-(3-((((1S,3S)-3- aminocyclohexyl)- methyl)amino)-1-(2- fluoro-4-(4-hydroxy- 4-methylpiperidin-1- yl)phenyl)-1H-pyr- azol-5-yl)-2-fluoro- benzonitrile 2,2,2- trifluoroacetate | MS-ESI: 521(M + 1) |
| 117 | 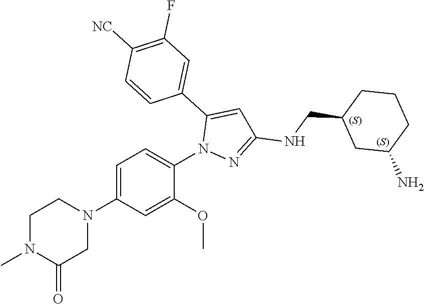 | 4-(3-((((1S,3S)-3- aminocyclohexyl)- methyl)amino)-1-(2- methoxy-4-(4-meth- yl-3-oxopiperazin-1- yl)phenyl)-1H-pyr- azol-5-yl)-2-fluoro- benzonitrile | MS-ESI: 532(M + 1) |
| 118 | 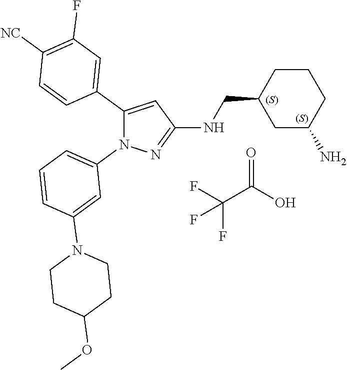 | 4-(3-((((1S,3S)-3- aminocyclohexyl)- methyl)amino)-1-(3- (4-methoxypiperi- din-1-yl)phenyl)-1H- pyrazol-5-yl)-2- fluorobenzonitrile 2,2,2-trifluoroacetate | MS-ESI: 503(M + 1) |
| 119 | 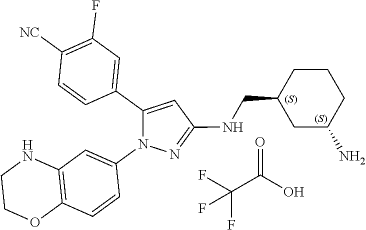 | 4-(3-((((1S,3S)-3- aminocyclohexyl)- methyl)amino)-1- (3,4-dihydro-2H- benzo[b][1,4]oxazin- 6-yl)-1H-pyrazol-5- yl)-2-fluorobenzo- nitrile 2,2,2-trifluoro- acetate | MS-ESI: 447(M + 1) |
| 120 | 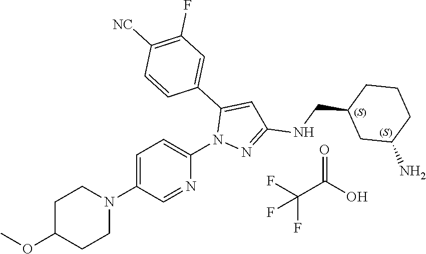 | 4-(3-((((1S,3S)-3- aminocyclohexyl)- methyl)amino)-1-(5- (4-methoxypiperi- din-1-yl)pyridin-2- yl)-1H-pyrazol-5-yl)- 2-fluorobenzonitrile 2,2,2-trifluoroacetate | MS-ESI: 504(M + 1) |

### Example 121

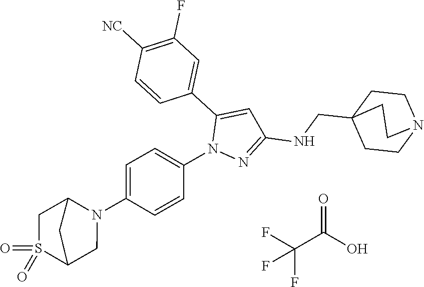 [SMILES: N#Cc1ccc(-c2cc(NCC34CCN(CC3)CC4)nn2-c2ccc(N3CC4CC3CS4(=O)=O)cc2)cc1F ; O=C(O)C(F)(F)F]

### 4-(1-(4-(2,2-Dioxido-2-thia-5-azabicyclo[2.2.1]heptan-5-yl)phenyl)-3-((quinuclidin-4-ylmethyl)amino)-1H-pyrazol-5-yl)-2-fluorobenzonitrile 2,2,2-trifluoroacetate

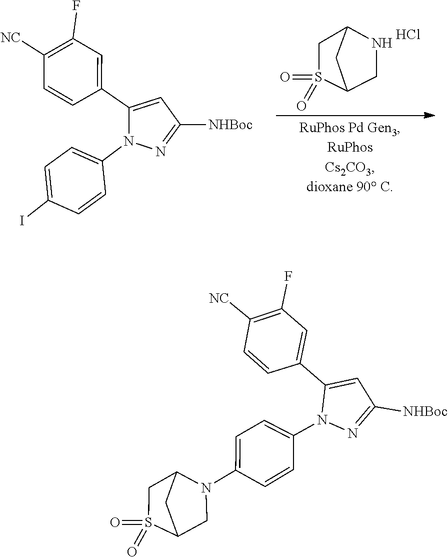 [SMILES: CC(C)(C)OC(=O)Nc1cc(-c2ccc(C#N)c(F)c2)n(-c2ccc(I)cc2)n1 ; O=S1(=O)CC2CC1CN2 ; Cl ; CC(C)(C)OC(=O)Nc1cc(-c2ccc(C#N)c(F)c2)n(-c2ccc(N3CC4CC3CS4(=O)=O)cc2)n1]

Tert-butyl (5-(4-cyano-3-fluorophenyl)-1-(4-(2,2-dioxido-2-thia-5-azabicyclo[2.2.1]heptan-5-yl)phenyl)-1H-pyrazol-3-yl) carbamate To a stirred solution of Intermediate E-6 (3.0 g, 5.95 mmol, 1.0 equiv) in dioxane (60 mL) were added 2-thia-5-azabicyclo[2.2.1]heptane 2,2-dioxide hydrochloride (1.31 g, 8.92 mmol, 1.5 equiv), RuPhos Palladacycle Gen.3 (0.50 g, 0.60 mmol, 0.10 equiv), RuPhos (0.28 g, 0.60 mmol, 0.1 equiv) and Cs₂CO₃ (3.88 g, 11.9 mmol, 2.0 equiv) in portions at room temperature under nitrogen atmosphere. The reaction mixture was stirred for 8 h at 90° C. under nitrogen atmosphere. The mixture was allowed to cool down to room temperature. The reaction mixture was diluted with water (100 mL) and extracted with EtOAc (3×100 mL). The combined organic layers were dried with anhydrous Na₂SO₄ and concentrated under vacuum. The residue was eluted from silica gel with PE/EtOAc (1:1). This resulted in 2.0 g (61%) of the title compound as a yellow solid. MS-ESI: 524 (M+1).

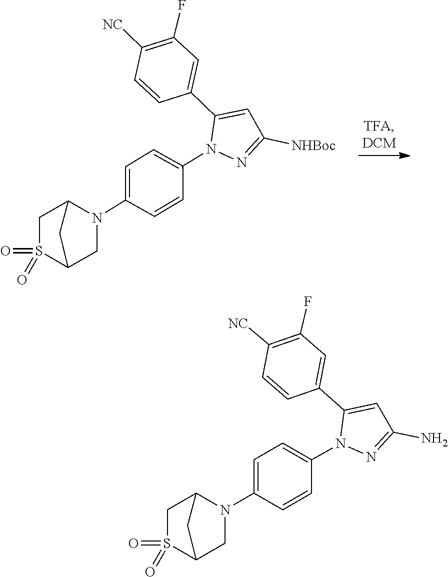 [SMILES: CC(C)(C)OC(=O)Nc1cc(-c2ccc(C#N)c(F)c2)n(-c2ccc(N3CC4CC3CS4(=O)=O)cc2)n1 ; N#Cc1ccc(-c2cc(N)nn2-c2ccc(N3CC4CC3CS4(=O)=O)cc2)cc1F]

4-(3-Amino-1-(4-(2,2-dioxido-2-thia-5-azabicyclo[2.2.1]heptan-5-yl)phenyl)-1H-pyrazol-5-yl)-2-fluorobenzonitrile A solution of tert-butyl (5-(4-cyano-3-fluorophenyl)-1-(4-(2,2-dioxido-2-thia-5-azabicyclo[2.2.1]heptan-5-yl)phenyl)-1H-pyrazol-3-yl) carbamate (1.97 g, 3.76 mmol, 1.0 equiv) in DCM (30 mL) and TFA (6 mL) was stirred for 2 h at room temperature. The resulting mixture was basified to pH 7-8 with sat. Na₂CO₃ (aq.) and extracted with DCM: MeOH (10:1, 3×100 mL). The combined organic layers were dried with anhydrous Na₂SO₄ and concentrated under vacuum. This resulted in 1.4 g (79%) of the title compound as a yellow solid. MS-ESI: 424 (M+1).

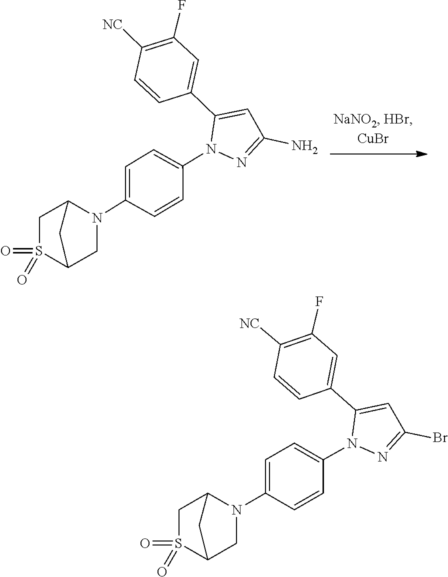 [SMILES: N#Cc1ccc(-c2cc(N)nn2-c2ccc(N3CC4CC3CS4(=O)=O)cc2)cc1F ; N#Cc1ccc(-c2cc(Br)nn2-c2ccc(N3CC4CC3CS4(=O)=O)cc2)cc1F]

4-(3-Bromo-1-(4-(2,2-dioxido-2-thia-5-azabicyclo[2.2.1]heptan-5-yl)phenyl)-1H-pyrazol-5-yl)-2-fluorobenzonitrile To a stirred solution of 4-(3-amino-1-(4-(2,2-dioxido-2-thia-5-azabicyclo[2.2.1]heptan-5-yl)phenyl)-1H-pyrazol-5-yl)-2-fluorobenzonitrile (150 mg, 0.35 mmol, 1.0 equiv) in MeCN (1.5 mL) was added HBr (aq.) (40% wt., 0.1 mL, 0.71 mmol, 2.0 equiv) dropwise for 5 min at 0° C. Then to above mixture was added NaNO₂(aq.) (36.7 mg, 0.53 mmol, 1.5 equiv) dropwise at 0° C. The resulting mixture was stirred for 30 min at 0° C. Then CuBr (102 mg, 0.71 mmol, 2.0 equiv) was added to the reaction solution at 0° C. The resulting mixture was stirred for 1 h at room temperature. The mixture was extracted with DCM (3×20 mL). The combined organic layers were dried with anhydrous Na₂SO₄ and concentrated under vacuum. The residue was purified by Prep-TLC (EtOAc). This resulted in 130 mg (75%) of the title compound as a yellow solid. MS-ESI: 487/489 (M+1).

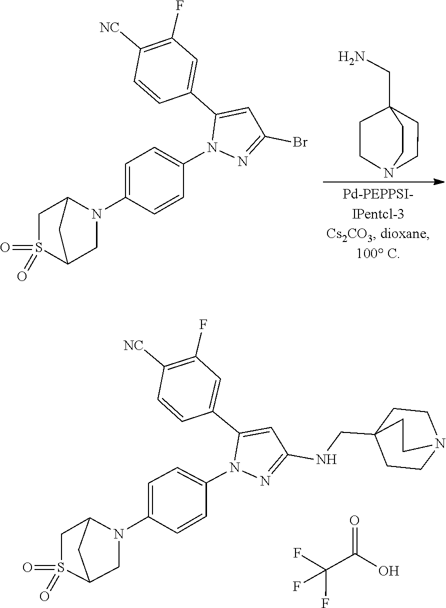 [SMILES: N#Cc1ccc(-c2cc(Br)nn2-c2ccc(N3CC4CC3CS4(=O)=O)cc2)cc1F ; NCC12CCN(CC1)CC2 ; N#Cc1ccc(-c2cc(NCC34CCN(CC3)CC4)nn2-c2ccc(N3CC4CC3CS4(=O)=O)cc2)cc1F ; O=C(O)C(F)(F)F]

4-(1-(4-(2,2-Dioxido-2-thia-5-azabicyclo[2.2.1]heptan-5-yl)phenyl)-3-((quinuclidin-4-ylmethyl)amino)-1H-pyrazol-5-yl)-2-fluorobenzonitrile 2,2,2-trifluoroacetate To a stirred solution of 4-(3-bromo-1-(4-(2,2-dioxido-2-thia-5-azabicyclo[2.2.1]heptan-5-yl)phenyl)-1H-pyrazol-5-yl)-2-fluorobenzonitrile (120 mg, 0.28 mmol, 1.0 equiv) in 1,4-dioxane (5 mL) were added quinuclidin-4-ylmethanamine (39.7 mg, 0.28 mmol, 1.0 equiv), Pd-PEPPSI-IHeptCl 3-chloropyridine (27.6 mg, 0.028 mmol, 0.1 equiv) and Cs₂CO₃ (277 mg, 0.85 mmol, 3.0 equiv) in portions at room temperature under nitrogen atmosphere. The resulting mixture was stirred for 2 h at 100° C. under nitrogen atmosphere. The mixture was allowed to cool down to room temperature. The residue was purified by Prep-TLC (DCM/MeOH 8:1). The crude product (60 mg) was purified by Prep-HPLC with the following conditions: Xselect CSH C18 OBD Column, 30*150 mm 5 μm; Mobile Phase A: water (0.05% TFA), Mobile Phase B: ACN; Flow rate: 60 mL/min; Gradient: 23% B to 33% B in 7 min; Detector UV: 254/220 nm; RT1(min) 6.48. This resulted in 30 mg (15.8%) of the title compound as a yellow solid. MS-ESI: 547 (M+1). ¹H NMR (400 MHZ, MeOD-d₄) δ 7.69 (dd, J=8.4, 7.2 Hz, 1H), 7.27-7.18 (m, 2H), 7.17-7.10 (m, 2H), 6.70 (d, J=8.8 Hz, 2H), 6.12 (s, 1H), 4.76 (s, 1H), 3.91 (s, 1H), 3.82 (d, J=10.4 Hz, 1H), 3.75 (dd, J=10.4, 3.2 Hz, 1H), 3.44-3.35 (m, 6H), 3.21 (s, 4H), 2.71 (d, J=12.0 Hz, 1H), 2.60 (d, J=12.0 Hz, 1H), 1.96-1.87 (m, 6H).

Examples in the following table were prepared using similar conditions as described for those of Example 121 from appropriate starting materials.

TABLE 24
Example Compounds.

| Ex. | | | |
|---|---|---|---|
| No. | Structure | IUPAC Name | Data |
| 122 | 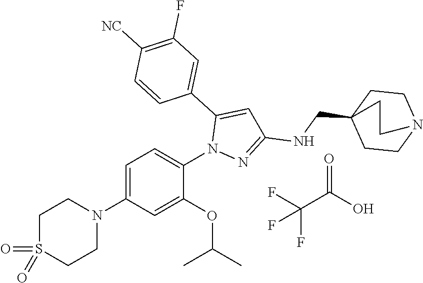 | 4-(1-(4-(1,1-Dioxido- thiomorpholino)-2- isopropoxyphenyl)- 3-((quinuclidin-4- ylmethyl)amino)-1H- pyrazol-5-yl)-2- fluorobenzonitrile 2,2,2-trifluoroacetate | MS-ESI: 593(M + 1) |
| 123 | 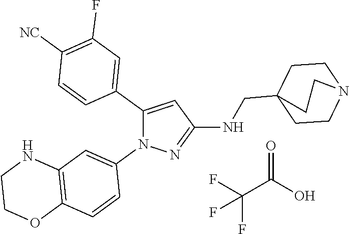 | 4-(1-(3,4-Dihydro- 2H-benzo[b][1,4]- oxazin-6-yl)-3- ((quinuclidin-4-yl- methyl)amino)-1H- pyrazol-5-yl)-2- fluorobenzonitrile 2,2,2-trifluoroacetate | MS-ESI: 459(M + 1) |
| 124 | 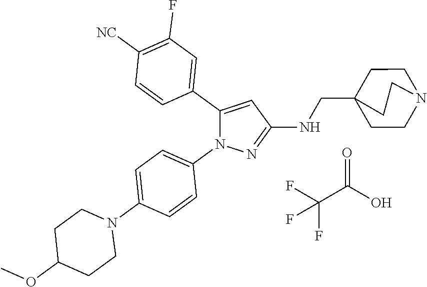 | 2-Fluoro-4-(1-(4-(4- methoxypiperidin-1- yl)phenyl)-3-((quinu- clidin-4-ylmethyl)- amino)-1H-pyrazol- 5-yl)benzo-nitrile 2,2,2-trifluoroacetate | MS-ESI: 515(M + 1) |
| 125 | 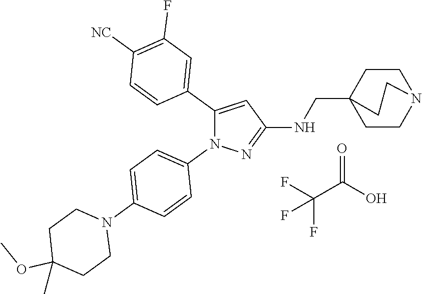 | 2-Fluoro-4-(1-(4-(4- methoxy-4-methyl- piperidin-1-yl)phen- yl)-3-((quinuclidin- 4-ylmethyl)amino)- 1H-pyrazol-5-yl)- benzonitrile 2,2,2- trifluoroacetate | MS-ESI: 529(M + 1) |
| 126 | 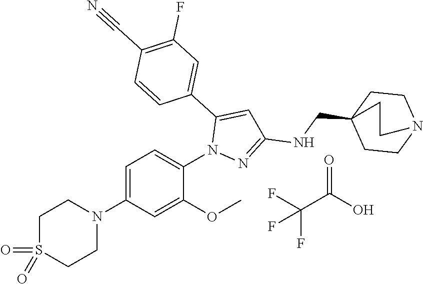 | 4-(1-(4-(1,1-Dioxido- thiomorpholino)-2- methoxyphenyl)-3- ((quinuclidin-4-yl- methyl)amino)-1H- pyrazol-5-yl)-2- fluorobenzonitrile 2,2,2-trifluoroacetate | MS-ESI: 565(M + 1) |

### Example 127

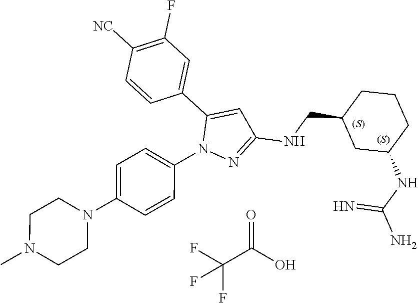

### 1-((1S,3S)-3-(((5-(4-cyano-3-fluorophenyl)-1-(4-(4-methylpiperazin-1-yl)phenyl)-1H-pyrazol-3-yl)amino)methyl)cyclohexyl)guanidine 2,2,2-trifluoroacetate

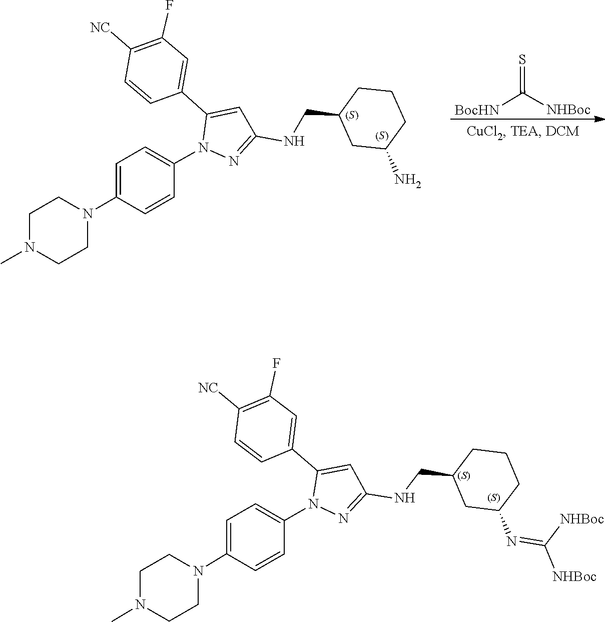

Tert-butyl N-{[(tert-butoxycarbonyl)amino]({[(1S,3S)-3-({[5-(4-cyano-3-fluorophenyl)-1-[4-(4-methylpiperazin-1-yl)phenyl]pyrazol-3-yl]amino}methyl)cyclohexyl]imino})methyl}carbamate To a stirred solution of Example 44 (200 mg, 0.41 mmol, 1.0 equiv) in DCM (5 mL) were added tert-butyl N-{[(tert-butoxycarbonyl)amino]methanethioyl}carbamate (79.3 mg, 0.29 mmol, 0.7 equiv), CuCl₂ (110 mg, 0.82 mmol, 2.0 equiv) and TEA (83 mg, 0.82 mmol, 2.0 equiv) in portions at room temperature. The resulting mixture was stirred for 2 h at room temperature. The residue was purified by Prep-TLC (DCM/MeOH 10:1). This resulted in 100 mg (33.4%) of the title compound as a yellow solid. MS-ESI: 730 (M+1).

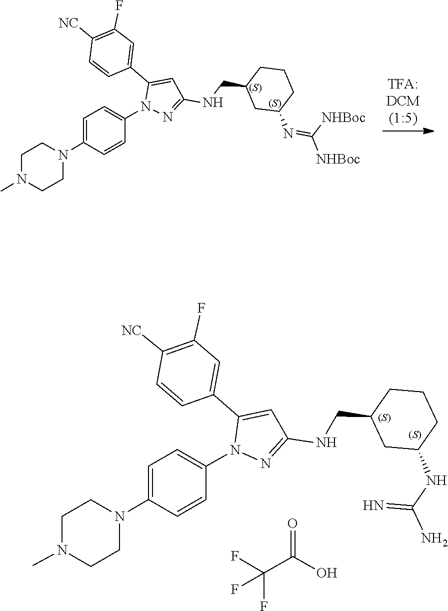

### 1-((1S,3S)-3-(((5-(4-cyano-3-fluorophenyl)-1-(4-(4-methylpiperazin-1-yl)phenyl)-1H-pyrazol-3-yl)amino)methyl)cyclohexyl)guanidine 2,2,2-trifluoroacetate

A solution of tert-butyl N-{[(tert-butoxycarbonyl)amino]({[(1S,3S)-3-({[5-(4-cyano-3-fluorophenyl)-1-[4-(4-methylpiperazin-1-yl)phenyl]pyrazol-3-yl]amino}methyl)cyclohexyl]imino})methyl}carbamate (100 mg, 0.14 mmol, 1.0 equiv) in DCM (2 mL) and TFA (0.4 mL) was stirred for 1 h at room temperature. The mixture was basified to pH 8 with sat. Na₂CO₃ (aq.) and extracted with DCM (3×30 mL). The combined organic layers were washed with brine (3×30 mL), dried over anhydrous Na₂SO₄ and concentrated under vacuum. The crude product was purified by Prep-HPLC with the following conditions: SunFire Prep C18 OBD Column, 19*150 mm, 5 μm; Mobile Phase A: water (0.05% TFA), Mobile Phase B: ACN; Flow rate: 20 mL/min; Gradient: 70% B to 90% B in 5.5 min, Detector UV: 210/254 nm; RT1(min): 5.3. This resulted in 13.3 mg (18.2%) of the title compound as a yellow solid. MS-ESI: 530 (M+1). ¹H NMR (400 MHZ, MeOD-d₄) δ 7.68 (t, J=7.6 Hz, 1H), 7.25-7.15 (m, 4H), 7.07 (d, J=9.2 Hz, 2H), 6.11 (s, 1H), 4.05-3.85 (m, 2H), 3.81 (s, 1H), 3.74-3.54 (m, 2H), 3.31-3.23 (m, 2H), 3.22-3.14 (m, 2H), 3.13-3.03 (m, 2H), 3.00 (s, 3H), 2.45-1.89 (m, 1H), 1.88-1.78 (m, 2H), 1.77-1.63 (m, 3H), 1.62-1.56 (m, 2H), 1.36-1.23 (m, 1H).

The example in the following table was prepared using similar conditions as described for those of Example 127 from appropriate starting materials.

TABLE 25
Example Compounds.

| Ex. | | | |
|---|---|---|---|
| No. | Structure | IUPAC Name | Data |
| 128 | 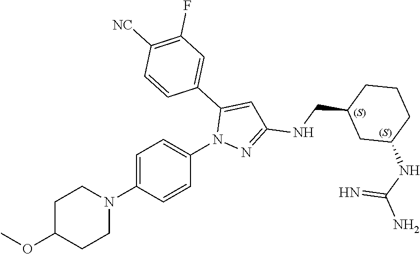 | 1-((1S,3S)-3-(((5-(4- cyano-3-fluorophen- yl)-1-(4-(4-methoxy- piperidin-1-yl)phen- yl)-1H-pyrazol-3-yl)- amino)methyl)cyclo- hexyl)guanidine | MS-ESI: 545(M + 1) |

### Example 129

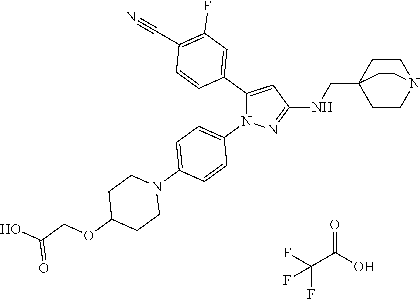 [SMILES: N#Cc1ccc(-c2cc(NCC34CCN(CC3)CC4)nn2-c2ccc(N3CCC(OCC(=O)O)CC3)cc2)cc1F ; O=C(O)C(F)(F)F]

### 2-((1-(4-(5-(4-Cyano-3-fluorophenyl)-3-((quinuclidin-4-ylmethyl)amino)-1H-pyrazol-1-yl)phenyl)piperidin-4-yl)oxy)acetic acid 2,2,2-trifluoroacetate

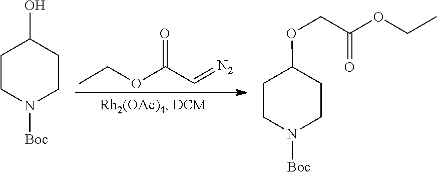

Tert-butyl 4-(2-ethoxy-2-oxoethoxy) piperidine-1-carboxylate To a stirred solution of tert-butyl 4-hydroxypiperidine-1-carboxylate (3.27 g, 16.2 mmol, 1.0 equiv) in DCM (50 mL) were added ethyl 2-diazoacetate (5.56 g, 48.7 mmol, 3.0 equiv) and Rh₂(OAc)₄ (718 mg, 1.63 mmol, 0.1 equiv) at 0° C. under nitrogen atmosphere. The reaction mixture was stirred for 16 h at room temperature under nitrogen atmosphere. The mixture was diluted with H₂O (200 mL) and extracted with DCM (3×200 mL). The combined organic layers were dried over anhydrous Na₂SO₄ and concentrated under vacuum. The residue was purified by Prep-TLC (EtOAc/PE 1:2). This resulted in 2.4 g (51.4%) of the title compound as a yellow oil. MS-ESI: 288 (M+1).

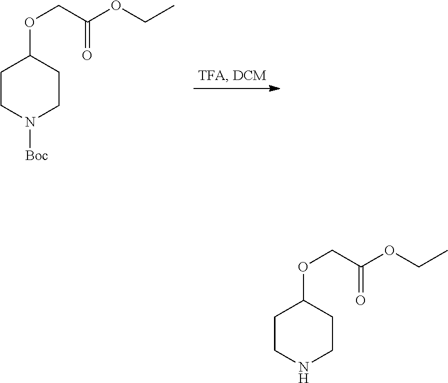 [SMILES: CCOC(=O)COC1CCN(C(=O)OC(C)(C)C)CC1 ; CCOC(=O)COC1CCNCC1]

Ethyl 2-(piperidin-4-yloxy)acetate A solution of tert-butyl 4-(2-ethoxy-2-oxoethoxy) piperidine-1-carboxylate (2.4 g, 8.35 mmol, 1.0 equiv) in DCM (25 mL) and TFA (5 mL) was stirred for 16 h at room temperature. The mixture was basified to pH 10 with sat. Na₂CO₃ aq. and extracted with DCM (3×100 mL). The combined organic layers were dried over anhydrous Na₂SO₄ and concentrated under vacuum. This resulted in 1.5 g (95.9%) of the title compound as a yellow oil. MS-ESI: 188 (M+1).

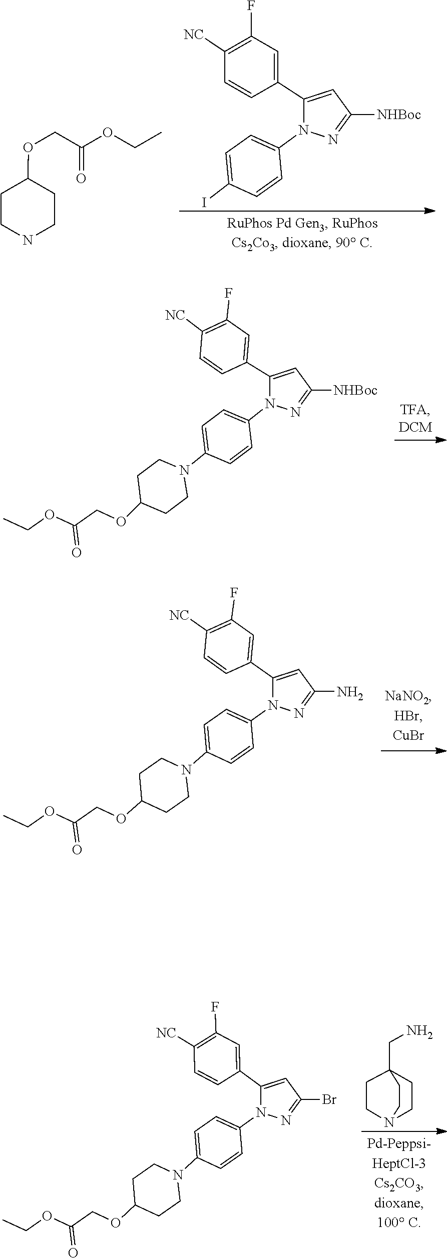

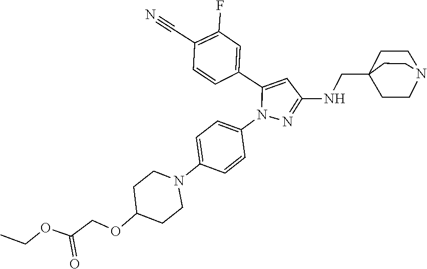 [SMILES: CCOC(=O)COC1CCN(c2ccc(-n3nc(NCC45CCN(CC4)CC5)cc3-c3ccc(C#N)c(F)c3)cc2)CC1]

Ethyl 2-((1-(4-(5-(4-cyano-3-fluorophenyl)-3-((quinuclidin-4-ylmethyl)amino)-1H-pyrazol-1-yl)phenyl)piperidin-4-yl)oxy)acetate Ethyl 2-(piperidin-4-yloxy)acetate was converted to ethyl 2-((1-(4-(5-(4-cyano-3-fluorophenyl)-3-((quinuclidin-4-ylmethyl)amino)-1H-pyrazol-1-yl)phenyl)piperidin-4-yl)oxy)acetate with methods similar to those used to obtain Example 121.

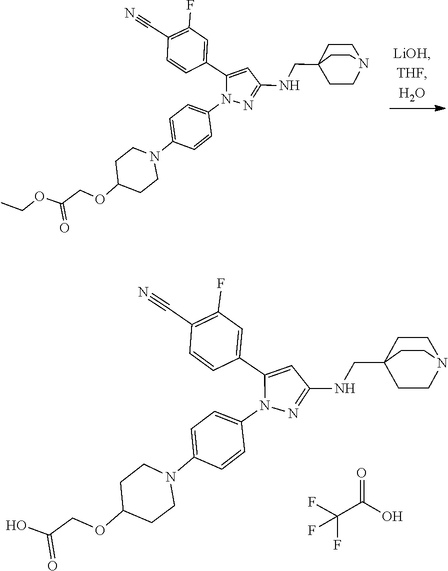 [SMILES: CCOC(=O)COC1CCN(c2ccc(-n3nc(NCC45CCN(CC4)CC5)cc3-c3ccc(C#N)c(F)c3)cc2)CC1 ; N#Cc1ccc(-c2cc(NCC34CCN(CC3)CC4)nn2-c2ccc(N3CCC(OCC(=O)O)CC3)cc2)cc1F ; O=C(O)C(F)(F)F]

2-((1-(4-(5-(4-Cyano-3-fluorophenyl)-3-((quinuclidin-4-ylmethyl)amino)-1H-pyrazol-1-yl)phenyl)piperidin-4-yl)oxy)acetic acid 2,2,2-trifluoroacetate To a stirred solution of ethyl 2-((1-(4-(5-(4-cyano-3-fluorophenyl)-3-((quinuclidin-4-ylmethyl)amino)-1H-pyrazol-1-yl)phenyl)piperidin-4-yl)oxy)acetate (65 mg, 0.111 mmol, 1.0 equiv) in THF (15 mL) and H₂O (5 mL) was added LiOH (7.96 mg, 0.33 mmol, 3.0 equiv). The reaction mixture was stirred for 2 h at room temperature. The resulting mixture was concentrated under vacuum. The crude product was purified by Prep-HPLC with the following conditions: X select CSH C18 OBD Column 30*150 mm 5 μm; Mobile Phase A: water (0.05% TFA), Mobile Phase B: ACN; Flow rate: 60 mL/min; Gradient: 14% B to 24% B in 8 min, 24% B; Detector UV: 254/220 nm; RT1(min): 7.68. This resulted in 27.5 mg (36.5%) of the title compound as a yellow solid. MS-ESI: 559 (M+1). ¹H NMR (400 MHZ, DMSO-d₆) δ 9.28 (s, 1H), 7.88 (t, J=7.6 Hz, 1H), 7.40-7.31 (m, 1H), 7.23-7.11 (m, 1H), 7.05-7.03 (m, 2H), 7.02-6.89 (m, 2H), 6.10 (s, 1H), 5.99-5.30 (m, 1H), 4.17-4.10 (m, 2H), 3.70-3.48 (m, 3H), 3.30-3.19 (m, 6H), 3.10-3.02 (m, 2H), 3.02-2.89 (m, 2H), 1.98-1.89 (m, 2H), 1.80-1.65 (m, 6H), 1.63-1.49 (m, 2H).

Examples in the following table were prepared using similar conditions as described in the Examples disclosed herein from appropriate starting materials.

TABLE 26
Example Compounds.

| Ex. | | |
|---|---|---|
| No. | Structure | IUPAC Name |
| 129 | 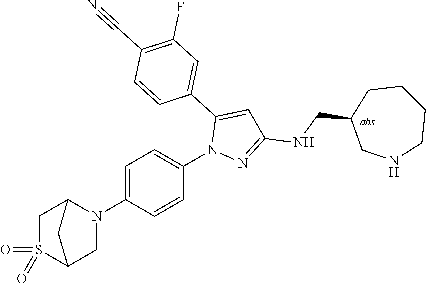 [SMILES: N#Cc1ccc(-c2cc(NC[C@H]3CCCCNC3)nn2-c2ccc(N3CC4CC3CS4(=O)=O)cc2)cc1F] | 4-[3-({[(3S)-azepan-3- yl]methyl}amino)-1-(4- {2,2-dioxo-2λ⁶-thia-5- azabicyclo[2.2.1]heptan- 5-yl}phenyl)-1H-pyr- azol-5-yl]-2-fluoro- benzonitrile |
| 130 | 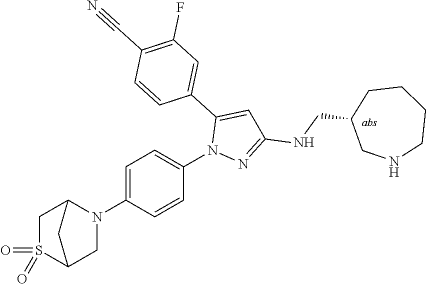 [SMILES: N#Cc1ccc(-c2cc(NC[C@@H]3CCCCNC3)nn2-c2ccc(N3CC4CC3CS4(=O)=O)cc2)cc1F] | 4-[3-({[(3R)-azepan-3- yl]methyl}amino)-1-(4- {2,2-dioxo-2λ⁶-thia-5- azabicyclo[2.2.1]heptan- 5-yl}phenyl)-1H-pyr- azol-5-yl]-2-fluoro- benzonitrile |
| 131 | 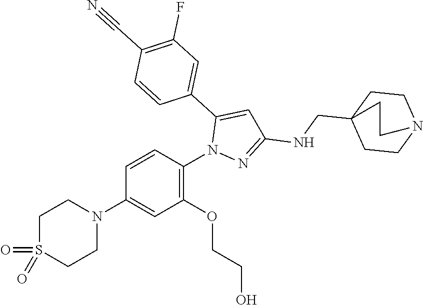 [SMILES: N#Cc1ccc(-c2cc(NCC34CCN(CC3)CC4)nn2-c2ccc(N3CCS(=O)(=O)CC3)cc2OCCO)cc1F] | 4-{3-[{{1-azabicyclo- [2.2.2]octan-4-yl}meth- yl)amino]-1-[4-(1,1- dioxo-1λ⁶-thiomorpho- lin-4-yl)-2-(2-hydroxy- ethoxy)phenyl]-1H- pyrazol-5-yl}-2-fluoro- benzonitrile |
| 132 | 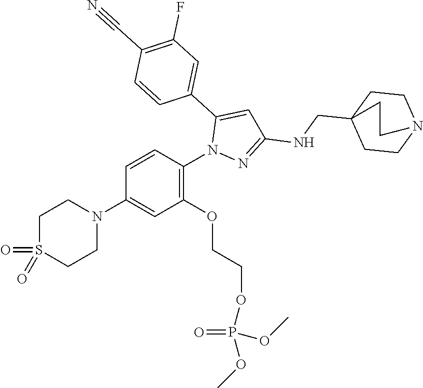 [SMILES: COP(=O)(OC)OCCOc1cc(N2CCS(=O)(=O)CC2)ccc1-n1nc(NCC23CCN(CC2)CC3)cc1-c1ccc(C#N)c(F)c1] | 2-(2-{3-[{{1-azabi- cyclo[2.2.2]octan-4-yl}- methyl)amino]-5-(4- cyano-3-fluorophenyl)- 1H-pyrazol-1-yl}-5- (1,1-dioxo-1λ⁶-thio- morpholin-4-yl)phen- oxy)ethyl dimethyl phosphate |
| 133 | 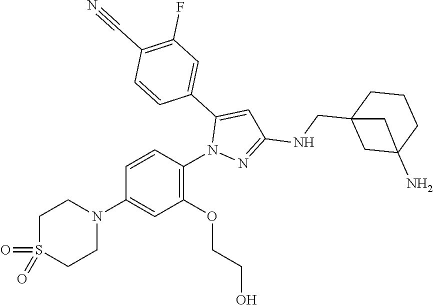 [SMILES: N#Cc1ccc(-c2cc(NCC34CCCC(N)(C3)C4)nn2-c2ccc(N3CCS(=O)(=O)CC3)cc2OCCO)cc1F] | 4-{3-[{{5-aminobi- cyclo[3.1.1]heptan-1- yl}methyl)amino]-1-[4- (1,1-dioxo-1λ⁶-thio- morpholin-4-yl)-2-(2- hydroxyethoxy)phenyl]- 1H-pyrazol-5-yl}-2- fluorobenzonitrile |
| 134 | 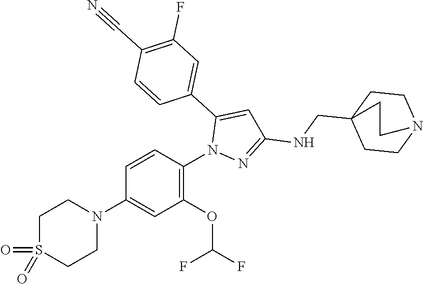 [SMILES: N#Cc1ccc(-c2cc(NCC34CCN(CC3)CC4)nn2-c2ccc(N3CCS(=O)(=O)CC3)cc2OC(F)F)cc1F] | 4-{3-[{{1-azabicyclo- [2.2.2]octan-4-yl}meth- yl)amino]-1-[2-(di- fluoromethoxy)-4-(1,1- dioxo-1λ⁶-thiomorpho- lin-4-yl)phenyl]-1H- pyrazol-5-yl}-2-fluoro- benzonitrile |
| 135 | 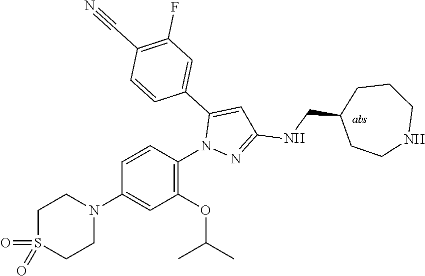 [SMILES: CC(C)Oc1cc(N2CCS(=O)(=O)CC2)ccc1-n1nc(NC[C@H]2CCCNCC2)cc1-c1ccc(C#N)c(F)c1] | 4-[3-({[(4S)-azepan-4- yl]methyl}amino)-1-[4- (1,1-dioxo-1λ⁶-thio- morpholin-4-yl)-2- (propan-2-yloxy)phen- yl]-1H-pyrazol-5-yl]- 2-fluorobenzonitrile |
| 136 | 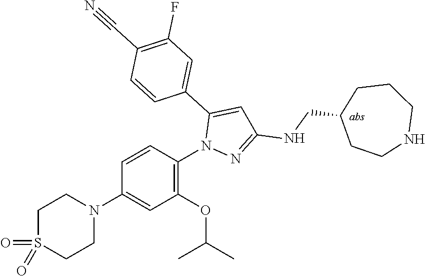 [SMILES: CC(C)Oc1cc(N2CCS(=O)(=O)CC2)ccc1-n1nc(NC[C@@H]2CCCNCC2)cc1-c1ccc(C#N)c(F)c1] | 4-[3-({[(4R)-azepan-4- yl]methyl}amino)-1-[4- (1,1-dioxo-1λ⁶-thio- morpholin-4-yl)-2- (propan-2-yloxy)phen- yl]-1H-pyrazol-5-yl]- 2-fluorobenzonitrile |
| 137 | 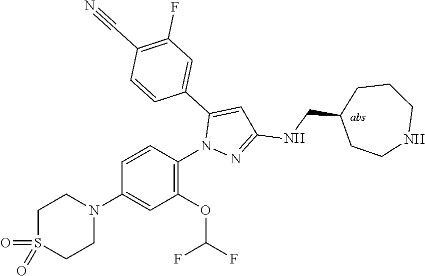 [SMILES: N#Cc1ccc(-c2cc(NC[C@H]3CCCNCC3)nn2-c2ccc(N3CCS(=O)(=O)CC3)cc2OC(F)F)cc1F] | 4-[3-({[(4S)-azepan-4- yl]methyl}amino)-1-[2- (difluoromethoxy)-4- (1,1-dioxo-1λ⁶-thio- morpholin-4-yl)phen- yl]-1H-pyrazol-5-yl]- 2-fluorobenzonitrile |
| 138 | 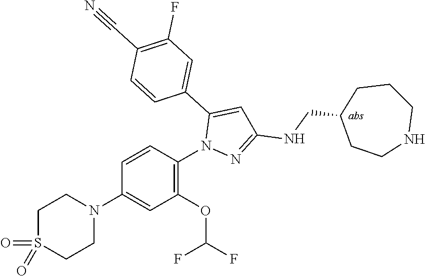 [SMILES: N#Cc1ccc(-c2cc(NC[C@@H]3CCCNCC3)nn2-c2ccc(N3CCS(=O)(=O)CC3)cc2OC(F)F)cc1F] | 4-[3-({[(4R)-azepan-4- yl]methyl}amino)-1-[2- (difluoromethoxy)-4- (1,1-dioxo-1λ⁶-thio- morpholin-4-yl)phen- yl]-1H-pyrazol-5-yl]-2- fluorobenzonitrile |
| 139 | 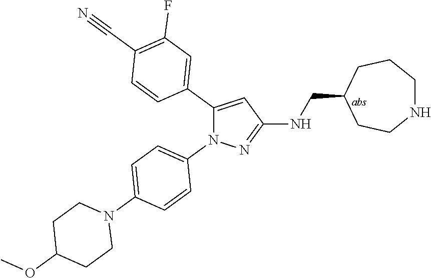 [SMILES: COC1CCN(c2ccc(-n3nc(NC[C@H]4CCCNCC4)cc3-c3ccc(C#N)c(F)c3)cc2)CC1] | 4-[3-({[(4S)-azepan-4- yl]methyl}amino)-1-[4- (4-methoxypiperidin-1- yl)phenyl]-1H-pyrazol- 5-yl]-2-fluorobenzo- nitrile |
| 140 | 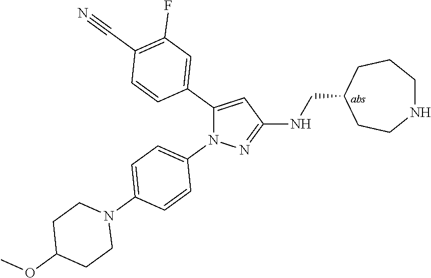 [SMILES: COC1CCN(c2ccc(-n3nc(NC[C@@H]4CCCNCC4)cc3-c3ccc(C#N)c(F)c3)cc2)CC1] | 4-[3-({[(4R)-azepan-4- yl]methyl}amino)-1-[4- (4-methoxypiperidin-1- yl)phenyl]-1H-pyrazol- 5-yl]-2-fluorobenzo- nitrile |
| 141 | 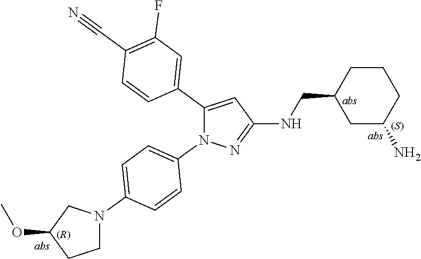 | 4-[3-({[(1S,3S)-3- aminocyclohexyl]meth- yl}amino)-1-{4-[(3R)- 3-methoxypyrrolidin-1- yl]phenyl}-1H-pyrazol- 5-yl]-2-fluorobenzo- nitrile |
| 142 | 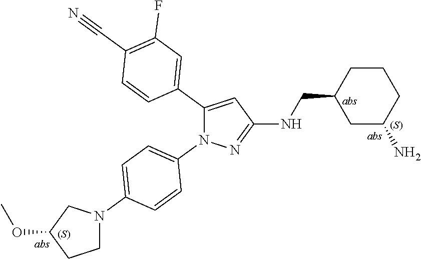 | 4-[3-({[(1S,3S)-3- aminocyclohexyl]meth- yl}amino)-1-{4-[(3S)-3- methoxypyrrolidin-1- yl]phenyl}-1H-pyrazol- 5-yl]-2-fluorobenzo- nitrile |
| 143 | 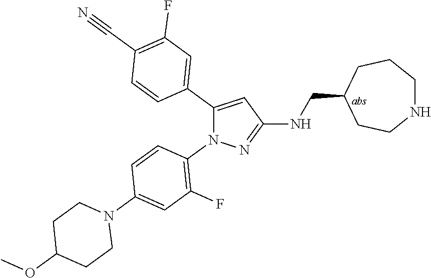 [SMILES: COC1CCN(c2ccc(-n3nc(NC[C@H]4CCCNCC4)cc3-c3ccc(C#N)c(F)c3)c(F)c2)CC1] | 4-[3-({[(4S)-azepan-4- yl]methyl}amino)-1-[2- fluoro-4-(4-methoxy- piperidin-1-yl)phenyl]- 1H-pyrazol-5-yl]-2- fluorobenzonitrile |
| 144 | 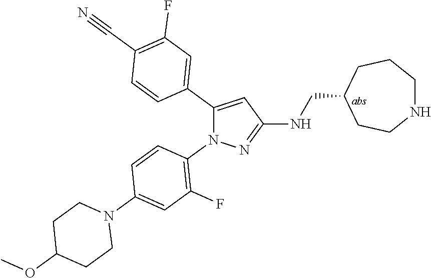 [SMILES: COC1CCN(c2ccc(-n3nc(NC[C@@H]4CCCNCC4)cc3-c3ccc(C#N)c(F)c3)c(F)c2)CC1] | 4-[3-({[(4R)-azepan-4- yl]methyl}amino)-1-[2- fluoro-4-(4-methoxy- piperidin-1-yl)phenyl]- 1H-pyrazol-5-yl]-2- fluorobenzonitrile |
| 145 | 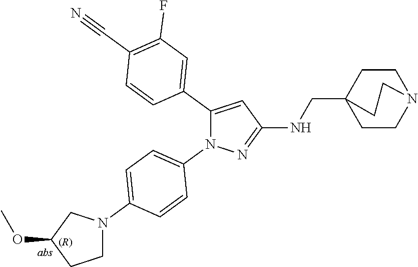 | 4-{3-[{{1-azabicyclo- [2.2.2]octan-4-yl}meth- yl)amino]-1-{4-[(3R)-3- methoxypyrrolidin-1- yl]phenyl}-1H-pyrazol- 5-yl}-2-fluorobenzo- nitrile |
| 146 | 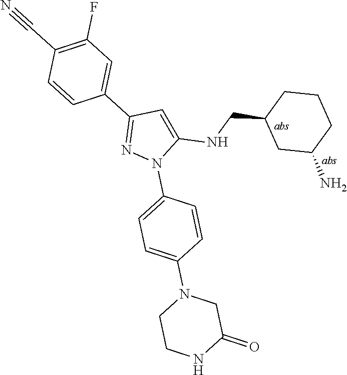 [SMILES: N#Cc1ccc(-c2cc(NC[C@H]3CCC[C@H](N)C3)n(-c3ccc(N4CCNC(=O)C4)cc3)n2)cc1F] | 4-[5-({[(1S,3S)-3- aminocyclohexyl]meth- yl}amino)-1-[4-(3-oxo- piperazin-1-yl)phenyl]- 1H-pyrazol-3-yl]-2- fluorobenzonitrile |
| 147 | 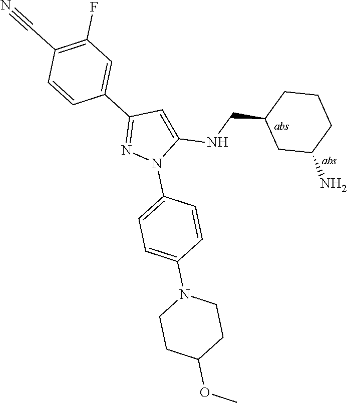 [SMILES: COC1CCN(c2ccc(-n3nc(-c4ccc(C#N)c(F)c4)cc3NC[C@H]3CCC[C@H](N)C3)cc2)CC1] | 4-[5-({[(1S,3S)-3- aminocyclohexyl]meth- yl}amino)-1-[4-(4- methoxypiperidin-1- yl)phenyl]-1H-pyrazol- 3-yl]-2-fluorobenzo- nitrile |
| 148 | 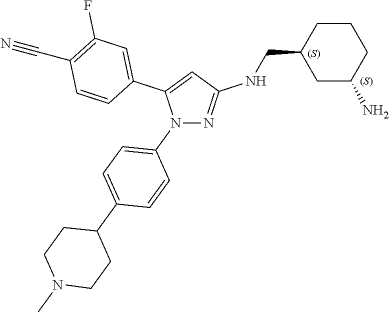 | 4-[3-({[(1S,3S)-3- aminocyclohexyl]meth- yl}amino)-1-[4-(1- methylpiperidin-4-yl)- phenyl]-1H-pyrazol-5- yl]-2-fluorobenzonitrile |
| 149 | 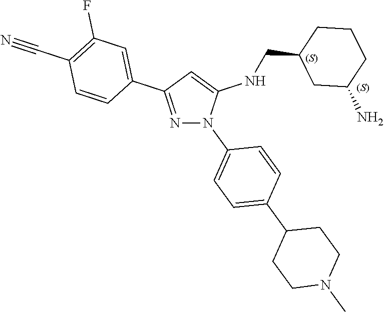 | 4-[3-({[(1S,3S)-3- aminocyclohexyl]meth- yl}amino)-1-[4-(1- methylpiperidin-4-yl)- phenyl]-1H-pyrazol-5- yl]-2-fluorobenzonitrile |
| 150 | 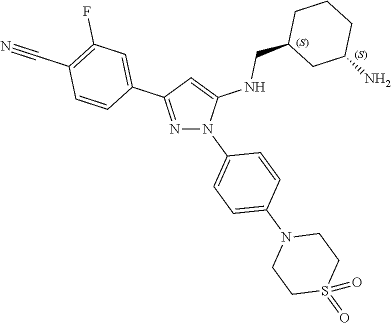 | 4-[5-({[(1S,3S)-3- aminocyclohexyl]meth- yl}amino)-1-[4-(1,1- dioxo-1λ⁶-thiomorpho- lin-4-yl)phenyl]-1H- pyrazol-3-yl]-2-fluoro- benzonitrile |
| 151 | 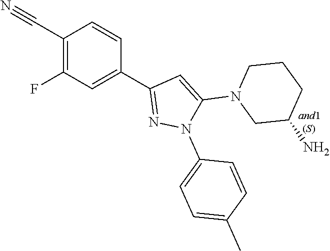 | rac-4-{5-[(3R)-3- aminopiperidin-1-yl]-1- (4-methylphenyl)-1H- pyrazol-3-yl}-2-fluoro- benzonitrile |
| 152 | 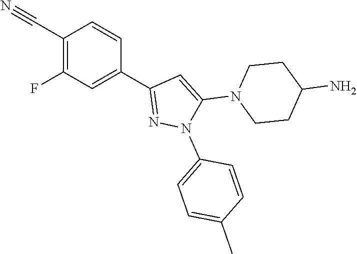 [SMILES: Cc1ccc(-n2nc(-c3ccc(C#N)c(F)c3)cc2N2CCC(N)CC2)cc1] | 4-[5-(4-aminopiperidin- 1-yl)-1-(4-methylphen- yl)-1H-pyrazol-3-yl]-2- fluorobenzonitrile |

TABLE 27
Analytical data.

| Ex. | | |
|---|---|---|
| No. | LC-MS | ¹H NMR |
| 3R | 406 [M + H]⁺ | (300 MHz, CD³OD) δ ppm: 7.70-7.65 (m, 1H), 7.23-7.16 (m, 4H), |
| | | 6.99-6.96 (m, 2H), 6.12 (s, 1H), 3.84 (s, 3H), 3.50-3.32 (m, 2H), |
| | | 3.28-3.14 (m, 2H), 2.94-2.91 (m, 1H), 2.77-2.73 (m, 1H), 2.19-2.16 |
| | | (m, 1H), 2.01-1.95 (m, 2H), 1.84-1.68 (m, 1H), 1.41-1.26 (m, 1H). |
| 3S | 406 [M + H]⁺ | (300 MHz, CD₃OD) δ ppm: 7.70-7.65 (m, 1H), 7.23-7.16 (m, 4H), |
| | | 6.99-6.96 (m, 2H), 6.12 (s, 1H), 3.84 (s, 3H), 3.50-3.32 (m, 2H), |
| | | 3.28-3.14 (m, 2H), 2.94-2.91 (m, 1H), 2.77-2.73 (m, 1H), 2.19-2.16 |
| | | (m, 1H), 2.01-1.95 (m, 2H), 1.84-1.68 (m, 1H), 1.41-1.26 (m, 1H). |
| 4 | 406 [M + H]⁺ | (400 MHz, CD₃OD) δ ppm: 7.70-7.67 (t, J = 7.4 Hz, 1H), 7.24-7.18 |
| | | (m, 4H), 6.99-6.96 (m, 2H), 6.13 (s, 1H), 3.84 (s, 3H), 3.63-3.60 (m, |
| | | 1H), 3.26-3.19 (m, 2H), 2.42-2.36 (m, 2H), 2.15-1.93 (m, 2H), 1.77- |
| | | 1.59 (m, 2H), 1.38-1.33 (m, 1H). |
| 4a | 406 [M + H]⁺ | (400 MHz, CD₃OD) δ ppm: 7.69-7.65 (t, J = 7.6 Hz, 1H), 7.23-7.16 |
| | | (m, 4H), 6.99-6.96 (m, 2H), 6.10 (s, 1H), 3.84 (s, 3H), 3.42-3.32 (m, |
| | | 1H), 3.20-3.17 (m, 2H), 2.33-2.24 (m, 2H), 2.03-1.91 (m, 1H), 1.90- |
| | | 1.82 (m, 1H), 1.61-1.47 (m, 2H), 1.21-1.13 (m, 1H). |
| 4b | 406 [M + H]+ | (400 MHz, CD₃OD) δ ppm: 7.69-7.65 (m, 1H), 7.23-7.16 (m, 4H), |
| | | 6.99-6.96 (m, 2H), 6.10 (s, 1H), 3.84 (s, 3H), 3.42-3.32 (m, 1H), |
| | | 3.20-3.18 (d, J = 6.8 Hz, 2H), 2.33-2.24 (m, 2H), 2.03-1.82 (m, 2H), |
| | | 1.61-1.47 (m, 2H), 1.21-1.13 (m, 1H). |
| 5 | 420 [M + H]⁺ | (400 MHz, DMSO-d₆) δ 7.89-7.86 (m, 1H), 7.86-7.56 (s, 1H), 7.42- |
| | | 7.34 (d, 1H), 7.21-7.08 (d, 2H), 7.01-6.89 (d, 2H), 6.15-6.05 (d, 2H), |
| | | 5.77-5.64 (m,1H), 3.85-3.72 (s, 3H), 3.10-2.92 (m, 2H), 2.18-1.89 |
| | | (m, 2H), 1.85-1.42 (m, 6H), 1.38-1.28 (m, 1H), 1.19-0.81 (m, 1H) |
| 5a | 420 [M + H]⁺ | (400 MHz, Chloroform-d) δ ppm: 7.91-7.78 (m, 1H), 7.50-7.30 (m, |
| | | 1H), 7.18-7.03 (m, 3H), 7.03-6.82 (m, 2H), 6.12-6.02 (m, 1H), 5.75- |
| | | 5.52 (m, 1H), 3.82-3.72 (m, 3H), 3.01-2.90 (m, 2H), 2.65-2.55 (m, |
| | | 1H), 2.00-1.82 (m, 1H), 1.82-1.55 (m, 4H), 1.28-1.20 (m, 1H), 1.09- |
| | | 0.91 (m, 1H), 0.85-0.69 (m, 2H). |
| 5b | 420 [M + H]⁺ | (400 MHz, Chloroform-d) δ ppm: 7.98-7.72 (m, 1H), 7.48-7.35 (m, |
| | | 1H), 7.20-7.19 (m, 3H), 7.02-6.69 (m, 2H), 6.19-5.95 (m, 1H), 5.80- |
| | | 5.50 (m, 1H), 3.85-3.69 (m, 3H), 3.05-2.91 (m, 2H), 2.85-2.70 (m, |
| | | 1H), 2.05-1.68 (m, 5H), 1.31-1.18 (m, 1H), 1.15-0.95 (m, 1H), 0.95- |
| | | 0.40 (m, 2H). |
| 7a | 444 (M + H)⁺ | (400 MHz, CD₃OD) δ 8.03 (s, 1H), 7.65-7.61 (m, 3H), 7.33-7.30 |
| | | (m, 1H), 7.23-7.19 (m, 1H), 7.16-7.13 (m, 1H), 6.14 (s, 1H), 4.11 (s, |
| | | 3H), 3.17-3.12 (m, 2H), 2.22-2.19 (m, 1H), 2.05-2.00 (m 1H), |
| | | 1.97-1.93 (m, 2H), 1.92-1.83 (m, 1H), 1.46-1.42 (m, 1H), 1.37- |
| | | 1.30 (m, 2H), 1.15-1.01 (m, 1H), 1.01-0.97 (m, 1H). |
| 7b | 444 (M + H)⁺ | (400 MHz, CD₃OD) δ 8.03 (s, 1H), 7.65-7.61 (m, 3H), 7.33-7.30 |
| | | (m, 1H), 7.23-7.19 (m, 1H), 7.16-7.12 (m, 1H), 6.14 (s, 1H), 4.11 (s, |
| | | 3H), 3.17-3.12 (m, 2H), 2.22-2.19 (m, 1H), 2.05-2.00 (m, 1H), |
| | | 1.97-1.93 (m, 2H), 1.92-1.83 (m, 1H), 1.46-1.30 (m, 3H), 1.15- |
| | | 1.01 (m, 1H), 0.98-0.90 (m, 1H). |
| 7c | 444 (M + H)⁺ | (400 MHz, CD₃OD) δ 8.03 (s, 1H), 7.65-7.62 (m, 3H), 7.33-7.30 |
| | | (m, 1H), 7.23-7.20 (m, 1H), 7.16-7.10 (m, 1H), 6.14 (s, 1H), 4.11 (s, |
| | | 3H), 3.50-3.45 (m, 1H), 3.25-3.20 (m, 1H), 2.14 (s, 1H), 1.90-1.63 |
| | | (m, 8H), 1.31 (s, 1H). |
| 7d | 444 (M + H)⁺ | (400 MHz, CD₃OD) δ 8.03 (s, 1H), 7.65-7.62 (m, 3H), 7.33-7.30 |
| | | (m, 1H), 7.23-7.19 (m, 1H), 7.16-7.13 (m, 1H), 6.14 (s, 1H), 4.11 (s, |
| | | 3H), 3.50-3.45 (m, 1H), 3.25-3.20 (m, 1H), 2.14 (s, 1H), 1.90-1.31 |
| | | (m, 9H). |
| 8 | 393 [M + H]⁺ | (400 MHz, CD₃OD) δ ppm: 7.70-7.67 (t, J = 7.4 Hz, 1H), 7.23-7.18 |
| | | (m, 4H), 7.01-6.97 (m, 2H), 6.11 (s, 1H), 3.84 (s, 3H), 3.49-3.33 (m, |
| | | 3H), 3.30-3.27 (m, 2H), 3.14-3.09 (m, 1H), 2.80-2.74 (m, 1H), 2.26- |
| | | 2.21 (m, 1H), 1.91-1.83 (m, 1H). |
| 9 | 430 (M + H)⁺ | (400 MHz, CD₃OD) δ 8.21 (s, 1H), 7.71-7.60 (m, 2H), 7.53 (d, J = |
| | | 2.1 Hz, 1H), 7.25-7.16 (m, 3H), 6.12 (s, 1H), 4.22 (s, 3H), 3.53- |
| | | 3.45 (m, 1H), 3.38 (s, 1H), 3.34-3.15 (m, 2H), 3.00-2.89 (m, 1H), |
| | | 2.79 (t, J = 12.1 Hz, 1H), 2.17 (s, 1H), 2.06-1.95 (m, 2H), 1.78- |
| | | 1.71 (m, 1H), 1.44-1.29 (m, 1H). |
| 9a | 430 (M + H)⁺ | (300 MHz, CD₃OD) δ ppm: 8.22 (s, 1H), 7.68-7.60 (m, 2H), 7.52 (s, |
| | | 1H), 7.26-7.16 (m, 3H), 6.13 (s, 1H), 4.22 (s, 3H), 3.49-3.45 (m, |
| | | 1H), 3.36-3.31 (m, 1H), 3.27-3.14 (m, 2H), 2.92-2.88 (m, 1H), 2.77- |
| | | 2.74 (m, 1H), 2.21-2.17 (m, 1H), 2.03-1.96 (m, 2H), 1.76-1.71 (m, |
| | | 1H), 1.41-1.29 (m, 1H). |
| 9b | 430 (M + H)⁺ | (300 MHz, CD₃OD) δ ppm: 8.21 (s, 1H), 7.67-7.60 (m, 2H), 7.51 (s, |
| | | 1H), 7.25-7.15 (m, 3H), 6.13 (s, 1H), 4.22 (s, 3H), 3.49-3.31 (m, |
| | | 2H), 3.28-3.14 (m, 2H), 2.91-2.88 (m, 1H), 2.78-2.73 (m, 1H), 2.23- |
| | | 2.07 (m, 1H), 2.03-1.96 (m, 2H), 1.79-1.61 (m, 1H), 1.38-1.25 (m, |
| | | 1H). |
| 10 | 430 (M + 1)⁺ | (400 MHz, DMSO-d₆) δ 7.43 (s, 1H), 6.84 (q, J = 8.4, 7.2 Hz, 2H), |
| | | 6.73 (s, 1H), 6.49-6.41 (m, 2H), 6.38 (d, J = 8.1 Hz, 1H), 5.34 (s, |
| | | 1H), 3.44 (s, 3H), 2.63 (d, J = 12.8 Hz, 2H), 2.39 (d, J = 6.5 Hz, |
| | | 2H), 2.20 (t, J = 12.6 Hz, 2H), 1.31-1.22 (m, 3H), 0.71-0.62 (m, |
| | | 2H). |
| 11RR | 420 [M + H]⁺ | (300 MHz, CD₃OD) δ ppm: 7.71-7.65 (t, J = 7.4 Hz, 1H), 7.23-7.18 |
| | | (m, 4H), 6.99-6.96 (d, J = 9 Hz, 2H), 6.12 (s, 1H), 3.84 (s, 3H), 3.48 |
| | | (s, 1H), 3.23-3.15 (m, 2H), 2.34-2.01 (m, 1H), 1.98-1.58 (m, 7H), |
| | | 1.55-1.23 (m, 1H) |
| 11SS | 420 [M + H]⁺ | (400 MHz, CD₃OD) δ ppm: 7.70-7.66 (m, 1H), 7.22-7.17 (m, 4H), |
| | | 6.99-6.97 (m, 2H), 6.11 (s, 1H), 3.84 (s, 3H), 3.48 (s, 1H), 3.23-3.15 |
| | | (m, 2H), 2.19-2.01 (m, 1H), 1.98-1.58 (m, 7H), 1.55-1.41 (m, 1H). |
| 12RR | 404 [M + H]⁺ | (400 MHz, CD₃OD) δ 7.69-7.65 (m, 1H), 7.29-7.09 (m, 6H), 6.11 |
| | | (s, 1H), 3.51-3.45 (m, 1H), 3.27-3.16 (m, 2H), 2.39 (s, 3H), 2.16- |
| | | 2.08 (m, 1H), 1.95-1.60 (m, 7H), 1.47-1.43 (m, 1H). |
| 21R | 416 (M + H)⁺ | (400 MHz, CD₃OD) δ 8.24 (s, 1H), 7.77-7.61 (m, 2H), 7.54 (d, J = |
| | | 2.0 Hz, 1H), 7.25-7.16 (m, 3H), 6.13 (s, 1H), 4.24 (s, 3H), 3.52- |
| | | 3.35 (m, 3H), 3.33-3.27 (m, 2H), 3.13 (dd, J = 11.7, 7.6 Hz, 1H), |
| | | 2.77-2.74 (m, 1H), 2.24-2.20 (m, 1H), 1.98-1.80 (m, 1H). |
| 21S | 416 (M + H)⁺ | (400 MHz, CD₃OD) δ 8.24 (s, 1H), 7.71-7.61 (m, 2H), 7.53 (dd, J = |
| | | 2.1, 0.8 Hz, 1H), 7.25-7.23 (m, 2H), 7.19 (dd, J = 8.1, 1.6 Hz, |
| | | 1H), 6.12(s, 1H), 4.22(s, 3H), 3.47-3.33 (m, 3H), 3.31-3.24 (m, 2H), |
| | | 3.12 (dd, J = 11.8, 7.7 Hz, 1H), 2.77 (dq, J = 14.6, 7.3 Hz, 1H), 2.31- |
| | | 2.18 (m, 1H), 1.87 (dq, J = 13.3, 8.1 Hz, 1H). |
| 32a | 434 [M + H]⁺ | (300 MHz, CD₃OD) δ ppm: 7.68-7.63 (m, 1H), 7.20-7.15 (m, 4H), |
| | | 6.97-6.94 (d, J = 9 Hz, 2H), 5.99 (s, 1H), 3.71 (s, 3H), 3.29-3.23 (m, |
| | | 1H), 3.16-3.02 (m, 2H), 2.54 (s, 3H), 2.00-1.93 (m, 1H), 1.83-1.22 |
| | | (m, 8H). |
| 32b | 434 [M + H]⁺ | (300 MHz, CD₃OD) δ ppm: 7.68-7.63 (m, 1H), 7.20-7.14 (m, 4H), |
| | | 6.97-6.94 (d, J = 8.1 Hz, 2H), 6.08 (s, 1H), 3.81 (s, 3H), 3.19-3.00 |
| | | (m, 3H), 2.68 (s, 3H), 2.28-2.25 (m, 1H), 2.14-2.10 (m, 1H), 1.98- |
| | | 1.80 (m, 3H), 1.48-1.25 (m, 2H), 1.12-0.87 (m, 2H). |
| 33a | 434 [M + H]⁺ | (300 MHz, CD₃OD) δ ppm: 7.68-7.63 (m, 1H), 7.20-7.15 (m, 4H), |
| | | 6.97-6.94 (m, 2H), 6.08 (s, 1H), 3.81 (s, 3H), 3.40-3.32 (m, 1H), |
| | | 3.26-3.11 (m, 2H), 2.68 (s, 3H), 2.08-2.03 (m, 1H), 1.93-1.60 (m, |
| | | 7H), 1.49-1.29 (m, 1H). |
| 33b | 434 [M + H]⁺ | (300 MHz, CD₃OD) δ ppm: 7.68-7.63 (m, 1H), 7.20-7.15 (m, 4H), |
| | | 6.97-6.94 (d, J = 9 Hz, 2H), 5.99 (s, 1H), 3.71 (s, 3H), 3.29-3.23 (m, |
| | | 1H), 3.16-3.02 (m, 2H),2.54 (s, 3H), 2.00-1.93 (m, 1H), 1.83-1.22 |
| | | (m, 8H). |
| 90 | MS-ESI: | 7.69 (dd, J = 8.2, 6.8 Hz, 1H), 7.28-7.18 (m, 2H), 7.18-7.11 (m, |
| | 507(M + 1) | 2H), 7.11-6.98 (m, 2H), 6.09 (s, 1H), 4.03-3.83 (m, 2H), 3.81-3.63 |
| | | (m, 2H), 3.26-2.92 (m, 8H), 2.92-2.76 (m, 2H), 2.15-1.85 (m, 4H), |
| | | 1.84-1.69 (m, 1H), 1.67-1.46 (m, 1H), 1.46-1.24 (m, 1H). |
| 91 | MS-ESI: | 7.69 (dd, J = 8.0, 6.4 Hz, 1H), 7.29-7.19 (m, 2H), 7.19-7.12 (m, |
| | 507(M + 1) | 2H), 7.12-7.02 (m, 2H), 6.09 (s, 1H), 4.00-3.82 (m, 2H), 3.79-3.68 |
| | | (m, 2H), 3.43-3.36 (m, 1H), 3.30-3.21 (m, 1H), 3.21-3.01 (m, 6H), |
| | | 2.92-2.83 (m, 2H). 2.25-1.91 (m, 4H), 1.91-1.74 (m, 1H), 1.73-1.55 |
| | | (m, 1H), 1.52-1.29 (m, 1H). |
| 92 | MS-ESI: | 7.70 (dd, J = 8.0, 6.4 Hz, 1H), 7.31 (t, J = 8.8 Hz, 1H), 7.28-7.20 |
| | 521(M + 1) | (m, 2H), 6.87 (dd, J = 8.8, 2.4 Hz, 1H), 6.76 (dd, J = 13.8, 2.4 Hz, |
| | | 1H), 6.14 (s, 1H), 3.68-3.56 (m, 2H), 3.55-3.43 (m, 2H), 3.40 (s, |
| | | 3H), 3.28-3.16 (m, 2H), 3.16-3.05 (m, 2H), 2.18-2.07 (m, 1H), |
| | | 2.07-1.96 (m, 2H), 1.96-1.55 (m, 9H), 1.54-1.40 (m, 1H). |
| 93 | MS-ESI: | 7.82-7.65 (m, 1H), 7.41-7.24 (m, 6H), 6.16 (s, 1H), 4.05 (d, J = 11.6 |
| | 503(M + 1) | Hz, 2H), 4.01-3.87 (m, 1H), 3.59-3.41 (m, 3H), 3.23 (d, J = 6.4 Hz, |
| | | 2H), 3.11 (s, 3H), 2.14 (s, 1H), 1.97-1.85 (m, 1H), 1.85-1.52 (m, |
| | | 10H), 1.50-1.39 (m, 1H). |
| 94 | MS-ESI: | 7.73 (t, J = 7.4 Hz, 1H), 7.53-7.38 (m, 2H), 7.38-7.18 (m, 4H), 6.15 |
| | 531(M + 1) | (s, 1H), 3.93-3.69 (m, 4H), 3.58-3.36 (m, 3H), 3.29-3.13 (m, 2H), |
| | | 2.24-2.02 (m, 3H), 2.02-1.85 (m, 3H), 1.85-1.78 (m, 2H), 1.78-1.57 |
| | | (m, 4H), 1.57-1.41 (m, 1H), 1.21 (d, J = 6.0 Hz, 6H). |
| 95 | MS-ESI: | 7.74 (t, J = 7.4 Hz, 1H), 7.53 (d, J = 8.8 Hz, 2H), 7.37 (d, J = 8.8 |
| | 529(M + 1) | Hz, 2H), 7.31-7.21 (m, 2H), 6.15 (s, 1H), 3.92 (t, J = 6.8 Hz, 2H), |
| | | 3.77-3.66 (m, 2H), 3.61-3.52 (m, 2H), 3.51-3.44 (m, 1H), 3.23 (d, J = |
| | | 5.6 Hz, 2H), 2.19-2.11 (m, 1H), 2.10-1.97 (m, 4H), 1.96-1.85 (m, |
| | | 2H), 1.84-1.75 (m, 3H), 1.74-1.68 (m, 2H), 1.67-1.56 (m, 4H), 1.52- |
| | | 1.41 (m, 1H). |
| 96 | MS-ESI: | 7.69 (t, J = 8.4 Hz, 1H), 7.26-7.19 (m, 4H), 7.03 (d, J = 8 Hz, 2H), |
| | 475(M + 1) | 6.01 (s, 1H), 3.85 (t, J = 4.8 Hz, 4H), 3.49-3.43 (m, 1H), 3.32-3.10 |
| | | (m, 6H), 2.21-2.01 (m, 1H), 1.83-1.80 (m, 1H), 1.79-1.64 (m, 6H), |
| | | 1.63-1.48 (m, 1H). |
| 97 | MS-ESI: | 7.73 (t, J = 7.4 Hz 1H), 7.52-7.11 (m, 6H), 6.14 (s, 1H), 4.96-4.92 |
| | 517(M + 1) | (m, 1H), 3.86-3.67 (m, 3H), 3.61 (q, J = 6.8 Hz, 2H), 3.53-3.43 (m, |
| | | 2H), 3.28-3.14 (m, 2H), 2.25-2.05 (m, 3H), 2.05-1.55 (m, 9H), |
| | | 1.54-1.37 (m, 1H), 1.24 (t, J = 6.8 Hz, 3H) |
| 98 | MS-ESI: | 7.71 (t, J = 7.6 Hz, 1H), 7.29-7.19 (m, 6H), 6.12 (s, 1H), 3.74-3.63 |
| | 503(M + 1) | (m, 1H), 3.62-3.45 (m, 3H), 3.44 (s, 3H), 3.27-3.13 (m, 4H), 2.19- |
| | | 1.85 (m, 4H), 1.84-1.54 (m, 7H), 1.53-1.43 (m, 2H). |
| 99 | MS-ESI: | 7.87 (t, J = 7.6 Hz, 1H), 7.37 (d, J = 10.8 Hz, 1H), 7.15 (dd, J = 8.0, |
| | 491(M + 1) | 1.2 Hz, 1H), 7.03 (d, J = 8.8 Hz, 2H), 6.91 (d, J = 8.8 Hz, 2H), 6.06 |
| | | (s, 1H), 5.52 (t, J = 6.0 Hz, 1H), 3.65-3.51 (m, 4H), 3.06-2.89 (m, |
| | | 3H), 2.73-2.59 (m, 4H), 2.08-1.88 (m, 1H), 1.65-1.35 (m, 7H), 1.23- |
| | | 1.05 (m, 1H). |
| 100 | MS-ESI: | 7.87 (t, J = 7.6 Hz, 1H), 7.37 (dd, J = 10.4, 1.6 Hz, 1H), 7.16 (dd, J = |
| | | 8.4, 1.6 Hz, 1H), 7.03 (d, J = 8.8 Hz, 2H), 6.91 (d, J = 8.8 Hz, |
| | | 2H), 6.06 (s, 1H), 5.52 (t, J = 5.8 Hz, 1H), 3.59-3.53 (m, 4H), 3.62- |
| | 491(M + 1) | 2.92 (m, 3H), 2.71-2.61 (m, 4H), 2.05-1.84 (m, 1H), 1.69-1.22 (m, |
| | | 7H), 1.21-1.06 (m, 1H). |
| 101 | MS-ESI: | 7.74 (t, J = 7.4 Hz, 1H), 7.62 (d, J = 8.8 Hz, 2H), 7.41 (d, J = 8.8 |
| | 503(M + 1) | Hz, 2H), 7.30 (dd, J = 10.8, 1.6 Hz, 1H), 7.24 (dd, J = 8.0, 1.6 Hz, |
| | | 1H), 6.16 (s, 1H), 3.84-3.63 (m, 2H), 3.63-3.43 (m, 3H), 3.27-3.14 |
| | | (m, 2H), 2.24-1.95 (m, 3H), 1.99-1.85 (m, 3H), 1.85-1.55 (m, 6 H), |
| | | 1.53-1.41 (m, 1H), 1.37 (s, 3H). |
| 102 | MS-ESI: | 7.74 (t, J = 7.4 Hz, 1H), 7.53 (d, J = 8.8 Hz, 2H), 7.37 (d, J = 9.2 |
| | 517(M + 1) | Hz, 2H), 7.31-7.20 (m, 2H), 6.15 (s, 1H), 3.70-3.57 (m, 2H), 3.56- |
| | | 3.43 (m, 3H), 3.29 (s, 3H), 3.26-3.17 (m, 2H), 2.22-2.07 (m, 3H), |
| | | 2.00-1.86 (m, 3H), 1.86-1.77 (m, 2H), 1.78-1.56 (m, 4H), 1.56-1.42 |
| | | (m, 1H), 1.30 (s, 3H). |
| 103 | MS-ESI: | 7.70 (dd, J = 7.6, 6.8 Hz, 1H), 7.27-7.18 (m, 2H), 7.15 (d, J = 8.8 |
| | 498(M + 1) | Hz, 2H), 7.04 (d, J = 8.8 Hz, 2H), 6.12 (s, 1H), 3.57-3.42 (m, 3H), |
| | | 3.27-3.12 (m, 4H), 3.07-2.94 (m, 1H), 2.21-2.03 (m, 3H), 2.02-1.85 |
| | | (m, 3H), 1.84-1.76 (m, 2H), 1.76-1.55 (m, 4H), 1.53-1.40 (m, 1H). |
| 104 | MS-ESI: | 7.68 (dd, J = 8.0, 6.8 Hz, 1H), 7.24-7.18 (m, 2H), 7.16 (d, J = 9.2 |
| | 488(M + 1) | Hz, 2H), 6.98 (d, J = 8.8 Hz, 2H), 6.09 (s, 1H), 3.88 (s, 2H), 3.57- |
| | | 3.51 (m, 2H), 3.50-3.39 (m, 3H), 3.25-3.13 (m, 2H), 2.19-2.02 (m, |
| | | 1H), 1.90-1.81 (m, 2H), 1.80-1.53 (m, 6H), 1.52-1.35 (m, 1H). |
| 105 | MS-ESI: | 7.65 (t, J = 7.6 Hz, 1H), 7.22-7.06 (m, 4H), 6.96 (d, J = 8.8 Hz, |
| | 502(M + 1) | 2H), 6.06 (s, 1H), 3.86 (s, 2H), 3.61-3.54 (m, 2H), 3.53-3.45 (m, |
| | | 2H), 3.18-3.09 (m, 2H), 3.03 (s, 3H), 2.16-1.94 (m, 1H), 1.93-1.58 |
| | | (m, 7H), 1.57-1.46 (m, 1H), 1.45-1.31 (m, 1H). |
| 106 | MS-ESI: | 7.69 (dd, J = 8.2, 6.8 Hz, 1H), 7.39 (t, J = 8.8 Hz, 1H), 7.30-7.21 |
| | 541(M + 1) | (m, 2H), 6.98-6.80 (m, 2H), 6.12 (s, 1H), 4.02-3.86 (m, 4H), 3.20- |
| | | 3.12 (m, 4H), 3.12-3.02 (m, 3H), 3.02-2.96 (m, 1H), 2.96-2.81 (m, |
| | | 2H). 2.09-1.81 (m, 4H), 1.75-1.59 (m, 1H), 1.53-1.29 (m, 2H) |
| 107 | MS-ESI: | 7.77-7.61 (m, 1H), 7.38 (t, J = 8.8 Hz, 1H), 7.31-7.16 (m, 2H), 7.01- |
| | 541(M + 1) | 6.91 (m, 1H), 6.88 (dd, J = 13.2, 2.8 Hz, 1H), 6.12 (s, 1H), 4.02- |
| | | 3.85 (m, 4H), 3.21-3.12 (m, 5H), 3.12-3.02 (m, 3H), 3.02-2.90 (m, |
| | | 2H), 2.11-1.83 (m, 4H), 1.83-1.65 (m, 1H), 1.58-1.46 (m, 1H), 1.46- |
| | | 1.30 (m, 1H). |
| 108 | MS-ESI: | 7.70 (dd, J = 8.0, 6.8 Hz, 1H), 7.39 (t, J = 8.8 Hz, 1H), 7.29-7.21 |
| | 529(M + 1) | (m, 2H), 6.94 (dd, J = 8.8, 2.4 Hz, 1H), 6.88 (dd, J = 13.2, 2.4 Hz, |
| | | 1H), 6.17 (s, 1H), 4.14 (dd, J = 12.8, 3.2 Hz, 1H), 4.06-3.91 (m, |
| | | 5H), 3.91-3.79 (m, 1H), 3.45-3.32 (m, 3H), 3.30-3.24 (m, 1H), 3.22- |
| | | 3.05 (m, 5H), 3.02 (t, J = 12.0 Hz, 1H). |
| 109 | MS-ESI: | 7.68 (t, J = 7.6 Hz, 1H), 7.39 (t, J = 8.8 Hz, 1H), 7.23 (d, J = 9.2 Hz, |
| | 520(M + 1) | 2H), 6.83 (dd, J = 8.8, 2.4 Hz, 1H), 6.75 (dd, J = 13.6, 2.8 Hz, 1H), |
| | | 6.12 (s, 1H), 3.92 (s, 2H), 3.72-3.59 (m, 2H), 3.57-3.49 (m, 2H), |
| | | 3.24-3.10(m, 3H), 3.05 (s, 3H), 2.15-1.98 (m, 1H), 1.83-1.53 (m, |
| | | 6H), 1.55-1.42(m, 1H), 1.41-1.28 (m, 1H) |
| 110 | MS-ESI: | 7.73 (dd, J = 8.0, 6.8 Hz, 1H), 7.28 (dd, J = 10.0, 1.2 Hz, 1H), 7.22 |
| | 541(M + 1) | (dd, J = 8.0, 1.6 Hz, 1H), 7.19-7.08 (m, 2H), 6.97-6.88 (m, 1H), |
| | | 6.10 (s, 1H), 3.75-3.59 (m, 4H), 3.43-3.35 (m, 1H), 3.32-3.24 (m, |
| | | 5H), 3.24-3.01 (m, 4H), 2.27-1.94 (m, 4H), 1.93-1.72 (m, 1H), 1.72- |
| | | 1.55 (m, 1H), 1.48-1.30 (m, 1H). |
| 111 | MS-ESI: | 7.73 (dd, J = 8.0, 6.8 Hz, 1H), 7.28 (dd, J = 10.4, 1.2 Hz, 1H), 7.22 |
| | 541(M + 1) | (dd, J = 8.0, 1.6 Hz, 1H), 7.18-7.01 (m, 2H), 6.96-6.80 (m, 1H), |
| | | 6.09 (s, 1H), 3.79-3.59 (m, 4H), 3.41-3.35 (m, 1H), 3.31-3.21 (m, |
| | | 5H), 3.20-3.09 (m, 4H), 2.25-1.94 (m, 4H), 1.94-1.78 (m, 1H), 1.78- |
| | | 1.59 (m, 1H), 1.48-1.27 (m, 1H). |
| 112 | MS-ESI: | 7.67 (dd, J = 8.4, 6.8 Hz, 1H), 7.29 (d, J = 8.4 Hz, 1H), 7.26-7.13 |
| | 553(M + 1) | (m, 2H), 6.71 (dd, J = 8.8, 2.8 Hz, 1H), 6.65 (d, J = 2.4 Hz, 1H), |
| | | 6.09 (s, 1H), 4.01-3.89 (m, 4H), 3.59 (s, 3H), 3.43-3.35 (m, 1H), |
| | | 3.31-3.25 (m, 1H), 3.24-3.03 (m, 8H), 2.22-1.92 (m, 4H), 1.92-1.75 |
| | | (m, 1H), 1.71-1.55 (m, 1H), 1.49-1.25 (m, 1H). |
| 113 | MS-ESI: | 7.68 (dd, J = 8.0, 6.4 Hz, 1H), 7.32-7.17 (m, 3H), 6.72 (dd, J = 8.6, |
| | 553(M + 1) | 2.6 Hz, 1H), 6.65 (d, J = 2.8 Hz, 1H), 6.18-5.95 (m,1H), 4.09-3.82 |
| | | (m, 4H), 3.67-3.49 (m, 3H), 3.46-3.34 (m, 1H),3.24-2.91 (m, 9H), |
| | | 2.24-1.91 (m, 4H), 1.91-1.72 (m, 1H), 1.72-1.53 (m, 1H), 1.52-1.28 |
| | | (m, 1H) |
| 114 | MS-ESI: | 7.71 (t, J = 7.4 Hz, 1H), 7.47-7.36 (m, 1H), 7.33-7.21 (m, 2H), 6.97- |
| | 533(M + 1) | 6.87 (m, 1H), 6.86-6.81 (m, 1H), 6.20 (s, 1H), 3.81-3.66 (m, 2H), |
| | | 3.61 (s, 3H), 3.59-3.51 (m, 1H), 3.50-3.43 (m, 1H), 3.41 (s, 3H), |
| | | 3.31-3.23 (m, 2H), 3.22-3.14 (m, 2H), 2.22-2.02 (m, 3H), 1.96-1.76 |
| | | (m, 5H), 1.75-1.57 (m, 4H), 1.53-1.39 (m, 1H). |
| 115 | MS-ESI: | 7.71 (t, J = 4.0 Hz, 1H), 7.54 (d, J = 8.8 Hz, 1H), 7.33-7.22 (m, 2H), |
| | 553(M + 1) | 7.11 (dd, J = 8.4, 2.0 Hz, 1H), 7.04 (d, J = 2.4 Hz, 1H), 6.21 (s, 1H), |
| | | 3.73-3.41 (m, 8H), 3.22 (d, J = 7.2 Hz, 2H), 2.19-2.07 (m, 1H), |
| | | 2.06-1.79 (m, 7H), 1.77-1.56 (m, 4H), 1.52-1.42 (m, 1H), 1.34 (s, |
| | | 3H). |
| 116 | MS-ESI: | 7.70 (dd, J = 7.4 Hz, 1H), 7.35-7.27 (m, 1H), 7.23 (d, J = 8.8 Hz, |
| | 521(M + 1) | 2H), 6.87 (d, J = 9.2 Hz, 1H), 6.76 (d, J = 13.6 Hz, 1H), 6.13 (s, |
| | | 1H), 3.55-3.42 (m, 3H), 3.31-3.12 (m, 4H), 2.17-1.98 (m, 1H), |
| | | 1.96-1.54 (m, 11H), 1.53-1.38 (m, 1H), 1.28 (s, 3H). |
| 117 | MS-ESI: | 7.64 (t, J = 4.0 Hz, 1H), 7.32-7.15 (m, 3H), 6.61 (dd, J = 8.8, 2.8 |
| | 532(M + 1) | Hz, 1H), 6.54 (d, J = 2.4 Hz, 1H), 6.07 (s, 1H), 3.93 (s, 2H), 3.67- |
| | | 3.47 (m, 7H), 3.20-3.09 (m, 3H), 3.05 (s, 3H), 2.11-1.97 (m, 1H), |
| | | 1.80-1.67 (m, 2H), 1.67-1.53 (m, 4H), 1.52-1.41 (m, 1H), 1.40-1.29 |
| | | (m, 1H). |
| 118 | MS-ESI: | 7.74 (t, J = 4.0 Hz, 1H), 7.48 (t, J = 8.2 Hz 1H), 7.44-7.35 (m, 2H), |
| | 503(M + 1) | 7.31 (dd, J = 10.4, 1.6 Hz, 1H), 7.23 (dd, J = 8.0, 1.6 Hz, 1H), 7.12- |
| | | 7.07 (m, 1H), 6.17 (s, 1H), 3.71-3.55 (m, 3H), 3.53-3.44 (m, 1H), |
| | | 3.42 (s, 3H),3.40-3.34 (m, 2H), 3.28-3.18 (m, 2H), 2.21-2.08 (m, |
| | | 3H), 1.99-1.85 (m, 3H), 1.84-1.78 (m, 2H), 1.77-1.57 (m, 4H), 1.53- |
| | | 1.41 (m, 1H). |
| 119 | MS-ESI: | 7.70 (dd, J = 8.0 Hz, 6.8 Hz, 1H), 7.26-7.22 (m, 2H), 6.71 (d, J = |
| | 447(M + 1) | 8.4 Hz, 1H), 6.52 (d, J = 2.4 Hz, 1H), 6.42 (dd, J = 8.4 Hz, 2.4 Hz, |
| | | 1H), 6.10 (s, 1H), 4.22 (t, J = 4.4 Hz, 2H), 3.49-3.46 (m, 1H), 3.37 |
| | | (t, J = 4.4 Hz, 2H), 3.22-3.18 (m, 2H), 2.12-2.10 (m, 1H), 1.91-1.83 |
| | | (m, 2H), 1.81-1.57 (m, 5H), 1.52-1.42 (m, 1H). |
| 120 | MS-ESI: | 8.00 (s, 1H), 7.70 (t, J = 7.2 Hz, 1H), 7.58-7.45 (m, 1H), 7.34 (d, J = |
| | 504(M + 1) | 8.8 Hz, 1H), 7.23 (d, J = 10.4 Hz, 1H), 7.17 (d, J = 8.0 Hz, 1H), |
| | | 6.11 (s, 1H), 3.68-3.54 (m, 2H), 3.53-3.43 (m, 2H), 3.40 (s, 3H), |
| | | 3.29-3.15 (m, 2H), 3.14-3.01 (m, 2H), 2.22-2.09 (m, 1H), 2.10-1.96 |
| | | (m, 2H), 1.95-1.84 (m, 1H), 1.83-1.76 (m, 2H), 1.75-1.55 (m, 6H), |
| | | 1.53-1.36 (m, 1H) |
| 122 | MS-ESI: | 7.71 (dd, J = 8.1, 6.8 Hz, 1H), 7.41 (d, J = 8.8 Hz, 1H), 7.34-7.25 |
| | 593(M + 1) | (m, 2H), 6.74 (dd, J = 8.8, 2.4 Hz, 1H), 6.62-6.58 (m, 1H), 6.19 (s, |
| | | 1H), 4.52-4.38 (m, 1H), 3.97-3.90 (m, 4H), 3.45-3.35 (m, 6H), 3.21 |
| | | (s, 2H), 3.19-3.12 (m, 4H), 1.96-1.87 (m, 6H), 1.26-0.75 (m, 6H). |
| 123 | MS-ESI: | 7.67 (t, J = 7.4 Hz, 1H), 7.28-7.13 (m, 2H), 6.67 (d, J = 8.4 Hz, 1H), |
| | 459(M + 1) | 6.48 (d, J = 2.0 Hz, 1H), 6.37 (dd, J = 8.4, 2.0 Hz, 1H), 6.06 (s, 1H), |
| | | 4.19 (t, J = 4.4 Hz, 2H), 3.48-3.34 (m, 8H), 3.17 (s, 2H), 1.89 (t, J = |
| | | 8.0 Hz, 6H). |
| 124 | MS-ESI: | 7.72 (t, J = 8.0 Hz, 1H), 7.56-7.13 (m, 6H), 6.13 (s, 1H), 3.85-3.68 |
| | 515(M + 1) | (m, 2H), 3.68-3.51 (m, 1H), 3.46-3.33 (m, 10H), 3.23 (s, 2H), 2.27- |
| | | 2.10 (m, 2H), 2.01-1.71 (m, 8H). |
| 125 | MS-ESI: | 7.73 (t, J = 7.4 Hz, 1H), 7.58 (d, J = 8.8 Hz, 2H), 7.36-7.15 (m, |
| | 529(M + 1) | 4H), 6.13 (s, 1H), 3.64-4.53 (m, 4H), 4.52-3.35 (m, 6H), 3.29 (s, |
| | | 3H), 3.23 (s, 2H), 2.09 (d, J = 13.6 Hz, 2H), 2.01-1.82 (m, 8H), 1.29 |
| | | (s, 3H). |
| 126 | MS-ESI: | 7.70-7.58 (m, 1H), 7.31-7.17 (m, 3H), 6.71 (dd, J = 8.8 Hz, 2.8 Hz, |
| | 565(M + 1) | 1H), 6.64 (d, J = 2.4 Hz, 1H), 6.09 (s, 1H), 4.01-3.88 (m, 4H), 3.57 |
| | | (s, 3H), 3.44-3.36 (m, 6H), 3.21-3.12 (m, 6H), 1.99-1.82 (m, 6H). |
| 128 | MS-ESI: | 7.68 (t, J = 4.4 Hz, 1H), 7.22-7.14 (m, 2H), 7.08 (d, J = 8.8 Hz, |
| | 545(M + 1) | 2H), 6.97 (d, J = 9.2 Hz, 2H), 6.08 (s, 1H), 3.82-3.74 (m, 1H), 3.63- |
| | | 3.49 (m, 2H), 3.46-3.86 (m, 1H), 3.37 (s, 3H), 3.35-3.32 (m, 1H), |
| | | 3.21-3.06 (m, 2H), 3.04-2.93 (m, 2H), 2.69-1.89 (m, 3H), 1.85-1.74 |
| | | (m, 2H), 1.77-1.38 (m, 7H), 1.33-1.20 (m, 1H). |

### Biological Activity Assay

The activity of the Examples above may be illustrated in the following assays. Compounds listed above, which may not yet have been made and/or tested, are predicted to have activity in these assays.

Assaying the inhibition of KDM1A can be determined in vitro, in cultured cells, and in animals. There are a variety of spectrophotometric methods to detect the results of demethylation of methylated lysines, viz., detecting the products of KDM1A demethylase oxidative activity on a peptide fragment of at least 18 amino acids representing the N-terminus of the histone H3 substrate that contains a monomethyl at the fourth lysine residue. Hydrogen peroxide, one product of the KDM1A demethylase reaction, reacts with horseradish peroxidase and dihydroxyphenoxazine (ADHP) to produce the fluorescent compound resorufin (excitation=530-560 nm: emission=590 nm). The KDM1A demethylase enzyme activity can be obtained from mammalian cells or tissues expressing KDM1A from an endogenous or recombinant gene and purified or assayed from a whole cell extract. These methods can be used to determine the concentration of the disclosed compounds can inhibit fifty percent of the enzyme activity (IC₅₀). In one aspect, the disclosed compounds exhibit inhibition fifty percent of the KDM1A enzyme activity at a concentration of less than 500 nM, less than 100 nM, less than 50 nM or less than 10 nM.

The association of KDM1A with other proteins can be determined by a variety of both in vitro and in vivo methods known to one skilled in the art. For example, the disruption of KDM1A with associated proteins can be determined in an electromobility shift assay (EMSA). In various aspects, the disruption of the physical association of KDM1A with CoREST protein by the disclosed compounds can be observed using EMSA. In another example, the disruption of KDM1A with associated proteins can be determined by immunoprecipitation followed by separation of the co-precipitated proteins by mass spectroscopy or by get electrophoresis. In another example, the disruption of KDM1A association with CoREST can be determined by the ability of KDM1A to act on a nucleosomal substrate containing K4 or K9 methylated histone H3, a substrate that requires the presence of both KDM1A and CoREST. The disclosed compounds could be used to assay inhibition of CoREST association with KDM1A using nucleosomal substrate; such compounds may not inhibit KDM1A enzymatic activity as determined by the use of the histone H3 K4 methylated peptide substrate.

The inhibition of KDM1A can be determined in a cell-based assay. For example, KDM1A is an essential enzyme and prolonged inhibition of KDM1A will result in cell death, thus cell growth inhibition, arrest of cell growth or cell death can be assayed. In another aspect, genes induced by androgens and estrogens require KDM1A activity; inhibition by the disclosed compounds of KDM1A will abrogate the induction of gene expression in cells treated with androgens or estrogens. These effects can be measured, e.g., using quantitative PCR of mRNA to measure the magnitude of gene expression for androgen- and estrogen-dependent genes. KDM1A activity is required for the repression of transcription of specific genes. Inhibition of KDM1A by the disclosed compounds could de-repress the expression such genes in cell. These genes include MEIS1, CD86, VEG-A, AIM1, HMOX1, VIM, SKAP1, BMP, EOMES, FOXA2, HNF4, SOX17, GH, PSA, pS2, GREB1, GR-1b, PRL, TSHB, SYN1, HBG, SCN1A, SCN2a, and SCN3A the expression of which can be assayed using quantitative PCR of mRNA before and at various time following the treatment of cells with the disclosed compounds. In another aspect, KDM1A is a regulator of leukemic stem cell potential and is required for oncogenic transformation of myeloid cells to acute myeloid leukemia (AML) by MLL-AF9. Inhibition of KDM1A in MLL-AF9-transformed cells grown in culture overcomes the arrest in differentiation to resulting in a more mature cell expressing the CD11b surface antigen, a monocytic cell antigen. Thus, inhibition of KDM1A can be assayed using an AML cell line such as THP-1 grown in culture quantifying the proportion of cells newly expressing the CD11b antigen using fluorescence activated cell sorting (FACS). A similar assay using FACS to count cells displaying the CD14 or CD86 can be also used, each of which are characteristic of more mature cells along the macrophage/monocytic lineage. Other cells lines derived from patients with acute myeloid leukemia such as MV4; 11 or MOLM-13 cells can be used for this assay. Other markers of differentiation along the macrophage/monocyte lineage can be similarly assayed by FACS such as CD14 and CD86. Other AML cell lines such as MPLM-13 or MV4; 11 can be assayed for the induction of either specific genes mentioned above or the differentiation markers as well as cell growth or apoptosis by Annexin V staining and FACS enumeration.

The selectivity of the disclosed compounds for KDM1A can be determined by assaying the IC₅₀ of the disclosed compounds for other FAD-dependent aminoxidases such as monoamine oxidase A (MAO-A), monoamine oxidase B (MAO-B), IL4I1, KDM1B, or SMOX. As such, a disclosed compound would inhibit KDM1A with an IC₅₀ that is 50-fold, or 100-fold or 250-fold or 500-fold less than for MAO-A or MAO-B.

The histone demethylase assay can be performed essentially as described in Shi, Y. et al. Cell 199, 941-953 (2004). Briefly, bulk histones, histone peptides or nucleosomes are incubated with purified human recombinant KDM1A, in the histone demethylase activity (HDM) assay buffer 1 (50 mM Tris pH 8.5, 50 mM KCl, 5 mM MgCl, 0.5% BSA, and 5% glycerol) from 30 minutes to 4 hours at 37° C. A typical reaction is conducted in 100 microliters in which either 20 micrograms of purified bulk histones or 3 micrograms of modified histone peptides are used as substrates. Different amounts of KDM1A ranging from 1-20 micrograms are used in the reaction along with, as necessary, other co-factors such as FAD or CoREST, depending on the chosen substrate. The reaction mixture is analyzed by SDS-PAGE and Western blotting using histone methyl-specific antibodies or by formaldehyde formation assay to examine the removal and conversion of the methyl group to formaldehyde, or by mass spectrometry in the case of peptide substrates to identify the demethylated histone peptide.

Bulk histones (e.g., 4 mg) are incubated with the indicated amounts of recombinant proteins or complexes in histone demethylase (HDM) assay buffer A (50 mM Tris pH 8.5, 50 mM KCl, 5 mM MgCl, 5% glycerol, 0.2 mM phenylmethylsulphonyl fluoride and 1 mM dithiothreitol) in a final volume of 10 mL for 12-16 h at 37° C. For nucleosomes (0.3 mg) or mononucleosome (0.3 mg), HDM buffer A containing 0.1% NP40 can be used. The reaction mixture can then be analyzed by SDS-PAGE followed by Western blotting. Antibodies against mono- or di-methyl K4 in histone H3 and acetyl-K9/K14 of histone H3 are used to detect the degree of methylation and acetylation, respectively. Western blots are then quantified by densitometry or by intensity of luminescence.

Alternatively, a standard flurogenic assay can be used in which the methylated histone substrate is tethered to the bottom of a 96 well plate (or to beads resting in the plate) using biotin conjugated to the histone methylated substrate and strepavidin (SA) on beads or SA attached to the plate to secure the biotinylated substrate. After incubation of the KDM1A enzyme in histone demethylase buffer A, the demethylated histone substrate can be detected using antibodies specific for demethylated H3K4 substrate conjugated to a fluor or some other agent that can be detected. A variation on that assay method would employ an antibody directed against the methylated version of the histone in which the amount of substrate is quantified before and after incubation with the enzyme. Yet another version of a similar assay would employ a fluorescence resonance energy transfer (FRET) system of detection in which the antibody recognizing the methylated version is conjugated or otherwise linked to an entity, e.g., a bead or a large carrier molecule on which a fluorophore (donor) is attached, and the fluorophore (acceptor) is bound to an entity linked to the substrate.

Alternatively, the production of H₂O₂ during the KDM1A reaction can be detected fluometrically. In this system, the production of H₂O₂ is detected in the HDM assay buffer after exposure to substrate, co-factor and enzyme using ADHP (10-Acetyl-3, 7-dihydroxyphenoxazine) as a fluorogenic substrate for horse radish peroxidase (HRP). ADHP (also known as Amplex Red Reagent) is the most stable and sensitive fluorogenic substrate for HRP. The florescent product is resorufin. Sensitivity can be as low as 10-15 M of target protein. The signal is read using a fluorescence microplate reader at excitation and emission wavelengths of 530-560 nm and 590 nm, respectively.

Additionally, the KDM1A reaction can include other factors which may influence the activity of KDM1A. Such factors might include CoREST, NuRD complexes, DNMT1, HDAC1, HDAC2, and HDAC3, for example, as proteins known to associate with KDM1A or KDM1A-containing complexes. Interactions that influence any aspect of the KDM1A activity including specificity for template, substrate, Kₘ, K_{cat}, or sensitivity to FAD concentrations can be assayed. For example, an in vitro interaction assay between KDM1A and CoREST can be performed adding recombinant KDM1A (e.g., 10 mg) and CoREST (e.g., 5 mg) mixed and incubated for 1 h at 4-8° C., fractionated by Superdex 200 gel filtration column in a buffer containing 20 mM Tris-HCl pH 7.9, 500 mM KCl, 10% glycerol, 0.2 mM EDTA, 1 mM dithiothreitol, 0.1% Nonidet P40 and 0.2 mM phenylmethylsulphonyl fluoride, and then analyzed by silver staining.

For co-immunoprecipitation of mononucleosomes with KDM1A and CoREST, nucleosomes (1.5 mg) can be digested with micrococcal nuclease and incubated with recombinant KDM1A (e.g., 1 mg), CoREST (e.g., 500 ng) or both proteins in HDM buffer A containing 0.1% NP40 for 1 h at 4-8° C. Antibodies directed against KDM1A or CoREST attached to an affinity resin are added and after extensive washing with HDM buffer A containing 0.1% NP40, the bound proteins are eluted with a wash buffer. KDM1A activity can be assayed in the eluate or the concentration of KDM1A can be determined by quantitative Western blotting.

Compounds were tested in a 10-dose IC₅₀ mode fluorescence coupling enzyme assay with 3-fold serial dilution in duplicate starting at 100 μM. The production of FAD-dependent H₂O₂ as a result of demethylase activity of LSD1 on 10 μM histone H3₍₁₋₂₁₎K4me2 peptide substrate was measured by coupling with HRP and Amplex Red to yield resorufin (fluorescence measured at Ex/Em=535/590 nm on EnVision, Perkin Elmer).

TABLE 28
LSD1 Data

| Ex. No. | LSD1 IC₅₀ (nM) |
|---|---|
| 1 | D |
| 2 | A |
| 3 | A |
| 3R | A |
| 3S | A |
| 4 | A |
| 4a | A |
| 4b | A |
| 5 | A |
| 5a | A |
| 5b | A |
| 7 | A |
| 7a | B |
| 7b | A |
| 7c | A |
| 7d | A |
| 8 | A |
| 9 | A |
| 9a | C |
| 9b | A |
| 10 | A |
| 11RR | B |
| 11SS | A |
| 12RR | B |
| 12SS | A |
| 13RR | A |
| 13SS | A |
| 14RR | A |
| 14SS | A |
| 15 | A |
| 16 | A |
| 17 | D |
| 18 | C |
| 19 | A |
| 20 | A |
| 21R | B |
| 21S | A |
| 22 | A |
| 22a | A |
| 22b | A |
| 23 | A |
| 24 | A |
| 24a | A |
| 24b | A |
| 25 | C |
| 25a | A |
| 25b | A |
| 26 | A |
| 26a | A |
| 26b | A |
| 27 | A |
| 27a | A |
| 27b | A |
| 28 | B |
| 29 | C |
| 29a | B |
| 29b | A |
| 31a | A |
| 31b | A |
| 32a | A |
| 32b | C |
| 33a | B |
| 33b | A |
| 34 | A |
| 36 | C |
| 37 | A |
| 38 | A |
| 39 | A |
| 40 | C |
| 41 | A |
| 42 | C |
| 43 | A |
| 44 | B |
| 45 | C |
| 46 | A |
| 49 | C |
| 50 | A |
| 51 | C |
| 52 | A |
| 53 | A |
| 54 | A |
| 55 | A |
| 56 | A |
| 57 | A |
| 58 | A |
| 59 | A |
| 60 | C |
| 61 | A |
| 62 | B |
| 63 | A |
| 64 | C |
| 65 | A |
| 66 | A |
| 67 | B |
| 68 | A |
| 69 | A |
| 70 | A |
| 71 | A |
| 72 | C |
| 73 | A |
| 74 | C |
| 76 | A |
| 77 | C |
| 78 | A |
| 79 | C |
| 80 | B |
| 82 | C |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | C |
| 106 | A |
| 107 | A |
| 108 | A |
| 109 | A |
| 110 | A |
| 111 | A |
| 112 | A |
| 113 | A |
| 114 | A |
| 115 | C |
| 116 | A |
| 117 | A |
| 118 | C |
| 119 | A |
| 120 | A |
| 121 | A |
| 122 | C |
| 123 | A |
| 124 | A |
| 125 | A |
| 126 | A |
| 127 | A |
| 128 | A |
| 129 | C |

A = < 500 nM;
B = 500-1000 nM;
C = 1000-10000 nM;
D = >10000 nM
ND = Not determined

### Determination of IC₅₀ in MV4;11 Cell Line

Cell growth inhibition against MV4;11 cells was confirmed for the synthetic compounds. The Cell Titer-Glo Luminescent Cell Viability Assay (Promega, USA) was used to determine the number of viable cells in culture based on quantitation of the ATP present, which signals the presence of metabolically active cells. This assay was run in a 96-well plate format arrayed in a 10-point dose curve, ranging from 0 to 50 UM with 4-fold dilutions for each compound. Cells are seeded on Day 0 at 3750 cells per well. At Day 5, a portion of each well is assayed for viability according to the Cell Titer-Glo protocol. Briefly, cells/plates were centrifuged at 1100×g for 5 minutes. 100 μL of media was removed from each well and replaced with 100 μL of CellTiter-Glo. The plates were shaken at room temperature for 10 minutes and measured on a luminescence plate reader. Another portion of each well from the Day 5 plate is used to seed a new 96 plate containing fresh compound and media set up in the equivalent 10-point dose curve array in order to continue the cell growth inhibition assay for another 5 days. A final timepoint is collected at Day 10 using the Cell Titer-Glo protocol. Luminescence was detected on an En Vision® Multilabel Plate Reader (Perkin Elmer, Waltham, Mass.) and IC₅₀ determination was made using Graph Pad Prism software.

TABLE 29
Cell Titer Data

| | Cell Titer-Glo | Cell Titer-Glo |
|---|---|---|
| Ex. No. | IC₅₀ (5 d) | IC₅₀ (10 d) |
| 1 | D | |
| 2 | A | |
| 3 | A | |
| 4 | C | |
| 4a | A | |
| 4b | B | |
| 5 | A | |
| 5a | B | |
| 5b | A | |
| 7 | A | |
| 7a | C | |
| 7b | B | |
| 7c | A | A |
| 7d | A | A |
| 8 | A | |
| 9 | A | |
| 9a | A | |
| 9b | A | |
| 10 | A | |
| 11RR | C | |
| 11SS | B | A |
| 12RR | C | A |
| 12SS | A | A |
| 13RR | C | A |
| 13SS | ND | A |
| 14RR | A | A |
| 14SS | ND | A |
| 15 | A | A |
| 16 | A | A |
| 17 | C | C |
| 18 | B | B |
| 19 | A | A |
| 20 | ND | A |
| 21R | B | |
| 21S | A | |
| 22 | C | A |
| 22a | A | A |
| 22b | B | A |
| 23 | A | A |
| 24 | A | A |
| 24a | A | A |
| 24b | A | A |
| 25 | B | A |
| 25a | A | A |
| 25b | C | A |
| 26 | A | A |
| 26a | A | A |
| 26b | A | A |
| 27 | A | A |
| 27a | B | A |
| 27b | A | A |
| 28 | C | C |
| 29 | C | C |
| 29a | C | A |
| 29b | C | A |
| 31a | C | A |
| 31b | B | A |
| 32a | C | |
| 32b | C | |
| 33a | C | |
| 33b | C | |
| 34 | C | |
| 36 | B | A |
| 37 | A | A |
| 38 | C | |
| 39 | C | |
| 40 | ND | D |
| 41 | A | A |
| 42 | C | A |
| 43 | ND | A |
| 44 | A | A |
| 45 | B | A |
| 46 | A | A |
| 49 | C | A |
| 50 | A | A |
| 51 | C | A |
| 52 | A | A |
| 53 | B | A |
| 54 | A | A |
| 55 | B | A |
| 56 | A | A |
| 57 | C | A |
| 58 | A | A |
| 59 | C | A |
| 60 | C | A |
| 61 | A | A |
| 62 | C | A |
| 63 | A | A |
| 64 | C | A |
| 65 | B | A |
| 66 | A | A |
| 67 | A | A |
| 68 | A | A |
| 69 | ND | A |
| 70 | C | A |
| 71 | A | A |
| 72 | B | A |
| 73 | B | A |
| 74 | C | A |
| 75 | B | A |
| 76 | B | A |
| 77 | C | C |
| 78 | C | C |
| 82 | ND | D |

A = < 500 nM;
B = 500-1000 nM;
C = 1000-10000 nM;
D = >10000 nM
ND = Not determined

### LSD1 Inhibition for the Treatment of SCD

In an in vitro assay system, human hematopoietic stem cells (CD34⁺) from a single donor are cultured to differentiate into red cells over the course of 18 days using standard culture methods with eythrogenic growth factors. Three test articles were co-cultured beginning on Day 7: the compound described in Example 11SS, a reversible inhibitor of LSD1; 4-(2-(4-aminopiperidin-1-yl)-5-(4-methoxyphenyl)-1-methyl-6-oxo-1,6-dihydropyrimidin-4-yl)-2-fluorobenzonitrile, a potent reversible inhibitor of LSD1; and 2-(((trans)-2-(4-(benzyloxy)phenyl)cyclopropyl)amino)-1-(4-methylpiperazin-1-yl) ethanone dihydrochloride (RN-1), a commercially available potent reversible inhibitor of LSD1. Cells were harvested at Day 18 and total HbF was assayed by HPLC, e.g., using a C20 reverse phase column. 4-(2-(4-aminopiperidin-1-yl)-5-(4-methoxyphenyl)-1-methyl-6-oxo-1,6-dihydropyrimidin-4-yl)-2-fluorobenzonitrile and RN-1 both induce HbF at 3 nM but inhibit red cell maturation at 10 nM. Conversely, a higher concentration of compound 11SS was required to induce HbF, and red cell maturation was not arrested until concentrations reached 300 nM.

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references cited in this specification are hereby incorporated by reference. The discussion of the references herein is intended merely to summarize the assertions made by their authors and no admission is made that any reference constitutes prior art relevant to patentability. Applicant reserves the right to challenge the accuracy and pertinence of the cited references.

1. A compound of structural Formula I: 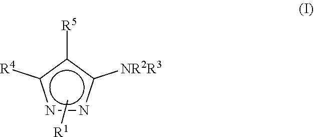
or a salt or tautomer thereof, wherein:
R¹ is chosen from cycloalkyl, heterocycloalkyl, aryl, and heteroaryl, any of which is optionally substituted with one R⁶, and one or more R⁷;
R² and R³ are independently chosen from H, cycloalkyl, heterocycloalkyl, aryl, heteroaryl, (cycloalkyl)alkyl, (heterocycloalkyl)alkyl, (aryl)alkyl, and (heteroaryl)alkyl, any of which is optionally substituted with one or more R⁸,
or R² and R³, together with the intervening nitrogen, combine to form heterocycloalkyl which is optionally substituted with one or more R⁸;
R⁴ is chosen from cycloalkyl, heterocycloalkyl, aryl, and heteroaryl, any of which is optionally substituted with one or more R⁹;
R⁵ is chosen from H, halo, and cyano;
R⁶ is chosen from cycloalkyl, heterocycloalkyl, aryl, heteroaryl, (cycloalkyl)carbonyl, (heterocycloalkyl)carbonyl, (aryl)carbonyl, and (heteroaryl)carbonyl, any of which is optionally substituted with one or more R¹⁰;
each R⁷ is independently chosen from halo, cyano, hydroxy, alkyl, alkoxy, (hydroxy)alkyl, haloalkoxy, and (hydroxy)alkoxy;
each R⁸ is independently chosen from —N(R¹¹)₂, —NHR¹¹, —C(NH)NHR¹¹, —NHC(═NH)—R¹¹, —NHC(═NH)NHR¹¹, —CONHR¹¹, —CH₂NHR¹¹, —CH₂C(═NH)NHR¹¹, —CH₂NHC(═NH)—R¹¹, —CH₂NHC(═NH)NHR¹¹, —CH₂CONHR¹¹, halo, cyano, hydroxy, alkyl, and alkoxy;
each R⁹ is independently chosen from halo, cyano, hydroxy, alkyl, and alkoxy; and
each R¹⁰ is independently chosen from halo, cyano, hydroxy, oxo, alkyl, alkoxy, (hydroxy)alkyl, (hydroxy)alkoxy, alkylthio, and alkylsulfonyl; and
each R¹¹ is independently chosen from H and alkyl.

2. The compound of claim 1, having structural Formula (IIa) or (IIb): 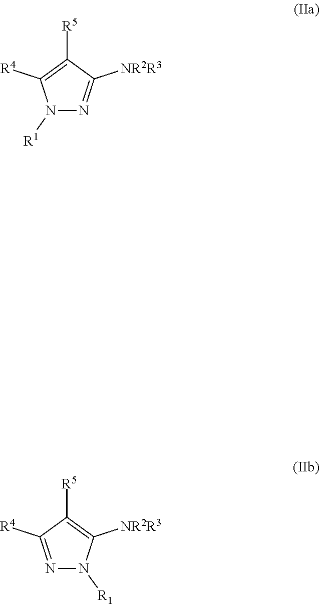
or a salt thereof.

3. The compound of claim 1, or a salt or tautomer thereof, wherein R⁵ is chosen from H, F, and Cl.

4. The compound of claim 3, or a salt or tautomer thereof, wherein R⁵ is chosen from H and Cl.

5. The compound of claim 4, or a salt or tautomer thereof, wherein R⁵ is H.

6. The compound of claim 1, or a salt or tautomer thereof, wherein R⁴ is chosen from (C₃₋₇) cycloalkyl, 4- to 7-membered heterocycloalkyl, (C₆₋₁₀) aryl, and 5- to 10-membered heteroaryl, any of which is optionally substituted with one or more R⁹.

7. The compound of claim 6, or a salt or tautomer thereof, wherein R⁴ is chosen from phenyl, pyridinyl, pyridazinyl, pyrimidinyl, and pyrazinyl, any of which is optionally substituted with one or more R⁹.

8. The compound of claim 7, or a salt or tautomer thereof, wherein R⁴ is 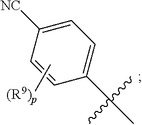
each R⁹ is independently chosen from cyano, halo, hydroxy, alkyl, and alkoxy; and
p is chosen from 0, 1, and 2.

9. The compound of claim 8, or a salt or tautomer thereof, wherein R⁴ is 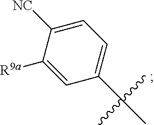
and
R^{9a} is chosen from H, cyano, halo, hydroxy, alkyl, and alkoxy.

10. The compound of claim 9, or a salt or tautomer thereof, wherein R^{9a} is chosen from H, cyano, F, Cl, Br, hydroxy, C₁₋₆alkyl, and C₁₋₆alkoxy.

11. The compound of claim 10, or a salt or tautomer thereof, wherein R^{9a} is chosen from H, cyano, F, Cl, hydroxy, methyl, and methoxy.

12. The compound of claim 11, or a salt or tautomer thereof, wherein R^{9a} is chosen from H, F, Cl, and hydroxy.

13. The compound of claim 12, or a salt or tautomer thereof, wherein R^{9a} is chosen from H and F.

14. The compound of claim 1, or a salt or tautomer thereof, wherein R⁴ is chosen from

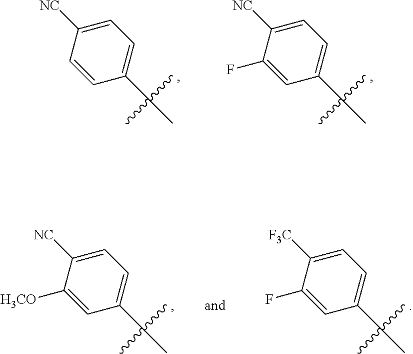 [SMILES: *c1ccc(C#N)cc1 ; *c1ccc(C#N)c(F)c1 ; *c1ccc(C#N)c(OC)c1 ; *c1ccc(C(F)(F)F)c(F)c1]

15. The compound of claim 14, or a salt or tautomer thereof, wherein R⁴ is

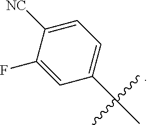 [SMILES: *c1ccc(C#N)c(F)c1]

16. The compound of claim 1, having structural Formula (IIIa) or (IIIb): 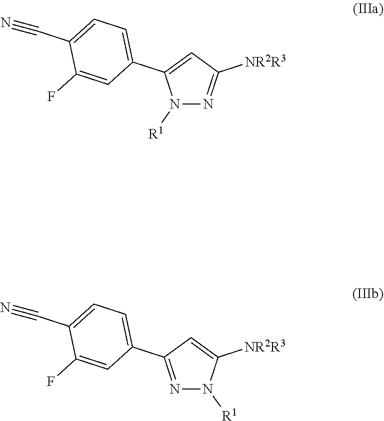
or a salt thereof.

17. The compound of claim 1, or a salt or tautomer thereof, wherein R² is H.

18. The compound of claim 1, or a salt or tautomer thereof, wherein R³ is chosen from H, cycloalkyl, heterocycloalkyl, aryl, heteroaryl, (cycloalkyl)C₁₋₆alkyl, (heterocycloalkyl)C₁₋₆alkyl, (aryl)C₁₋₆alkyl, and (heteroaryl)C₁₋₆alkyl, any of which is optionally substituted with one or more R⁸.

19. The compound of claim 18, or a salt or tautomer thereof, wherein R³ is chosen from H, cycloalkyl, heterocycloalkyl, aryl, heteroaryl, (cycloalkyl)methyl, (heterocycloalkyl)methyl, (aryl)methyl, and (heteroaryl)methyl, any of which is optionally substituted with one or more R⁸.

20. The compound of claim 19, or a salt or tautomer thereof, wherein R³ is chosen from H, C₃₋₁₀cycloalkyl, 4- to 10-membered heterocycloalkyl, C₆₋₁₀aryl, 5- to 10-membered heteroaryl, (C₃₋₁₀cycloalkyl)methyl, (4- to 10-membered heterocycloalkyl)methyl, (C₆₋₁₀aryl)methyl, and (5- to 10-membered heteroaryl)methyl, any of which is optionally substituted with one or more R⁸.

21. The compound of claim 20, or a salt or tautomer thereof, wherein R³ is chosen from (C₃₋₁₀cycloalkyl)methyl and (4- to 10-membered heterocycloalkyl)methyl, any of which is optionally substituted with one or more R⁸.

22. The compound of claim 1, or a salt or tautomer thereof, wherein each R⁸ is independently chosen from —NHR¹¹ and NHC(═NH)NHR¹¹.

23. The compound of claim 1, or a salt or tautomer thereof, wherein R¹¹ is hydrogen.

24. The compound of claim 1, or a salt or tautomer thereof, wherein R³ is chosen from

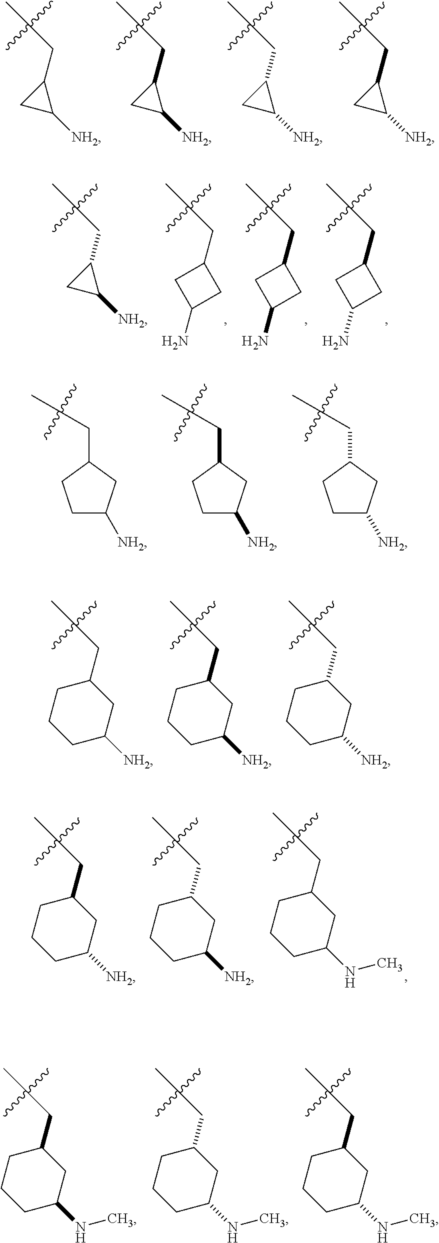

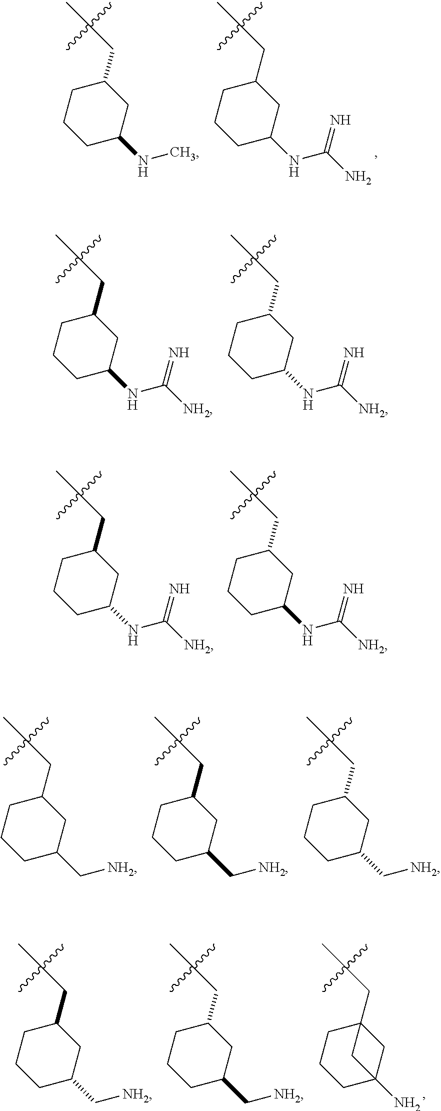

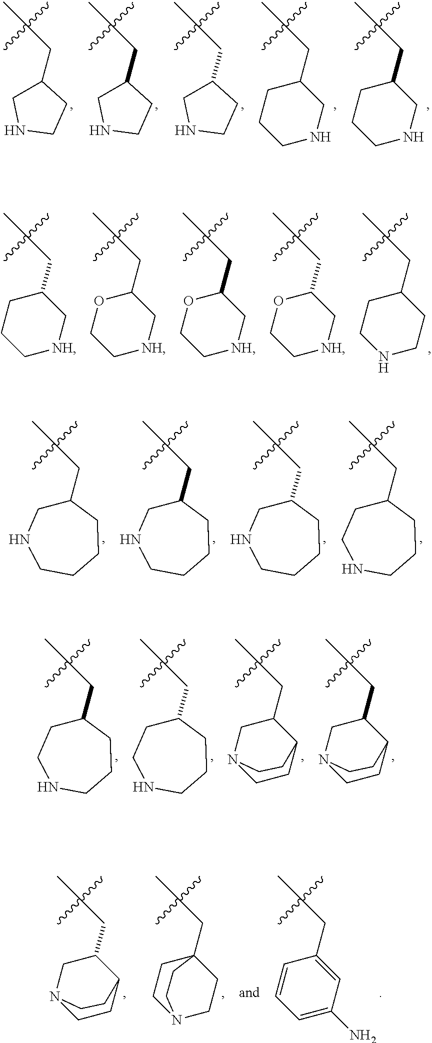

25. The compound of claim 1, or a salt or tautomer thereof, wherein R² and R³, together with the intervening nitrogen, combine to form heterocycloalkyl chosen from pyrrolidinyl, piperidinyl, azepanyl, octahydroquinolinyl, octahydro-1H-pyrrolo[3,2-c]pyridin-5-yl, 2,6-diazaspiro[3.4]octan-6-yl, 2,7-diazaspiro[4.4]nonan-2-yl, 1,8-diazaspiro[4.5]decan-8-yl, 2,7-diazaspiro[4.5]decan-7-yl, 3,9-diazaspiro[5.5]undecan-3-yl, 9-oxa-3,7-diazabicyclo[3.3.1]nonan-3-yl, and (3aR, 8aS)-decahydropyrrolo[3,4-d]azepin-6-yl, any of which is optionally substituted with one or more R⁸.

26. The compound of claim 25, or a salt or tautomer thereof, wherein R² and R³, together with the intervening nitrogen, combine to form heterocycloalkyl chosen from pyrrolidinyl, piperidinyl, azepanyl, and octahydroquinolinyl, any of which is optionally substituted with one or more R⁸.

27. The compound of claim 25, or a salt or tautomer thereof, wherein R² and R³, together with the intervening nitrogen, combine to form

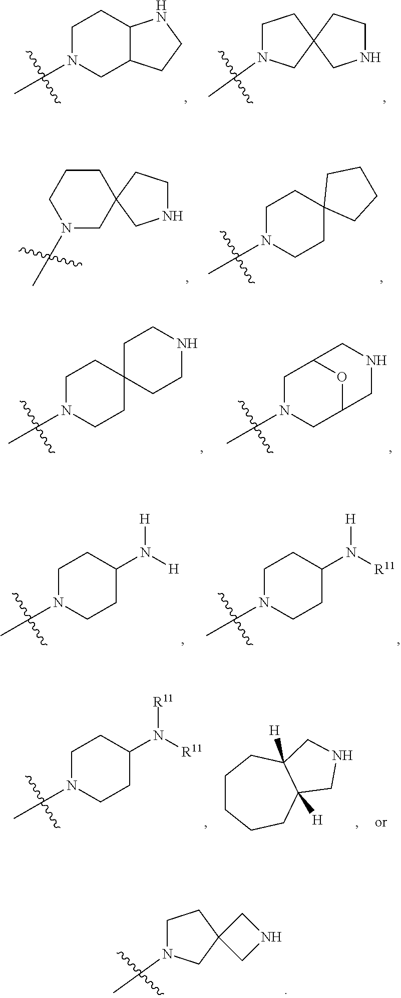

28. The compound of claim 1, or a salt or tautomer thereof, wherein R¹ is chosen from (C₃₋₇) cycloalkyl, 4- to 7-membered heterocycloalkyl, (C₆₋₁₀) aryl, and 5- to 10-membered heteroaryl, any of which is optionally substituted with one R⁶ and one or more R⁷, wherein R⁶ is optionally substituted with one or more R¹⁰.

29. The compound of claim 28, or a salt or tautomer thereof, wherein R¹ is chosen from (C₆₋₁₀) aryl and 5- to 10-membered heteroaryl, either of which is optionally substituted with one R⁶ and one or more R⁷, wherein R⁶ is optionally substituted with one or more R¹⁰.

30. The compound of claim 29, or a salt or tautomer thereof, wherein R¹ is chosen from 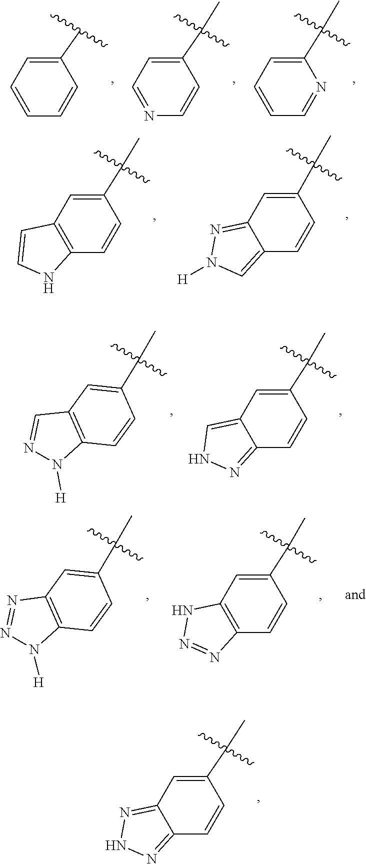
each of which is optionally substituted with one R⁶ and one or more R⁷, wherein R⁶ is optionally substituted with one or more R¹⁰.

31. The compound of claim 30, or a salt or tautomer thereof, wherein R¹ is phenyl and is optionally substituted with one R⁶ and one or more R⁷, wherein R⁶ is optionally substituted with one or more R¹⁰.

32. The compound of claim 31, or a salt or tautomer thereof, wherein R¹ optionally substituted with one R⁶ and one or more R⁷ wherein R⁶ is optionally substituted with one or more R¹⁰ is chosen from 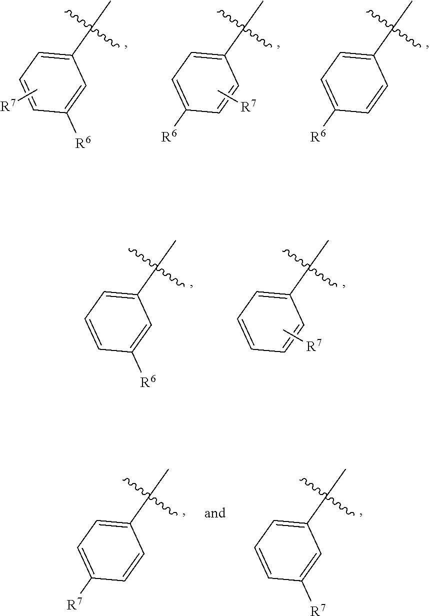
wherein
R⁶ is chosen from cycloalkyl, heterocycloalkyl, aryl, heteroaryl, (cycloalkyl)carbonyl, (heterocycloalkyl)carbonyl, (aryl)carbonyl, and (heteroaryl)carbonyl, any of which is optionally substituted with one or more R¹⁰; and
R⁷ is independently chosen from halo, cyano, hydroxy, alkyl, alkoxy, (hydroxy)alkyl, haloalkoxy, and (hydroxy)alkoxy.

33. The compound of claim 1, or a salt or tautomer thereof, wherein R⁶ is chosen from cycloalkyl, heterocycloalkyl, aryl, and heteroaryl, any of which is optionally substituted with one or more R¹⁰.

34. The compound of claim 33, or a salt or tautomer thereof, wherein R⁶ is heterocycloalkyl optionally substituted with one or more R¹⁰.

35. The compound of claim 34, or a salt or tautomer thereof, wherein R⁶ is chosen from pyrrolidin-1-yl, piperidin-1-yl, piperazin-1-yl, thiomorpholin-4-yl, 1-oxidothiomorpholino, 1,1-dioxidothiomorpholino, 3,3-dioxido-3-thia-6-azabicyclo[3.1.1]heptan-6-yl, 1-oxa-8-azaspiro[4.5]decan-8-yl, and 3-oxopiperazin-1-yl, any of which is optionally substituted with one or more R¹⁰.

36. The compound of claim 1, or a salt or tautomer thereof, wherein each R¹⁰ is independently chosen from cyano, hydroxy, alkyl, (hydroxy)alkyl, and alkoxy.

37. The compound of claim 36, or a salt or tautomer thereof, wherein each R¹⁰ is independently chosen from methyl, methoxy, ethoxy, isopropoxy, hydroxy, and —CH₂CH₂OH.

38. The compound of claim 1, or a salt or tautomer thereof, wherein R⁷ is chosen from methyl, isopropyl, F, Cl, —CH₂CH₂OH, —OCH₂CH₂OH, methoxy, ethoxy, isopropoxy, and (1,3-difluoropropan-2-yl)oxy.

39. The compound of claim 1, or a salt or tautomer thereof, wherein R¹ optionally substituted with one R⁶ and one or more R⁷ wherein R⁶ is optionally substituted with one or more R¹⁰ is chosen from

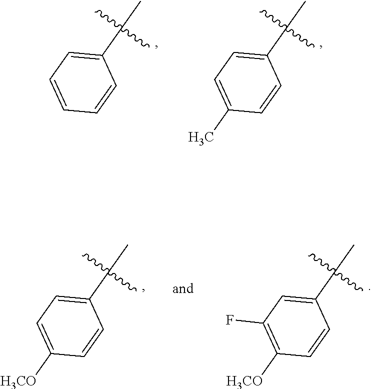

40. A compound chosen from: 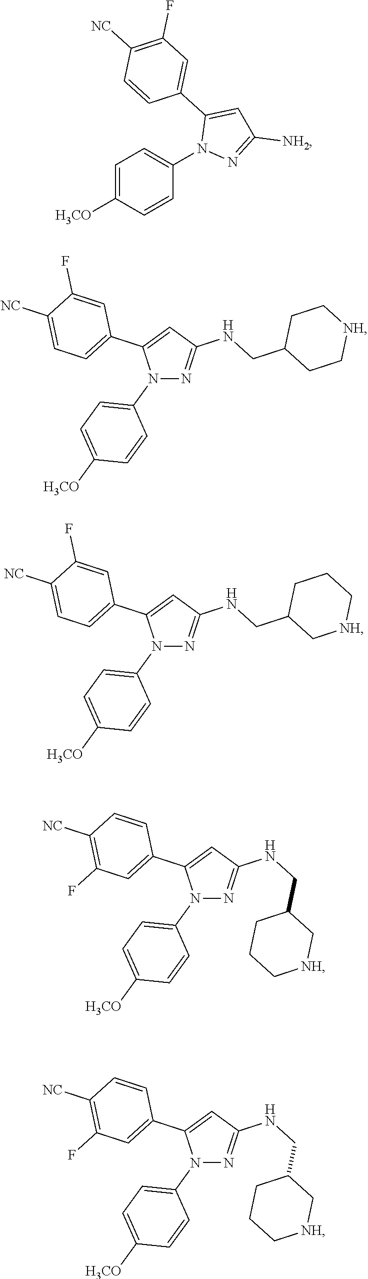 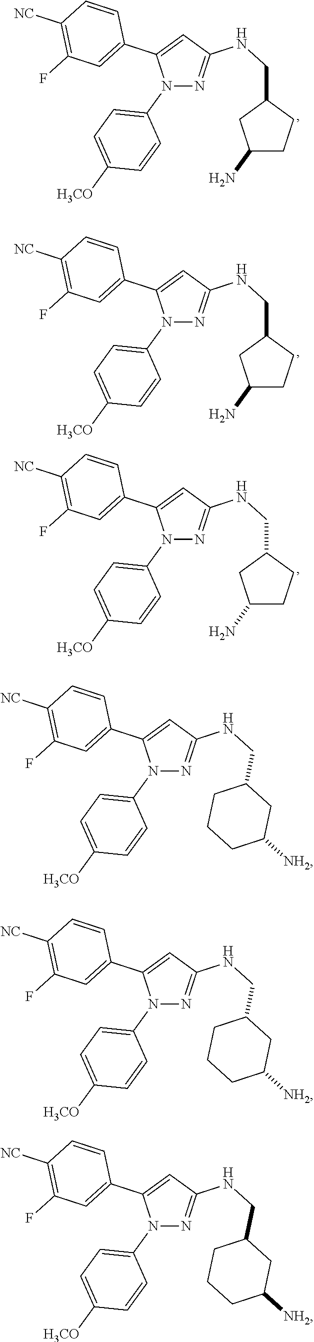 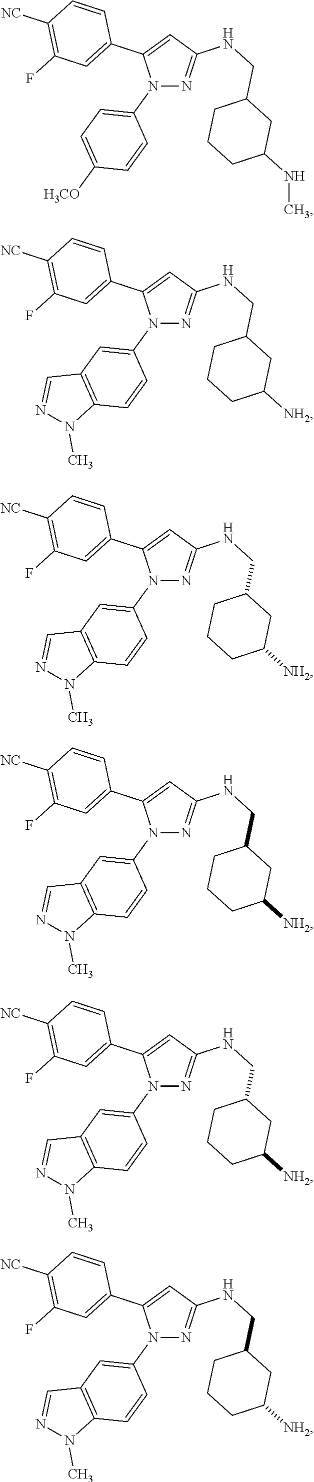 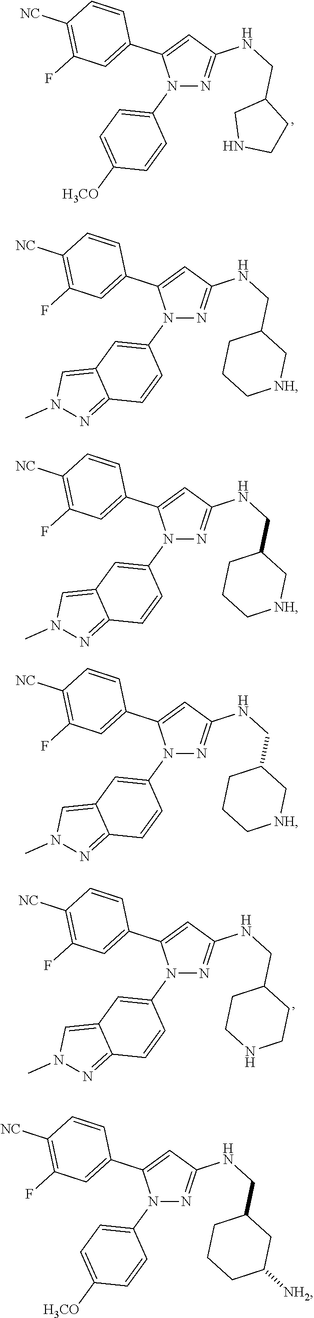 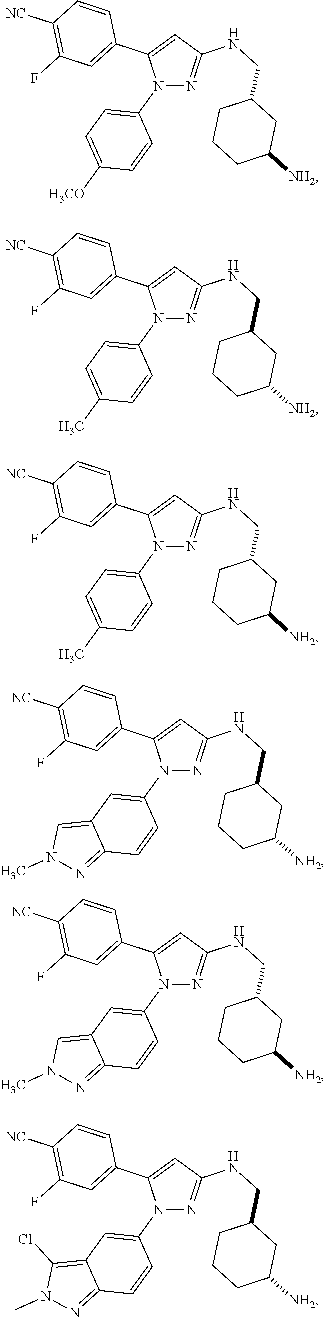 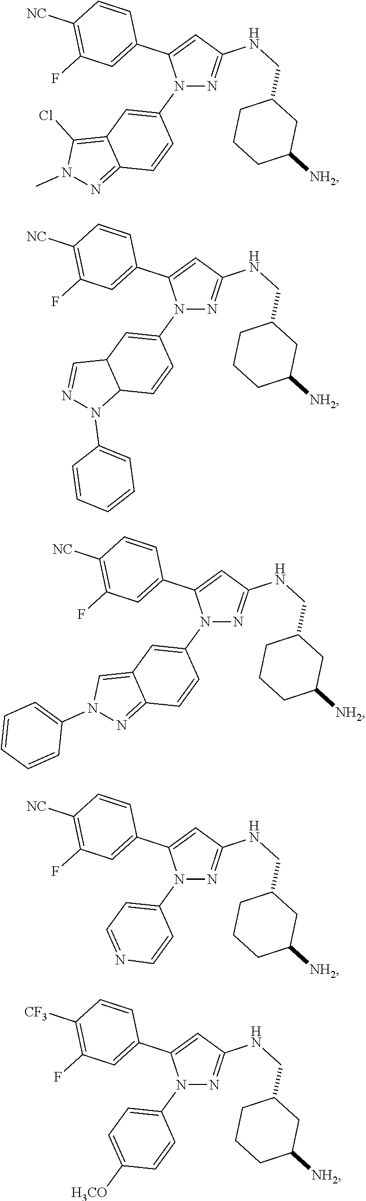 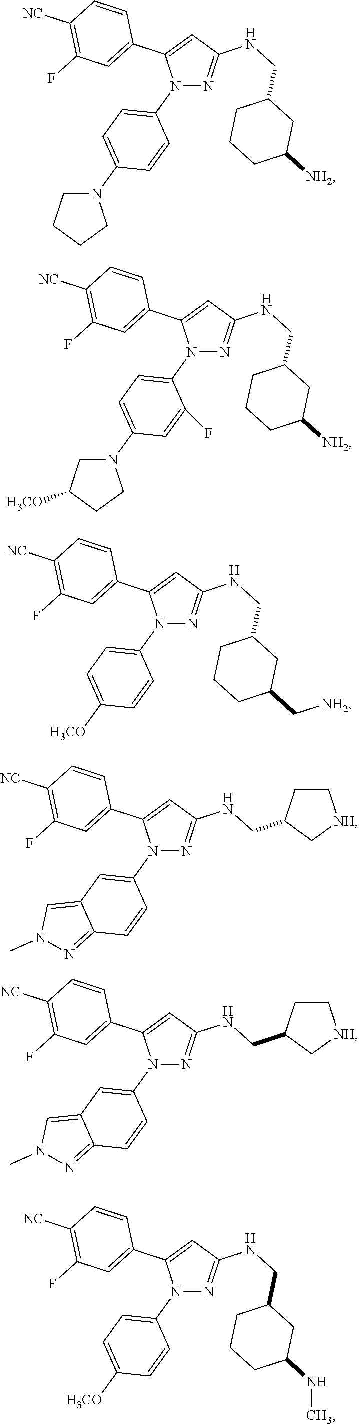 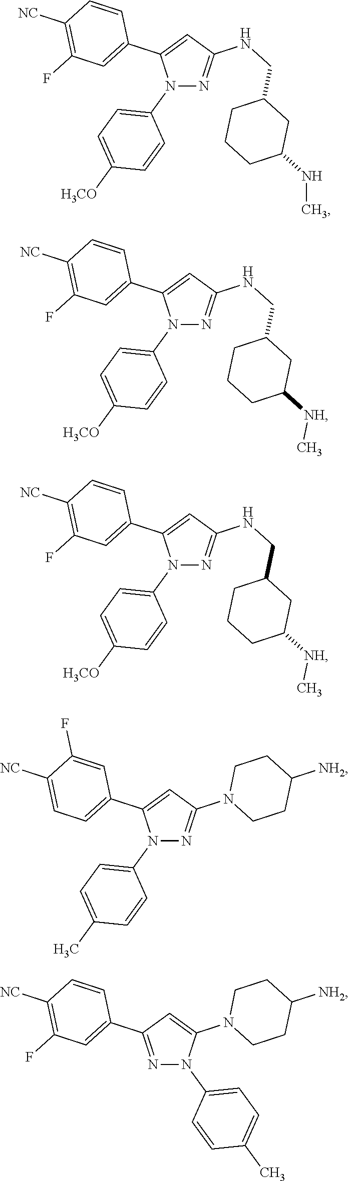 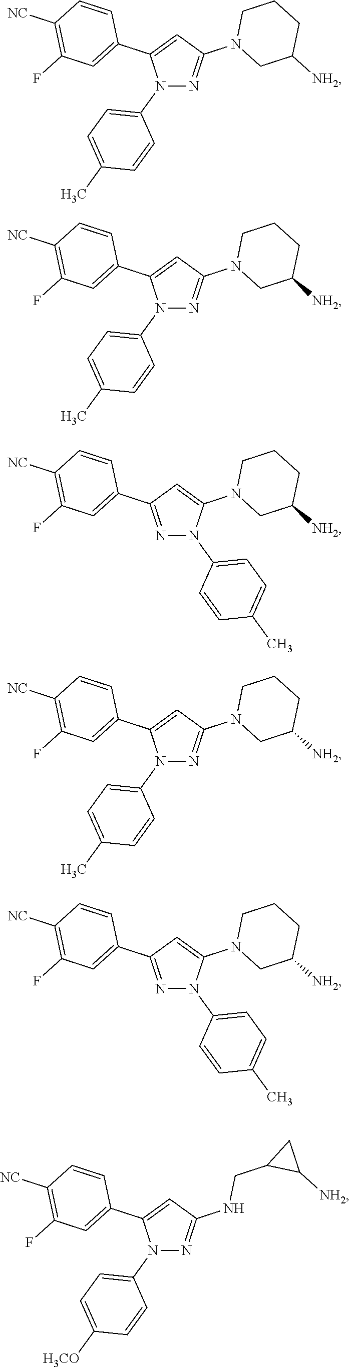 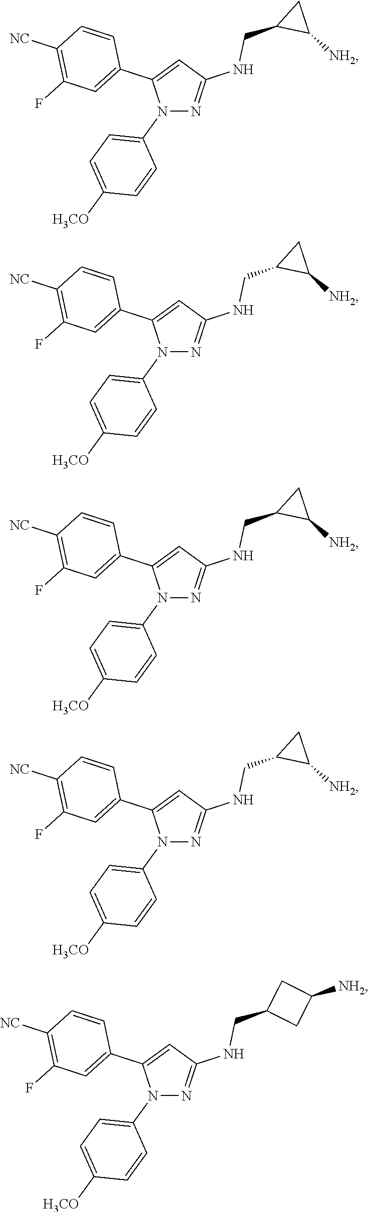 [SMILES: COc1ccc(-n2nc(NC[C@H]3C[C@@H]3N)cc2-c2ccc(C#N)c(F)c2)cc1 ; COc1ccc(-n2nc(NC[C@@H]3C[C@H]3N)cc2-c2ccc(C#N)c(F)c2)cc1 ; COc1ccc(-n2nc(NC[C@H]3C[C@H]3N)cc2-c2ccc(C#N)c(F)c2)cc1 ; COc1ccc(-n2nc(NC[C@@H]3C[C@@H]3N)cc2-c2ccc(C#N)c(F)c2)cc1 ; COc1ccc(-n2nc(NC[C@H]3C[C@@H](N)C3)cc2-c2ccc(C#N)c(F)c2)cc1] 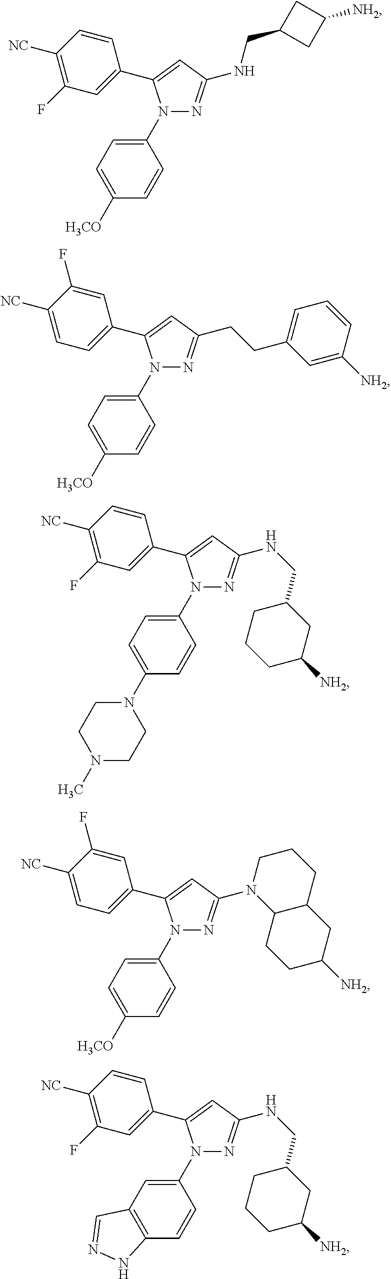 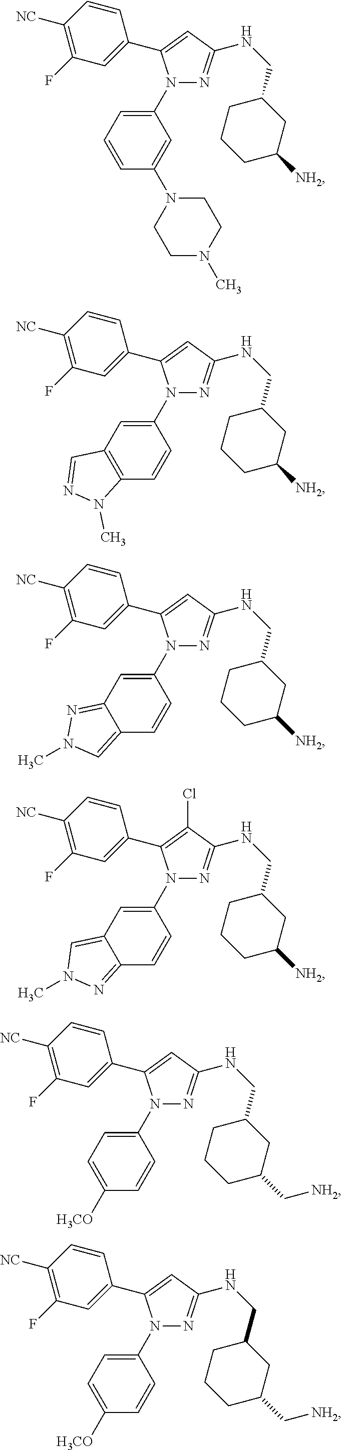 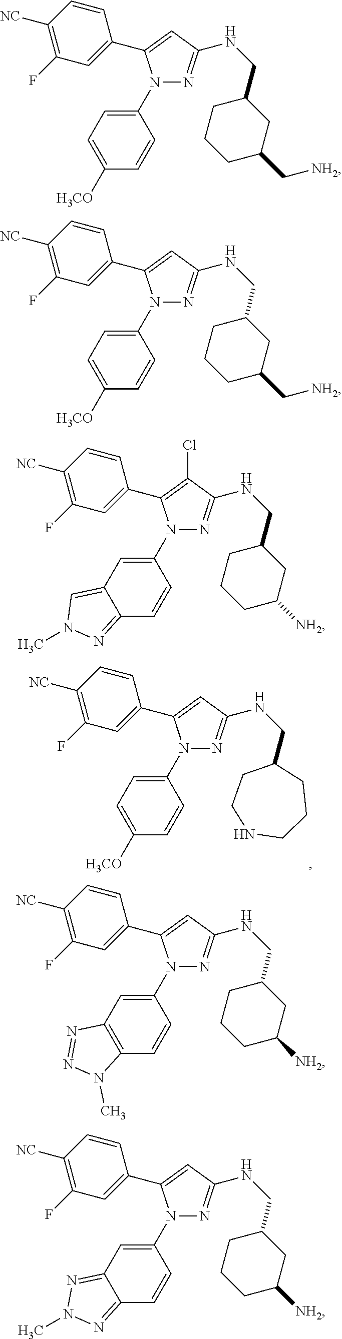 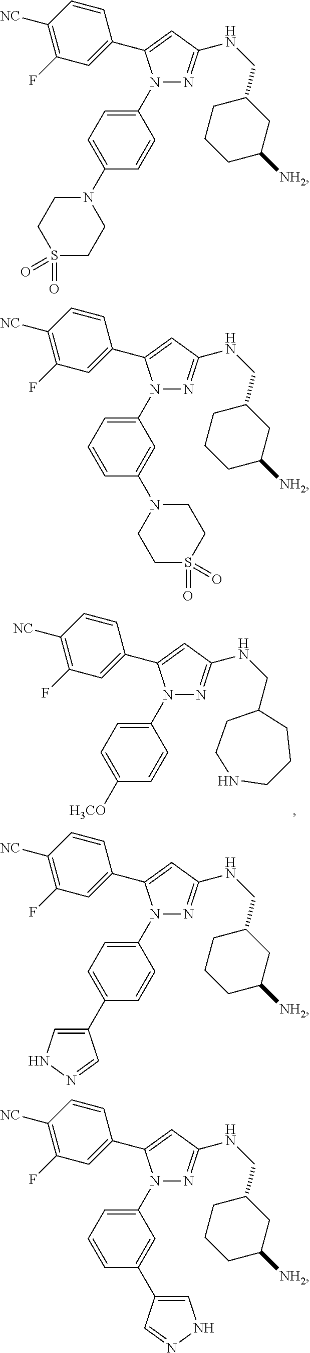 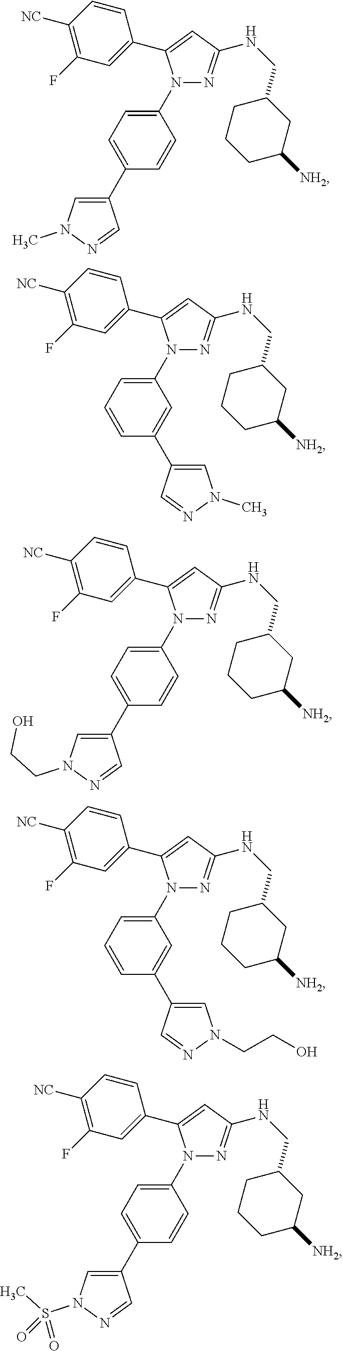 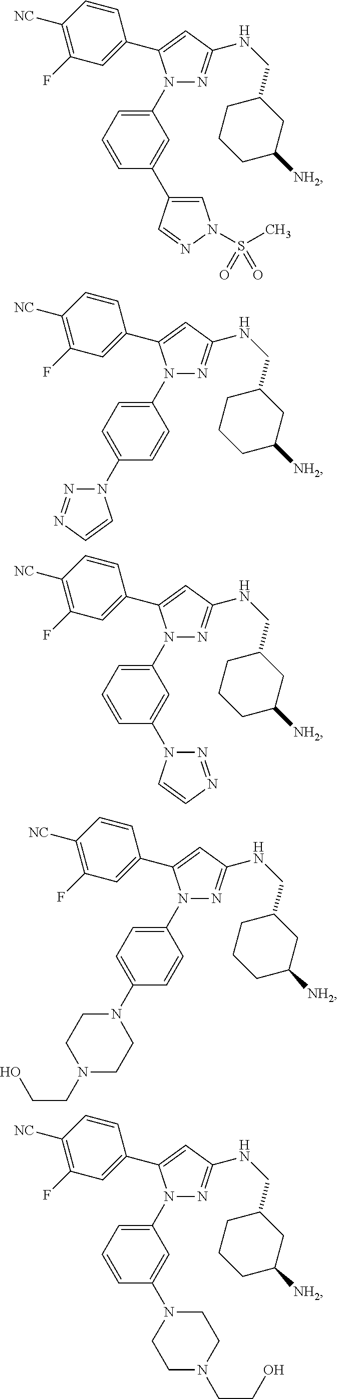 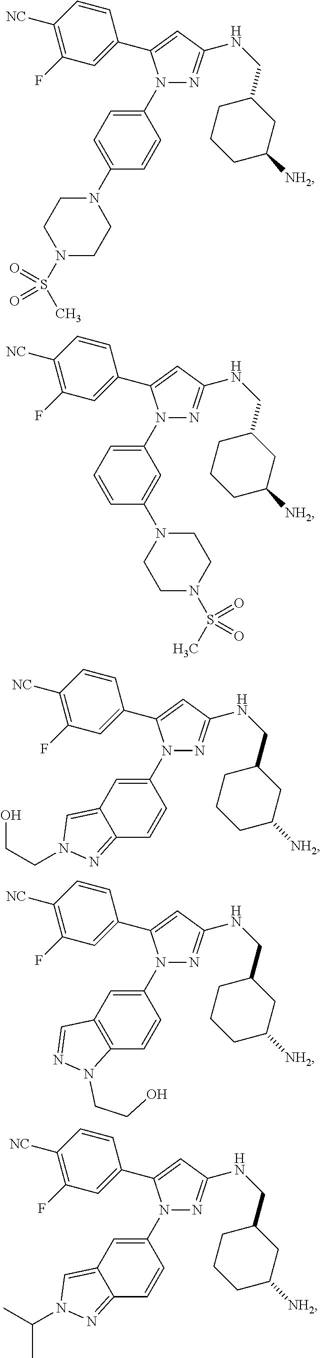 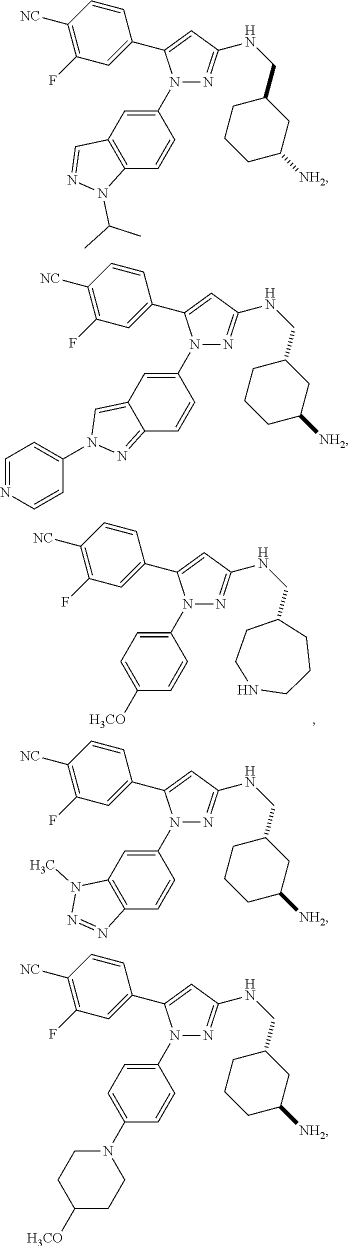 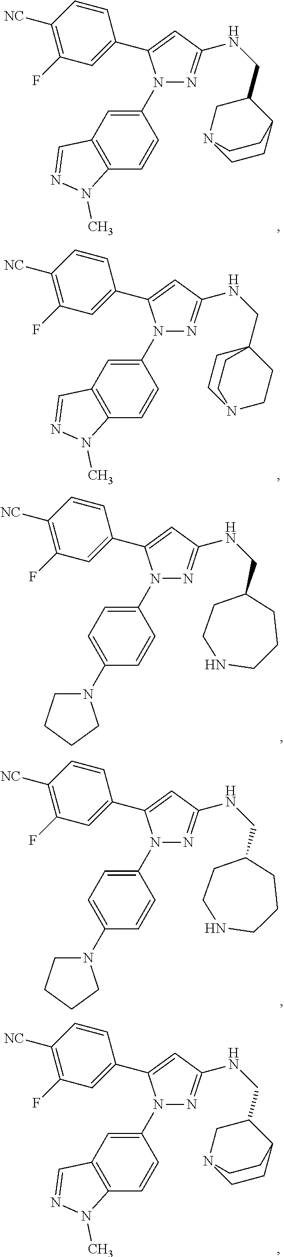 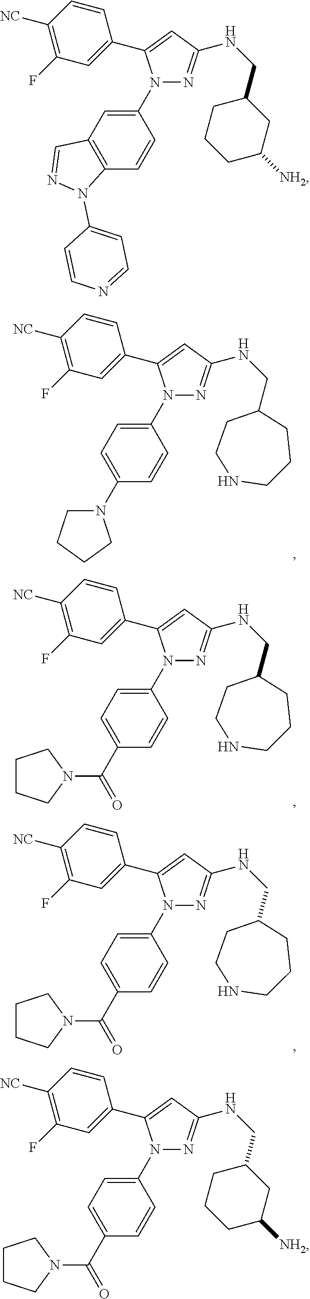 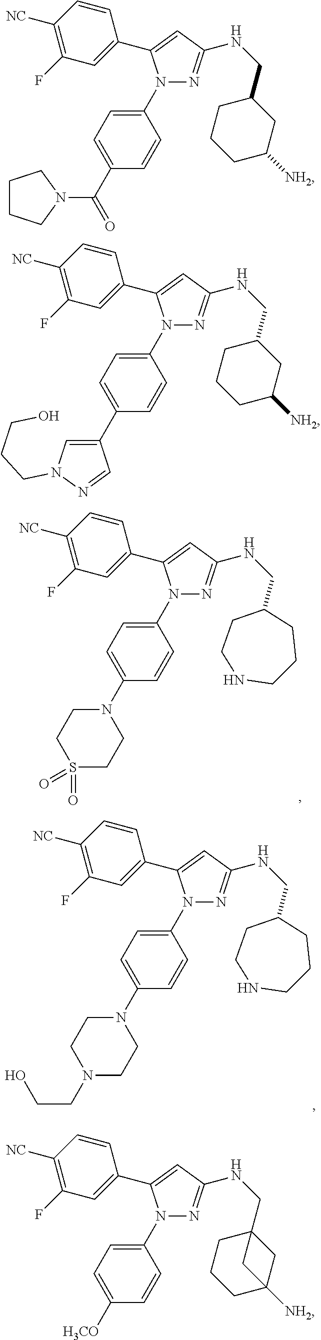 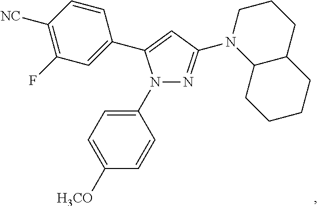 [SMILES: COc1ccc(-n2nc(N3CCCC4CCCCC43)cc2-c2ccc(C#N)c(F)c2)cc1] 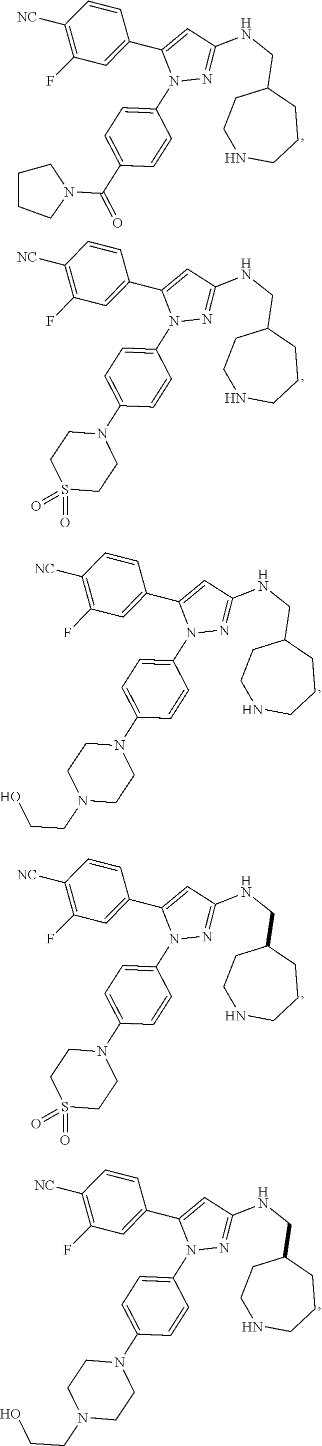 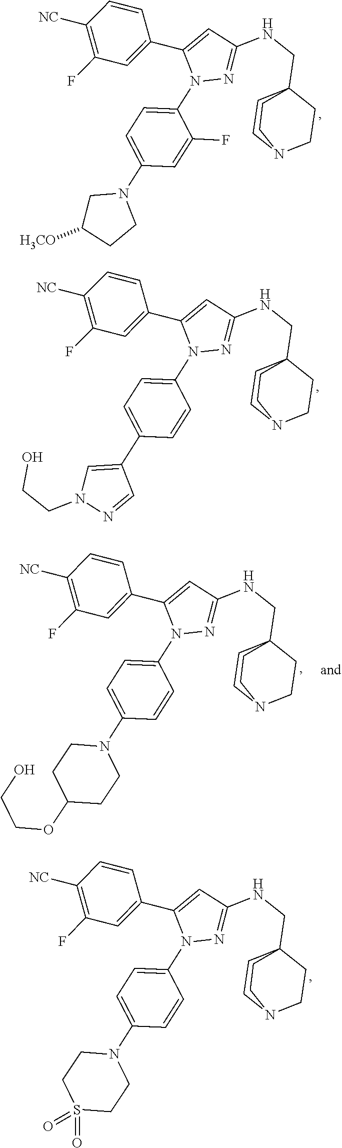
or a salt or tautomer thereof.

41. A compound chosen from: 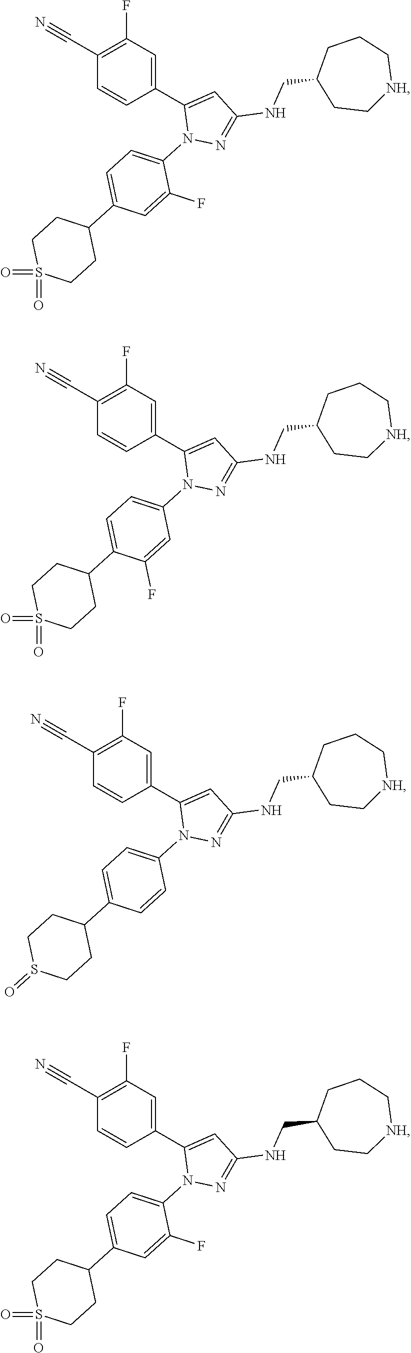 [SMILES: N#Cc1ccc(-c2cc(NC[C@@H]3CCCNCC3)nn2-c2ccc(C3CCS(=O)(=O)CC3)cc2F)cc1F ; N#Cc1ccc(-c2cc(NC[C@@H]3CCCNCC3)nn2-c2ccc(C3CCS(=O)(=O)CC3)c(F)c2)cc1F ; N#Cc1ccc(-c2cc(NC[C@@H]3CCCNCC3)nn2-c2ccc(C3CCS(=O)CC3)cc2)cc1F ; N#Cc1ccc(-c2cc(NC[C@H]3CCCNCC3)nn2-c2ccc(C3CCS(=O)(=O)CC3)cc2F)cc1F] 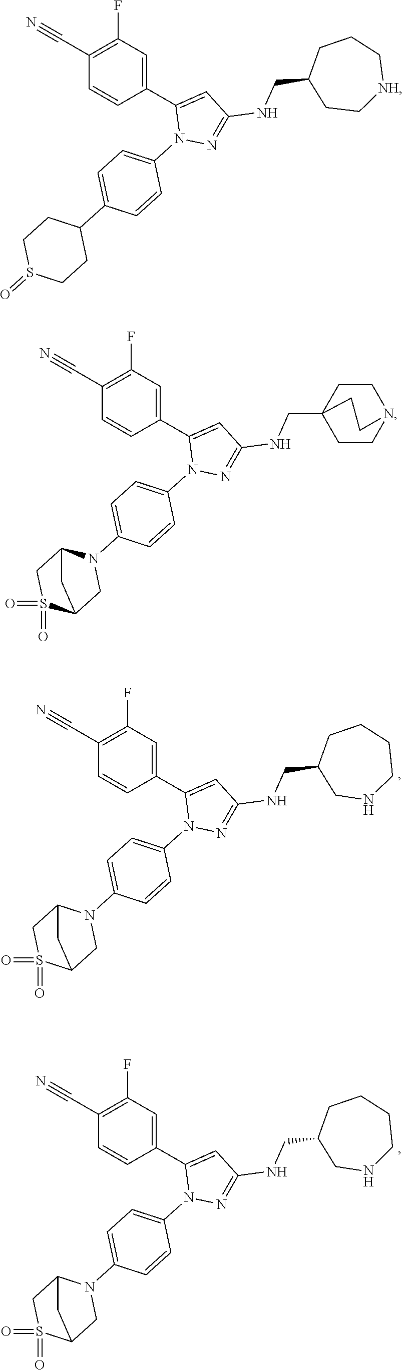 [SMILES: N#Cc1ccc(-c2cc(NC[C@H]3CCCNCC3)nn2-c2ccc(C3CCS(=O)CC3)cc2)cc1F ; N#Cc1ccc(-c2cc(NCC34CCN(CC3)CC4)nn2-c2ccc(N3C[C@@H]4C[C@H]3CS4(=O)=O)cc2)cc1F ; N#Cc1ccc(-c2cc(NC[C@H]3CCCCNC3)nn2-c2ccc(N3CC4CC3CS4(=O)=O)cc2)cc1F ; N#Cc1ccc(-c2cc(NC[C@@H]3CCCCNC3)nn2-c2ccc(N3CC4CC3CS4(=O)=O)cc2)cc1F] 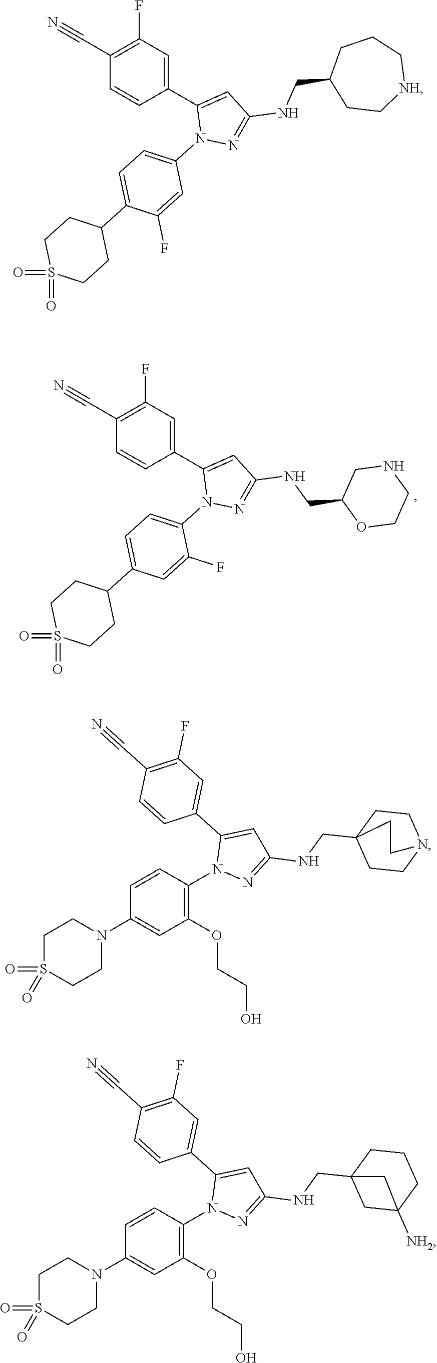 [SMILES: N#Cc1ccc(-c2cc(NC[C@H]3CCCNCC3)nn2-c2ccc(C3CCS(=O)(=O)CC3)c(F)c2)cc1F ; N#Cc1ccc(-c2cc(NC[C@H]3CNCCO3)nn2-c2ccc(C3CCS(=O)(=O)CC3)cc2F)cc1F ; N#Cc1ccc(-c2cc(NCC34CCN(CC3)CC4)nn2-c2ccc(N3CCS(=O)(=O)CC3)cc2OCCO)cc1F ; N#Cc1ccc(-c2cc(NCC34CCCC(N)(C3)C4)nn2-c2ccc(N3CCS(=O)(=O)CC3)cc2OCCO)cc1F] 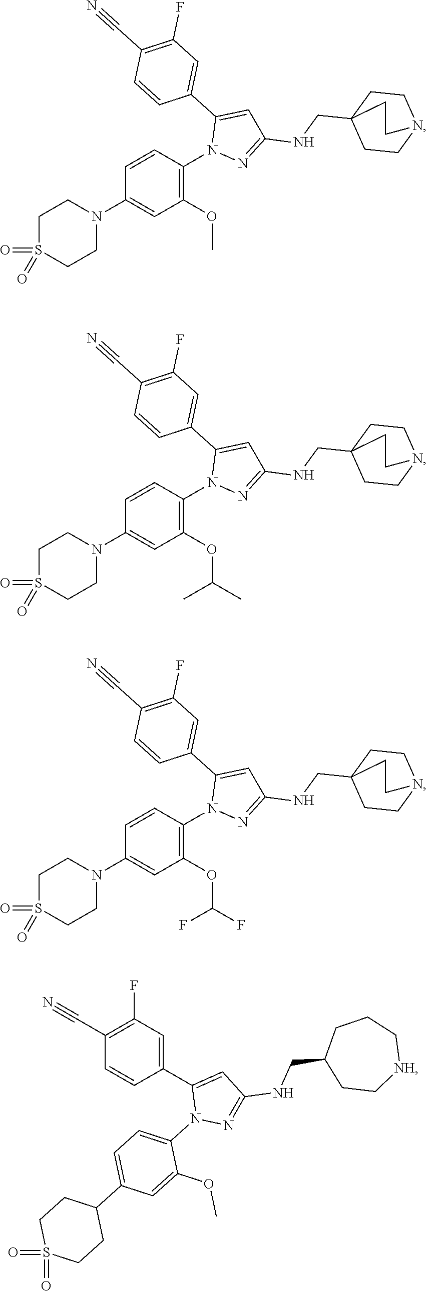 [SMILES: COc1cc(N2CCS(=O)(=O)CC2)ccc1-n1nc(NCC23CCN(CC2)CC3)cc1-c1ccc(C#N)c(F)c1 ; CC(C)Oc1cc(N2CCS(=O)(=O)CC2)ccc1-n1nc(NCC23CCN(CC2)CC3)cc1-c1ccc(C#N)c(F)c1 ; N#Cc1ccc(-c2cc(NCC34CCN(CC3)CC4)nn2-c2ccc(N3CCS(=O)(=O)CC3)cc2OC(F)F)cc1F ; COc1cc(C2CCS(=O)(=O)CC2)ccc1-n1nc(NC[C@H]2CCCNCC2)cc1-c1ccc(C#N)c(F)c1] 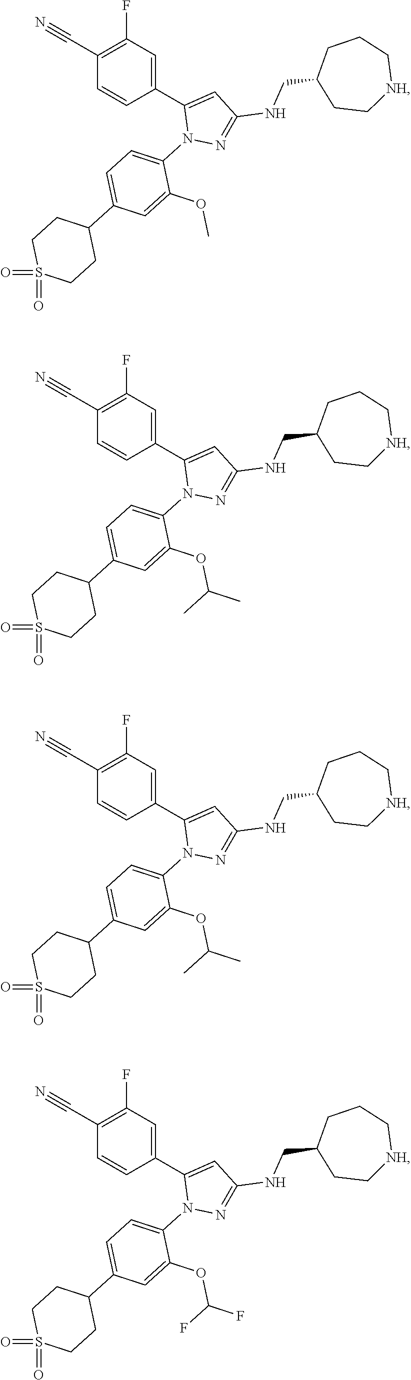 [SMILES: COc1cc(C2CCS(=O)(=O)CC2)ccc1-n1nc(NC[C@@H]2CCCNCC2)cc1-c1ccc(C#N)c(F)c1 ; CC(C)Oc1cc(C2CCS(=O)(=O)CC2)ccc1-n1nc(NC[C@H]2CCCNCC2)cc1-c1ccc(C#N)c(F)c1 ; CC(C)Oc1cc(C2CCS(=O)(=O)CC2)ccc1-n1nc(NC[C@@H]2CCCNCC2)cc1-c1ccc(C#N)c(F)c1 ; N#Cc1ccc(-c2cc(NC[C@H]3CCCNCC3)nn2-c2ccc(C3CCS(=O)(=O)CC3)cc2OC(F)F)cc1F] 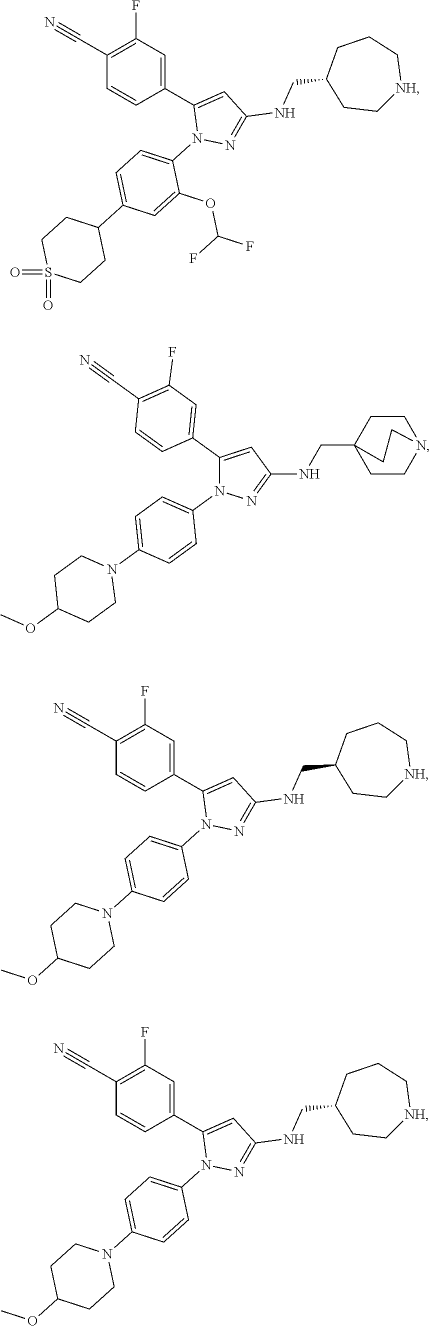 [SMILES: N#Cc1ccc(-c2cc(NC[C@@H]3CCCNCC3)nn2-c2ccc(C3CCS(=O)(=O)CC3)cc2OC(F)F)cc1F ; COC1CCN(c2ccc(-n3nc(NCC45CCN(CC4)CC5)cc3-c3ccc(C#N)c(F)c3)cc2)CC1 ; COC1CCN(c2ccc(-n3nc(NC[C@H]4CCCNCC4)cc3-c3ccc(C#N)c(F)c3)cc2)CC1 ; COC1CCN(c2ccc(-n3nc(NC[C@@H]4CCCNCC4)cc3-c3ccc(C#N)c(F)c3)cc2)CC1] 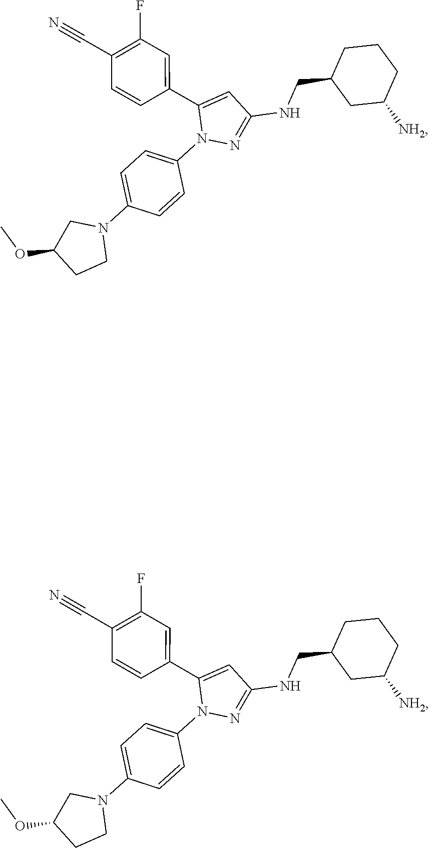 [SMILES: CO[C@@H]1CCN(c2ccc(-n3nc(NC[C@H]4CCC[C@H](N)C4)cc3-c3ccc(C#N)c(F)c3)cc2)C1 ; CO[C@H]1CCN(c2ccc(-n3nc(NC[C@H]4CCC[C@H](N)C4)cc3-c3ccc(C#N)c(F)c3)cc2)C1] 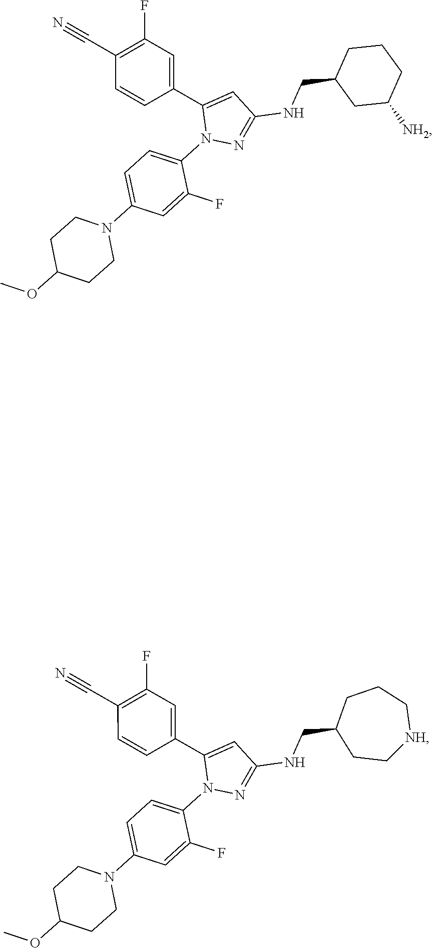 [SMILES: COC1CCN(c2ccc(-n3nc(NC[C@H]4CCC[C@H](N)C4)cc3-c3ccc(C#N)c(F)c3)c(F)c2)CC1 ; COC1CCN(c2ccc(-n3nc(NC[C@H]4CCCNCC4)cc3-c3ccc(C#N)c(F)c3)c(F)c2)CC1] 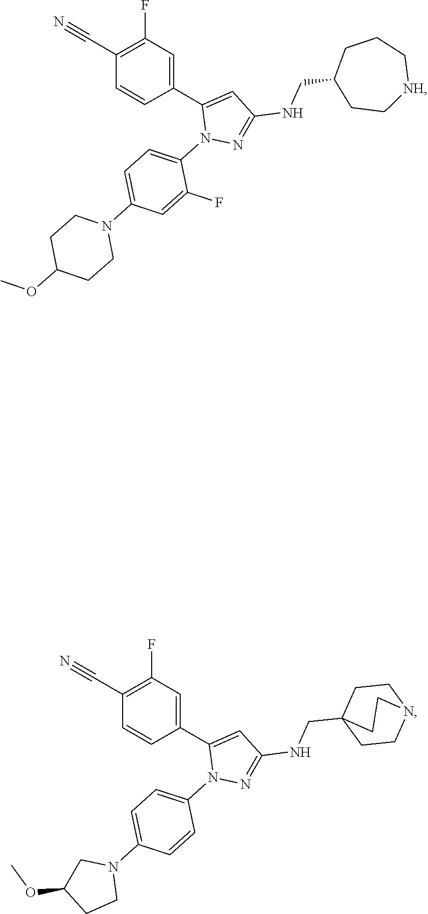 [SMILES: COC1CCN(c2ccc(-n3nc(NC[C@@H]4CCCNCC4)cc3-c3ccc(C#N)c(F)c3)c(F)c2)CC1 ; CO[C@@H]1CCN(c2ccc(-n3nc(NCC45CCN(CC4)CC5)cc3-c3ccc(C#N)c(F)c3)cc2)C1] 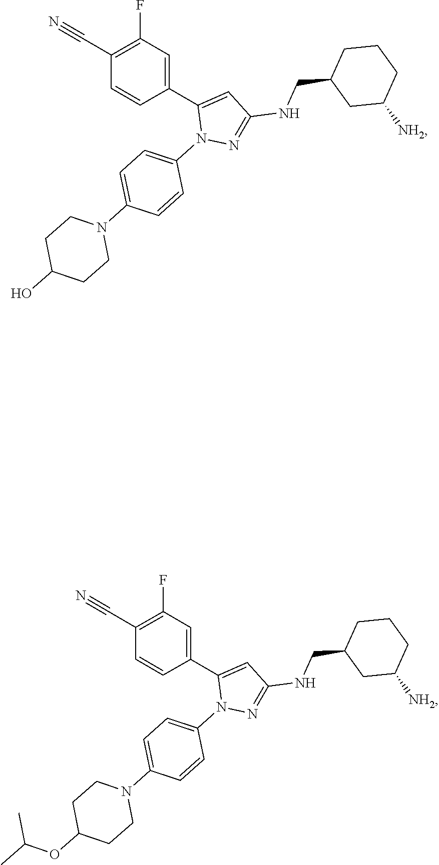 [SMILES: N#Cc1ccc(-c2cc(NC[C@H]3CCC[C@H](N)C3)nn2-c2ccc(N3CCC(O)CC3)cc2)cc1F ; CC(C)OC1CCN(c2ccc(-n3nc(NC[C@H]4CCC[C@H](N)C4)cc3-c3ccc(C#N)c(F)c3)cc2)CC1] 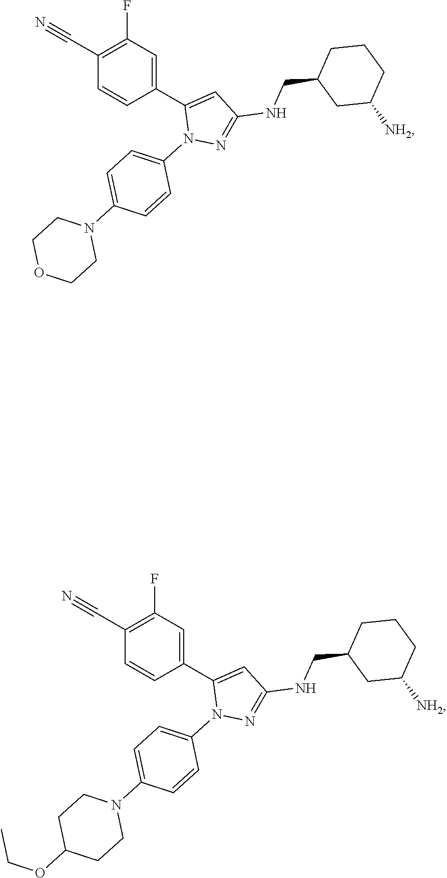 [SMILES: N#Cc1ccc(-c2cc(NC[C@H]3CCC[C@H](N)C3)nn2-c2ccc(N3CCOCC3)cc2)cc1F ; CCOC1CCN(c2ccc(-n3nc(NC[C@H]4CCC[C@H](N)C4)cc3-c3ccc(C#N)c(F)c3)cc2)CC1] 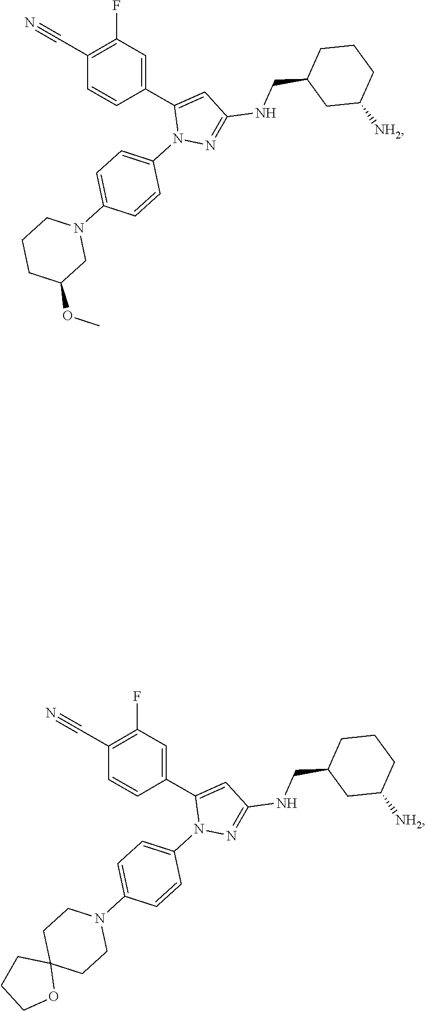 [SMILES: CO[C@H]1CCCN(c2ccc(-n3nc(NC[C@H]4CCC[C@H](N)C4)cc3-c3ccc(C#N)c(F)c3)cc2)C1 ; N#Cc1ccc(-c2cc(NC[C@H]3CCC[C@H](N)C3)nn2-c2ccc(N3CCC4(CCCO4)CC3)cc2)cc1F] 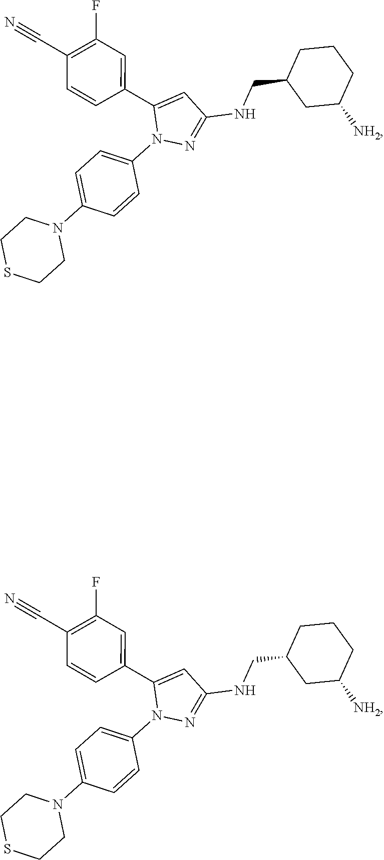 [SMILES: N#Cc1ccc(-c2cc(NC[C@H]3CCC[C@H](N)C3)nn2-c2ccc(N3CCSCC3)cc2)cc1F ; N#Cc1ccc(-c2cc(NC[C@@H]3CCC[C@H](N)C3)nn2-c2ccc(N3CCSCC3)cc2)cc1F] 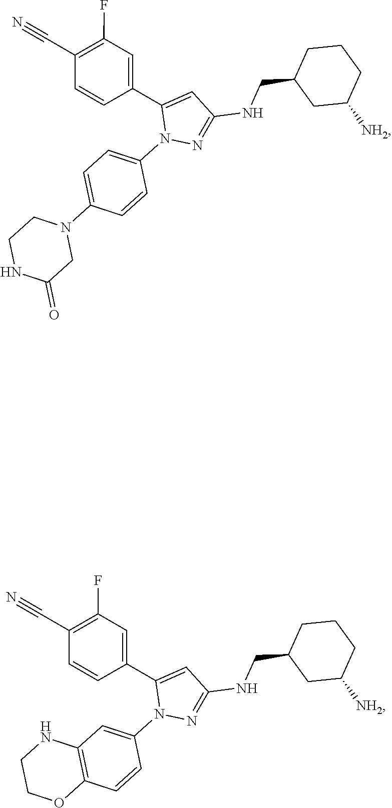 [SMILES: N#Cc1ccc(-c2cc(NC[C@H]3CCC[C@H](N)C3)nn2-c2ccc(N3CCNC(=O)C3)cc2)cc1F ; N#Cc1ccc(-c2cc(NC[C@H]3CCC[C@H](N)C3)nn2-c2ccc3c(c2)NCCO3)cc1F] 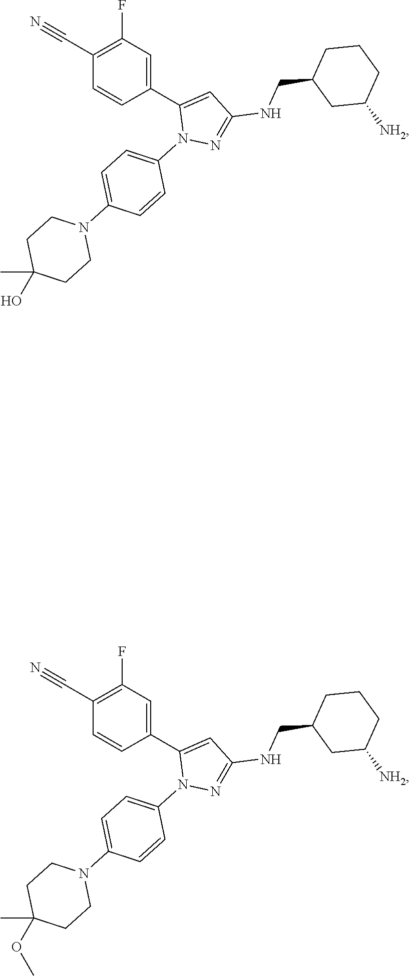 [SMILES: CC1(O)CCN(c2ccc(-n3nc(NC[C@H]4CCC[C@H](N)C4)cc3-c3ccc(C#N)c(F)c3)cc2)CC1 ; COC1(C)CCN(c2ccc(-n3nc(NC[C@H]4CCC[C@H](N)C4)cc3-c3ccc(C#N)c(F)c3)cc2)CC1] 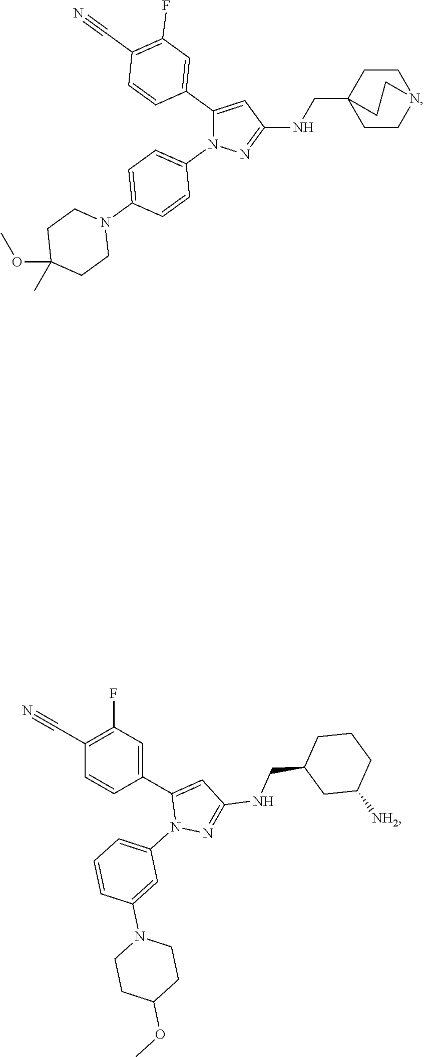 [SMILES: COC1(C)CCN(c2ccc(-n3nc(NCC45CCN(CC4)CC5)cc3-c3ccc(C#N)c(F)c3)cc2)CC1 ; COC1CCN(c2cccc(-n3nc(NC[C@H]4CCC[C@H](N)C4)cc3-c3ccc(C#N)c(F)c3)c2)CC1] 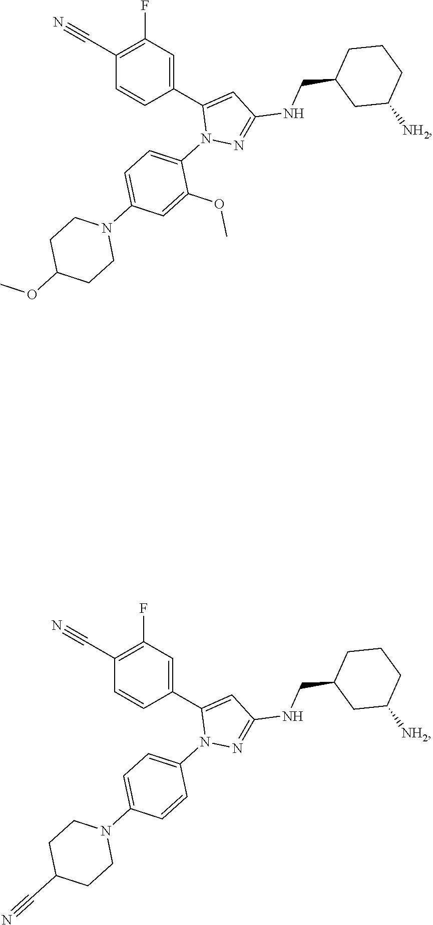 [SMILES: COc1cc(N2CCC(OC)CC2)ccc1-n1nc(NC[C@H]2CCC[C@H](N)C2)cc1-c1ccc(C#N)c(F)c1 ; N#Cc1ccc(-c2cc(NC[C@H]3CCC[C@H](N)C3)nn2-c2ccc(N3CCC(C#N)CC3)cc2)cc1F] 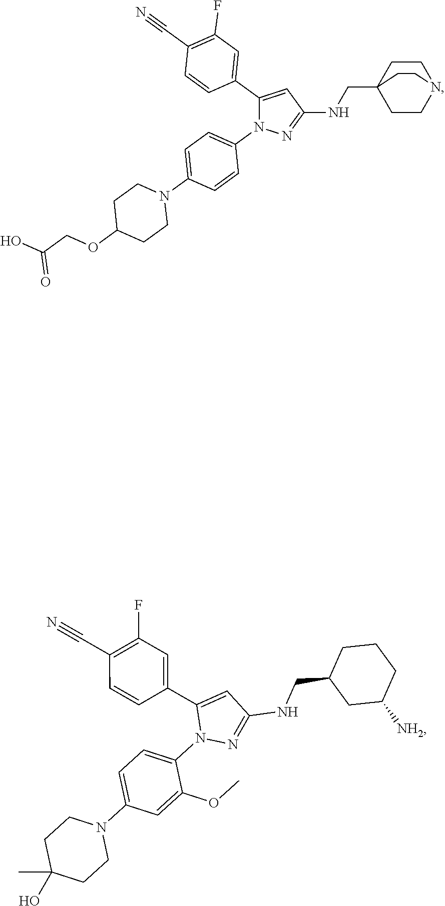 [SMILES: N#Cc1ccc(-c2cc(NCC34CCN(CC3)CC4)nn2-c2ccc(N3CCC(OCC(=O)O)CC3)cc2)cc1F ; COc1cc(N2CCC(C)(O)CC2)ccc1-n1nc(NC[C@H]2CCC[C@H](N)C2)cc1-c1ccc(C#N)c(F)c1] 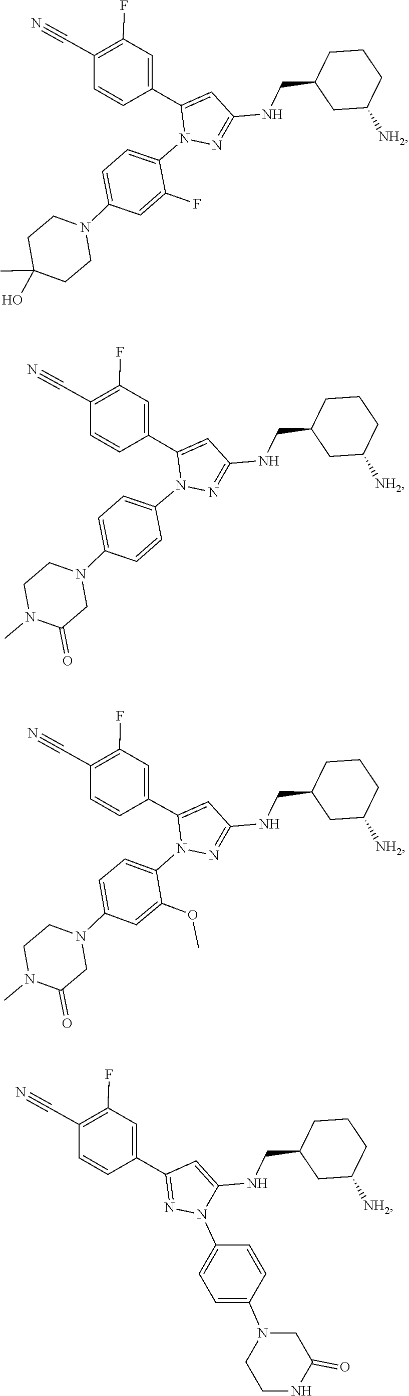 [SMILES: CC1(O)CCN(c2ccc(-n3nc(NC[C@H]4CCC[C@H](N)C4)cc3-c3ccc(C#N)c(F)c3)c(F)c2)CC1 ; CN1CCN(c2ccc(-n3nc(NC[C@H]4CCC[C@H](N)C4)cc3-c3ccc(C#N)c(F)c3)cc2)CC1=O ; COc1cc(N2CCN(C)C(=O)C2)ccc1-n1nc(NC[C@H]2CCC[C@H](N)C2)cc1-c1ccc(C#N)c(F)c1 ; N#Cc1ccc(-c2cc(NC[C@H]3CCC[C@H](N)C3)n(-c3ccc(N4CCNC(=O)C4)cc3)n2)cc1F] 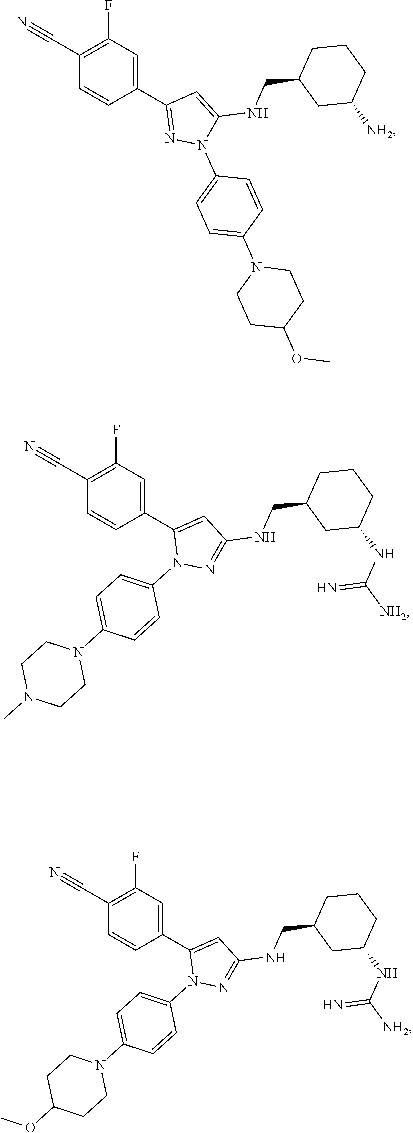 [SMILES: COC1CCN(c2ccc(-n3nc(-c4ccc(C#N)c(F)c4)cc3NC[C@H]3CCC[C@H](N)C3)cc2)CC1 ; CN1CCN(c2ccc(-n3nc(NC[C@H]4CCC[C@H](NC(=N)N)C4)cc3-c3ccc(C#N)c(F)c3)cc2)CC1 ; COC1CCN(c2ccc(-n3nc(NC[C@H]4CCC[C@H](NC(=N)N)C4)cc3-c3ccc(C#N)c(F)c3)cc2)CC1] 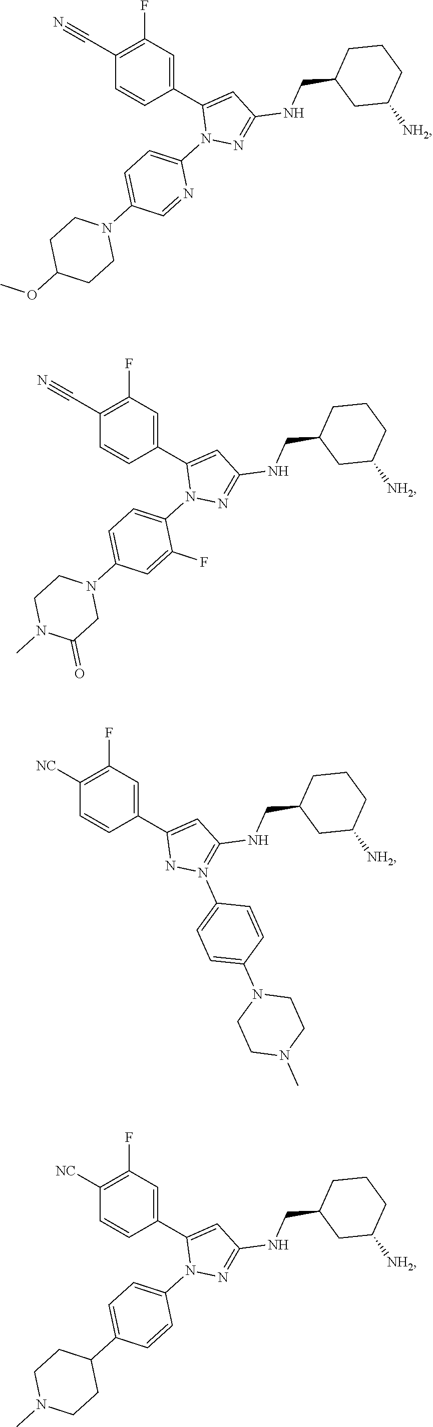 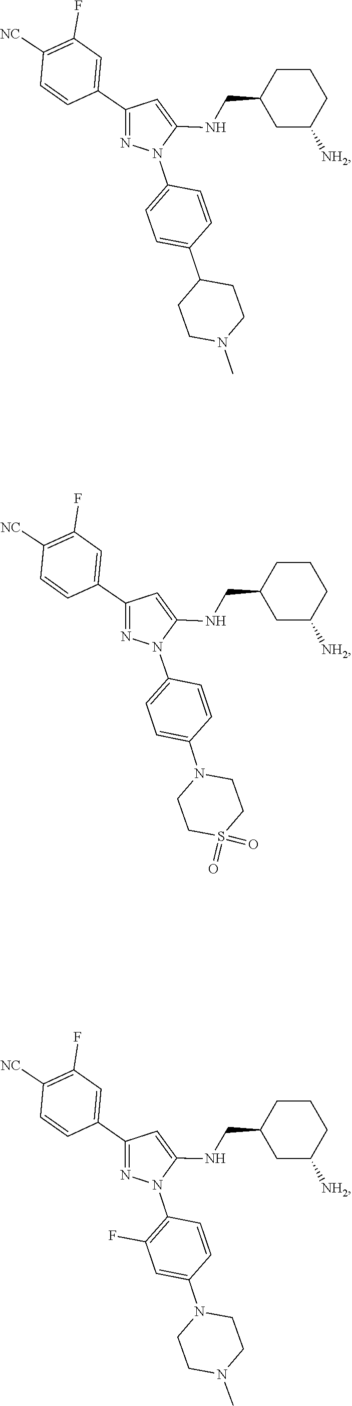 [SMILES: CN1CCC(c2ccc(-n3nc(-c4ccc(C#N)c(F)c4)cc3NC[C@H]3CCC[C@H](N)C3)cc2)CC1 ; N#Cc1ccc(-c2cc(NC[C@H]3CCC[C@H](N)C3)n(-c3ccc(N4CCS(=O)(=O)CC4)cc3)n2)cc1F ; CN1CCN(c2ccc(-n3nc(-c4ccc(C#N)c(F)c4)cc3NC[C@H]3CCC[C@H](N)C3)c(F)c2)CC1] 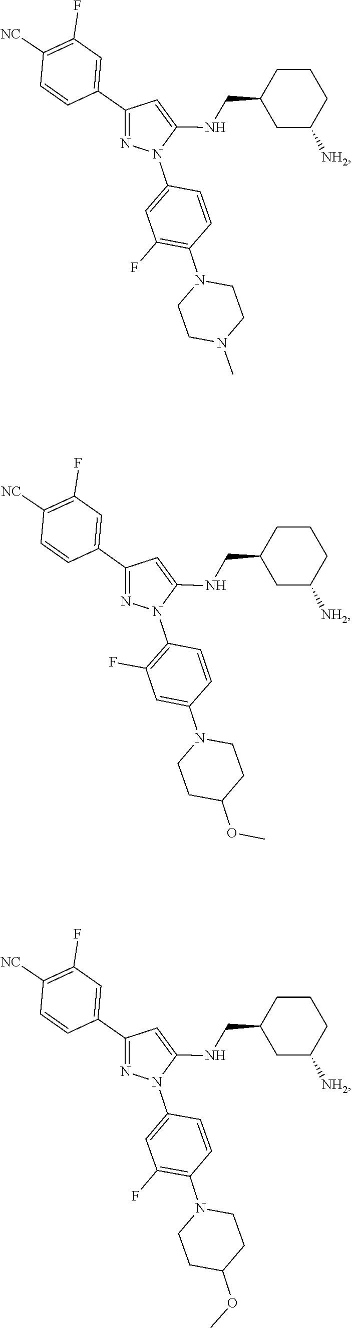 [SMILES: CN1CCN(c2ccc(-n3nc(-c4ccc(C#N)c(F)c4)cc3NC[C@H]3CCC[C@H](N)C3)cc2F)CC1 ; COC1CCN(c2ccc(-n3nc(-c4ccc(C#N)c(F)c4)cc3NC[C@H]3CCC[C@H](N)C3)c(F)c2)CC1 ; COC1CCN(c2ccc(-n3nc(-c4ccc(C#N)c(F)c4)cc3NC[C@H]3CCC[C@H](N)C3)cc2F)CC1] 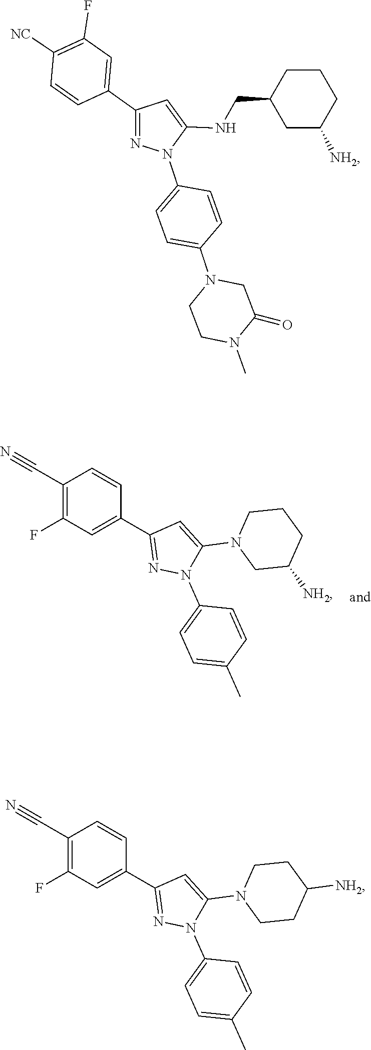 [SMILES: CN1CCN(c2ccc(-n3nc(-c4ccc(C#N)c(F)c4)cc3NC[C@H]3CCC[C@H](N)C3)cc2)CC1=O ; Cc1ccc(-n2nc(-c3ccc(C#N)c(F)c3)cc2N2CCC[C@H](N)C2)cc1 ; "and" ; Cc1ccc(-n2nc(-c3ccc(C#N)c(F)c3)cc2N2CCC(N)CC2)cc1]
or a salt or tautomer thereof.

42. A pharmaceutical composition comprising a compound of claim 1, or a salt or tautomer thereof, together with a pharmaceutically acceptable carrier.

43. The pharmaceutical composition of claim 42, formulated for oral administration.

44. The pharmaceutical composition of claim 42, additionally comprising another therapeutic agent.

45. A method of inhibition of KDM1A comprising contacting KDM1A with a compound of claim 1, or a salt or tautomer thereof.

46. A method of treatment of a KDM1A-mediated disease comprising the administration of a therapeutically effective amount of a compound of claim 1, or a salt or tautomer thereof.

47. The method of claim 46, wherein the disease is cancer.

48. The method of claim 47, wherein the cancer is chosen from Ewing's sarcoma, multiple myeloma, T-cell leukemia, Wilm's tumor, small-cell lung cancer, bladder cancer, prostate cancer, breast cancer, head/neck cancer, colon cancer, and ovarian cancer.

49. The method of claim 46, wherein the disease is a myeloid disease.

50. The method of claim 49, wherein the myeloid disease is chosen from chronic neutrophilic leukemia, myelofibrosis, polycythemia vera, essential thrombocythemia, myelodysplastic syndrome, acute myelogenous leukemia, and chronic myelogenous leukemia.

51. The method of claim 46, wherein the disease is an inflammatory disease.

52. The method of claim 51, wherein the inflammatory disease is chosen from inflammatory bowel disease, rheumatoid arthritis, or systemic lupus erythematosus.

53. A method of treatment of a disease affecting beta-globin or a hemoglobinopathy comprising the administration of a therapeutically effective amount of a compound of claim 1, or a salt or tautomer thereof.

54. A method for achieving an effect in a patient comprising the administration of a therapeutically effective amount of a compound of claim 1, or a salt or tautomer thereof, to a patient, wherein the effect is chosen from an elevation of red blood cell count, an elevation of the red blood cell count of red cells containing fetal hemoglobin, an elevation in the total concentration of fetal hemoglobin in red cells, an elevation in the total concentration of fetal hemoglobin in reticulocytes, an increase in the transcription of the gamma globin gene in bone marrow-derived red cell precursors, a reduction in the number of sickle cell crises a patient experiences over a unit period of time, a halt to or prevention of tissue damage caused by sickling cells, a reduction in the proportion of red cells that undergo sickling under physiological conditions of relative hypoxia as measured using patient blood in an in vitro assay, an increase in the amount of histone 3 lysine methylation at lysine position, and/or a decrease in the amount of histone 3 methylation at lysine position 9 near or at the gamma globin promoter as assayed by ChIP using cells derived from a treated patient.

55. A method of inhibiting at least one KDM1A function comprising the step of contacting KDM1A with a compound of claim 1, or a salt or tautomer thereof, wherein the inhibition is measured by phenotype of red cells or their precursors either cultured or in vivo in humans or mouse or transgenic mice containing the human beta globin locus or portions thereof, the ability of cancer cells to proliferate, become differentiated, or induced to undergo apoptosis, the expression of specific genes known to be regulated by KDM1A activity, a change in the histone methylation states, a change in the methylation state of proteins known to be demethylated by KDM1A, expression of KDM1A-regulated genes, or binding of KDM1A with a natural binding partner such as CoREST, NuRD, DNMT1 or HDACs.

56. A method of inhibition of KDM1A comprising contacting KDM1A with a pharmaceutical composition of claim 42.

57. A method of treatment of a KDM1A-mediated disease comprising the administration of a therapeutically effective amount of a pharmaceutical composition of claim 42, to a patient in need thereof.

58. A method of treatment of a disease affecting beta-globin or a hemoglobinopathy comprising the administration of a therapeutically effective amount of a pharmaceutical composition of claim 42 to a patient in need thereof.

59. A method for achieving an effect in a patient comprising the administration of a therapeutically effective amount of a pharmaceutical composition of claim 42, to a patient, wherein the effect is chosen from an elevation of red blood cell count, an elevation of the red blood cell count of red cells containing fetal hemoglobin, an elevation in the total concentration of fetal hemoglobin in red cells, an elevation in the total concentration of fetal hemoglobin in reticulocytes, an increase in the transcription of the gamma globin gene in bone marrow-derived red cell precursors, a reduction in the number of sickle cell crises a patient experiences over a unit period of time, a halt to or prevention of tissue damage caused by sickling cells, a reduction in the proportion of red cells that undergo sickling under physiological conditions of relative hypoxia as measured using patient blood in an in vitro assay, an increase in the amount of histone 3 lysine methylation at lysine position 4, and/or a decrease in the amount of histone 3 methylation at lysine position 9 near or at the gamma globin promoter as assayed by ChIP using cells derived from a treated patient.

60. A method of inhibiting at least one KDM1A function comprising the step of contacting KDM1A with a pharmaceutical composition of claim 42, wherein the inhibition is measured by phenotype of red cells or their precursors either cultured or in vivo in humans or mouse or transgenic mice containing the human beta globin locus or portions thereof, the ability of cancer cells to proliferate, become differentiated, or induced to undergo apoptosis, the expression of specific genes known to be regulated by KDM1A activity, a change in the histone methylation states, a change in the methylation state of proteins known to be demethylated by KDM1A, expression of KDM1A-regulated genes, or binding of KDM1A with a natural binding partner.
